SEQUENCE LISTING (12 sequences)

SEQ ID NO: 1 - protein, 316 aa, Homo sapiens
MSYYHHHHHHDYDIPTTENLYFQGAMDPSSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRGT

SEQ ID NO: 2 - protein, 298 aa, Homo sapiens
MEEYMPTEHHHHHHENLYFQGTSMDPSSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQE

SEQ ID NO: 3 - protein, 2031 aa, Homo sapiens [sequence omitted]

SEQ ID NO: 4 - protein, 2006 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDADGSFGTPPGYGCAADRAEEQRRHQDGLPYIDDSPSSSPHLSSKGRGSRDALVSGALKSTKASELDLEKGLEMRKWVLSGILASEETYLSHLEALLLPMKPLKAAATTSQPVLTSQQIETIFFKVPELYEIHKESYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 5 - protein, 1530 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDAEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 6 - protein, 2031 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDADGSFGTPPGYGCAADRAEEQRRHQDGLPYIDDSPSSSPHLSSKGRGSRDALVSGALKSTKASELDLEKGLEMRKWVLSGILASEETYLSHLEALLLPMKPLKAAATTSQPVLTSQQIETIFFKVPELYEIHKESYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSKALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 7 - protein, 2006 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDADGSFGTPPGYGCAADRAEEQRRHQDGLPYIDDSPSSSPHLSSKGRGSRDALVSGALKSTKASELDLEKGLEMRKWVLSGILASEETYLSHLEALLLPMKPLKAAATTSQPVLTSQQIETIFFKVPELYEIHKESYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 8 - protein, 1530 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDAEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 9 - protein, 420 aa, Homo sapiens
MHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARYIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVKGVEACQLGTDDYDIPTTHHHHHH

SEQ ID NO: 10 - protein, 381 aa, Homo sapiens
MKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLGKGVEACQLGTDDYDIPTTHHHHHH

SEQ ID NO: 11 - protein, 534 aa, Homo sapiens
MAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRHNQTSLYKKAGSAAALFNFKKEPFTPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKT

SEQ ID NO: 12 - protein, 12 aa, Homo sapiens
EAIYAAPFAKKK

(19) United States
(12) Patent Application Publication
(10) Pub. No.: US 2012/0225057 A1
(43) Pub. Date: Sep. 6, 2012

(54) METHODS AND COMPOSITIONS FOR THE TREATMENT OF MYELOPROLIFERATIVE DISEASES AND OTHER PROLIFERATIVE DISEASES

(75) Inventors: Daniel L. Flynn, Lawrence, KS (US); Michael D. Kaufman, Lawrence, KS (US)

(73) Assignee: DECIPHERA PHARMACEUTICALS, LLC, Lawrence, KS (US)

(21) Appl. No.: 13/228,288

(22) Filed: Sep. 8, 2011

Related U.S. Application Data
(63) Continuation-in-part of application No. 12/829,561, filed on Jul. 2, 2010.
Application No. 12/829,561 is a division of application No. 11/870,388, filed on Oct. 10, 2007, now Pat. No. 7,790,756.
(60) Provisional application No. 60/850,834, filed on Oct. 11, 2006.

(51) Int. Cl. A61K 31/4709; A61K 31/4439; A61K 31/506; A61K 31/437; A61K 31/498; A61K 31/454; A61K 31/5025; A61P 35/02; A61P 35/00; A61P 35/04; A61P 27/02; A61P 37/00; A61P 29/00; A61P 25/00; A61P 19/04; A61P 11/06; A61P 19/02; A61P 19/06; A61P 31/00; A61P 11/00; A61P 9/10; A61P 17/06; A61P 19/08; A61P 37/06; A61P 1/00; A61P 1/04; A61K 39/395; A61K 38/16; C12N 9/99; A61K 31/4725

(52) U.S. Cl. 424/133.1; 514/314; 514/310; 514/341; 514/272; 514/300; 514/249; 514/252.18; 514/326; 514/252.19; 514/248; 514/253.06; 424/142.1; 514/12.2; 435/184

(57) ABSTRACT

Compounds of the present invention, alone and in combination with other active agents, find utility in the treatment of hyperproliferative diseases, mammalian cancers and especially human cancers including but not limited to for example malignant melanomas, myeloproliferative diseases, chronic myelogenous leukemia, acute lymphocytic leukemia, a disease caused by c-ABL kinase, oncogenic forms thereof, aberrant fusion proteins thereof and polymorphs thereof.

### CROSS REFERENCE TO RELATED APPLICATIONS

This application is related to U.S. Ser. No. 60/850,834 filed Oct. 11, 2006 and U.S. Ser. No. 11/870,388 filed Oct. 11, 2007, and claims the benefit of U.S. Ser. No. 12/829,561 filed Jul. 2, 2010, the disclosures of which are incorporated herein by reference for all purposes.

### FIELD OF THE INVENTION

The present invention relates to novel kinase inhibitors and modulator compounds useful for the treatment of various diseases. More particularly, the invention is concerned with combinations of such compounds with known kinase inhibitors, and methods of treating diseases. Preferrably, the compounds and combinations are useful for the modulation of kinase activity of c-ABL, c-KIT, TIE-2, TRK-A, TRK-B, TRK-C, VEGFR, PDGFR, FLT-3, c-MET, the HER family, cFMS, RET, oncogenic forms thereof, and aberrant fusion proteins and disease polymorphs thereof.

### BACKGROUND OF THE INVENTION

Several members of the protein kinase family have been clearly implicated in the pathogenesis of various proliferative and myeloproliferative diseases and thus represent important targets for treatment of these diseases. Some of the proliferative diseases relevant to this invention include cancer, rheumatoid arthritis, atherosclerosis, and retinopathies. Important examples of kinases which have been shown to cause or contribute to the pathogenesis of these diseases include c-ABL kinase and the oncogenic fusion protein BCR-ABL kinase, c-KIT kinase, c-MET, the HER family of kinases, PDGF receptor kinase, VEGF receptor kinases, FLT-3 kinase, TIE-2 kinase, the TRK family of kinases, RET kinase, and c-FMS kinase.

c-ABL kinase is an important non-receptor tyrosine kinase involved in cell signal transduction. This ubiquitously expressed kinase—upon activation by upstream signaling factors including growth factors, oxidative stress, integrin stimulation, and ionizing radiation—localizes to the cell plasma membrane, the cell nucleus, and other cellular compartments including the actin cytoskeleton (Van Etten, Trends Cell Biol. (1999) 9: 179). There are two normal isoforms of Abl kinase: ABL-1A and ABL-1B. The N-terminal half of c-ABL kinase is important for autoinhibition of the kinase domain catalytic activity (Pluk et al, Cell (2002) 108: 247). Details of the mechanistic aspects of this autoinhibition have recently been disclosed (Nagar et al, Cell (2003) 112: 859). The N-terminal myristolyl amino acid residue of ABL-1B has been shown to intramolecularly occupy a hydrophobic pocket formed from alpha-helices in the C-lobe of the kinase domain. Such intramolecular binding induces a novel binding area for intramolecular docking of the SH2 domain and the SH3 domain onto the kinase domain, thereby distorting and inhibiting the catalytic activity of the kinase. Thus, an intricate intramolecular negative regulation of the kinase activity is brought about by these N-terminal regions of c-ABL kinase. An aberrant dysregulated form of c-Abl is formed from a chromosomal translocation event, referred to as the Philadelphia chromosome (P. C. Nowell et al, Science (1960) 132: 1497; J. D. Rowley, Nature (1973) 243: 290). This abnormal chromosomal translocation leads aberrant gene fusion between the ABL kinase gene and the breakpoint cluster region (BCR) gene, thus encoding an aberrant protein called BCR-ABL (G. Q. Daley et al, Science (1990) 247: 824; M. L. Gishizky et al, Proc. Natl. Acad. Sci. USA (1993) 90: 3755; S. Li et al, J. Exp. Med. (1999) 189: 1399). The bcr-Abl fusion protein does not include the regulatory myristolylation site (B. Nagar et al, Cell (2003) 112: 859) and as a result functions as an oncoprotein which causes chronic myeloid leukemia (CML). CML is a malignancy of pluripotent hematopoietic stem cells. The p210 form of BCR-ABL is seen in 95% of patients with CML, and in 20% of patients with acute lymphocytic leukemia. A p185 form has also been disclosed and has been linked to being causative of up to 10% of patients with acute lymphocytic leukemia. It will be appreciated by one skilled in the art that “p210 form”, “p190 form” and “p185 form” each describe a closely related group of fusion proteins, and that Sequence ID's used herein are merely representative of each form and are not meant to restrict the scope solely to those sequences.

c-KIT (KIT, CD117, stem cell factor receptor) is a 145 kDa transmembrane tyrosine kinase protein that acts as a type-III receptor (Pereira et al. J Carcin. (2005), 4: 19). The c-KIT proto-oncogene, located on chromosome 4q11-21, encodes the c-KIT receptor, whose ligand is the stem cell factor (SCF, steel factor, c-KIT ligand, mast cell growth factor, Morstyn G, et al. Oncology (1994) 51(2):205. Yarden Y, et al. Embo J (1987) 6(11):3341). The receptor has tyrosine-protein kinase activity and binding of the ligands leads to the autophosphorylation of c-KIT and its association with substrates such as phosphatidylinositol 3-kinase (Pi3K). Tyrosine phosphorylation by protein tyrosine kinases is of particular importance in cellular signaling and can mediate signals for major cellular processes, such as proliferation, differentiation, apoptosis, attachment, and migration. Defects in c-KIT are a cause of piebaldism, an autosomal dominant genetic developmental abnormality of pigmentation characterized by congenital patches of white skin and hair that lack melanocytes. Gain-of-function mutations of the c-KIT gene and the expression of phosphorylated c-KIT are found in most gastrointestinal stromal tumors and mastocytosis. Activating c-KIT mutations have been identified in a subset of melanoma patients (Guo, J. J. Clin. Oncol. (2011) 29 (21): 2904). Further, almost all gonadal seminomas/dysgerminomas exhibit c-KIT membranous staining, and several reports have clarified that some (10-25%) have a c-KIT gene mutation (Sakuma, Y. et al. Cancer Sci (2004) 95:9, 716). C-KIT defects have also been associated with testicular tumors including germ cell tumors (GCT) and testicular germ cell tumors (TGCT).

The role of c-KIT expression has been studied in hematologic and solid tumors, such as acute leukemias (Cortes J. et al. Cancer (2003) 97(11):2760) and gastrointestinal stromal tumors (GIST, Fletcher C. D. et al. Hum Pathol (2002) 33(5):459). The clinical importance of c-KIT expression in malignant tumors relies on studies with Gleevec® (imatinib mesylate, STI571, Novartis Pharma AG Basel, Switzerland) that specifically inhibits tyrosine kinase receptors (Lefevre G. et al. J Biol Chem (2004) 279(30):31769). Moreover, a clinically relevant breakthrough has been the finding of anti-tumor effects of this compound in GIST, a group of tumors regarded as being generally resistant to conventional chemotherapy (de Silva C M, Reid R: Pathol Oncol Res (2003) 9(1):13-19). GIST most often become Gleevec resistant and molecularly targeted small therapies that target c-KIT mutations remain elusive.

The role of TRK kinases in acute myeloid leukemia has been documented. TRK gene expression in AML leukemic cell lines (HEL, K5672, HL60) was first reported in 1996 by Kaebisch and coworkers. This study also demonstrated expression of TRK-A in 44% (n=59) of AML patients. Actively translated TRK receptors were demonstrated by Western blotting and performance of in vitro kinase assays (Kaebisch, A., Brokt, S., Seay, U., Lohmeyer, J., Jaeger, U., and Pralle, H. Br. J. Haematol. (1996). 95: 102-109). More recently, a causal role for TRK in AML disease and disease progression has been disclosed (Li, A., Beutel, Rhein, M., et al. Blood (2009) 113: 2028-2037). High affinity TRK receptors were shown to be present in 55% of 94 analyzed acute leukemia patients, including 43/82 of leukemic blasts from AML patients. In contrast, TRK receptors were not detected on the surface of normal mononuclear cells. In 50% of AML cells expressing surface TRK-B receptors and the cognate ligand BDNF were also coexpressed establishing an autocrine loop within these AML patient cells. Significantly, constitutive TRK kinase activation (phosphorylation) was observed in all AML patient blast cells expressing a TRK receptor. AML patients whose blasts expressed TRK-A receptors exhibited a shorter median survival compared with patients not expressing TRK-A (312 vs. 547 days). Patients expressing both TRK-B and its autocrine ligand BDNF had a significantly shorter overall survival at three years (8% in TRKB/NBDNF+ patients vs. 30% in patients not expressing these markers). A TRK inhibitor or siRNA induced apoptosis in 65+% of TRK positive AML patient cells. A significant proportion of AML patients co-expressed TRK receptors and oncogenic FLT-3 ITD kinase, establishing that certain patient populations present with activation of both the TRK and FLT-3 signaling pathways (Li, A., Beutel, Rhein, M., et al. Blood (2009) 113: 2028-2037).

AML1-ETO is the most frequent chromosomal translocation in AML patients, both in adult and childhood AML (Xiao, A., Greaves, M. F., Buffler, P. et al. Leukemia (2001) 15: 1906-1913). The AML1-ETO fusion protein functions as a transcriptional activator to up-regulate expression of TRK-A in hematopoietic stem/progenitor cells (Mulloy, J. C., Jankovic, V., Wunderlich, M. et al. Proc. Natl. Acad. Sci. USA (2005) 102: 4016-4021).

This study examined a large number of AML samples and found that those patient cells expressing the AML1-ETO fusion expressed significantly higher levels of TRK-A.

Patient AML cells do not require the concomitant autocrine expression of the TRK ligand NGF to be functionally relevant. If overexpressed, TRK-A receptors may be constitutively activated in the absence of NGF ligand (Li, A., Beutel, Rhein, M., et al. Blood (2009) 113: 2028-2037). Additionally, it has been shown that the TRK ligands (growth factors) NGF and BDNF are both expressed by stromal cells in bone marrow, and activate bone marrow myeloid progenitor cells via a paracrine mechanism (Auffray, I., Chevalier, S. Froger, J., et al. Blood (1996) 88: 1608-1618; Labouyrie, E., Dubus, P., Groppi, A. et al. Am. J. Pathol. (1999) 154: 405-415.

While the predominant linkage of TRK receptor kinases to AML has been demonstrated by expression of wild-type activated TRK and/or coincident autocrine upregulation of the TRK ligand BDNF, there have also been reports of mutated forms of TRK associated with AML patient cells. Reuther and coworkers reported an activating mutant form of TRK-A containing a 75 amino acid deletion mutation in the extracellular domain. This form of TRK-A, called ΔTRK-A, was constitutively phosphorylated (activated) and transformed the 32D myeloid progenitor cell line (Reuther, G. W., Lambert, Q. T., Caligiuri, M. A., and Der, C. J. Mol. Cell. Biology (2000). 20: 8655-8666). ΔTRK-A, when expressed in 32D myeloid cells, caused an aggressive leukemogenesis when evaluated in vivo in mice (Meyer, J., Rhein, M. Schiedlmeier, B. et al. Leukemia (2007) 21: 2171-2180.

Finally, it has been disclosed that the TRK-A inhibitor AZ23 blocked NGF-induced proliferation of AML cell lines and also blocked TRK-A mediated-phosphorylation of ERK and AKT. AZ23 significantly decreased leukemic burden after oral administration by 70% after three weeks of dosing in an AML-xenograft model (Ghisoli, M. L., Fang, W., Graham, T. C. et al. 50^{th} ASH Annual Meeting (2008) December 6-9. Abstract #3789).

TIE-2 kinase is expressed in primitive hematopoietic stem cells (CD34+ CD38−) (Sato, A. Iwama, A. Takakura, N., Nishio, H., Yancopoulos, G. D., and Suda, T. Int. Immunol. (1998) 10: 1217-1227; Buhring, H. J., Seiffert, M., Bock, R. A. Scheding, S., Thiel, A., Scheffold, A., Kanz, L., and Brugger, W. Ann. New York Acad. Sci. (1999) 872: 25-38) and Ang-1 (the ligand for TIE-2 receptors) promotes adhesion of TIE-2+ cells and synergizes with stem cell factor to promote proliferation and differentiation of progenitor cells into myeloid cells (Takakura, N., Huang, X. L., Naruse, T. Hamaguchi, I., Dumont, D. J., Yancopoulos, G. D. and Suda, T. Immunity (1998) 9: 677-686). Ang-1/TIE-2 interactions in the bone marrow also enhance the ability of hematopoietic stem cells to remain quiescent and protected from myelosuppressive stress (Arai et al., 2004). Thus, blockade of TIE-2 may render these quiescent leukemic stem cells more susceptible to apoptosis (Arai, F., Hirao, A., Ohmura, M. et al. Cell (2004) 118: 149-161).

In addition to the direct role of TIE-2/Ang-1 in the adherence and proliferation of myeloid progenitor cells in the bone marrow, the TIE-2/Ang-1/Ang-2 signaling pathway also contributes to and maintains high microvessel density in the bone marrow niche that is significantly increased in AML patients. In this angiogenic role, TIE-2 is expressed on bone marrow endothelial cells to maintain the highly vascularized bone marrow niche (Holash, J., Maisonpierre, P. C., Compton, D. et al. Science (1999) 284: 1994-1998; Hussong, J. W., Rodgers, G. M., and Shami, P. J. Blood (2000) 95: 309-313; Padro, T., Ruiz, S., Bieker, R., et al. Blood (2000) 95: 2637-2644).

The ligand for the TIE-2 receptor kinase, Ang-1, is expressed in bone marrow stromal cells and acts in a paracrine manner to stimulate TIE-2 positive myeloid progenitor cells and endothelial cells (Sato, A. Iwama, A. Takakura, N., Nishio, H., Yancopoulos, G. D., and Suda, T. Int. Immunol. (1998) 10: 1217-1227).

Increased expression of both TIE-2 and its activating ligand Ang-1 were observed in circulating peripheral leukemic cells in 11 of 17 myeloid leukemia patients, including 7 of 11 samples from AML patients. Elevation in Ang-1 message was most prevalent in peripheral blood samples containing >20% AML myeloid blasts (Muller, A., Lange, K., Gaiser, T., Hofmann, M., Bartels, H., Feller, A. C., and Merz, H. Leukemia Research (2002) 26: 163-168.

A correlative study was reported by Hou and coworkers, in which expression of Ang-2 correlated with poor prognosis in AML patients (Hou, H-A., Chou, W-C., Lin, L-I., Tang, J-L., Tseung, M-H., Huang, C-F., Yao, M., Chen, C-Y., Tsay, W., and Tien, H-F. Leukemia Research (2008) 32: 904-912). In this study of 126 newly diagnosed de novo AML patients, high pre-treatment levels of Ang-2 in the bone marrow correlated, as an independent prognostic factor, with unfavorable overall survival. Only 7.2% of AML patients with high Ang-2 levels remained alive at 60 months compared to 46.5% of patients with low Ang-2 expression levels. Moreover, Ang-1 and Ang-2 levels correlated positively with peripheral blast count.

c-MET is a unique receptor tyrosine kinase (RTK) located on chromosome 7p and activated via its natural ligand hepatocyte growth factor. c-MET is found mutated in a variety of solid tumors (Ma P. C. et al. Cancer Metastasis (2003) 22:309). Mutations in the tyrosine kinase domain are associated with hereditary papillary renal cell carcinomas (Schmidt L et al. Nat. Genet. (1997)16:68; Schmidt L, et al. Oncogene (1999) 18:2343), whereas mutations in the sema and juxtamembrane domains are often found in small cell lung cancers (SCLC; Ma P. C. et al. Cancer Res (2003) 63:6272). Many activating mutations are also found in breast cancers (Nakopoulou et al. Histopath (2000) 36(4): 313). The panoply of tumor types for which c-MET mediated growth has been implicated suggests this is a target ideally suited for modulation by specific c-MET small molecule inhibitors.

The TPR-MET oncogene is a transforming variant of the c-MET RTK and was initially identified after treatment of a human osteogenic sarcoma cell line transformed by the chemical carcinogen N-methyl-N-nitro-N-nitrosoguanidine (Park M. et al. Cell (1986) 45:895). The TPR-MET fusion oncoprotein is the result of a chromosomal translocation, placing the TPR3 locus on chromosome 1 upstream of a portion of the c-MET gene on chromosome 7 encoding only for the cytoplasmic region. Studies suggest that TPR-MET is detectable in experimental cancers (e.g. Yu J. et al. Cancer (2000) 88:1801). Dimerization of the Mᵣ 65,000 TPR-MET oncoprotein through a leucine zipper motif encoded by TPR leads to constitutive activation of the c-MET kinase (Zhen Z. et al. Oncogene (1994) 9:1691). TPR-MET activates wild-type c-MET RTK and can activate crucial cellular growth pathways, including the Ras pathway (Aklilu F. et al. Am J Physiol (1996) 271:E277) and the phosphatidylinositol 3-kinase (PI3K)/AKT pathway (Ponzetto C. et al. Mol Cell Biol (1993) 13:4600). Conversely, in contrast to c-MET RTK, TPR-MET is ligand independent, lacks the CBL binding site in the juxtamembrane region in c-MET, and is mainly cytoplasmic. c-MET immunohistochemical expression seems to be associated with abnormal β-catenin expression, and provides good prognostic and predictive factors in breast cancer patients.

The majority of small molecule kinase inhibitors that have been reported have been shown to bind in one of three ways. Most of the reported inhibitors interact with the ATP binding domain of the active site and exert their effects by competing with ATP for occupancy. Such inhibitors are referred to as Type 1 kinase inhibitors. Other inhibitors have been shown to bind to a separate hydrophobic region of the protein known as the “DFG-in-conformation” pocket, and still others have been shown to bind to both the ATP domain and the “DFG-in-conformation” pocket. Examples specific to inhibitors of Raf kinases can be found in Lowinger et al, Current Pharmaceutical Design (2002) 8: 2269-2278; Dumas, J. et al., Current Opinion in Drug Discovery & Development (2004) 7: 600-616; Dumas, J. et al, WO 2003068223 A1 (2003); Dumas, J., et al, WO 9932455 A1 (1999), and Wan, P. T. C., et al, Cell (2004) 116: 855-867.

Physiologically, kinases are regulated by a common activation/deactivation mechanism wherein a specific activation loop sequence of the kinase protein binds into a specific pocket on the same protein which is referred to as the switch control pocket (see WO 2004/081084 and WO2007/008917 for further details). Such binding occurs when specific amino acid residues of the activation loop are modified for example by phosphorylation, oxidation, or nitrosylation. The binding of the activation loop into the switch pocket results in a conformational change of the protein into its active form (Huse, M. and Kuriyan, J. Cell (109) 275-282).

### SUMMARY OF THE INVENTION

Compounds of formula Ia find utility in the treatment of hyperproliferative diseases, including autoimmune diseases and other diseases characterized by hypervascularization or proliferation of myeloid cells, mast cells, fibroblasts, synoviocytes, or monocytes; mammalian cancers and especially human cancers including but not limited to melanomas; a disease caused by c-ABL kinase, oncogenic forms thereof, aberrant fusion proteins thereof including BCR-ABL kinase and polymorphs thereof; a disease caused by FLT-3 kinase, oncogenic forms thereof, aberrant fusion proteins thereof and polymorphs thereof; a disease caused by TIE-2 kinase, oncogenic forms thereof, aberrant fusion proteins thereof and polymorphs thereof; a disease caused by the TRK family of kinases, oncogenic forms thereof, aberrant fusion proteins thereof and polymorphs thereof; a disease caused by cMET kinase, oncogenic forms thereof, aberrant fusion proteins thereof including TPR-MET; a disease caused by KDR kinase or PDGFR kinases; a disease caused by HER kinases, oncogenic forms thereof and polymorphs thereof; a disease caused by RET kinase, oncogenic forms thereof, aberrant fusion proteins thereof; a disease caused by c-FMS kinase, oncogenic forms thereof and polymorphs thereof; a disease caused by a c-KIT kinase, oncogenic forms thereof, aberrant fusion proteins thereof and polymorphs thereof; and diseases caused by any of the foregoing kinases, oncogenic forms thereof, and aberrant fusion proteins thereof, including but not limited to, chronic myelogenous leukemia, acute lymphocytic leukemia, acute myeloid leukemia, other myeloproliferative disorders, a disease caused by metastasis of primary solid tumors to secondary sites, glioblastomas, ovarian cancer, pancreatic cancer, prostate cancer, lung cancers, mesothelioma, hypereosinophilic syndrome, a disease caused or maintained by pathological vascularization, ocular diseases characterized by hyperproliferation leading to blindness including various retinopathies, i.e. diabetic retinopathy and age-related macular degeneration, non small cell lung cancer, breast cancers, kidney cancers, colon cancers, cervical carcinomas, medullary thyroid carcinoma, melanomas, autoimmune diseases including rheumatoid arthritis, multiple sclerosis, lupus, asthma, human inflammation, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic obstructive pulmonary disease, bone resorptive diseases, bone cancer, graft-versus-host reaction, Crohn's disease, ulcerative colitis, inflammatory bowel disease, pyresis, gastrointestinal stromal tumors, mastocytosis, mast cell leukemia, and combinations thereof.

### DETAILED DESCRIPTION OF THE INVENTION

The following descriptions refer to various compounds, stereo-, regioisomers and tautomers of such compounds and individual moieties of the compounds thereof.

Carbocyclyl refers to carbon rings taken from cyclopropyl, cyclobutyl, cyclopentyl, cyclohexyl, cycloheptanyl, cyclooctanyl, norboranyl, norborenyl, bicyclo[2.2.2]octanyl, and bicyclo[2.2.2]octenyl;

Halogen refers to fluorine, chlorine, bromine and iodine;

Aryl refers to monocyclic or fused bicyclic ring systems characterized by delocalized π electrons (aromaticity) shared among the ring carbon atoms of at least one carbocyclic ring; preferred aryl rings are taken from phenyl, naphthyl, tetrahydronaphthyl, indenyl, and indanyl;

Heteroaryl refers to monocyclic or fused bicyclic ring systems characterized by delocalized π electrons (aromaticity) shared among the ring carbon or heteroatoms including nitrogen, oxygen, or sulfur of at least one carbocyclic or heterocyclic ring; heteroaryl rings are taken from, but not limited to, pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyridinyl, pyrimidinyl, pyrazinyl, pyridazinyl, triazinyl, indolyl, indolinyl, isoindolyl, isoindolinyl, indazolyl, benzofuranyl, benzothienyl, benzothiazolyl, benzothiazolonyl, benzoxazolyl, benzoxazolonyl, benzisoxazolyl, benzisothiazolyl, benzimidazolyl, benzimidazolonyl, benztriazolyl, imidazopyridinyl, pyrazolopyridinyl, imidazolonopyridinyl, thiazolopyridinyl, thiazolonopyridinyl, oxazolopyridinyl, oxazolonopyridinyl, isoxazolopyridinyl, isothiazolopyridinyl, triazolopyridinyl, imidazopyrimidinyl, pyrazolopyrimidinyl, imidazolonopyrimidinyl, thiazolopyridiminyl, thiazolonopyrimidinyl, oxazolopyridiminyl, oxazolonopyrimidinyl, isoxazolopyrimidinyl, isothiazolopyrimidinyl, triazolopyrimidinyl, dihydropurinonyl, pyrrolopyrimidinyl, purinyl, pyrazolopyrimidinyl, phthalimidyl, phthalimidinyl, pyrazinylpyridinyl, pyridinopyrimidinyl, pyrimidinopyrimidinyl, cinnolinyl, quinoxalinyl, quinazolinyl, quinolinyl, isoquinolinyl, phthalazinyl, benzodioxyl, benzisothiazoline-1,1,3-trionyl, dihydroquinolinyl, tetrahydroquinolinyl, dihydroisoquinolyl, tetrahydroisoquinolinyl, benzoazepinyl, benzodiazepinyl, benzoxapinyl, and benzoxazepinyl;

Heterocyclyl refers to monocyclic rings containing carbon and heteroatoms taken from oxygen, nitrogen, or sulfur and wherein there is not delocalized π electrons (aromaticity) shared among the ring carbon or heteroatoms; heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl;

Poly-aryl refers to two or more monocyclic or fused aryl bicyclic ring systems characterized by delocalized π electrons (aromaticity) shared among the ring carbon atoms of at least one carbocyclic ring wherein the rings contained therein are optionally linked together;

Poly-heteroaryl refers to two or more monocyclic or fused bicyclic systems characterized by delocalized π electrons (aromaticity) shared among the ring carbon or heteroatoms including nitrogen, oxygen, or sulfur of at least one carbocyclic or heterocyclic ring wherein the rings contained therein are optionally linked together, wherein at least one of the monocyclic or fused bicyclic rings of the poly-heteroaryl system is taken from heteroaryl as defined broadly above and the other rings are taken from either aryl, heteroaryl, or heterocyclyl as defined broadly above;

Poly-heterocyclyl refers to two or more monocyclic or fused bicyclic ring systems containing carbon and heteroatoms taken from oxygen, nitrogen, or sulfur and wherein there is not delocalized π electrons (aromaticity) shared among the ring carbon or heteroatoms wherein the rings contained therein are optionally linked, wherein at least one of the monocyclic or fused bicyclic rings of the poly-heteroaryl system is taken from heterocyclyl as defined broadly above and the other rings are taken from either aryl, heteroaryl, or heterocyclyl as defined broadly above;

Lower alkyl refers to straight or branched chain C1-C6alkyls;

Substituted in connection with a moiety refers to the fact that a further substituent may be attached to the moiety to any acceptable location on the moiety.

The term salts embraces pharmaceutically acceptable salts commonly used to form alkali metal salts of free acids and to form addition salts of free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, and heterocyclyl containing carboxylic acids and sulfonic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, 2-hydroxyethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid. Suitable pharmaceutically-acceptable salts of free acid-containing compounds of formula Ia include metallic salts and organic salts. More preferred metallic salts include, but are not limited to appropriate alkali metal (group Ia) salts, alkaline earth metal (group IIa) salts and other physiological acceptable metals. Such salts can be made from aluminum, calcium, lithium, magnesium, potassium, sodium and zinc. Preferred organic salts can be made from primary amines, secondary amines, tertiary amines and quaternary ammonium salts, including in part, tromethamine, diethylamine, tetra-N-methylammonium, N,N′-dibenzylethylenediamine, chloroprocaine, choline, diethanolamine, ethylenediamine, meglumine (N-methylglucamine) and procaine.

Structural, chemical and stereochemical definitions are broadly taken from IUPAC recommendations, and more specifically from Glossary of Terms used in Physical Organic Chemistry (IUPAC Recommendations 1994) as summarized by P. Miiller, Pure Appl. Chem., 66, 1077-1184 (1994) and Basic Terminology of Stereochemistry (IUPAC Recommendations 1996) as summarized by G. P. Moss Pure and Applied Chemistry, 68, 2193-2222 (1996). Specific definitions are as follows:

Atropisomers are defined as a subclass of conformers which can be isolated as separate chemical species and which arise from restricted rotation about a single bond.

Regioisomers or structural isomers are defined as isomers involving the same atoms in different arrangements.

Enantiomers are defined as one of a pair of molecular entities which are mirror images of each other and non-superimposable.

Diastereomers or diastereoisomers are defined as stereoisomers other than enantiomers. Diastereomers or diastereoisomers are stereoisomers not related as mirror images. Diastereoisomers are characterized by differences in physical properties, and by some differences in chemical behavior towards achiral as well as chiral reagents.

Tautomerism is defined as isomerism of the general form

G-X-Y=Z [symbol] X=Y-Z-G

where the isomers (called tautomers) are readily interconvertible; the atoms connecting the groups X, Y, Z are typically any of C, H, O, or S, and G is a group which becomes an electrofuge or nucleofuge during isomerization. The commonest case, when the electrofuge is H⁺, is also known as “prototropy”.

A pharmaceutically active agent or an additional agent is defined as a therapeutic agent that is used in combination with a compound of formula Ia of the present invention. The pharmaceutically active agent may be administered in combination with a compound of formula Ia in separate unit dosage forms or together in a single unit dosage form. If administered as separate unit dosage forms, the compound of formula Ia and the pharmaceutically active agent(s) may be administered simultaneously, sequentially or within a period of time from one another normally within five hours from one another. The pharmaceutically active agent(s) may be administered with a compound of formula Ia as part of an alternating dosing combination. In such an alternating dosing combination, a compound of formula Ia is dosed to a patient for a period of time ranging from two weeks to six months, followed by administration of the pharmaceutically active agent(s) for a second period of time ranging from two weeks to six months.

Tautomers are defined as isomers that arise from tautomerism, independent of whether the isomers are isolable.

### 1. First Aspect of the Invention—Compounds, Methods, Preparations and Adducts

The invention includes compounds of the formula Ia:

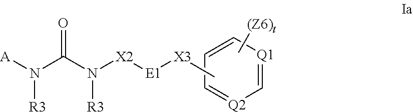

wherein Q1 and Q2 are each individually and independently selected from the group consisting of N and C—Z6, provided that both Q1 and Q2 are not simultaneously C—Z6;
E1 is selected from the group consisting cyclopropyl, cyclobutyl, cyclopentyl, cyclohexyl, pyrrolidinyl piperidinyl, phenyl, thienyl, oxazolyl, thiazolyl, isoxazolyl, isothiazolyl, pyrrolyl, pyrazolyl, oxadiazolyl, thiadiazolyl, furyl, imidazolyl, pyridyl, pyrimidinyl and naphthyl and wherein the E1 ring is substituted with one or more R16 moieties and wherein the E1 ring is substituted with one or more R18 moieties;
wherein A is selected from the group consisting of phenyl, C3-C8-carbocyclyl, pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyrazinyl, pyridazinyl, triazinyl, pyridinyl, pyrimidinyl, and G4;
G1 is a heteroaryl taken from the group consisting of pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyrazinyl, pyridazinyl, triazinyl, pyridinyl, and pyrimidinyl;
G2 is a fused bicyclic heteroaryl taken from the group consisting of indolyl, indolinyl, isoindolyl, isoindolinyl, indazolyl, benzofuranyl, benzothienyl, benzothiazolyl, benzothiazolonyl, benzoxazolyl, benzoxazolonyl, benzisoxazolyl, benzisothiazolyl, benzimidazolyl, benzimidazolonyl, benztriazolyl, imidazopyridinyl, pyrazolopyridinyl, imidazolonopyridinyl, thiazolopyridinyl, thiazolonopyridinyl, oxazolopyridinyl, oxazolonopyridinyl, isoxazolopyridinyl, isothiazolopyridinyl, triazolopyridinyl, imidazopyrimidinyl, pyrazolopyrimidinyl, imidazolonopyrimidinyl, thiazolopyridiminyl, thiazolonopyrimidinyl, oxazolopyridiminyl, oxazolonopyrimidinyl, isoxazolopyrimidinyl, isothiazolopyrimidinyl, triazolopyrimidinyl, dihydropurinonyl, pyrrolopyrimidinyl, purinyl, pyrazolopyrimidinyl, phthalimidyl, phthalimidinyl, pyrazinylpyridinyl, pyridinopyrimidinyl, pyrimidinopyrimidinyl, cinnolinyl, quinoxalinyl, quinazolinyl, quinolinyl, isoquinolinyl, phthalazinyl, benzodioxyl, benzisothiazoline-1,1,3-trionyl, dihydroquinolinyl, tetrahydroquinolinyl, dihydroisoquinolyl, tetrahydroisoquinolinyl, benzoazepinyl, benzodiazepinyl, benzoxapinyl, and benzoxazepinyl;
G3 is a non-fused bicyclic heteroaryl taken from the group consisting of pyridylpyridiminyl pyrimidinylpyrimidinyl, oxazolylpyrimidinyl, thiazolylpyrimidinyl, imidazolylpyrimidinyl, isoxazolylpyrimidinyl, isothiazolylpyrimidinyl, pyrazolylpyrimidinyl, triazolylpyrimidinyl, oxadiazoylpyrimidinyl, thiadiazoylpyrimidinyl, morpholinylpyrimidinyl, dioxothiomorpholinylpyrimidinyl, and thiomorpholinylpyrimidinyl;
G4 is a heterocyclyl taken from the group consisting of oxetanyl, azetadinyl, tetrahydrofuranyl, pyrrolidinyl, oxazolinyl, oxazolidinyl, imidazolonyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl;
The A ring is substituted at any substitutable position with one A1 moiety, wherein
A1 is selected from the group consisting of A2, A3 and A4;
A2 is selected from the group consisting of

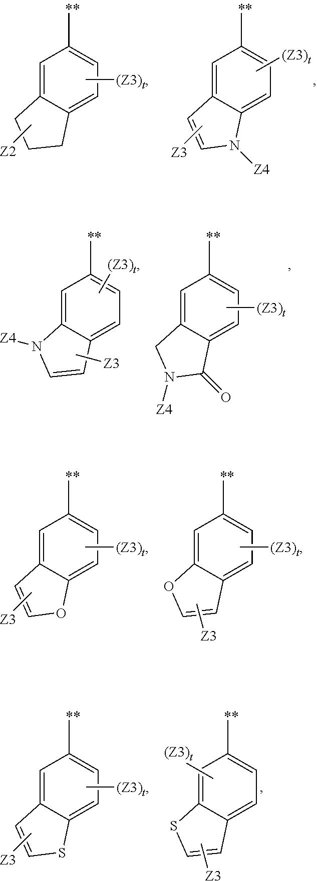

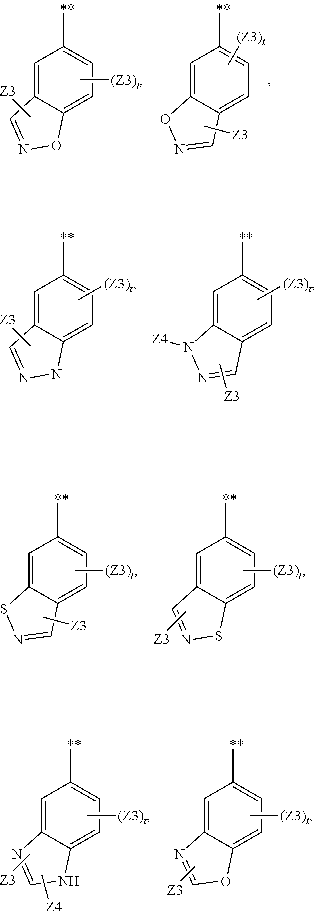

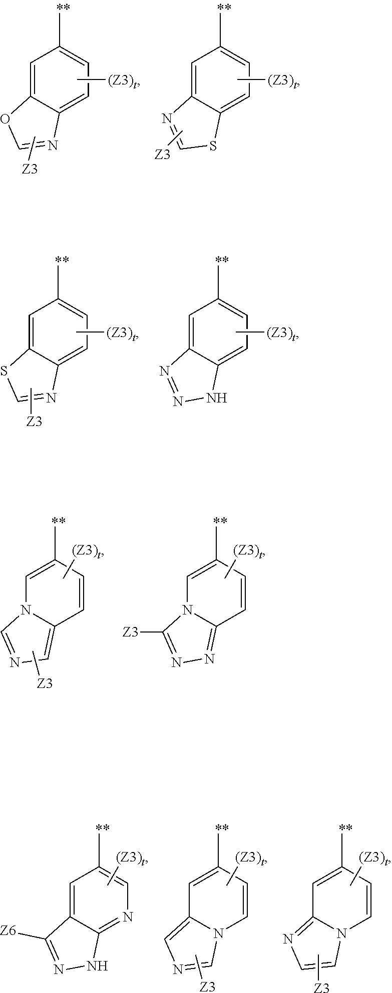

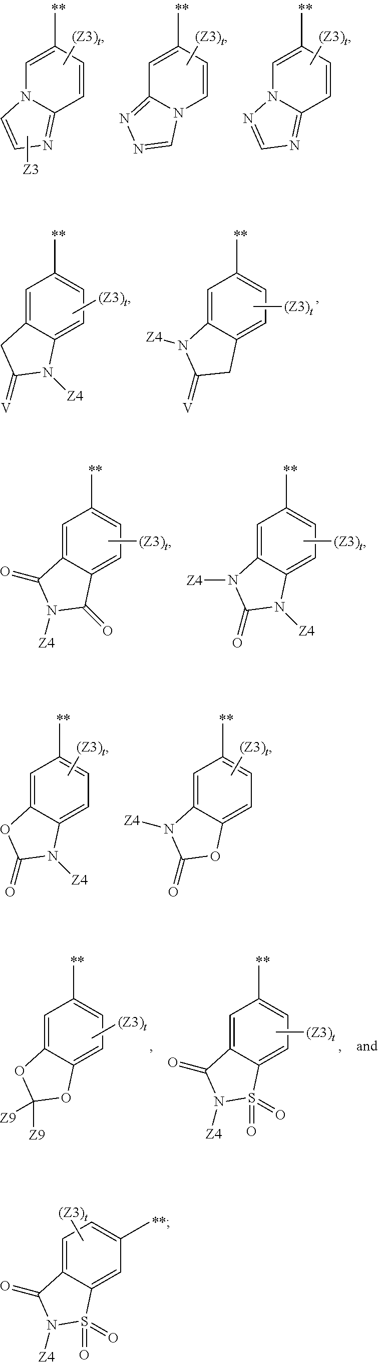

A3 is selected from the group consisting of

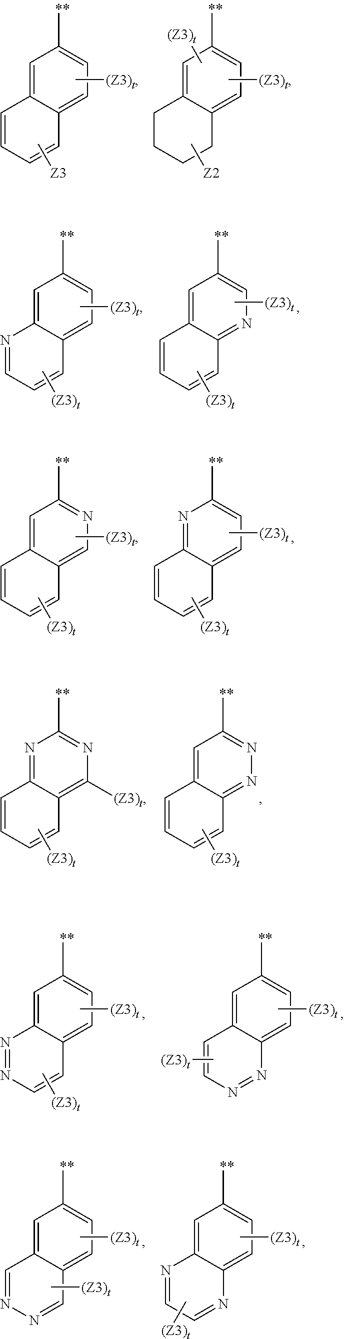

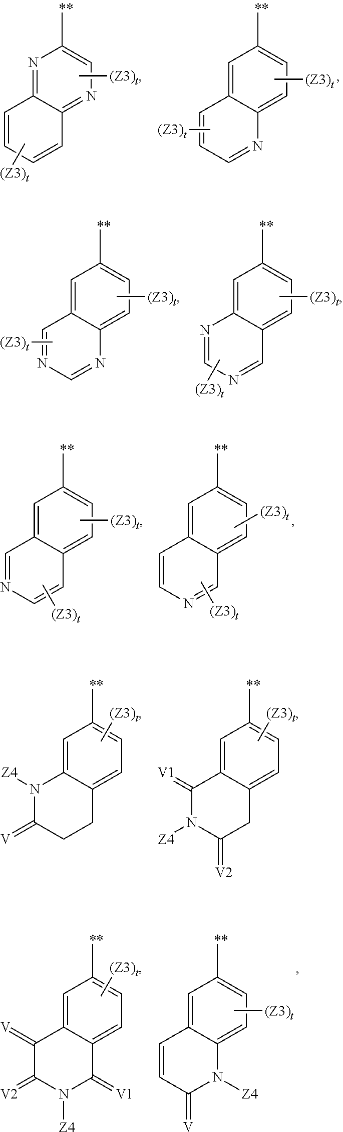

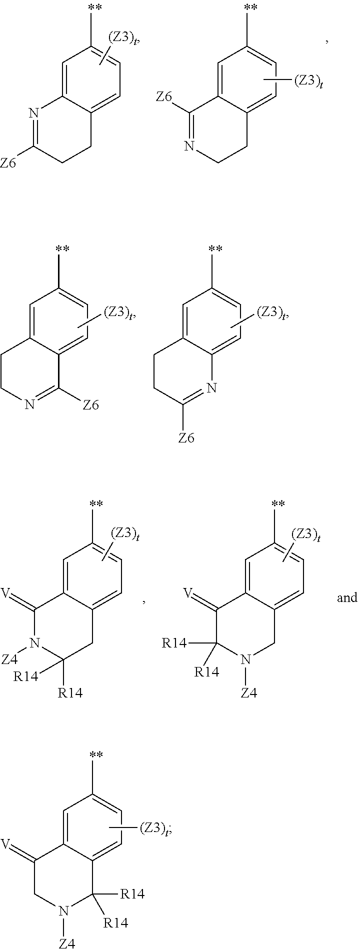

A4 is selected from the group consisting of

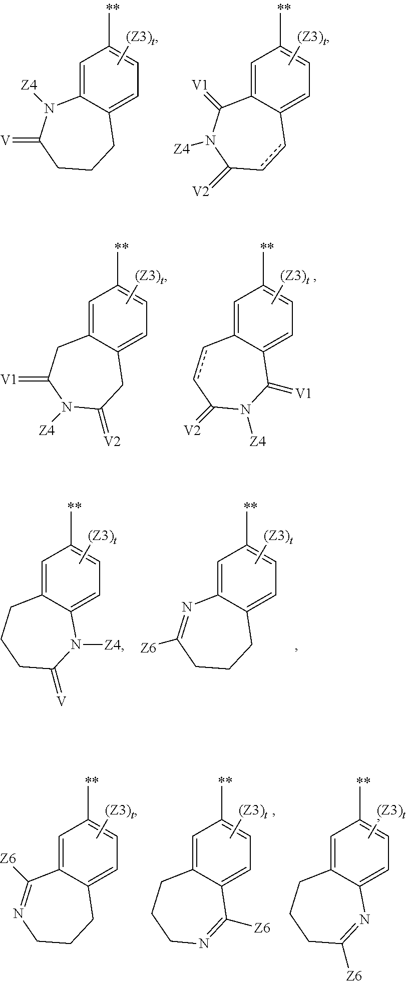

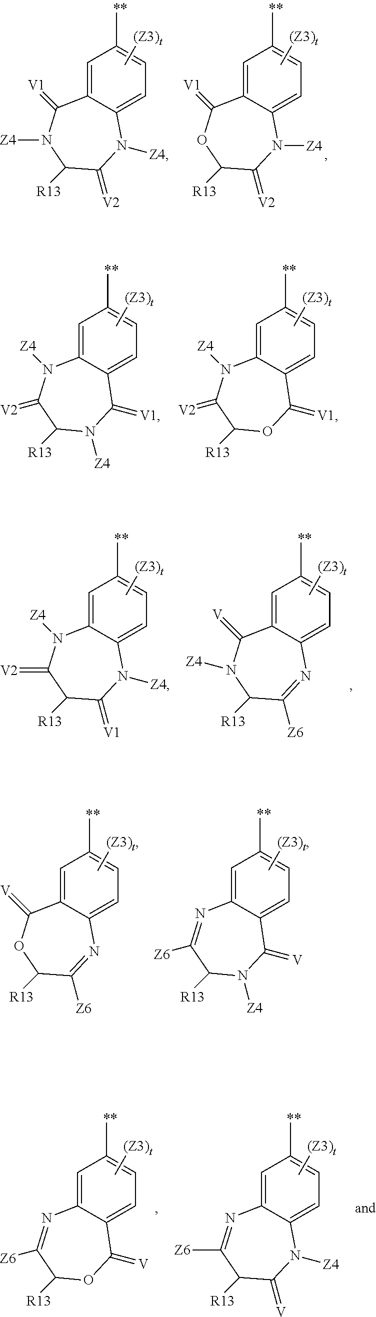

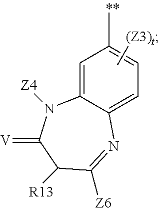

and wherein the symbol (**) is the point of attachment to the A ring of formula Ia; and wherein - - - - indicates either a saturated or unsaturated bond;
the A ring is optionally substituted with one or more R2 moieties;
X2 is selected from the group consisting of C1-C6 alkyl, C2-C6 branched alkyl, and a direct bond wherein E1 is directly linked to the NR3 group of formula Ia;
X3 is selected from the group consisting of —C(═O)—, —O—, —O—(CH₂)ₙ—, —S—(CH₂)ₙ—, —NR3-(CH₂)ₙ—, —O—(CH₂)_{q}—O—, —O—(CH₂)_{q}—NR3-, —N(R3)-(CH₂)_{q}—N(R3)-, —(CH₂)ₙ—N(R4)-C(═O)—, —(CH₂)ₙ—N(R4)-C(═O)(CH₂)ₙ—, —(CH₂)ₙ—C(═O)N(R4)-, —(CH₂)ₚ—, C2-C5alkenyl, C2-C5alkynyl, and C3-C6cycloalkyl and wherein the carbon atoms of —(CH₂)ₙ—, —(CH₂)_{q}—, —(CH₂)ₚ—, C2-C5alkenyl, and C2-C5alkynyl moieties of X3 may be further substituted by one or more C1-C6alkyl;
V, V1, and V2 are each independently and respectively selected from the group consisting of O and H₂;
each Z2 is independently and individually selected from the group consisting of hydrogen, aryl, C1-C6alkyl, C3-C8carbocyclyl, hydroxyl, hydroxyC1-C6alkyl-, cyano, (R3)₂N—, (R4)₂N—, (R4)₂NC1-C6alkyl-, (R4)₂NC2-C6alkylN(R4)-(CH₂)ₙ—, (R4)₂NC2-C6alkylO(CH₂)ₙ—, (R3)₂NC(O)—, (R4)₂NC(O)—, (R4)₂NC(O)C1-C6alkyl-, carboxyl, carboxyC1-C6alkyl-, C1-C6alkoxycarbonyl-, C1-C6alkoxycarbonylC1-C6alkyl-, (R3)₂NSO₂—, (R4)₂NSO₂—, —SO₂R5, —SO₂R8, —(CH₂)ₙN(R4)C(O)R8, —C(O)R8, ═O, ═NOH, ═N(OR6), —(CH₂)ₙG1, —(CH₂)ₙG4, —(CH₂)ₙ—O—(CH₂)ₙG1, —(CH₂)ₙ—O—(CH₂)ₙG4, —(CH₂)ₙNR3(CH₂)ₙ-aryl, —(CH₂)ₙNR3(CH₂)ₙG1, (CH₂)ₙNR3(CH₂)ₙG4, —(CH₂)ₙNHC(O)NHS(O)₂R8, —(CH₂)ₙNHS(O)₂NHC(O)R8, —C(O)NHS(O)₂R8, —(CH₂)ₙNHC(O)(CH₂)ₙR5, —(CH₂)ₙNHS(O)₂R5, (CH₂)ₙC(O)NH(CH₂)_{q}R5, —(CH₂)ₙC(O)R5, —(CH₂)ₙOC(O)R5, and —(CH₂)ₙR5;
in the event that Z2 contains an alkyl or alkylene moiety, such moieties may be further substituted with one or more C1-C6alkyls;
each Z3 is independently and individually selected from the group consisting of H, C1-C6alkyl, branched C3-C7alkyl, C3-C8carbocyclyl, halogen, fluoroC1-C6alkyl wherein the alkyl moiety can be partially or fully fluorinated, cyano, hydroxyl, methoxy, oxo, (R3)₂NC(O)—, (R4)₂NC(O)—, —N(R4)C(O)R⁸, (R3)₂NSO₂—, (R4)₂NSO₂—, —N(R4)SO₂R⁵, —N(R4)SO₂R⁸, —(CH₂)ₙN(R3)₂, —(CH₂)ₙN(R4)₂, —O(CH₂)_{q}N(R4)₂, —O(CH₂)_{q}O—C1-C6alkyl, —N(R3)(CH₂)_{q}O—C1-C6alkyl, —N(R3)(CH₂)_{q}N(R4)₂, O(CH₂)_{q}R⁵, —NR³(CH₂)_{q}R⁵, —C(O)R⁵, —C(O)R⁸, —R⁵, and nitro;
in the event that Z3 contains an alkyl or alkylene moiety, such moieties may be further substituted with one or more C1-C6alkyls;
each Z4 is independently and individually selected from the group consisting of H, C1-C6alkyl, hydroxyC2-C6alkyl, C1-C6alkoxyC2-C6alkyl-, (R4)₂N—C2-C6alkyl-, (R4)₂N—C2-C6alkylN(R4)-C2-C6alkyl-, (R4)₂N—C2-C6alkyl-O—C2-C6alkyl-(R4)₂NC(O)—C1-C6alkyl-, carboxyC1-C6alkyl-, C1-C6alkoxycarbonylC1-C6alkyl-, —C2-C6alkylN(R4)C(O)R8, R8-C(═NR3)-, —SO₂R8, —COR8, —(CH₂)ₙG1, —(CH₂)ₙG4, —(CH₂)_{g}—O(CH₂)(CH₂)G1, CH₂)O(CH₂)ₙG4, —(CH₂)_{q}NR3(CH₂)ₙG1, (CH₂)_{q}NR3(CH₂)ₙG4, —(CH₂)_{q}NHC(O)(CH₂)ₙR5, —(CH₂)_{q}C(O)NH(CH₂)_{q}R5, (CH₂)_{q}C(O)R5, —(CH₂)_{q}OC(O)R5, —(CH₂)_{q}R5, —(CH₂)_{q}NR4(CH₂)_{q}R5, and —(CH₂)_{q}—O—(CH₂)_{q}R5;
in the event that Z4 contains an alkyl or alkylene moiety, such moieties may be further substituted with one or more C1-C6alkyls;
each Z6 is independently and individually selected from the group consisting of H, C1-C6alkyl, branched C3-C7alkyl, hydroxyl, hydroxyC1-C6alkyl, hydroxyC2-C6 branched alkyl-, C1-C6alkoxy, C1-C6alkoxyC1-C6alkyl-, C1-C6alkoxyC2-C6 branched alkyl-, branched C2-C6alkoxy-, C1-C6alkylthio, (R3)₂N—, —N(R3)COR8, (R4)₂N—, —R5, —N(R4)C(O)R8, —N(R3)SO₂R6, —C(O)N(R3)₂, —C(O)N(R4)₂, —C(O)R5, —SO₂NHR4, halogen, fluoroC1-C6alkyl wherein the alkyl is fully or partially fluorinated, cyano, fluoroC1-C6alkoxy wherein the alkyl is fully or partially fluorinated, —O(CH₂)_{q}N(R4)₂, —N(R3)(CH₂)_{q}N(R4)₂, —O(CH₂)_{q}O—C1-C6alkyl, O(CH₂)_{q}N(R4)₂, —N(R3)(CH₂)_{q}O—C1-C6alkyl, —N(R3)(CH₂)_{q}N(R4)₂, —O(CH₂)_{q}R5, —N(R3)(CH₂)_{q}R5, —(NR3)ᵣR17, —(O)ᵣR17, —(S)ᵣR17, —(CH₂)ₙR17, —(CH₂)ₙG1, (CH₂)ₙG4, —(CH₂)_{q}—O—(CH₂)ₙG1, —(CH₂)_{q}—O—(CH₂)ₙG4, —(CH₂)_{q}N(R3)(CH₂)ₙG1, and —(CH₂)_{q}NR3(CH₂)ₙG4;
each R2 is selected from the group consisting of Z3-substituted aryl, Z3-substituted G1, Z3-substituted G4, C1-C6alkyl, branched C3-C8alkyl, R19 substituted C3-C8carbocyclyl, hydroxylC1-C6alky, hydroxyl branched C3-C6alkyl-, hydroxyl substituted C3-C8carbocyclyl-, cyanoC1-C6alkyl-, cyano substituted branched C3-C6alkyl-, cyano substituted C3-C8carbocyclyl-, (R4)₂NC(O)C1-C6alkyl-, (R4)₂NC(O) substituted branched C3-C6alkyl-, (R4)₂NC(O) substituted C3-C8carbocyclyl-, fluoroC1-C6alkyl wherein the alkyl is fully or partially fluorinated, halogen, cyano, C1-C6alkoxy, and fluoroC1-C6alkoxy wherein the alkyl group is fully or partially fluorinated;
each R3 is independently and individually selected from the group consisting of H, C1-C6alkyl, branched C3-C7alkyl, C3-C7cycloalkyl, and Z3-substituted phenyl-;
each R4 is independently and individually selected from the group consisting of H, C1-C6alkyl, hydroxyC1-C6alkyl-, dihydroxyC1-C6alkyl-, C1-C6alkoxyC1-C6alkyl-, branched C3-C7alkyl-, branched hydroxyC1-C6alkyl-, branched C1-C6alkoxyC1-C6alkyl-, branched dihydroxyC2-C6alkyl-, —(CH₂)ₚN(R7)₂, —(CH₂)ₚR5, (CH₂)ₚC(O)N(R7)₂, —(CH₂)ₙC(O)R5, —(CH₂)ₙC(O)OR3, C3-C8carbocyclyl, hydroxyl substituted C3-C8carbocyclyl-, alkoxy substituted C3-C8carbocyclyl-, dihydroxyl substituted C3-C8carbocyclyl-, and —(CH₂)ₙR17;
each R5 is independently and individually selected from the group consisting of

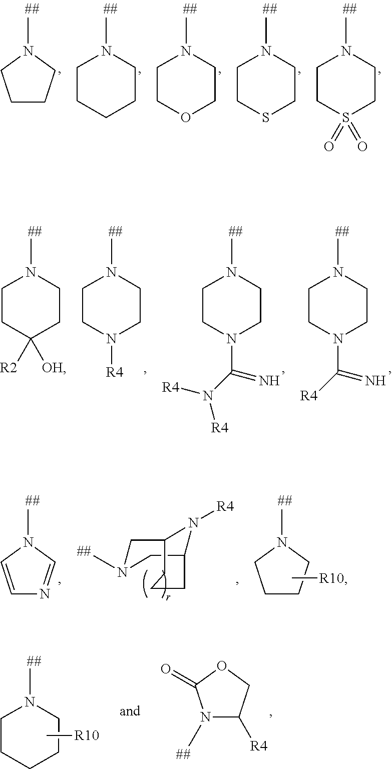

and wherein the symbol (##) is the point of attachment of the R5 moiety;
each R6 is independently and individually selected from the group consisting of C1-C6alkyl, branched C3-C7alkyl, C3-C8carbocyclyl, phenyl, G1, and G4;
each R7 is independently and individually selected from the group consisting of H, C1-C6alkyl, hydroxyC2-C6alkyl-, dihydroxyC2-C6alkyl-, C2-C6alkoxyC2-C6alkyl-, branched C3-C7alkyl-, branched hydroxyC2-C6 alkyl-, branched C2-C6alkoxyC2-C6alkyl-, branched dihydroxyC2-C6alkyl-, —(CH₂)_{q}R5, —(CH₂)ₙC(O)R5, (CH₂)ₙC(O)OR3, C3-C8carbocyclyl, hydroxyl substituted C3-C8carbocyclyl-, alkoxy substituted C3-C8carbocyclyl-, dihydroxy substituted C3-C8carbocyclyl, and —(CH₂)ₙR17;
each R8 is independently and individually selected from the group consisting of C1-C6alkyl, branched C3-C7alkyl, fluoroC1-C6alkyl wherein the alkyl moiety is partially or fully fluorinated, C3-C8carbocyclyl, Z3-substituted phenyl-, Z3-substituted phenylC1-C6alkyl-, Z3-substituted G1-, Z3-substituted G1-C1-C6alkyl-, Z2-substituted G4-, Z2-substituted G4-C1-C6alkyl-, OH, C1-C6alkoxy, N(R3)₂, N(R4)₂, and R5;
each R9 is independently and individually selected from the group consisting of H, F, C1-C6alkyl, branched C3-C7alkyl, C3-C7cycloalkyl, phenyl, phenyl-C1-C6alkyl-, —(CH₂)ₙG1, and —(CH₂)ₙG4;
each R10 is independently and individually selected from the group consisting of CO₂H, CO₂C1-C6alkyl, —C(O)N(R4)₂, OH, C1-C6alkoxy, and —N(R4)₂;
each R13 is independently and individually selected from the group consisting of H, C1-C6alkyl, branched C3-C7alkyl, carbocyclyl, hydroxyC2-C7alkyl, C1-C6alkoxyC2-C7alkyl-, (R4)₂NC(O)—, (R4)₂NC(O)C1-C6alkyl-, carboxyC1-C6alkyl-, C1-C6alkoxycarbonyl-, C1-C6alkoxycarbonylC1-C6alkyl-, (R4)₂N—C2-C6alkyl-, (R4)₂N—C2-C6alkylN(R4)(CH₂)_{q}—, R5-C2-C6alkylN(R4)(CH₂)_{q}—, (R4)₂N—C2-C6alkylO(CH₂)_{q}—, R5-C2-C6alkylO(CH₂)_{q}—, —(CH₂)_{q}N(R4)C(O)R8, aryl, arylC1-C6alkyl, aryloxyC2-C6alkyl-, arylaminoC2-C6alkyl-, C1-C6alkoxycarbonylC1-C6alkyl-, —C2-C6alkylN(R4)C(O)R8, R8C(═NR3)-, —SO₂R8, —COR8, —(CH₂)ₙG1, —(CH₂)ₙ-G4, —(CH₂)ₙ—O—(CH₂)ₙG1, —(CH₂)ₙ—O—(CH₂)ₙG4, —(CH₂)ₙN(R3)(CH₂)ₙG1, and —(CH₂)ₙN(R3)(CH₂)ₙG4;
each R14 is independently and respectively selected from the group consisting of H, C1-C6alkyl, branched C3-C6alkyl, and C3-C7carbocyclyl;
each R16 is independently and individually selected from the group consisting of C1-C6alkyl, branched C3-C7alkyl, C3-C8 carbocyclyl, halogen, fluoro C1-C6alkyl wherein the alkyl moiety can be partially or fully fluorinated, cyano, hydroxyl, C1-C6alkoxy, fluoroC1-C6alkoxy wherein the alkyl moiety can be partially or fully fluorinated, —N(R3)₂, —N(R4)₂, and nitro;
each R17 is taken from the group comprising phenyl, naphthyl, pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyrazinyl, pyridazinyl, triazinyl, oxetanyl, azetadinyl, tetrahydrofuranyl, oxazolinyl, oxazolidinyl, pyranyl, thiopyranyl, tetrahydropyranyl, dioxalinyl, azepinyl, oxepinyl, diazepinyl, pyrrolidinyl, and piperidinyl;
wherein R17 can be further substituted with one or more Z2, Z3 or Z4 moieties;
R18 is independently and individually selected from the group consisting of hydrogen, C1-C6alkyl, branched C3-C7alkyl, C3-C8carbocyclyl, halogen, fluoroC1-C6alkyl wherein the alkyl moiety can be partially or fully fluorinated, cyano, hydroxyl, C1-C6alkoxy, fluoroC1-C6alkoxy wherein the alkyl moiety can be partially or fully fluorinated, —N(R3)₂, —N(R4)₂, C2-C3alkynyl, and nitro;
R19 is H or C1-C6alkyl;
wherein two R3 or R4 moieties are independently and individually taken from the group consisting of C1-C6alkyl and branched C3-C6alkyl, hydroxyalkyl, and alkoxyalkyl and are attached to the same nitrogen atom, said moieties may cyclize to form a C3-C7 heterocyclyl ring;
and n is 0-6; p is 1-4; q is 2-6; r is 0 or 1; t is 1-3, v is 1 or 2;
with the proviso that compounds of formula Ia can not be

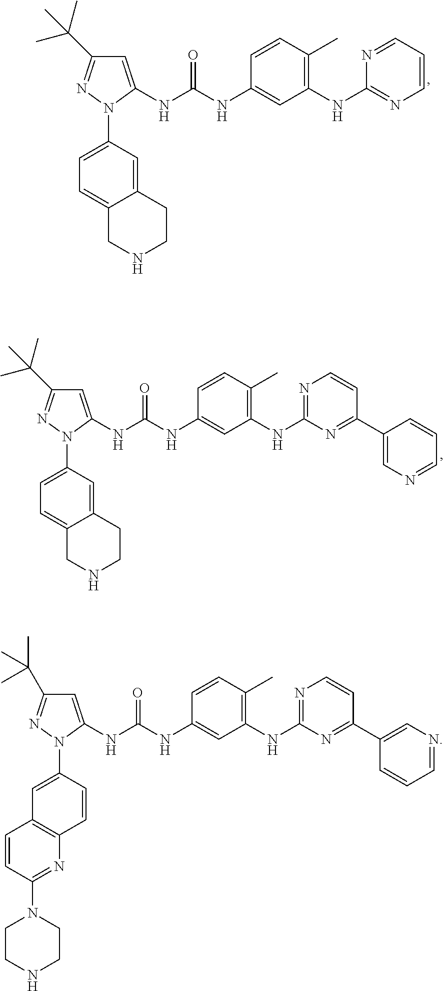

In one embodiment, the compounds of formula Ia are of the formula Ia′:

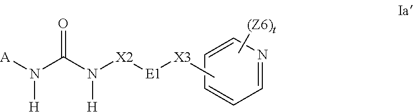

or a pharmaceutically acceptable salt thereof,
wherein
E1 is phenyl and wherein the E1 ring is substituted with one to three R16 moieties and one to three R18 moieties;
A is selected from the group consisting of pyrazolyl and imidazolyl;
G1 is a heteroaryl taken from the group consisting of pyrazolyl, imidazolyl, pyrrolyl, furyl, thienyl, oxazolyl, thiazolyl, isoxazolyl, isothiazolyl, oxadiazolyl, thiadiazolyl, triazolyl, tetrazolyl, pyrazinyl, pyridazinyl, triazinyl, pyridinyl, and pyrimidinyl;
G4 is a heterocyclyl taken from the group consisting of oxetanyl, azetadinyl, tetrahydrofuranyl, pyrrolidinyl, oxazolinyl, oxazolidinyl, imidazolonyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl;
the A ring is substituted at any substitutable position with one A1 moiety, wherein A1 is selected from the group consisting of:

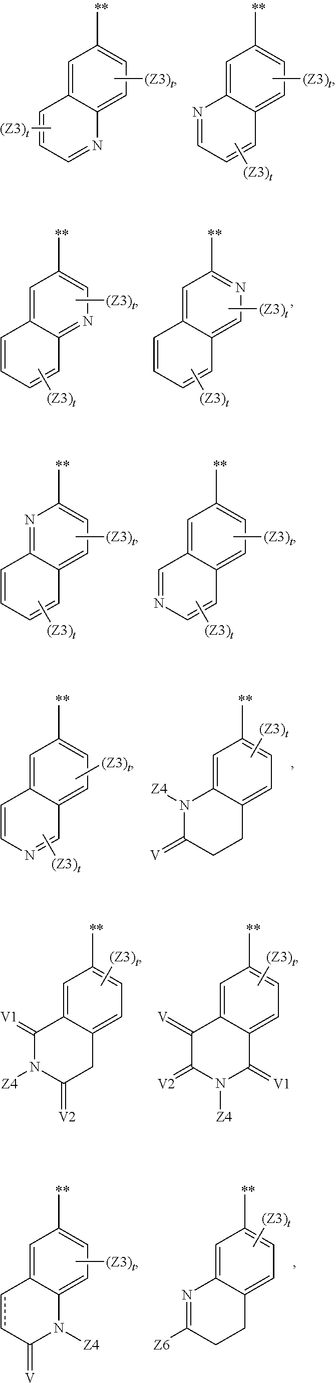

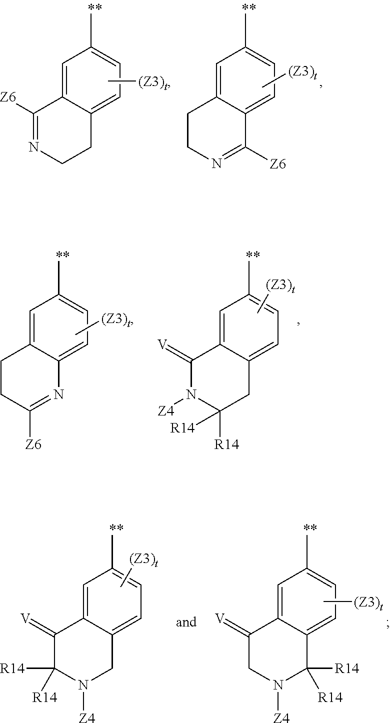

and wherein the symbol (**) is the point of attachment to the A ring of formula Ia;
and wherein - - - - indicates either a saturated or unsaturated bond;
the A ring is optionally substituted with one or more R2 moieties;
X2 is a direct bond, wherein E1 is directly linked to the NH group of formula Ia;

### X3 is —O—;

V, V1 and V2 are each independently O or represent two hydrogens attached to the methylene carbon to which the V, V1, and V2 is attached;
each Z3 is independently and individually selected from the group consisting of H, C1-C6alkyl, branched C3-C7alkyl, C3-C8carbocyclyl, halogen, fluoroC1-C6alkyl wherein the alkyl moiety can be partially or fully fluorinated, cyano, hydroxyl, methoxy, oxo, (R3)₂NC(O)—, (R4)₂NC(O)—, —N(R4)C(O)R8, (R3)₂NSO₂—, (R4)₂NSO₂—, —N(R4)SO₂R5, —N(R4)SO₂R8, —(CH₂)N(R3)₂, —(CH₂)ₙN(R4)₂, —O(CH₂)_{q}N(R4)₂, —O(CH₂)_{q}O—C1-C6alkyl, —N(R3)(CH₂)_{q}O—C1-C6alkyl, —N(R3)(CH₂)_{q}N(R4)₂, O(CH₂)_{q}R5, —N(R3)(CH₂)_{q}R5, —C(O)R5, —C(O)R8, —R5, and nitro;
in the event that Z3 contains an alkyl or alkylene moiety, such moieties may be further substituted by one or more C1-C6alkyl;
each Z4 is independently and individually selected from the group consisting of H, C1-C6alkyl, hydroxyC2-C6alkyl, C1-C6alkoxyC2-C6alkyl, (R4)₂N—C2-C6alkyl, (R4)₂N—C2-C6alkylN(R4)-C2-C6alkyl, (R4)₂N—C2-C6alkyl-O—C2-C6alkyl, (R4)₂NC(O)—C1-C6alkyl, carboxyC1-C6alkyl-, C1-C6alkoxycarbonylC1-C6alkyl-, —C2-C6alkylN(R4)C(O)R8, R8-C(═NR3)-, —SO₂R8, —C(O)R8, and —(CH₂)_{q}R5;
in the event that Z4 contains an alkyl or alkylene moiety, such moieties may be further substituted by one or more C1-C6alkyl;
each Z6 is independently and individually selected from the group consisting of —C(O)N(R3)₂, —C(O)N(R4)₂, —(CH₂)ₙG1, (R4)₂N—, (R3)₂N—, —N(R3)C(O)R8, —N(R4)C(O)R8, H, C1-C6alkyl, branched C3-C7alkyl, hydroxyl, hydroxyC1-C6alkyl, hydroxyC2-C6 branched alkyl, C1-C6alkoxy, C1-C6alkoxyC1-C6alkyl-, C1-C6alkoxyC2-C6 branched alkyl-, C2-C6 branched alkoxy-, C1-C6alkylthio-, —R5, —N(R3)SO₂R6, —C(O)R5, —SO₂N(R4)₂, —SO₂N(R5)₂, halogen, fluoroC1-C6alkyl wherein the alkyl is fully or partially fluorinated, cyano, fluoroC1-C6alkoxy wherein the alkyl is fully or partially fluorinated, —O(CH₂)_{q}N(R4)₂, —N(R3)(CH₂)_{q}N(R4)₂, —O(CH₂)_{q}O—C1-C6alkyl, —O(CH₂)_{q}N(R4)₂, —N(R3)(CH₂)_{q}O—C1-C6alkyl, N(R3)(CH₂)_{q}N(R4)₂, —O(CH₂)_{q}R5, and —N(R3)(CH₂)_{q}R5, —(NR3)ᵣR17, —(O)ᵣR17, —(S)ᵣR17, —(CH₂)ₙR17, —R17, —(CH₂)ₙG4, —(CH₂)ₙ—O—(CH₂)ₙG1, —(CH₂)ₙ—O—(CH₂)ₙG4, —(CH₂)ₙN(R3)(CH₂)ₙG1, and —(CH₂)ₙN(R3)(CH₂)ₙG4;
each R2 is selected from the group consisting of branched C3-C8alkyl, C1-C6alkyl, fluoroC1-C6alkyl wherein the alkyl is fully or partially fluorinated, R19 substituted C3-C8carbocyclyl, Z3-substituted aryl, Z3-substituted G1-, Z3-substituted G4-, hydroxyC1-C6alkyl-, hydroxy branched C3-C6alkyl-, hydroxy substituted C3-C8carbocyclyl-, cyanoC1-C6alkyl-, cyano substituted branched C3-C6alkyl, cyano substituted C3-C8carbocyclyl, (R4)₂NC(O)C1-C6alkyl-, (R4)₂NC(O) substituted branched C3-C6alkyl-, (R4)₂NC(O) substituted C3-C8carbocyclyl-, halogen, cyano, C1-C6alkoxy, and fluoroC1-C6alkoxy wherein the alkyl is fully or partially fluorinated;
wherein each R3 is independently and individually selected from the group consisting of H, C1-C6alkyl, branched C3-C7alkyl, C3-C8carbocyclyl, and Z3-substituted phenyl;
each R4 is independently and individually selected from the group consisting of H, C1-C6alkyl, hydroxyC1-C6alkyl-, dihydroxyC1-C6alkyl-, C1-C6alkoxyC1-C6alkyl-, branched C3-C7alkyl-, branched hydroxyC1-C6alkyl-, branched C1-C6alkoxyC1-C6alkyl-, branched dihydroxyC2-C6alkyl-, —(CH₂)ₚN(R7)₂, —(CH₂)ₚR5, (CH₂)ₚC(O)N(R7)₂, —(CH₂)ₙC(O)R5, —(CH₂)ₙC(O)OR3, C3-C8carbocyclyl, hydroxy substituted C3-C8carbocyclyl-, alkoxy substituted C3-C8carbocyclyl-, dihydroxy substituted C3-C8carbocyclyl-, and —(CH₂)ₙR17;
each R5 is independently and individually selected from the group consisting of

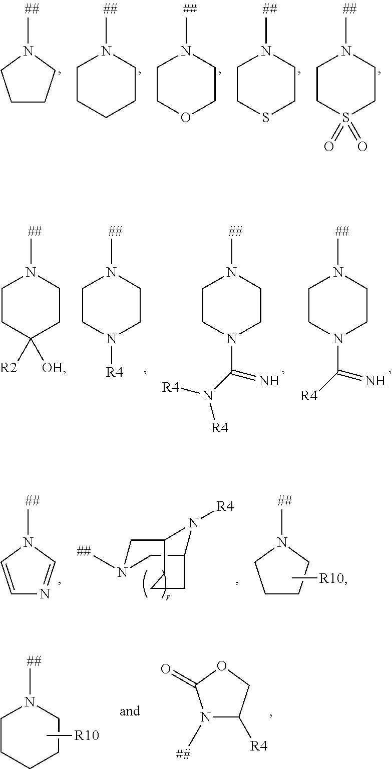

and wherein the symbol (##) is the point of attachment of the R5 moiety;
each R6 is independently and individually selected from the group consisting of C1-C6alkyl, branched C3-C7alkyl, C3-C8carbocyclyl, phenyl, G1, and G4;
each R7 is independently and individually selected from the group consisting of H, C1-C6alkyl, hydroxyC2-C6alkyl-, dihydroxyC2-C6alkyl-, C2-C6alkoxyC2-C6alkyl-, branched C3-C7alkyl-, branched hydroxyC2-C6alkyl-, branched C2-C6alkoxyC2-C6alkyl-, branched dihydroxyC2-C6alkyl-, —(CH₂)_{q}R5, —(CH₂)ₙC(O)R5, (CH₂)ₙC(O)OR3, C3-C8carbocyclyl, hydroxy substituted C3-C8carbocyclyl-, alkoxy substituted C3-C8carbocyclyl-, dihydroxy substituted C3-C8carbocyclyl, and —(CH₂)ₙR17;
each R8 is independently and individually selected from the group consisting of C1-C6alkyl, branched C3-C7alkyl, fluoroC1-C6alkyl wherein the alkyl moiety is partially or fully fluorinated, C3-C8carbocyclyl, Z3-substituted phenyl-, Z3-substituted phenylC1-C6alkyl-, Z3-substituted G1, Z3-substituted G1-C1-C6alkyl-, Z2-substituted G4, Z2-substituted G4-C1-C6alkyl-, OH, C1-C6alkoxy, N(R3)₂, N(R4)₂, and R5;
each R10 is independently and individually selected from the group consisting of CO₂H, CO₂C1-C6alkyl, —C(O)N(R4)₂, OH, C1-C6alkoxy, and —N(R4)₂;
each R14 is independently and respectively selected from the group consisting of H, C1-C6alkyl, branched C3-C6alkyl, and C3-C8carbocyclyl;
R16 is independently and individually selected from the group consisting of halogen, C1-C6alkyl, branched C3-C7alkyl, C3-C8carbocyclyl, fluoroC1-C6alkyl wherein the alkyl moiety can be partially or fully fluorinated, cyano, hydroxyl, C1-C6alkoxy, fluoroC1-C6alkoxy wherein the alkyl moiety can be partially or fully fluorinated, —N(R3)₂, —N(R4)₂, C2-C3alkynyl, and nitro;
each R17 is selected from the group consisting of phenyl, naphthyl, pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyrazinyl, pyridazinyl, triazinyl, oxetanyl, azetadinyl, tetrahydrofuranyl, oxazolinyl, oxazolidinyl, pyranyl, thiopyranyl, tetrahydropyranyl, dioxalinyl, azepinyl, oxepinyl, diazepinyl, pyrrolidinyl, and piperidinyl;
wherein R17 can be further substituted with one or more Z2, Z3 or Z4 moieties;
R18 is independently and individually selected from the group consisting of hydrogen, C1-C6alkyl, branched C3-C7alkyl, C3-C8carbocyclyl, halogen, fluoroC1-C6alkyl wherein the alkyl moiety can be partially or fully fluorinated, cyano, hydroxyl, C1-C6alkoxy, fluoroC1-C6alkoxy wherein the alkyl moiety can be partially or fully fluorinated, —N(R3)₂, —N(R4)₂, C2-C3alkynyl, and nitro;
R19 is H or C1-C6alkyl;
n is 0-6; p is 1-4; q is 2-6; r is 0 or 1; t is 1-3; and v is 1 or 2.
1.1 Compounds of Formula Ia which Exemplify Preferred a and X2-E1 Moieties

In a preferred embodiment of compounds of formula Ia, said compounds have structures of formula I-Ib:

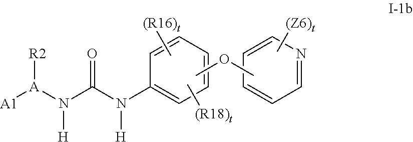

wherein the A ring is pyrazolyl.
1.1.1 Compounds of Formula I-1b which Exemplify Preferred A1 Moieties

In a preferred embodiment of compounds of formula I-1b, said compounds have structures of formula I-1c:

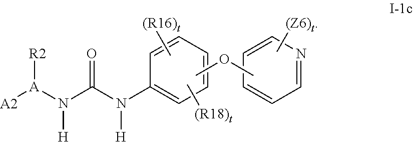

1.1.2 Compounds of Formula Ib which Exemplify Preferred A1 Moieties

In a preferred embodiment of compounds of formula I-1b, said compounds have structures of formula I-1d

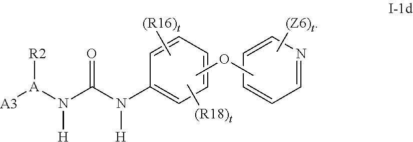

1.1.3 Compounds of Formula I-1b which Exemplify Preferred A1 Moieties

In a preferred embodiment of compounds of formula I-1b, said compounds have structures of formula I-1e

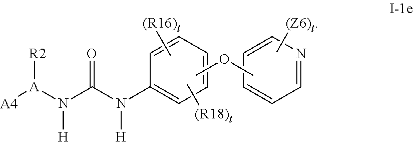

### 1.1.4 More Preferred Compounds of Section 1.1

In a preferred embodiment of compounds from Section 1.1, said compounds have structures of formula I-1f:

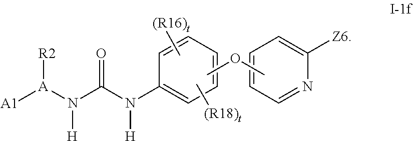

1.1.5 Compounds of Section 1.1.4 with Preferred R16 Moieties

In a preferred embodiment of compounds from Section 1.1.4, said compounds have structures of formula I-1g:

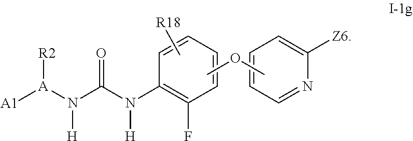

1.1.6 Compounds of Section 1.1.5 with a More Preferred A1 Moieties

In a more preferred embodiment of compounds from Section 1.1.5, said compounds have structures of formula I-1h:

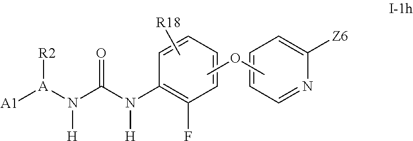

wherein A1 is selected from the group consisting of

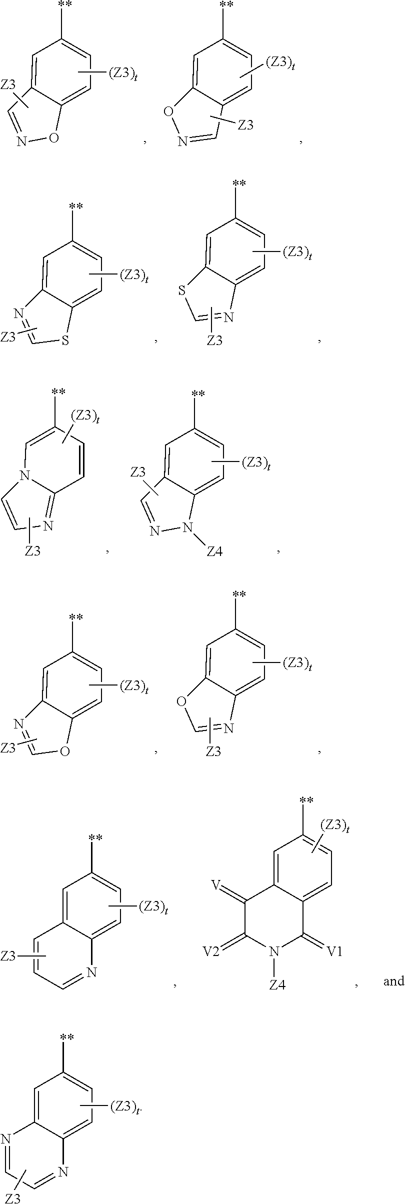

1.1.7 Compounds of Section 1.1.5 with a More Preferred Z6 Moieties

In a more preferred embodiment of compounds from Section 1.1.5, said compounds have structures of formula I-1i:

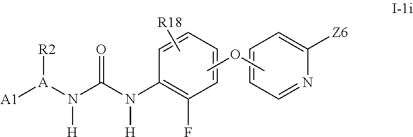

wherein Z6 is —C(O)NHR4, —NHR4 or R19 substituted pyrazole.
1.2 Compounds of Formula Ia which Exemplify Preferred A and X2-E1 Moieties

In a preferred embodiment of compounds of formula Ia, said compounds have structures of formula I-2a:

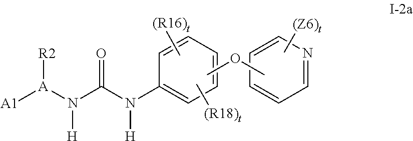

wherein the A ring is isoxazolyl.
1.2.1 Compounds of Formula I-2a which Exemplify Preferred A1 Moieties

In a preferred embodiment of compounds of formula I-2a, said compounds have structures of formula I-2b:

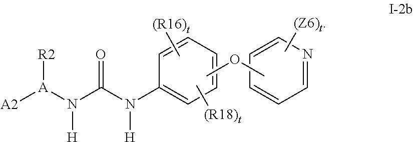

1.2.2 Compounds of Formula I-2a which Exemplify Preferred A1 Moieties

In a preferred embodiment of compounds of formula I-2a, said compounds have structures of formula I-2c:

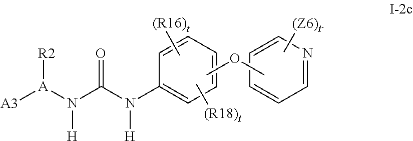

1.2.3 Compounds of Formula I-2a which Exemplify Preferred A1 Moieties

In a preferred embodiment of compounds of formula I-2a, said compounds have structures of formula I-2d:

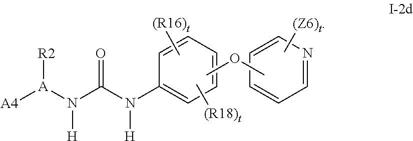

### 1.2.4 More Preferred Compounds of Section 1.2

In a preferred embodiment of compounds from Section 1.2, said compounds have structures of formula I-2e:

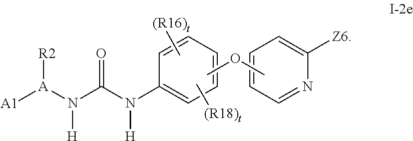

1.2.5 Compounds of Section 1.2.4 with Preferred R16 Moieties

In a preferred embodiment of compounds from Section 1.2.4, said compounds have structures of formula I-2f:

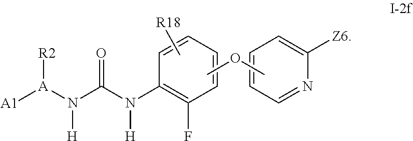

1.2.6 Compounds of Section 1.2.5 with a More Preferred A1 Moieties

In a more preferred embodiment of compounds from Section 1.2.5, said compounds have structures of formula I-2g:

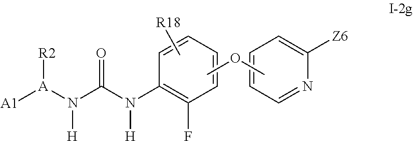

wherein A1 is selected from the group consisting of

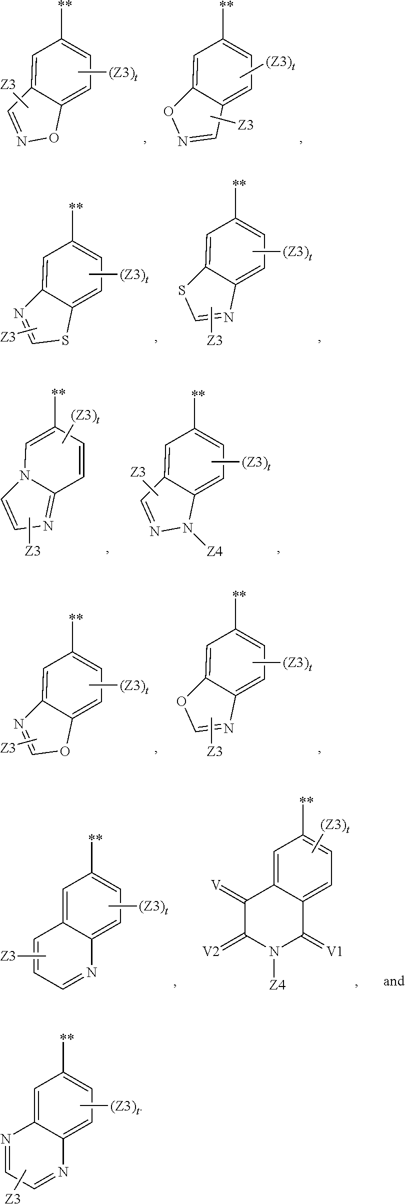

1.2.7 Compounds of Section 1.2.5 with a More Preferred Z6 Moieties

In a more preferred embodiment of compounds from Section 1.2.5, said compounds have structures of formula I-2h:

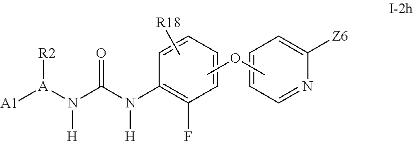

wherein Z6 is —C(O)NHR4, —NHR4 or R19 substituted pyrazole.
1.3 Compounds of Formula Ia which Exemplify Preferred A and X2-E1 Moieties

In a preferred embodiment of compounds of formula Ia, said compounds have structures of formula I-3a:

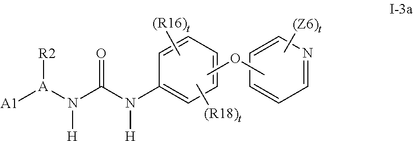

wherein the A ring is thienyl.
1.3.1 Compounds of Formula I-3a which Exemplify Preferred A1 Moieties

In a preferred embodiment of compounds of formula I-3a, said compounds have structures of formula I-3b:

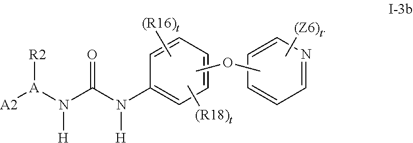

1.3.2 Compounds of Formula Ix which Exemplify Preferred A1 Moieties

In a preferred embodiment of compounds of formula I-3a, said compounds have structures of formula I-3c:

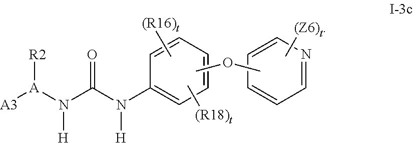

1.3.3 Compounds of Formula I-3a which Exemplify Preferred A1 Moieties

In a preferred embodiment of compounds of formula I-3a, said compounds have structures of formula I-3d:

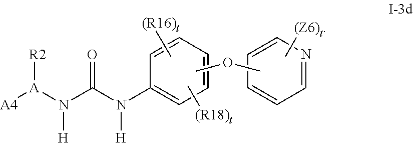

### 1.3.4 More Preferred Compounds of Section 1.3

In a preferred embodiment of compounds from Section 1.3, said compounds have structures of formula I-3e:

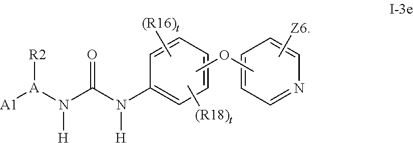

1.3.5 Compounds of Section 1.3.4 with Preferred R16 Moieties

In a preferred embodiment of compounds from Section 1.3.4, said compounds have structures of formula I-3f:

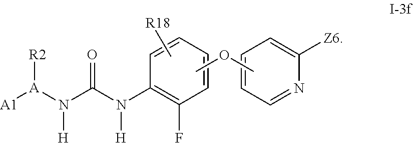

1.3.6 Compounds of Section 1.3.5 with a More Preferred A1 Moieties

In a more preferred embodiment of compounds from Section 1.3.5, said compounds have structures of formula I-3g:

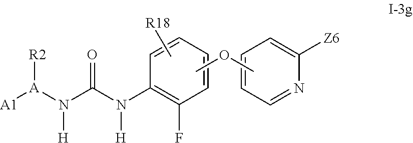

wherein A1 is selected from the group consisting of

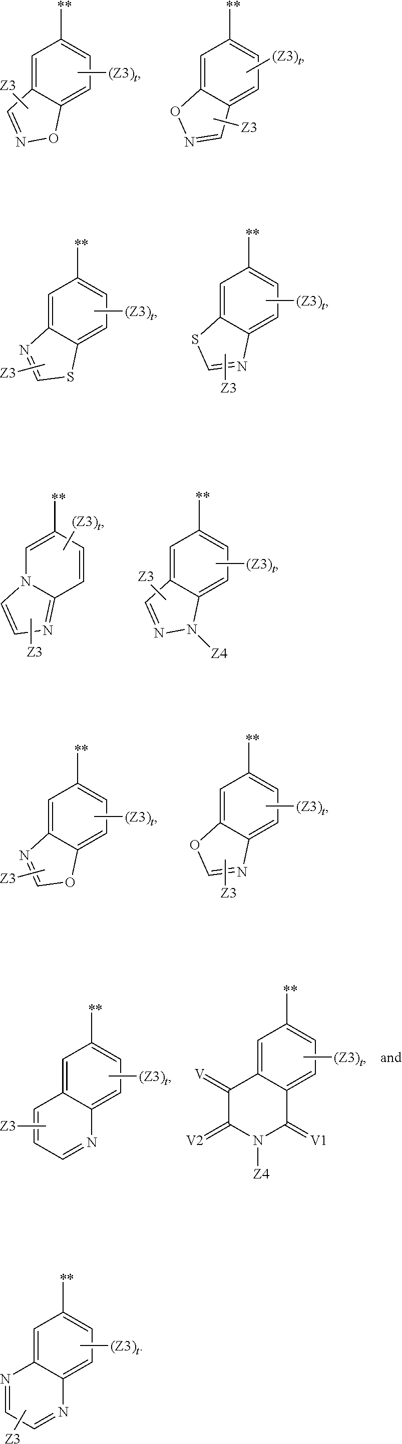

1.3.7 Compounds of Section 1.3.5 with a More Preferred Z6 Moieties

In a more preferred embodiment of compounds from Section 1.3.5, said compounds have structures of formula I-3h:

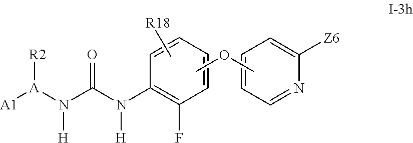

wherein Z6 is —C(O)NHR4, —NHR4 or R19 substituted pyrazole.
1.4 Compounds of Formula Ia which Exemplify Preferred A and X2-E1 Moieties

In a preferred embodiment of compounds of formula Ia, said compounds have structures of formula I-4-a:

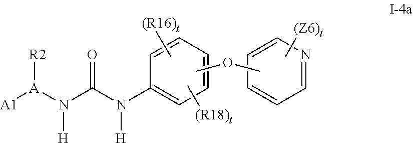

wherein the A ring is furyl.
1.4.1 Compounds of Formula Iii which Exemplify Preferred A1 Moieties

In a preferred embodiment of compounds of formula I-4a, said compounds have structures of formula I-4b:

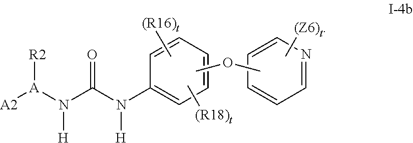

1.4.2 Compounds of Formula Iii which Exemplify Preferred A1 Moieties

In a preferred embodiment of compounds of formula I-4a, said compounds have structures of formula I-4c:

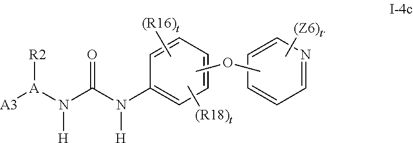

1.4.3 Compounds of Formula Im which Exemplify Preferred A1 Moieties

In a preferred embodiment of compounds of formula I-4a, said compounds have structures of formula I-4d:

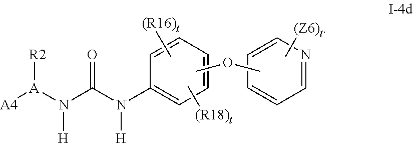

### 1.4.4 More Preferred Compounds of Section 1.4

In a preferred embodiment of compounds from Section 1.4, said compounds have structures of formula I-4e:

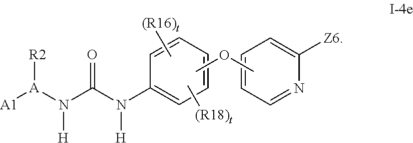

1.4.5 Compounds of Section 1.4.4 with Preferred R16 Moieties

In a preferred embodiment of compounds from Section 1.4.4, said compounds have structures of formula I-4f:

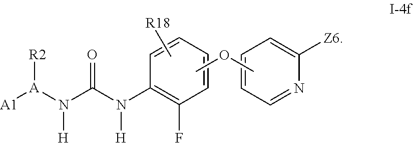

1.4.6 Compounds of Section 1.4.5 with a More Preferred A1 Moieties

In a more preferred embodiment of compounds from Section 1.4.5, said compounds have structures of formula I-4g:

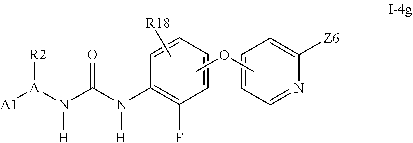

wherein A1 is selected from the group consisting of

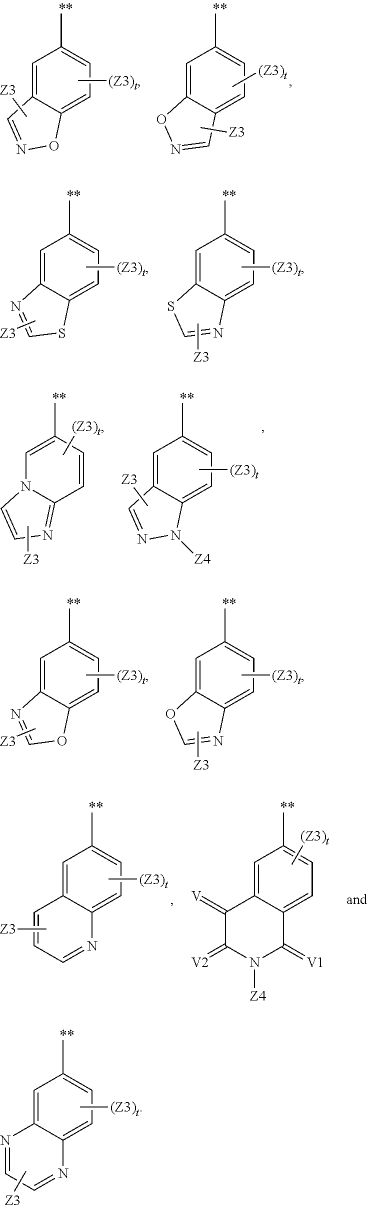

1.4.7 Compounds of Section 1.4.5 with a More Preferred Z6 Moieties

In a more preferred embodiment of compounds from Section 1.4.5, said compounds have structures of formula I-4h:

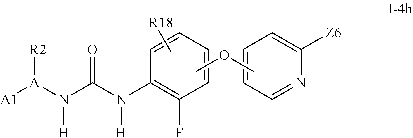

wherein Z6 is —C(O)NHR4, —NHR4 or R19 substituted pyrazole.
1.5 Compounds of Formula Ia which Exemplify Preferred A and X2-E1 Moieties

In a preferred embodiment of compounds of formula Ia, said compounds have structures of formula I-5a:

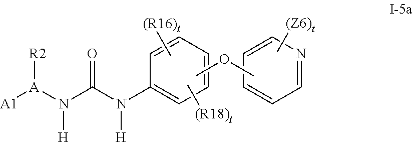

wherein the A ring is pyrrolyl.
1.5.1 Compounds of Formula I-5a which Exemplify Preferred A1 Moieties

In a preferred embodiment of compounds of formula I-5a, said compounds have structures of formula I-5b:

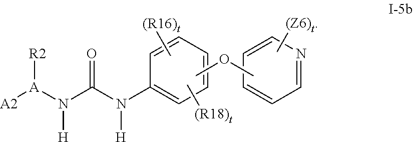

1.5.2 Compounds of Formula I-5a which Exemplify Preferred A1 Moieties

In a preferred embodiment of compounds of formula I-5a, said compounds have structures of formula I-5c:

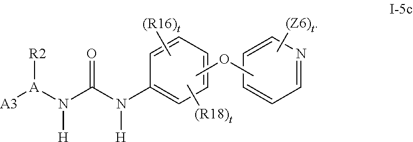

1.5.3 Compounds of Formula I-5a which Exemplify Preferred A1 Moieties

In a preferred embodiment of compounds of formula I-5a, said compounds have structures of formula I-5d:

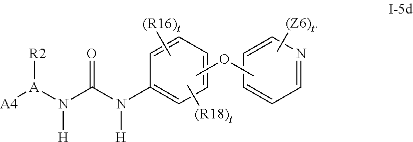

### 1.5.4 More Preferred Compounds of Section 1.5

In a preferred embodiment of compounds from Section 1.5, said compounds have structures of formula I-5e:

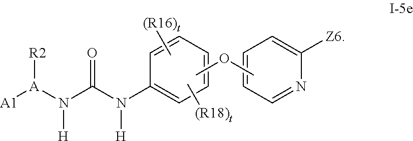

1.5.5 Compounds of Section 1.5.4 with Preferred R16 Moieties

In a preferred embodiment of compounds from Section 1.5.4, said compounds have structures of formula I-5f:

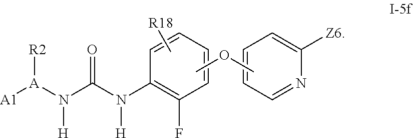

1.5.6 Compounds of Section 1.5.5 with a More Preferred A1 Moieties

In a more preferred embodiment of compounds from Section 1.5.5, said compounds have structures of formula I-5g:

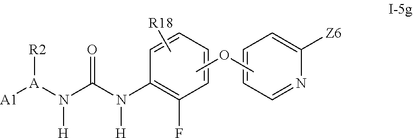

wherein A1 is selected from the group consisting of

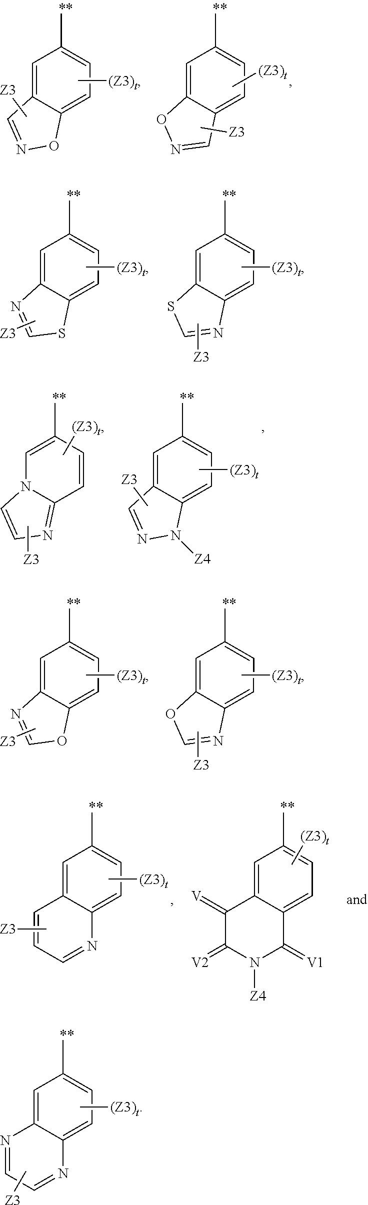

1.5.7 Compounds of Section 1.5.5 with a More Preferred Z6 Moieties

In a more preferred embodiment of compounds from Section 1.5.5, said compounds have structures of formula I-5h:

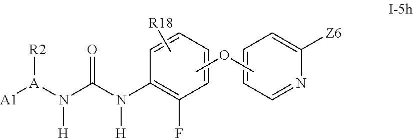

wherein Z6 is —C(O)NHR4, —NHR4 or R19 substituted pyrazole.
1.6 Compounds of Formula Ia which Exemplify Preferred A and X2-E1 Moieties

In a preferred embodiment of compounds of formula Ia, said compounds have structures of formula I-6a:

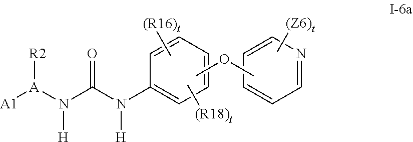

wherein the A ring is imidazolyl.
1.6.1 Compounds of Formula I-6a which Exemplify Preferred A1 Moieties

In a preferred embodiment of compounds of formula I-6a, said compounds have structures of formula I-6b:

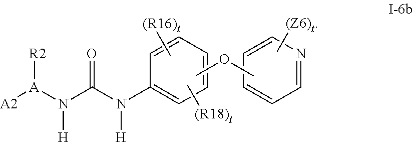

1.6.2 Compounds of Formula I-6a which Exemplify Preferred A1 Moieties

In a preferred embodiment of compounds of formula I-6a, said compounds have structures of formula I-6c:

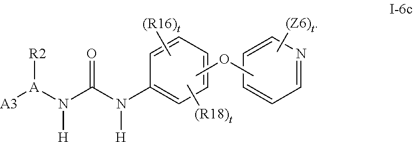

1.6.3 Compounds of Formula I-6a which Exemplify Preferred A1 Moieties

In a preferred embodiment of compounds of formula I-6a, said compounds have structures of formula I-6d:

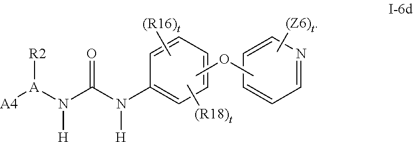

### 1.6.4 More Preferred Compounds of Section 1.6

In a preferred embodiment of compounds from Section 1.6, said compounds have structures of formula I-6e:

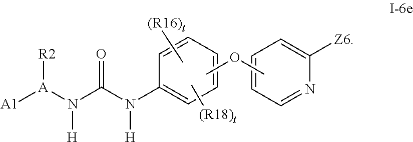

1.6.5 Compounds of Section 1.6.4 with Preferred R16 Moieties

In a preferred embodiment of compounds from Section 1.6.4, said compounds have structures of formula I-6f:

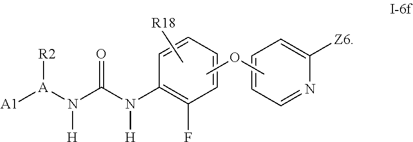

1.6.6 Compounds of Section 1.6.5 with a More Preferred A1 Moieties

In a more preferred embodiment of compounds from Section 1.6.5, said compounds have structures of formula I-6g:

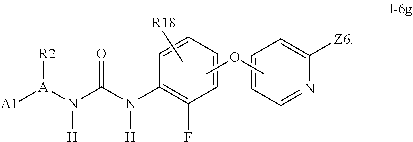

wherein A1 is selected from the group consisting of

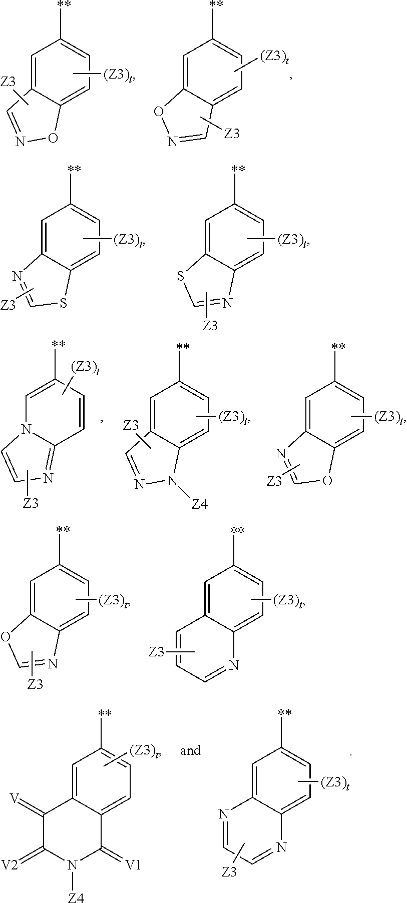

1.6.7 Compounds of Section 1.6.5 with a More Preferred Z6 Moieties

In a more preferred embodiment of compounds from Section 1.6.5, said compounds have structures of formula I-6h:

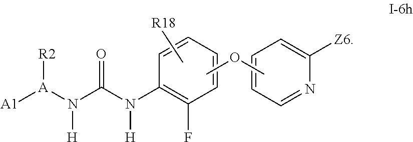

wherein Z6 is —C(O)NHR4, —NHR4 or R19 substituted pyrazole.
1.7 Compounds of Formula Ia which Exemplify Preferred A and X2-E1 Moieties

In a preferred embodiment of compounds of formula Ia, said compounds have structures of formula I-7a:

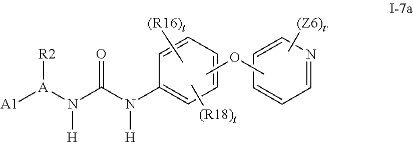

wherein the A ring is thiazolyl.
1.7.1 Compounds of Formula I-7a which Exemplify Preferred A1 Moieties

In a preferred embodiment of compounds of formula I-7a, said compounds have structures of formula I-7b:

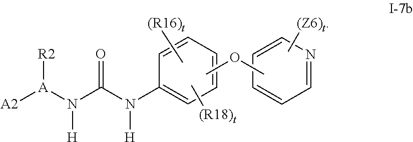

1.7.2 Compounds of Formula I-7a which Exemplify Preferred A1 Moieties

In a preferred embodiment of compounds of formula I-7a, said compounds have structures of formula I-7c:

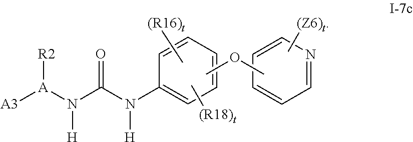

1.7.3 Compounds of Formula I-7a which Exemplify Preferred A1 Moieties

In a preferred embodiment of compounds of formula I-7a, said compounds have structures of formula I-7d:

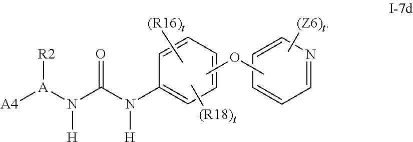

### 1.7.4 More Preferred Compounds of Section 1.7

In a preferred embodiment of compounds from Section 1.7, said compounds have structures of formula I-7e:

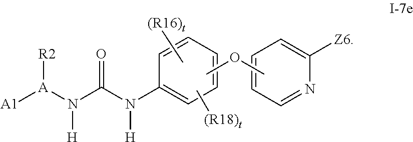

1.7.5 Compounds of Section 1.7.4 with Preferred R16 Moieties

In a preferred embodiment of compounds from Section 1.7.4, said compounds have structures of formula I-7f:

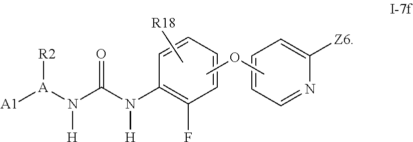

1.7.6 Compounds of Section 1.7.5 with a More Preferred A1 Moieties

In a more preferred embodiment of compounds from Section 1.7.5, said compounds have structures of formula I-7g:

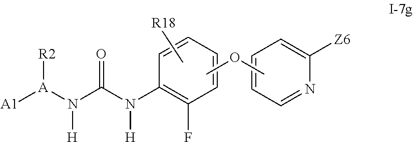

wherein A1 is selected from the group consisting of

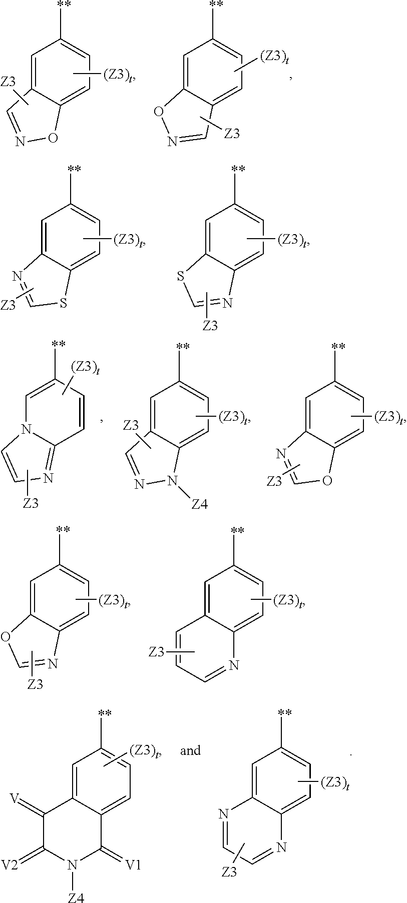

1.7.7 Compounds of Section 1.7.5 with a More Preferred Z6 Moieties

In a more preferred embodiment of compounds from Section 1.7.5, said compounds have structures of formula I-7h:

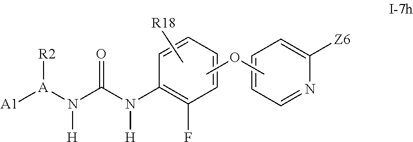

wherein Z6 is —C(O)NHR4, —NHR4 or R19 substituted pyrazole.
1.8 Compounds of Formula Ia which Exemplify Preferred A and X2-E1 Moieties

In a preferred embodiment of compounds of formula Ia, said compounds have structures of formula I-8a:

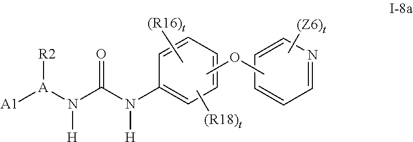

wherein the A ring is oxazolyl.
1.8.1 Compounds of Formula I-8a which exemplify preferred A1 Moieties

In a preferred embodiment of compounds of formula I-8a, said compounds have structures of formula I-8b:

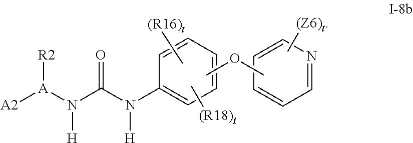

1.8.2 Compounds of Formula I-8a which Exemplify Preferred A1 Moieties

In a preferred embodiment of compounds of formula I-8a, said compounds have structures of formula I-8c:

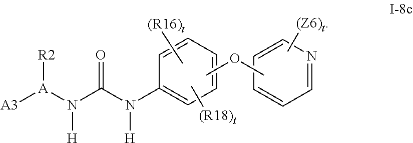

1.8.3 Compounds of Formula I-8a which Exemplify Preferred A1 Moieties

In a preferred embodiment of compounds of formula I-8a, said compounds have structures of formula I-8d:

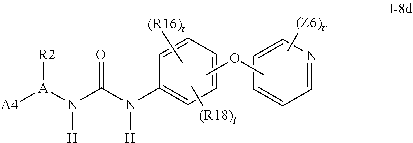

### 1.8.4 More Preferred Compounds of Section 1.8

In a preferred embodiment of compounds from Section 1.8, said compounds have structures of formula I-8e:

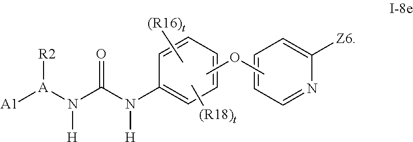

1.8.5 Compounds of Section 1.8.4 with Preferred R16 Moieties

In a preferred embodiment of compounds from Section 1.8.4, said compounds have structures of formula I-8f:

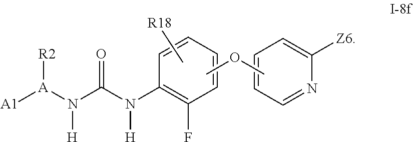

1.8.6 Compounds of Section 1.8.5 with a More Preferred A1 Moieties

In a more preferred embodiment of compounds from Section 1.8.5, said compounds have structures of formula I-8g:

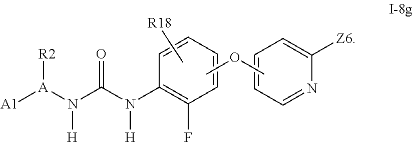

wherein A1 is selected from the group consisting of

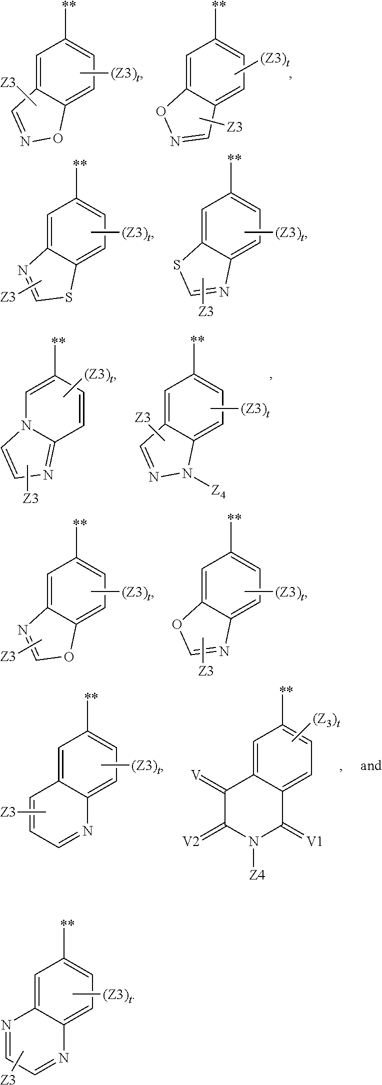

1.8.7 Compounds of Section 1.8.5 with a More Preferred Z6 Moieties

In a more preferred embodiment of compounds from Section 1.8.5, said compounds have structures of formula I-8h:

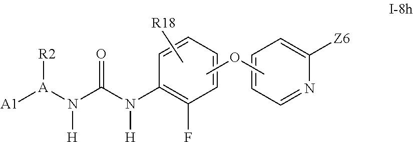

wherein Z6 is —C(O)NHR4, —NHR4 or R19 substituted pyrazole.
1.9 Compounds of Formula Ia which Exemplify Preferred A and X2-E1 Moieties

In a preferred embodiment of compounds of formula Ia, said compounds have structures of formula I-9a:

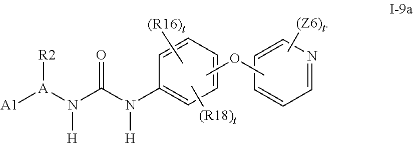

wherein the A ring is isothiazolyl.
1.9.1 Compounds of Formula I-9a which Exemplify Preferred A1 Moieties

In a preferred embodiment of compounds of formula I-9a, said compounds have structures of formula I-9b:

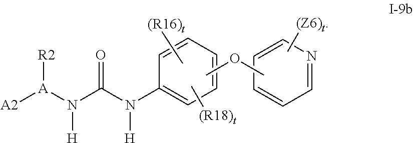

1.9.2 Compounds of Formula I-9a which Exemplify Preferred A1 Moieties

In a preferred embodiment of compounds of formula I-9a, said compounds have structures of formula I-9c:

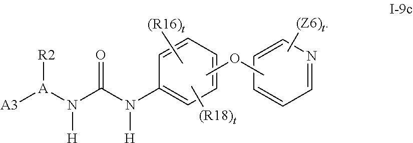

1.9.3 Compounds of Formula I-9a which Exemplify Preferred A1 Moieties

In a preferred embodiment of compounds of formula I-9a, said compounds have structures of formula I-9d:

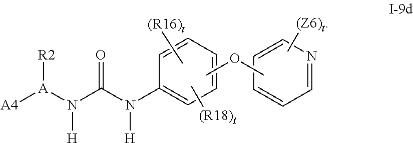

### 1.9.4 More Preferred Compounds of Section 1.9

In a preferred embodiment of compounds from Section 1.9, said compounds have structures of formula I-9e:

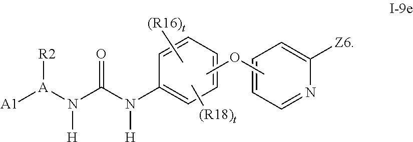

1.9.5 Compounds of Section 1.9.4 with Preferred R16 Moieties

In a preferred embodiment of compounds from Section 1.9.4, said compounds have structures of formula I-9f:

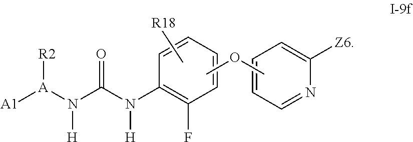

1.9.6 Compounds of Section 1.9.5 with a More Preferred A1 Moieties

In a more preferred embodiment of compounds from Section 1.9.5, said compounds have structures of formula I-9g:

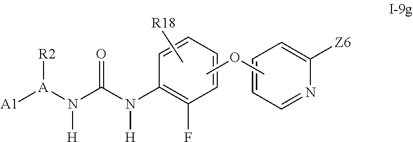

wherein A1 is selected from the group consisting of

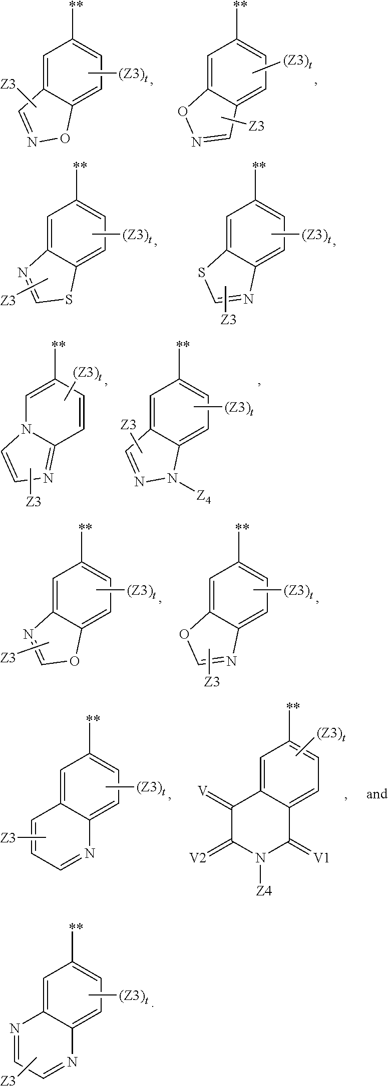

1.9.7 Compounds of Section 1.9.5 with a More Preferred Z6 Moieties

In a more preferred embodiment of compounds from Section 1.9.5, said compounds have structures of formula I-9h:

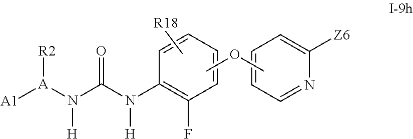

wherein Z6 is —C(O)NHR4, —NHR4 or R19 substituted pyrazole.
1.10 Compounds of Formula Ia which Exemplify Preferred A and X2-E1 Moieties

In a preferred embodiment of compounds of formula Ia, said compounds have structures of formula I-10a:

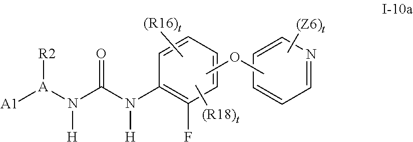

wherein the A ring is phenyl.
1.10.1 Compounds of Formula I-10a which Exemplify Preferred A1 Moieties

In a preferred embodiment of compounds of formula I-10a, said compounds have structures of formula I-10b:

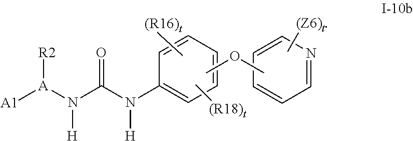

1.10.2 Compounds of Formula I-10a which Exemplify Preferred A1 Moieties

In a preferred embodiment of compounds of formula I-10a, said compounds have structures of formula I-10c:

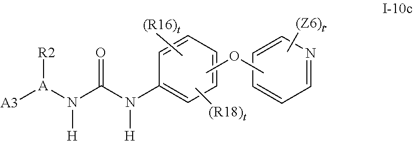

1.10.3 Compounds of Formula I-10a which Exemplify Preferred A1 Moieties

In a preferred embodiment of compounds of formula I-10a, said compounds have structures of formula I-10d:

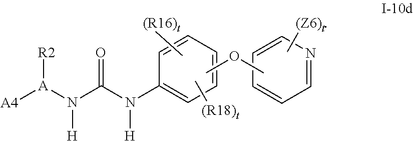

### 1.10.4 More Preferred Compounds of Section 1.10

In a preferred embodiment of compounds from Section 1.10, said compounds have structures of formula I-10e:

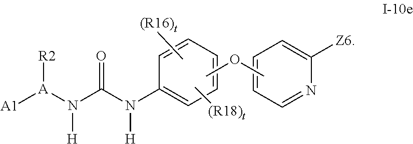

1.10.5 Compounds of Section 1.10.4 with Preferred R16 Moieties

In a preferred embodiment of compounds from Section 1.10.4, said compounds have structures of formula I-10f:

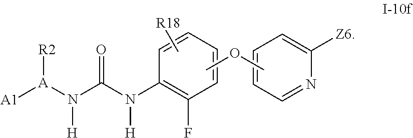

1.10.6 Compounds of Section 1.10.5 with a More Preferred A1 Moieties

In a more preferred embodiment of compounds from Section 1.10.5, said compounds have structures of formula I-10g:

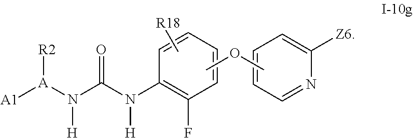

wherein A1 is selected from the group consisting of

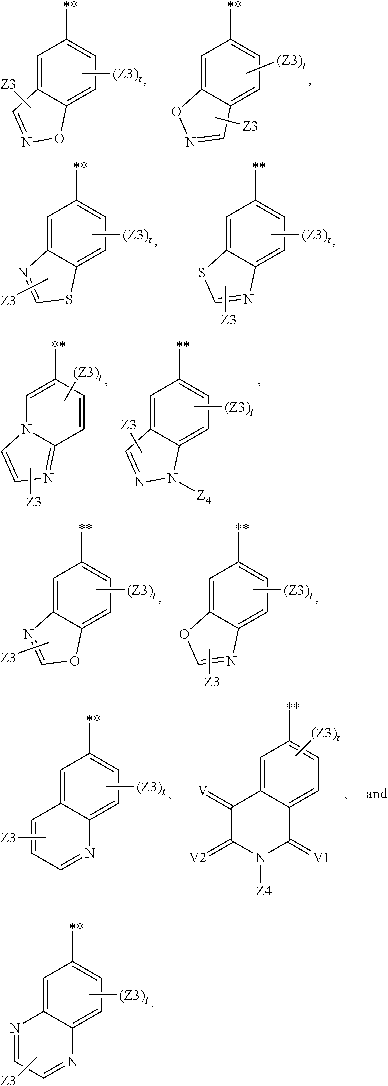

1.10.7 Compounds of Section 1.10.5 with a More Preferred Z6 Moieties

In a more preferred embodiment of compounds from Section 1.10.5, said compounds have structures of formula I-10h:

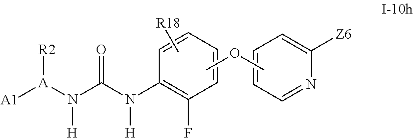

wherein Z6 is —C(O)NHR4, —NHR4 or R19 substituted pyrazole.
1.11 Compounds of Formula Ia which Exemplify Preferred A and X2-E1 Moieties

In a preferred embodiment of compounds of formula Ia, said compounds have structures of formula I-11a:

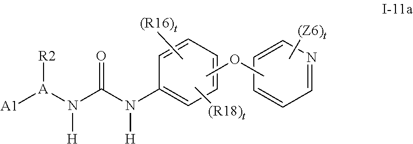

wherein the A ring is pyrimidinyl.
1.11.1 Compounds of Formula I-11a which exemplify preferred A1 Moieties

In a preferred embodiment of compounds of formula I-11a, said compounds have structures of formula I-11b:

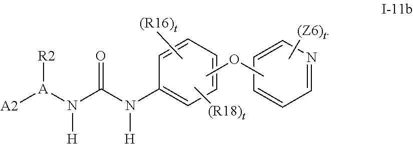

1.11.2 Compounds of Formula I-11a which Exemplify Preferred A1 Moieties

In a preferred embodiment of compounds of formula I-11a, said compounds have structures of formula I-11c:

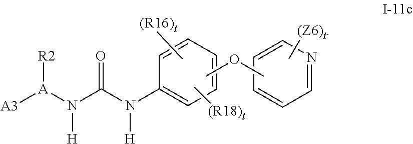

1.11.3 Compounds of Formula I-11a which Exemplify Preferred A1 Moieties

In a preferred embodiment of compounds of formula I-11a, said compounds have structures of formula I-11d:

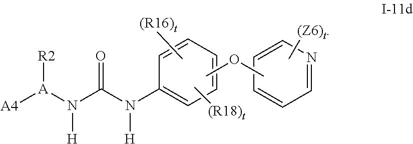

### 1.11.4 More Preferred Compounds of Section 1.11

In a preferred embodiment of compounds from Section 1.11, said compounds have structures of formula I-11e:

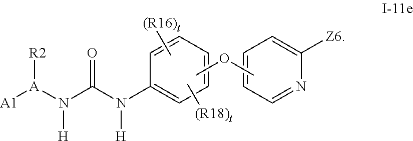

1.11.5 Compounds of Section 1.11.4 with Preferred R16 Moieties

In a preferred embodiment of compounds from Section 1.11.4, said compounds have structures of formula I-11f:

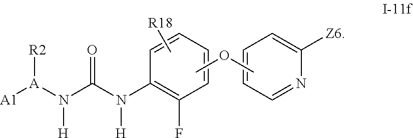

1.11.6 Compounds of Section 1.11.5 with a More Preferred A1 Moieties

In a more preferred embodiment of compounds from Section 1.11.5, said compounds have structures of formula I-11g:

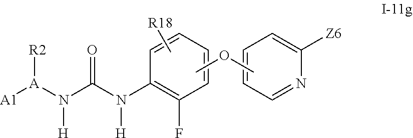

wherein A1 is selected from the group consisting of

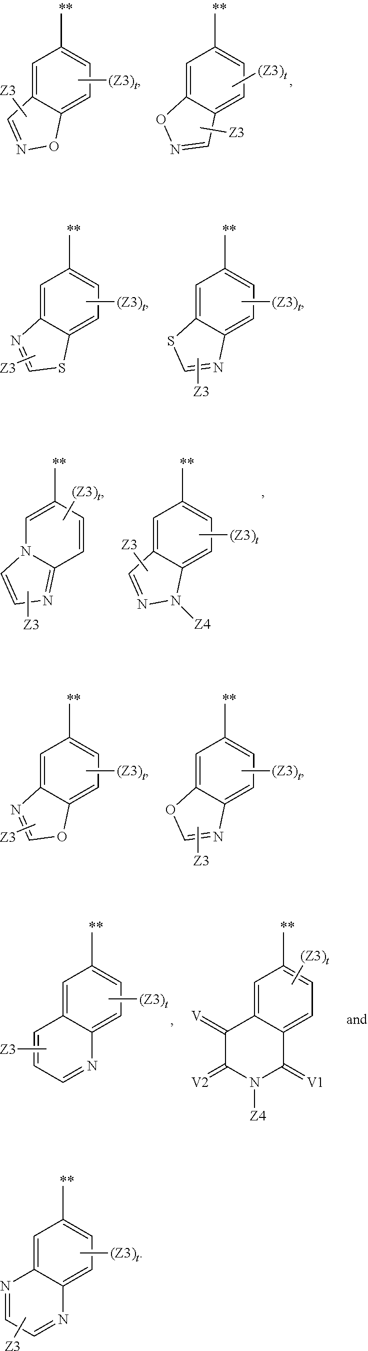

1.11.7 Compounds of Section 1.11.5 with a More Preferred Z6 Moieties

In a more preferred embodiment of compounds from Section 1.11.5, said compounds have structures of formula I-11h:

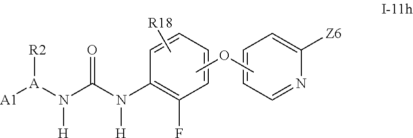

wherein Z6 is —C(O)NHR4, —NHR4 or R19 substituted pyrazole.
1.12 Compounds of Formula Ia which Exemplify Preferred A and X2-E1 Moieties

In a preferred embodiment of compounds of formula Ia, said compounds have structures of formula I-12a:

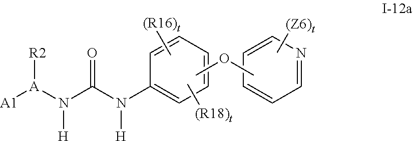

wherein the A ring is pyridinyl.
1.12.1 Compounds of Formula I-12a which Exemplify Preferred A1 Moieties

In a preferred embodiment of compounds of formula I-12a, said compounds have structures of formula I-12b:

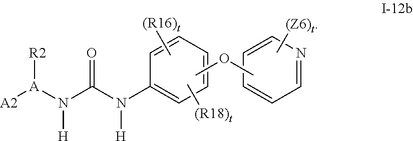

1.12.2 Compounds of Formula I-12a which Exemplify Preferred A1 Moieties

In a preferred embodiment of compounds of formula I-12a, said compounds have structures of formula I-12c:

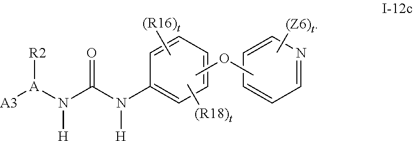

1.12.3 Compounds of Formula I-12a which Exemplify Preferred A1 Moieties

In a preferred embodiment of compounds of formula I-12a, said compounds have structures of formula I-12d:

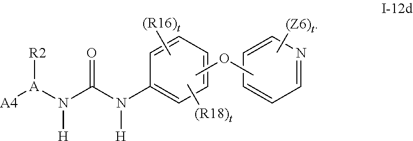

### 1.12.4 More Preferred Compounds of Section 1.12

In a preferred embodiment of compounds from Section 1.12, said compounds have structures of formula I-12e:

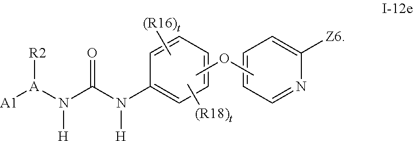

1.12.5 Compounds of Section 1.12.4 with Preferred R16 Moieties

In a preferred embodiment of compounds from Section 1.12.4, said compounds have structures of formula I-12f:

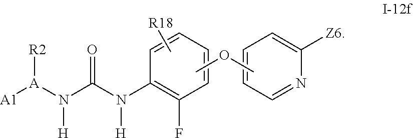

1.12.6 Compounds of Section 1.12.5 with a More Preferred A1 Moieties

In a more preferred embodiment of compounds from Section 1.12.5, said compounds have structures of formula I-12g:

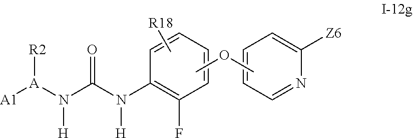

wherein A1 is selected from the group consisting of

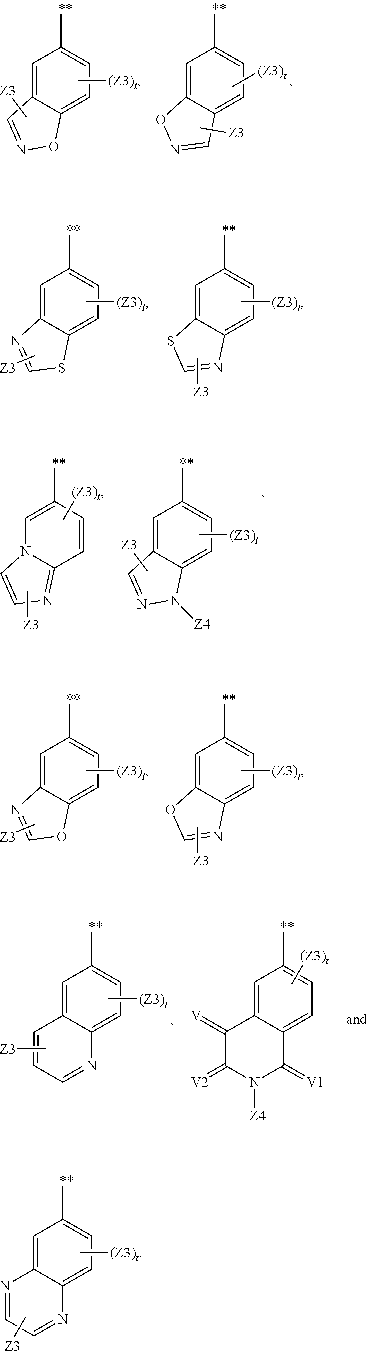

1.12.7 Compounds of Section 1.12.5 with a More Preferred Z6 Moieties compounds have structures of formula I-12h:

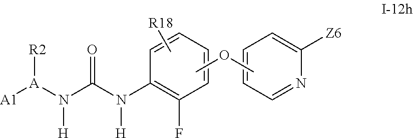

wherein Z6 is —C(O)NHR4, —NHR4 or R19 substituted pyrazole.

### 1.13 Methods

### 1.13a Methods of Protein Modulation

The invention includes methods of modulating kinase activity of a variety of kinases, e.g. c-ABL kinase, BCR-ABL kinase, FLT-3, TIE-2 kinase, the TRK family of kinases, c-KIT, PDGFR, VEGFR, c-MET, the HER family of kinases, RET kinase, and c-FMS kinase. The kinases may be wildtype kinases, oncogenic forms thereof, aberrant fusion proteins thereof or polymorphs of any of the foregoing. The method comprises the step of contacting the kinase species with a compound of formula Ia and especially those set forth in sections 1.1-1.12. The kinase species may be activated or unactivated, and the species may be modulated by phosphorylations, sulfation, fatty acid acylations glycosylations, nitrosylation, cystinylation (i.e. proximal cysteine residues in the kinase react with each other to form a disulfide bond) or oxidation. The kinase activity may be selected from the group consisting of catalysis of phospho transfer reactions, inhibition of phosphorylation, oxidation or nitrosylation of said kinase by another enzyme, enhancement of dephosphorylation, reduction or denitrosylation of said kinase by another enzyme, kinase cellular localization, and recruitment of other proteins into signaling complexes through modulation of kinase conformation.

### 1.13b Treatment Methods

The methods of the invention also include treating individuals suffering from a condition selected from the group consisting of cancer and hyperproliferative diseases. These methods comprise administering to such individuals a compound of formula Ia, and especially those of sections 1.1-1.12, said diseases including, but not limited to, a disease caused by c-ABL kinase, oncogenic forms thereof, aberrant fusion proteins thereof including BCR-ABL kinase and polymorphs thereof; a disease caused by FLT-3 kinase, oncogenic forms thereof, aberrant fusion proteins thereof and polymorphs thereof; a disease caused by TIE-2 kinase, oncogenic forms thereof, aberrant fusion proteins thereof and polymorphs thereof; a disease caused by the TRK family of kinases, oncogenic forms thereof, aberrant fusion proteins thereof and polymorphs thereof; a disease caused by cMET kinase, oncogenic forms thereof, aberrant fusion proteins thereof including TPR-MET; a disease caused by KDR kinase or PDGFR kinases; a disease caused by HER kinases, oncogenic forms thereof and polymorphs thereof; a disease caused by RET kinase, oncogenic forms thereof, aberrant fusion proteins thereof; a disease caused by c-FMS kinase, oncogenic forms thereof and polymorphs thereof; a disease caused by a c-KIT kinase, oncogenic forms thereof, aberrant fusion proteins thereof and polymorphs thereof; and diseases caused by any of the foregoing kinases, oncogenic forms thereof, and aberrant fusion proteins thereof, including but not limited to, chronic myelogenous leukemia, acute lymphocytic leukemia, acute myeloid leukemia, other myeloproliferative disorders, a disease caused by metastasis of primary solid tumors to secondary sites, glioblastomas, ovarian cancer, pancreatic cancer, prostate cancer, lung cancers, mesothelioma, hypereosinophilic syndrome, a disease caused or maintained by pathological vascularization, ocular diseases characterized by hyperproliferation leading to blindness including various retinopathies, i.e. diabetic retinopathy and age-related macular degeneration, non small cell lung cancer, breast cancers, kidney cancers, colon cancers, cervical carcinomas, papillary thyroid carcinoma, melanomas, autoimmune diseases including rheumatoid arthritis, multiple sclerosis, lupus, asthma, human inflammation, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic obstructive pulmonary disease, bone resorptive diseases, bone cancer, graft-versus-host reaction, Crohn's disease, ulcerative colitis, inflammatory bowel disease, pyresis, gastrointestinal stromal tumors, and combinations thereof. The administration method is not critical, and may be from the group consisting of oral, parenteral, inhalation, and subcutaneous.

### 1.14 Dosage

The methods of the present invention may be used to prevent, treat, or reduce the severity of cancer or hyperproliferative diseases. The exact amount required will vary from subject to subject, depending on the species, age, and general condition of the subject, the severity of the disease, the particular agent, its mode of administration, and the like. The compounds of formula Ia are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds of formula Ia and compositions described herein will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, body surface area, general health, sex, ethnicity and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Administration of a compound of formula Ia or an additional pharmaceutically active agent can be accomplished via any mode of administration for therapeutic agents. These modes include systemic or local administration such as oral, nasal, parenteral, transdermal, subcutaneous, vaginal, buccal, rectal or topical administration modes. In some instances, administration will result in the release of the compound of formula Ia or an additional pharmaceutically active agent described herein into the bloodstream.

In one embodiment, the compound of formula Ia or an additional pharmaceutically active agent described herein is administered orally.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, preferably in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using dissolution or suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, aqueous dextrose, glycerol, ethanol, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of formula Ia, it is often desirable to slow the absorption of the compound from subcutaneous injection or intramuscular injection, or to slow the rate of systemic absorption upon oral administration. This may be accomplished by the use of a liquid suspension of crystalline or amorphous material with poor water solubility. The rate of absorption of the compound then depends upon its rate of dissolution that, in turn, may depend upon crystal size and crystalline form. Modified or sustained release formulations, well known in the art, may also be utilized in formulations to control the rate of absorption of an orally administered compound. Alternatively, modified or sustained absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders or diluents such as starches, lactose, sucrose, glucose, mannitol, cellulose, saccharin, glycine, and silicic acid, b) binders such as, for example, magnesium aluminum silicate, starch paste, tragacanth, carboxymethylcellulose, methyl cellulose, alginates, gelatin, polyvinylpyrrolidinone, magnesium carbonate, natural sugars, corn sweeteners, sucrose, waxes and natural or synthetic gums such as acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators or disintegrants such as quaternary ammonium compounds, starches, agar, methyl cellulose, bentonite, xanthangum, algiic acid, and effervescent mixtures, g) wetting agents such as, for example, cetyl alcohol and glycerol monostearate, h) absorbents such as kaolin and bentonite clay, and i) lubricants such as talc, silica, stearic acid, calcium stearate, magnesium stearate, sodium oleate, sodium acetate, sodium chloride, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a modified or sustained manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The compound of formula Ia or pharmaceutically active agent can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the compound of formula Ia or pharmaceutically active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a modified or sustained manner. Examples of embedding compositions that can be used include polymeric substances and waxes.

The compound of formula Ia or pharmaceutically active agent described herein can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of the compound of formula Ia or pharmaceutically active agent to a form lipid layer encapsulating the drug, as described in U.S. Pat. No. 5,262,564.

The compound of formula Ia or pharmaceutically active agent described herein can also be delivered by the use of monoclonal antibodies as individual carriers to which the compound or pharmaceutically active agent described herein are coupled or conjugated. The compound of formula Ia or pharmaceutically active agent described herein can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the compound of formula Ia or pharmaceutically active agent described herein can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Furthermore, a compound of formula Ia and pharmaceutically active agents described herein may be coupled, absorbed, adsorbed, or conjugated to a medical device including but not limited to stents.

Parenteral injectable administration can be used for subcutaneous, intramuscular, intra-articular, or intravenous injections and infusions. Injectables can be prepared in conventional forms, either as liquid solutions or suspensions or solid forms suitable for dissolving in liquid prior to injection.

One embodiment, for parenteral administration employs the implantation of a slow-release or sustained-released system, according to U.S. Pat. No. 3,710,795, incorporated herein by reference.

The compositions can be sterilized or contain non-toxic amounts of adjuvants, such as preserving, stabilizing, wetting or emulsifying agents, solution promoters, salts for regulating the osmotic pressure, pH buffering agents, and other substances, including, but not limited to, sodium acetate or triethanolamine oleate. In addition, they can also contain other therapeutically valuable substances.

Dosage forms for topical or transdermal administration of a compound of formula Ia or pharmaceutically active agent include ointments, pastes, creams, lotions, gels, powders, solutions, sprays, inhalants or patches. The compound of formula Ia or pharmaceutically active agent described herein is admixed under sterile conditions with a pharmaceutically acceptable carrier and any needed preservatives or buffers as may be required. Ophthalmic formulation, ear drops, and eye drops are also contemplated as being within the scope of this invention. Furthermore, the compound of formula Ia or pharmaceutically active agent described herein can be administered in intranasal form via topical use of suitable intranasal vehicles. Additionally, the present invention contemplates the use of transdermal patches or via other transdermal routes, using those forms of transdermal skin patches and formulations well known to those of ordinary skill in that art. Transdermal patches have the added advantage of providing controlled delivery of a compound of formula Ia or pharmaceutically active agent to the body. Such dosage forms can be made by dissolving or dispensing the compound of formula Ia or pharmaceutically active agent in the proper medium. Absorption enhancers can also be used to increase the flux of the compound of formula Ia or pharmaceutically active agent across the skin. The rate can be controlled by either providing a rate controlling membrane or by dispersing the compound of formula Ia or pharmaceutically active agent in a polymer matrix or gel.

Compositions can be prepared according to conventional mixing, granulating or coating methods, respectively, and the present pharmaceutical compositions can contain from about 0.1% to about 99%, preferably from about 1% to about 70% of the compound of formula Ia or pharmaceutically active agent described herein by weight or volume.

The dosage regimen utilizing the compound of formula Ia or pharmaceutically active agent described herein can be selected in accordance with a variety of factors including type, species, age, weight, body surface area, sex, ethnicity, and medical condition of the subject; the severity of the condition to be treated; the route of administration; the renal or hepatic function of the subject; and the particular compound of formula Ia or pharmaceutically active agent described herein employed. A person skilled in the art can readily determine and prescribe the effective amount of the drug useful for treating or preventing a proliferative disorder.

Effective dosage amounts of the compound of formula Ia or pharmaceutically active agent described herein, when administered to a subject, range from about 0.05 to about 3,500 mg of compound of formula Ia or pharmaceutically active agent described herein per day. Unit dosage compositions for in vivo or in vitro use can contain about 0.01, 0.5, 1.0, 2.5, 5.0, 10.0, 15.0, 25.0, 50.0, 100.0, 250.0, 500.0 or 1000.0 mg of the compound of formula Ia or pharmaceutically active agent described herein. In one embodiment, the unit dosage compositions are in the form of a tablet that can be scored. The amount of a compound of formula Ia or pharmaceutically active agent described herein that is effective in the treatment or prevention of cancer or hyperproliferative disease can be determined by clinical techniques that are known to those of skill in the art. In addition, in vitro and in vivo assays can optionally be employed to help identify optimal dosage ranges. The precise dose to be employed can also depend on the route of administration, and the seriousness of the proliferative disorder being treated and can be decided according to the judgment of the practitioner and each subject's circumstances in view of, e.g., published clinical studies. Suitable effective dosage amounts, however, can range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, or about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one compound of formula Ia or pharmaceutically active agent described herein is administered, the effective dosage amounts correspond to the total amount administered.

In some embodiments, daily dosages of a compound of formula Ia or a pharmaceutically active agent range from about 1 mg/kg to about 100 mg/kg. In another embodiment daily dosages of a compound of formula Ia or a pharmaceutically active agent range from about 1 mg/kg to about 10 mg/kg. In some embodiments, the total daily dose of a compound of formula Ia or a pharmaceutically active agent is selected from about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, and about 10 mg/kg.

In some embodiments, the total daily dose of a compound of formula Ia or a pharmaceutically active agent is administered once daily. In other embodiments, the total daily dose of a compound of formula Ia or a pharmaceutically active agent is administered in two doses per day. In other embodiments, the total daily dose of a compound of formula Ia or a pharmaceutically active agent is administered in three doses per day. In other embodiments, the total daily dose of a compound of formula Ia or a pharmaceutically active agent is administered in four doses per day.

The dosage regimen utilizing the compound of formula Ia or pharmaceutically active agent described herein can be selected in accordance with a variety of factors including type, species, age, weight, body surface area, sex, ethnicity, and medical condition of the subject; the severity of the cancer or hyperproliferative disorder to be treated; the route of administration; the renal or hepatic function of the subject; and the particular compound of formula Ia or pharmaceutically active agent described herein employed. A person skilled in the art can readily determine and prescribe the effective amount of the compound of formula Ia or pharmaceutically active agent required to prevent, counter or arrest the progress of the proliferative disorder.

The compound of formula Ia or pharmaceutically active agent described herein can be administered in a single daily dose, or the total daily dosage can be administered in divided doses of two, three or four times daily. When administered in the form of a transdermal delivery system, the dosage administration can be continuous rather than intermittent throughout the dosage regimen. Dosage strengths of topical preparations including creams, ointments, lotions, aerosol sprays and gels, contain the compound or pharmaceutically active agent described herein ranging from about 0.1% to about 15%, w/w or w/v.

### 1.15 Combination

Depending upon the particular condition, or disease, to be treated, additional pharmaceutically active agents, which are normally administered to treat that condition, may be administered in combination with compounds of formula Ia. As used herein, additional pharmaceutically active agents that are normally administered to treat a particular disease, or condition, are known as “appropriate for the disease, or condition, being treated”.

Those additional pharmaceutically active agents may be administered separately from a compound of formula Ia as part of a multiple dosage regimen. Alternatively, those pharmaceutically active agents may be part of a single dosage form, mixed together with a compound of formula Ia in a single composition. If administered as part of a multiple dosage regime, the two or more pharmaceutically active agents may be administered simultaneously, sequentially or within a period of time from one another normally within five hours from one another.

In another embodiment, the pharmaceutically active agent(s) may be administered with a compound of formula Ia as part of an alternating dosing combination. In such an alternating dosing combination, a compound of formula Ia is dosed to a patient for a period of time ranging from two weeks to six months, followed by administration of the additional pharmaceutically active agent(s) for a second period of time ranging from two weeks to six months. This alternating dosing combination schedule may be repeated multiple times and the time period for dosing of the compound of formula Ia and the time period for dosing of the pharmaceutically active agent(s) may be adjusted. A drug holiday, wherein no compound of formula Ia or pharmaceutically active agent(s) is dosed, may optionally be implemented between the alternate dosing time periods of the compound of formula Ia and the pharmaceutically active agent(s).

As used herein, the term “combination,” “combined,” and related terms refers to the simultaneous, sequential, or alternating administration of a compound of formula Ia or pharmaceutically active agent(s) in accordance with this invention. For example, a compound of formula Ia may be administered with another pharmaceutically active agent simultaneously or sequentially in separate unit dosage forms or together in a single unit dosage form. Accordingly, the present invention provides a single unit dosage form comprising a compound of formula Ia, an additional pharmaceutically active agent, and a pharmaceutically acceptable carrier, adjuvant, or vehicle.

In certain embodiments, a combination of one additional pharmaceutically active agent and a compound of formula Ia are described. In some embodiments, two or more pharmaceutically active agents may be administered with a compound of formula Ia. In other embodiments, a combination of three or more additional pharmaceutically active agents may be administered with a compound of formula Ia.

In some embodiments, the additional pharmaceutically active agent is selected from taxanes such as taxol, taxotere or their analogues; alkylating agents such as cyclophosphamide, isosfamide, melphalan, hexamethylmelamine, thiotepa or dacarbazine; antimetabolites such as pyrimidine analogues, for instance 5-fluorouracil, cytarabine, capecitabine, azacitibine, and gemcitabine or its analogues such as 2-fluorodeoxycytidine; folic acid analogues such as methotrexate, idatrexate, trimetrexate, or pralatrexate; spindle poisons including vinca alkaloids such as vinblastine, vincristine, vinorelbine and vindesine, or their synthetic analogues such as navelbine, or estramustine and a taxoid; platinum compounds such as cisplatin; epipodophyllotoxins such as etoposide or teniposide; steroids such as prednisone; antibiotics such as daunorubicin, doxorubicin, bleomycin or mitomycin, enzymes such as L-asparaginase, topoisomerase inhibitors such as topotecan or pyridobenzoindole derivatives; and various agents such as procarbazine, mitoxantrone; biological response modifiers or growth factor inhibitors such as interferons or interleukins; inhibitors of growth factors, for example Bevacizumab and Ranibizumab; HSP-90 inhibitors, for example 17-AAG (Geldanamycin), 17-DMAG (Alvespimycin), NVP-BEP800, and BIIB021; small molecular deubiquitinase (DUB) inhibitors such as WP1130; chemokine receptor antagonists including CXCR4 antagonists; kinase inhibitors including Cetuximab, Imatinib, Trastuzumab, Gefitinib, Pegaptanib, Sorafenib, Regorafenib, Dasatinib, Bosutinib, Ponatinib, Sunitinib, Erlotinib, Nilotinib, Lapatinib, Panitumumab, Pazopanib, Crizotinib, AT-9283, Bafetinib (INNO-406), Danusertib (PHA-739358), KW-2449, Sarcatinib (AZD0530), Tozasertib (VX-680), Lestaurtinib (CEP-701), Tandutinib, Linifinib (ABT-869), Vatalinib, Axitinib, Dovitinib, Cediranib, Vandetinib, Zelboraf® (Vemurafenib), Cabozantinib (XL-184), the JAK inhibitor CP-690,550, and the SYK inhibitor Fostamatinib. In other embodiments, the other pharmaceutically active agent in addition to a compound of formula Ia is Imatinib. In other embodiments, the other pharmaceutically active agent in addition to a compound of formula Ia is Dasatinib.

Other examples of pharmaceutically active agents include, without limitation: agents for the treatment of Alzheimer's Disease such as Aricept® and Excelon®; agents for the treatment of HIV such as ritonavir; pharmaceutically active agents for the treatment of Parkinson's Disease such as L-DOPA/carbidopa, entacapone, ropinrole, pramipexole, bromocriptine, pergolide, trihexephendyl, and amantadine; pharmaceutically active agents for the treatment of Multiple Sclerosis (MS) such as beta interferon (e.g., Avonex® and Rebif®), Copaxone®, mitoxantrone, and Natalizumab; pharmaceutically active agents for the treatment of asthma such as albuterol and Singulair®; pharmaceutically active agents for the treatment of schizophrenia such as zyprexa, risperdal, seroquel, and haloperidol; pharmaceutically active agents for the treatment of inflammation such as corticosteroids, methotrexate, azathioprine, cyclophosphamide, and sulfasalazine; pharmaceutically active agents for the treatment of TNF-mediated disease including Humira®, Enbrel®, and Remicade®; pharmaceutically active agents for the treatment of IL-1-mediated disease including IL-1 receptor antagonists such as but not limited to Kineret® and Rilonacept; pharmaceutically active agents for the treatment of IL-6-mediated disease including IL-6 receptor inhibitors such as but not limited to toxiclizumab; pharmaceutically active agents for the treatment of CD-20-mediated disease including anti-CD20 agents such as but not limited to Rituxin®;

Other classes of pharmaceutically active agents include immunomodulatory and immunosuppressive agents such as Vervoy®, abatacept, cyclosporin, tacrolimus, ridaforolimus, rapamycin, mycophenolate mofetil, interferons, corticosteroids, cyclophophamide, azathioprine, and sulfasalazine; bone resorptive inhibitory agents including denosumab and bisphosphonates including zoledronic acid; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, compounds of formula Ia, or a pharmaceutically acceptable composition thereof, are administered in combination with a monoclonal antibody or an siRNA therapeutic.

Those additional pharmaceutically active agents may be administered separately from a compound of formula Ia as part of a multiple dosage regimen. Alternatively, those pharmaceutically active agents may be part of a single dosage form, mixed together with a compound of formula Ia in a single composition. If administered as part of a multiple dosage regime, the compounds of formula Ia and two or more pharmaceutically active agents may be administered simultaneously, sequentially or within a period of time from one another normally within five hours from one another.

In another embodiment, the additional pharmaceutically active agent(s) may be administered with a compound of formula Ia as part of an alternating dosing combination. In such an alternating dosing combination, a compound of formula Ia is dosed to a patient for a period of time ranging from two weeks to six months, followed by administration of the additional pharmaceutically active agent(s) for a second period of time ranging from two weeks to six months. This alternating dosing combination schedule may be repeated multiple times and the time period for dosing of the compound of formula Ia and the time period for dosing of the additional pharmaceutically active agent(s) may be adjusted. A drug holiday, wherein no compound of formula Ia or additional pharmaceutically active agent(s) is dosed, may optionally be implemented between the alternate dosing time periods of the compound of formula Ia and the additional pharmaceutically active agent(s).

The amount of both a compound of formula Ia and additional pharmaceutically active agent(s) (in those compositions which comprise additional pharmaceutically active agents as described above) that may be combined with the carrier materials to produce a single dosage form will vary depending upon the host treated and the particular mode of administration. This dosage form can be formulated so that the dosage amount of the compound of formula Ia and the dosage amount of the additional pharmaceutically active agent are independently between 0.01-100 mg/kg body weight.

In those compositions which comprise an additional pharmaceutically active agent, that additional pharmaceutically active agent and the compound of formula Ia may act synergistically. Therefore, the amount of additional pharmaceutically active agent in such compositions will be less than that required in a monotherapy utilizing only that pharmaceutically active agent. In such compositions a dosage of between 0.01-100 mg/kg body weight of the additional pharmaceutically active agent can be administered.

The amount of additional pharmaceutically active agent present in the compositions of this invention will be no more than the amount that would normally be administered in a composition comprising that pharmaceutically active agent as the only active agent. In some embodiments, the amount of additional pharmaceutically active agent in the presently disclosed compositions will range from about 50% to 100% of the amount normally present in a composition comprising that agent as the only pharmaceutically active agent.

In some embodiments, the compositions comprise an amount of a compound of formula Ia wherein the other pharmaceutically active agent is an anticancer agent. In another embodiment, the amount of the compound of formula Ia and the other anticancer agent is at least about 0.01% of the combined combination chemotherapy agents by weight of the composition. When intended for oral administration, this amount can be varied from about 0.1% to about 80% by weight of the composition. Some oral compositions can comprise from about 4% to about 50% of the compound of formula Ia and the other anticancer agent by weight of the composition. Other compositions of the present invention are prepared so that a parenteral dosage unit contains from about 0.01% to about 2% by weight of the composition.

The present methods for treating or preventing cancer or a hyperproliferative disease in a subject in need thereof can further comprise administering an additional pharmaceutically active agent that is a prophylactic or therapeutic agent to be administered with a compound of formula Ia. The other prophylactic or therapeutic agent includes, but is not limited to, an anti-inflammatory agent, an anti-renal failure agent, an anti-diabetic agent, an anti-cardiovascular disease agent, an antiemetic agent, a hematopoietic colony stimulating factor, an anxiolytic agent, and an opioid or non-opioid analgesic agent.

In a further embodiment, the compound of formula Ia or additional pharmaceutically active agent can be administered prior to, concurrently with, or after an antiemetic agent, or on the same day, or within 1 hour, 2 hours, 12 hours, 24 hours, 48 hours or 72 hours of each other.

In another embodiment, the compound of formula Ia or additional pharmaceutically active agent described herein can be administered prior to, concurrently with, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In still another embodiment, the compound of formula Ia or additional pharmaceutically active agent described herein can be administered prior to, concurrently with, or after an opioid or non-opioid analgesic agent, or on the same day, or within 1 hour, 2 hours, 12 hours, 24 hours, 48 hours or 72 hours of each other.

In yet another embodiment, the compound of formula Ia or additional pharmaceutically active agent described herein can be administered prior to, concurrently with, or after an anxiolytic agent, or on the same day, or within 1 hour, 2 hours, 12 hours, 24 hours, 48 hours or 72 hours of each other.

Effective amounts of the other pharmaceutically active agents are well known to those skilled in the art. However, it is well within the skilled artisan's purview to determine the pharmaceutically active agent's optimal effective amount range. In one embodiment of the invention, where another pharmaceutically active agent is administered to a subject, the effective amount of the compound of formula Ia described herein is less than its effective amount would be where the other pharmaceutically active agent is not administered. In this case, without being bound by theory, it is believed that the compound of formula Ia described herein and the other pharmaceutically active agent act synergistically to treat or prevent cancer or hyperproliferative disease.

Antiemetic agents useful in the methods of the present invention include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, and tropisetron.

Hematopoietic colony stimulating factors useful in the methods of the present invention include, but are not limited to, filgrastim, sargramostim, molgramostim and epoietin alfa.

Opioid analgesic agents useful in the methods of the present invention include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

Non-opioid analgesic agents useful in the methods of the present invention include, but are not limited to, acetaminophen, acetaminophen plus codeine, aspirin, celecoxib, rofecoxib, diclofenac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

Anxiolytic agents useful in the methods of the present invention include, but are not limited to, buspirone, and benzodiazepines such as diazepam, lorazepam, oxazapam, chlorazepate, clonazepam, chlordiazepoxide and alprazolam.

### 1.16 Pharmaceutical Preparations

The compounds of formula Ia, especially those of sections 1.1-1.12, may form a part of a pharmaceutical composition by combining one or more such compounds with a pharmaceutically acceptable carrier. Additionally, the compositions may include an additive selected from the group consisting of adjuvants, excipients, diluents, and stabilizers.

### 1.17 Most Preferred Compounds

1-(3-tert-butyl-1-(1,2,3,4-tetrahydroisoquinolin-6-yl)-1H-pyrazol-5-yl)-3-(3-fluoro-4-(2-(methylcarbamoyl)pyridin-4-yloxy)phenyl)urea, 1-(3-tert-butyl-1-(quinolin-6-yl)-1H-pyrazol-5-yl)-3-(2-fluoro-4-(2-(methylcarbamoyl)pyridin-4-yloxy)phenyl)urea, 1-(3-tert-butyl-1-(1,2,3,4-tetrahydroisoquinolin-6-yl)-1H-pyrazol-5-yl)-3-(2-fluoro-4-(2-(methylcarbamoyl)pyridin-4-yloxy)phenyl)urea, 1-(3-tert-butyl-1-(quinolin-6-yl)-1H-pyrazol-5-yl)-3-(3-fluoro-4-(2-(methylcarbamoyl)pyridin-4-yloxy)phenyl)urea, 1-(3-tert-butyl-1-(2-(methylamino)quinolin-6-yl)-1H-pyrazol-5-yl)-3-(2-fluoro-4-(2-(methylcarbamoyl)pyridin-4-yloxy)phenyl)urea, 1-(1-(4-(2-amino-2-oxoethyl)naphthalen-2-yl)-3-tert-butyl-1H-pyrazol-5-yl)-3-(2-chloro-5-(5-fluoropyridin-3-yloxy)phenyl)urea, 1-(3-tert-butyl-1-(quinolin-6-yl)-1H-pyrazol-5-yl)-3-(2-fluoro-5-(pyridin-3-yloxy)phenyl)urea, 1-(3-tert-butyl-1-(1H-indazol-5-yl)-1H-pyrazol-5-yl)-3-(2-fluoro-4-(2-(methylcarbamoyl)pyridin-4-yloxy)phenyl)urea, 1-(3-tert-butyl-1-(quinolin-6-yl)-1H-pyrazol-5-yl)-3-(2,4-difluoro-5-(pyridin-3-yloxy)phenyl)urea, 1-(3-tert-butyl-1-(1,2,3,4-tetrahydroisoquinolin-6-yl)-1H-pyrazol-5-yl)-3-(2,4-difluoro-5-(pyridin-3-yloxy)phenyl)urea, 1-(3-tert-butyl-1-(quinolin-6-yl)-1H-pyrazol-5-yl)-3-(3-methyl-4-(2-(methylcarbamoyl)pyridin-4-yloxy)phenyl)urea, 1-(3-tert-butyl-1-(1H-indazol-5-yl)-1H-pyrazol-5-yl)-3-(2-fluoro-5-(pyridin-3-yloxy)phenyl)urea, 1-(5-tert-butyl-1-(quinolin-6-yl)-1H-pyrazol-3-yl)-3-(2-fluoro-4-(2-(methylcarbamoyl)pyridin-4-yloxy)phenyl)urea, 1-(3-tert-butyl-1-(quinolin-6-yl)-1H-pyrazol-5-yl)-3-(4-(2-carbamoylpyridin-4-yloxy)-2-fluorophenyl)urea, 1-(3-tert-butyl-1-(quinolin-6-yl)-1H-pyrazol-5-yl)-3-(2-fluoro-4-(2-(2-hydroxyethylamino)pyridin-4-yloxy)phenyl)urea, 1-(3-tert-butyl-1-(quinolin-6-yl)-1H-pyrazol-5-yl)-3-(4-chloro-5-(6-cyanopyridin-3-yloxy)-2-fluorophenyl)urea, 1-(3-tert-butyl-1-(quinolin-6-yl)-1H-pyrazol-5-yl)-3-(2-fluoro-4-(2-(methylamino)pyridin-4-yloxy)phenyl)urea, 1-(3-tert-butyl-1-(1H-indazol-5-yl)-1H-pyrazol-5-yl)-3-(5-(5-chloropyridin-3-yloxy)-2-fluorophenyl)urea, 1-(3-tert-butyl-1-(1,2,3,4-tetrahydroisoquinolin-6-yl)-1H-pyrazol-5-yl)-3-(5-(5-chloropyridin-3-yloxy)-2-fluorophenyl)urea, 1-(2-fluoro-4-(2-(methylcarbamoyl)pyridin-4-yloxy)phenyl)-3-(3-isopropyl-1-(quinolin-6-yl)-1H-pyrazol-5-yl)urea, 1-(3-ethyl-1-(quinolin-6-yl)-1H-pyrazol-5-yl)-3-(2-fluoro-4-(2-(methylcarbamoyl)pyridin-4-yloxy)phenyl)urea, 1-(2-fluoro-4-(2-(methylcarbamoyl)pyridin-4-yloxy)phenyl)-3-(1-(quinolin-6-yl)-3-(trifluoromethyl)-1H-pyrazol-5-yl)urea, 1-(3-cyclopentyl-1-(quinolin-6-yl)-1H-pyrazol-5-yl)-3-(2-fluoro-4-(2-(methylcarbamoyl)pyridin-4-yloxy)phenyl)urea, 1-(3-cyclobutyl-1-(quinolin-6-yl)-1H-pyrazol-5-yl)-3-(2-fluoro-4-(2-(methylcarbamoyl)pyridin-4-yloxy)phenyl)urea, 1-(3-tert-butyl-1-(quinolin-6-yl)-1H-pyrazol-5-yl)-3-(5-(6-cyanopyridin-3-yloxy)-2-fluorophenyl)urea, 1-(3-tert-butyl-1-(quinolin-6-yl)-1H-pyrazol-5-yl)-3-(3-fluoro-4-(2-(methylamino)pyridin-4-yloxy)phenyl)urea, 1-(3-tert-butyl-1-(quinolin-6-yl)-1H-pyrazol-5-yl)-3-(5-(5-chloropyridin-3-yloxy)-2-fluorophenyl)urea, 1-(3-tert-butyl-1-(quinolin-6-yl)-1H-pyrazol-5-yl)-3-(2-fluoro-5-(2-(methylthio)pyrimidin-4-yloxy)phenyl)urea, 1-(3-tert-butyl-1-(quinolin-6-yl)-1H-pyrazol-5-yl)-3-(2-fluoro-5-(6-(hydroxymethyl)pyridin-3-yloxy)phenyl)urea, 1-(3-tert-butyl-1-(1,2,3,4-tetrahydroisoquinolin-6-yl)-1H-pyrazol-5-yl)-3-(4-methyl-3-(pyridin-3-yloxy)phenyl)urea, 1-(4-(2-carbamoylpyridin-4-yloxy)-2-fluorophenyl)-3-(3-isopropyl-1-(quinolin-6-yl)-1H-pyrazol-5-yl)urea, 1-(3-tert-butyl-1-(quinolin-6-yl)-1H-pyrazol-5-yl)-3-(2-fluoro-5-(6-methylpyridin-3-yloxy)phenyl)urea, 1-(3-isopropyl-1-(quinolin-6-yl)-1H-pyrazol-5-yl)-3-(3-methyl-4-(2-(methylcarbamoyl)pyridin-4-yloxy)phenyl)urea, 1-(3-tert-butyl-1-(quinolin-6-yl)-1H-pyrazol-5-yl)-3-(2-fluoro-5-(2-(methylamino)pyrimidin-4-yloxy)phenyl)urea, 1-(2-fluoro-5-(6-methylpyridin-3-yloxy)phenyl)-3-(3-isopropyl-1-(quinolin-6-yl)-1H-pyrazol-5-yl)urea, 1-(2-fluoro-4-(2-(methylamino)pyridin-4-yloxy)phenyl)-3-(3-isopropyl-1-(quinolin-6-yl)-1H-pyrazol-5-yl)urea, 1-(3-ethyl-1-(quinolin-6-yl)-1H-pyrazol-5-yl)-3-(2-fluoro-4-(2-(methylamino)pyridin-4-yloxy)phenyl)urea, 1-(4-(2-carbamoylpyridin-4-yloxy)-2-fluorophenyl)-3-(3-ethyl-(quinolin-6-yl)-1H-pyrazol-5-yl)urea, 1-(3-tert-butyl-1-(quinolin-6-yl)-1H-pyrazol-5-yl)-3-(2-fluoro-5-(6-(methylcarbamoyl)pyridin-3-yloxy)phenyl)urea, 1-(3-tert-butyl-1-(1H-indazol-5-yl)-1H-pyrazol-5-yl)-3-(2-fluoro-5-(2-(methylamino)pyrimidin-4-yloxy)phenyl)urea, 1-(4-(2-carbamoylpyridin-4-yloxy)-2-fluorophenyl)-3-(5-chloro-2-(quinolin-6-yl)phenyl)urea, 1-(4-(2-carbamoylpyridin-4-yloxy)-2-fluorophenyl)-3-(4-chloro-2-(quinolin-6-yl)phenyl)urea, 1-(2-fluoro-4-(2-methoxypyridin-4-yloxy)phenyl)-3-(3-isopropyl-1-(quinolin-6-yl)-1H-pyrazol-5-yl)urea, 1-(1-(1H-indazol-5-yl)-3-isopropyl-1H-pyrazol-5-yl)-3-(4-(2-carbamoylpyridin-4-yloxy)-2-fluorophenyl)urea, 1-(3-tert-butyl-1-(2-methylquinolin-6-yl)-1H-pyrazol-5-yl)-3-(2-fluoro-4-(2-(methylcarbamoyl)pyridin-4-yloxy)phenyl)urea, 1-(2-fluoro-5-(6-(trifluoromethyl)pyridin-3-yloxy)phenyl)-3-(3-isopropyl-1-(quinolin-6-yl)-1H-pyrazol-5-yl)urea, 1-(4-(2-carbamimidoylpyridin-4-yloxy)-2-fluorophenyl)-3-(3-isopropyl-1-(quinolin-6-yl)-1H-pyrazol-5-yl)urea, 1-(3-tert-butyl-1-(1,2,3,4-tetrahydroisoquinolin-6-yl)-1H-pyrazol-5-yl)-3-(3-cyano-5-(pyridin-3-yloxy)phenyl)urea, 1-(5-(2-aminopyrimidin-4-yloxy)-2-fluorophenyl)-3-(3-tert-butyl-1-(quinolin-6-yl)-1H-pyrazol-5-yl)urea, 1-(3-tert-butyl-1-(2-methylquinolin-6-yl)-1H-pyrazol-5-yl)-3-(4-(2-carbamoylpyridin-4-yloxy)-2-fluorophenyl)urea, 1-(4-(2-carbamoylpyridin-4-yloxy)-3-methylphenyl)-3-(3-isopropyl-1-(quinolin-6-yl)-1H-pyrazol-5-yl)urea, 1-(4-(2-cyanopyridin-4-yloxy)-2-fluorophenyl)-3-(3-isopropyl-1-(quinolin-6-yl)-1H-pyrazol-5-yl)urea, 1-(2-fluoro-4-(2-(methylcarbamoyl)pyridin-4-yloxy)phenyl)-3-(3-isopropyl-1-(2-methylquinolin-6-yl)-1H-pyrazol-5-yl)urea, 1-(3-isopropyl-1-(quinolin-6-yl)-1H-pyrazol-5-yl)-3-(3-methyl-4-(2-(methylcarbamoyl)pyridin-4-yloxy)phenyl)urea, 1-(4-(2-carbamoylpyridin-4-yloxy)-2-fluorophenyl)-3-(3-isopropyl-1-(2-methylquinolin-6-yl)-1H-pyrazol-5-yl)urea, 1-(4-(2-(dimethylamino)pyridin-4-yloxy)-2-fluorophenyl)-3-(3-isopropyl-1-(quinolin-6-yl)-1H-pyrazol-5-yl)urea, 1-(3-tert-butyl-1-(quinolin-6-yl)-1H-pyrazol-5-yl)-3-(2-fluoro-4-(2-(hydroxymethyl)pyridin-4-yloxy)phenyl)urea, 1-(2-fluoro-4-(2-(isopropylamino)pyridin-4-yloxy)phenyl)-3-(3-isopropyl-1-(quinolin-6-yl)-1H-pyrazol-5-yl)urea, 1-(3-tert-butyl-1-(H-imidazo[1,2-a]pyridin-6-yl)-1H-pyrazol-5-yl)-3-(4-(2-carbamoylpyridin-4-yloxy)-2-fluorophenyl)urea, 1-(3-isopropyl-1-(quinolin-6-yl)-1H-pyrazol-5-yl)-3-(3-methyl-4-(2-(methylamino)pyridin-4-yloxy)phenyl)urea, 1-(3-tert-butyl-1-(quinolin-6-yl)-1H-pyrazol-5-yl)-3-(4-(2-carbamoylpyridin-4-yloxy)-3-methylphenyl)urea, 1-(5-(2-aminopyrimidin-4-yloxy)-2-fluorophenyl)-3-(3-isopropyl-1-(2-methylquinolin-6-yl)-1H-pyrazol-5-yl)urea, 1-(2-fluoro-4-(2-(methylamino)pyrimidin-4-yloxy)phenyl)-3-(3-isopropyl-1-(quinolin-6-yl)-1H-pyrazol-5-yl)urea, 1-(2-fluoro-5-(6-(methylcarbamoyl)pyridin-3-yloxy)phenyl)-3-(3-isopropyl-1-(quinolin-6-yl)-1H-pyrazol-5-yl)urea, 1-(2-fluoro-5-(6-(hydroxymethyl)pyridin-3-yloxy)phenyl)-3-(3-isopropyl-1-(quinolin-6-yl)-1H-pyrazol-5-yl)urea, 1-(3-tert-butyl-1-(quinoxalin-6-yl)-1H-pyrazol-5-yl)-3-(2-fluoro-4-(2-(methylcarbamoyl)pyridin-4-yloxy)phenyl)urea, 1-(4-(2-(1H-pyrazol-4-yl)pyridin-4-yloxy)-2-fluorophenyl)-3-(3-isopropyl-1-(quinolin-6-yl)-1H-pyrazol-5-yl)urea, 1-(1-(benzo[d]thiazol-6-yl)-3-isopropyl-1H-pyrazol-5-yl)-3-(2-fluoro-4-(2-(methylcarbamoyl)pyridin-4-yloxy)phenyl)urea, 1-(2-fluoro-4-(2-(methylcarbamoyl)pyridin-4-yloxy)phenyl)-3-(3-isopropyl-1-(1-methyl-1H-benzo[d]imidazol-5-yl)-1H-pyrazol-5-yl)urea, 1-(3-isopropyl-1-(quinolin-6-yl)-1H-pyrazol-5-yl)-3-(2-methyl-4-(2-(methylcarbamoyl)pyridin-4-yloxy)phenyl)urea, 1-(1-(H-imidazo[1,2-a]pyridin-6-yl)-3-isopropyl-1H-pyrazol-5-yl)-3-(2-fluoro-4-(2-(methylcarbamoyl)pyridin-4-yloxy)phenyl)urea, 1-(3-tert-butyl-1-(2-methylquinolin-6-yl)-1H-pyrazol-5-yl)-3-(2-fluoro-4-(2-(methylamino)pyridin-4-yloxy)phenyl)urea, 1-(1-(H-imidazo[1,2-a]pyridin-6-yl)-3-isopropyl-1H-pyrazol-5-yl)-3-(4-(2-carbamoylpyridin-4-yloxy)-2-fluorophenyl)urea, 1-(4-(2-acetamidopyridin-4-yloxy)-2-fluorophenyl)-3-(3-isopropyl-1-(quinolin-6-yl)-1H-pyrazol-5-yl)urea, 1-(4-(2-(ethylamino)pyridin-4-yloxy)-2-fluorophenyl)-3-(3-isopropyl-1-(quinolin-6-yl)-1H-pyrazol-5-yl)urea, 1-(4-(2-(1H-pyrazol-4-yl)pyridin-4-yloxy)-2-fluorophenyl)-3-(3-methyl-1-(quinolin-6-yl)-1H-pyrazol-5-yl)urea, 1-(3-isopropyl-1-(quinolin-6-yl)-1H-pyrazol-5-yl)-3-(3-methyl-4-(2-(1-methyl-1H-pyrazol-4-yl)pyridin-4-yloxy)phenyl)urea, 1-(2-fluoro-4-(2-(methylcarbamoyl)pyridin-4-yloxy)phenyl)-3-(3-isopropyl-1-(quinoxalin-6-yl)-1H-pyrazol-5-yl)urea, 1-(2-fluoro-4-(2-(1-methyl-1H-pyrazol-4-yl)pyridin-4-yloxy)phenyl)-3-(3-isopropyl-1-(quinolin-6-yl)-1H-pyrazol-5-yl)urea, 1-(4-(2-carbamoylpyridin-4-yloxy)-2-fluorophenyl)-3-(3-isopropyl-1-(quinoxalin-6-yl)-1H-pyrazol-5-yl)urea, 1-(3-tert-butyl-1-(quinolin-6-yl)-1H-pyrazol-5-yl)-3-(2-fluoro-4-(2-(isopropylamino)pyridin-4-yloxy)phenyl)urea, 1-(3-ethyl-1-(quinolin-6-yl)-1H-pyrazol-5-yl)-3-(2-fluoro-4-(2-(1-methyl-1H-pyrazol-4-yl)pyridin-4-yloxy)phenyl)urea, 1-(1-(benzo[d]oxazol-5-yl)-3-isopropyl-1H-pyrazol-5-yl)-3-(2-fluoro-4-(2-(methylcarbamoyl)pyridin-4-yloxy)phenyl)urea, 1-(1-(benzo[d]oxazol-5-yl)-3-tert-butyl-1H-pyrazol-5-yl)-3-(2-fluoro-4-(2-(methylcarbamoyl)pyridin-4-yloxy)phenyl)urea, 1-(1-(3-aminobenzo[d]isoxazol-5-yl)-3-tert-butyl-1H-pyrazol-5-yl)-3-(2-fluoro-4-(2-(methylcarbamoyl)pyridin-4-yloxy)phenyl)urea, 1-(5-(6-acetamidopyridin-3-yloxy)-2-fluorophenyl)-3-(3-isopropyl-1-(quinolin-6-yl)-1H-pyrazol-5-yl)urea, 1-(4-(2-(1H-pyrazol-4-yl)pyridin-4-yloxy)-3-methylphenyl)-3-(3-isopropyl-1-(quinolin-6-yl)-1H-pyrazol-5-yl)urea, 1-(3-tert-butyl-1-(1,2,3,4-tetrahydroisoquinolin-6-yl)-1H-pyrazol-5-yl)-3-(2-fluoro-4-(2-(1-methyl-1H-pyrazol-4-yl)pyridin-4-yloxy)phenyl)urea, 1-(4-(2-(1H-pyrazol-4-yl)pyridin-4-yloxy)-2-fluorophenyl)-3-(3-tert-butyl-1-(1,2,3,4-tetrahydroisoquinolin-6-yl)-1H-pyrazol-5-yl)urea, 1-(4-(2-(1H-pyrazol-4-yl)pyridin-4-yloxy)-2-fluorophenyl)-3-(3-tert-butyl-1-(1-oxo-1,2,3,4-tetrahydroisoquinolin-7-yl)-1H-pyrazol-5-yl)urea, 1-(4-(2-(1H-pyrazol-4-yl)pyridin-4-yloxy)-2-fluorophenyl)-3-(3-tert-butyl-1-(1,2,3,4-tetrahydroisoquinolin-7-yl)-1H-pyrazol-5-yl)urea, 1-(3-fluoro-4-(2-(isopropylamino)pyridin-4-yloxy)phenyl)-3-(3-isopropyl-1-(quinolin-6-yl)-1H-pyrazol-5-yl)urea, 1-(3-isopropyl-1-(quinolin-6-yl)-1H-pyrazol-5-yl)-3-(4-(2-(isopropylamino)pyridin-4-yloxy)-3-methylphenyl)urea, 1-(4-(2-(cyclopentylamino)pyridin-4-yloxy)-2-fluorophenyl)-3-(3-isopropyl-1-(quinolin-6-yl)-1H-pyrazol-5-yl)urea, 1-(2-fluoro-4-(2-(1-methyl-1H-pyrazol-4-yl)pyridin-4-yloxy)phenyl)-3-(3-methyl-1-(quinolin-6-yl)-1H-pyrazol-5-yl)urea, 1-(3-ethyl-1-(quinolin-6-yl)-1H-pyrazol-5-yl)-3-(2-fluoro-3-methyl-4-(2-(1-methyl-1H-pyrazol-4-yl)pyridin-4-yloxy)phenyl)urea, 1-(2,3-difluoro-4-(2-(1-methyl-1H-pyrazol-4-yl)pyridin-4-yloxy)phenyl)-3-(3-ethyl-1-(quinolin-6-yl)-1H-pyrazol-5-yl)urea, 1-(3-tert-butyl-1-(quinolin-6-yl)-1H-pyrazol-5-yl)-3-(3-(5-chloropyridin-3-yloxy)-5-cyanophenyl)urea, 1-(3-tert-butyl-1-(quinolin-6-yl)-1H-pyrazol-5-yl)-3-(3-cyano-5-(6-methylpyridin-3-yloxy)phenyl)urea, 1-(5-(4-(1H-pyrazol-4-yl)pyrimidin-2-yloxy)-2-fluorophenyl)-3-(3-isopropyl-(quinolin-6-yl)-1H-pyrazol-5-yl)urea, 1-(2-fluoro-4-((2-(1-methyl-1H-pyrazol-4-yl)pyridin-4-yl)oxy)phenyl)-3-(1-isopropyl-3-(quinolin-6-yl)-1H-pyrazol-4-yl)urea, 4-(3-fluoro-4-(3-(1-isopropyl-3-(quinolin-6-yl)-1H-pyrazol-4-yl)ureido)phenoxy)-N-methylpicolinamide, 1-(4-((2-(ethylamino)pyridin-4-yl)oxy)-2,3-difluorophenyl)-3-(3-isopropyl-1-(quinolin-6-yl)-1H-pyrazol-5-yl)urea, N-(4-(2-fluoro-4-(3-(3-isopropyl-1-(quinolin-6-yl)-1H-pyrazol-5-yl)ureido)phenoxy)pyridin-2-yl)acetamide, 4-(4-(3-(1-(4-(aminomethyl)naphthalen-2-yl)-3-(tert-butyl)-1H-pyrazol-5-yl)ureido)-3-fluorophenoxy)-N-methylpicolinamide, and 4-(3-fluoro-4-(3-(3-(1-hydroxy-2-methylpropan-2-yl)-1-(quinolin-6-yl)-1H-pyrazol-5-yl)ureido)phenoxy)-N-methylpicolinamide.

### 2. Synthesis of Compounds of the Present Invention

The compounds of the invention are available, for example, by the procedures and teachings of WO 2006/071940, filed Dec. 23, 2005, incorporated by reference, and by the general synthetic methods illustrated in the schemes below and the accompanying examples.

As indicated in Scheme 1, ureas of general formula 1 can be readily prepared by the union of amines of general formula 2 with isocyanates 3 or isocyanate surrogates 4 (trichloroethyl carbamates) or 5 (isopropenyl carbamates). Preferred conditions for the preparation of compounds of general formula 1 involve heating a solution of 4 or 5 with 2 in the presence of a tertiary base such as diisopropylethylamine, triethylamine or N-methylpyrrolidine in a solvent such as dimethylformamide, dimethylsulfoxide, tetrahydrofuran or 1,4-dioxane at a temperature between 50 and 100° C. for a period of time ranging from 1 hour to 2 days.

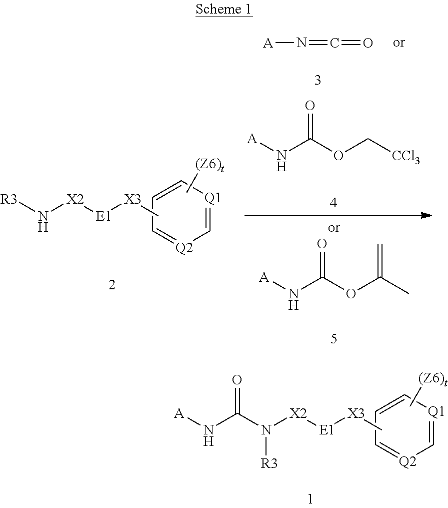

As shown in Scheme 2, isocyanates 3 can be prepared from amines A-NH₂ 6 with phosgene, or a phosgene equivalent such as diphosgene, triphosgene, or N,N-dicarbonylimidazole. Trichloroethyl carbamates 4 and isopropenyl carbamates 5 are readily prepared from amines A-NH₂ (6) by acylation with trichloroethyl chloroformate or isopropenyl chloroformate by standard conditions familiar to those skilled in the art. Preferred conditions for the preparation of 4 and 5 include include treatment of compound 6 with the appropriate chloroformate in the presence of pyridine in an aprotic solvent such as dichloromethane or in the presence of aqueous hydroxide or carbonate in a biphasic aqueous/ethyl acetate solvent system.

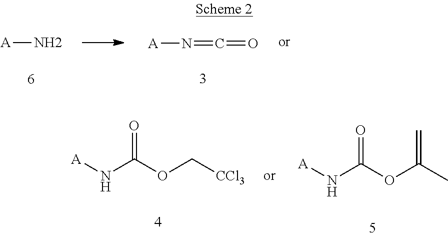

Additionally, compounds of formula 1 can also be prepared from carboxylic acids 7 by the intermediacy of in-situ generated acyl azides (Curtius rearrangement) as indicated in Scheme 3. Preferred conditions for Scheme 3 include the mixing of acid 7 with amine 2 and diphenylphosphoryl azide in a solvent such as 1,4-dioxane or dimethylformamide in the presence of base, such as triethylamine, and raising the temperature of the reaction to about 80-120° C. to affect the Curtius rearrangement.

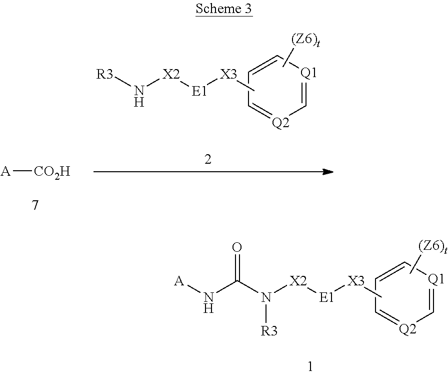

Many methods exist for the preparation of amines A-NH₂ 6 and acids A-CO₂ H 7, depending on the nature of the A-moiety. Many such methods have been described in detail in WO 2006/071940, and are incorporated by reference here. Preferred synthetic methods are outlined in the following schemes for the non-limiting examples wherein A is a 1-substituted-pyrazole (optionally substituted by R2) or A and A1 are linked by C—C bond.

As illustrated in Scheme 4, A1-substituted, pyrazole amines 10 (a preferred aspect of A-NH₂ 6, Scheme 2) are available by the condensation of hydrazines 8 and beta-keto nitriles 9. Preferred conditions for this transformation are by heating in ethanolic HCl. Hydrazines 8 are in turn available by the diazotization of amines 11 followed by reduction or, alternately from the hydrolysis of hydrazones 13 obtained by the palladium mediated coupling of benzophenone hydrazone with compounds of formula A1-X 12, wherein X represents a halogen or triflate moiety.

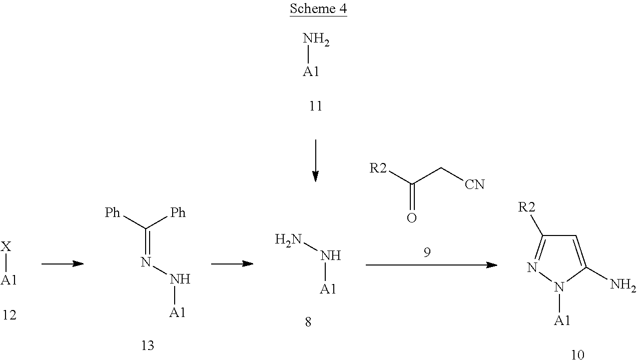

A non-limiting example of Scheme 4 is illustrated by the preparation of compound 19 (Scheme 5 and the accompanying examples). Thus commercially available 6-hydroxyquinoline 14 can be converted to trifluoromethanesulfonate 15 by treatment with triflic anhydride and pyridine. Reaction of 15 with benzophenone hydrazone in the presence of a palladium catalyst, preferably a catalyst containing the bis(diphenylphosphino)ferrocene ligand, provides the hydrazone 16. Reaction of 16 with ethanolic HCl at reflux provides the hydrazine 17, which can be combined with keto nitriles of general formula 18 by further heating in ethanolic HCl to provide quinoline pyrazole amines of formula 19. In another aspect of this synthetic sequence, hydrazone 16 can be converted directly to pyrazole 19 by the direct reaction with keto nitrile 18 upon heating in ethanolic HCl.

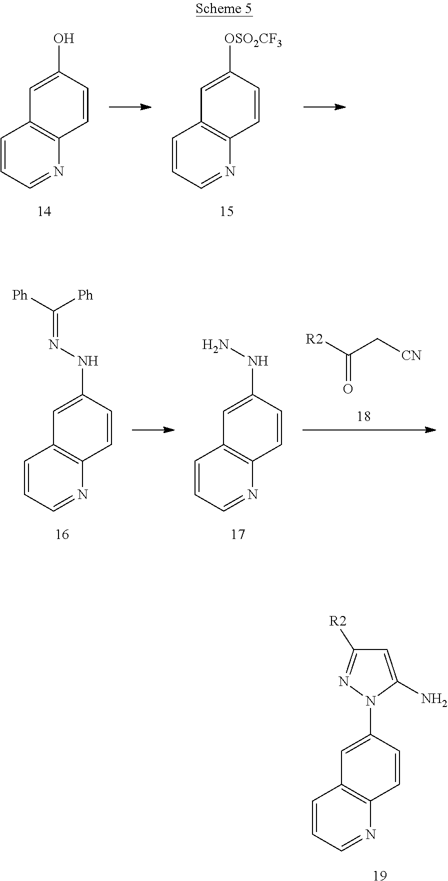

Another preferred method for constructing A1-substituted pyrazoles is illustrated by the general preparation of pyrazole acid 22 (Scheme 6), an aspect of A-CO₂ H 7 (Scheme 3). As indicated in Scheme 6, the union of a pyrazole 5-carboxylic ester 20 with A1-X 12, wherein X represents a halide, triflate, or boronic acid suitable for direct transition metal-catalyzed couplings with pyrazoles 20, provides A1-substituted pyrazole esters 21. Preferred conditions for such transformations involve mixing a boronic acid 11 [X═B(OH)₂] and esters 20 in dichloromethane with copper acetate and pyridine in the presence of crushed molecular sieves, with or without heating. Preferred esters for this transformation include ethyl, tert-butyl and benzyl esters. The esters 21 in turn can be converted to acids 22 by standard conditions familiar to those skilled in the art, such as saponification, acidic hydrolysis or hydrogenation.

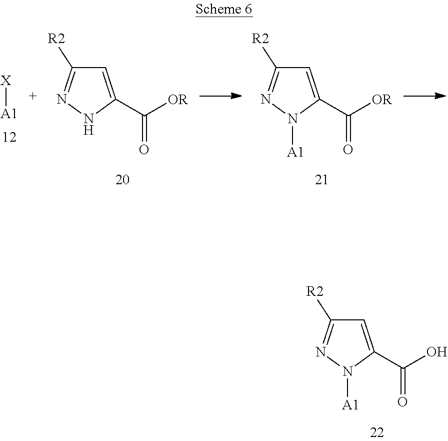

The synthesis of intermediates useful for the construction of compounds of formula 1 wherein A and A1 are linked by a C—C bond is shown in Scheme 7. In this case, palladium catalyzed reactions (for example, Suzuki or Stille reactions) of A1-X 12 with a complementary component 23 or 24 provides compounds 25 or 26, examples of general intermediates A-NH₂ 6 or A-CO₂H 7, respectively. In this synthetic sequence, the X— groups on the reactants 12 and 23 or 24 are moieties that undergo transition metal catalyzed cross coupling reactions, such as halides or triflates and boronic acids or esters, stannanes, silanes, organozincs or other organometallic moieties known by those skilled in the art to be suitable substrates for such processes. The X-groups in Scheme 7 are complementary moieties for cross coupling processes such that when A1-X 12 is a halide or triflate, A-X 23 or A-X 24 will be a complementary organometallic, such as a stannane or the like or a boronic acid or ester. Likewise, if A1-X 12 is an organometallic reagent or a boronic acid or ester, A-X will be a halide or triflate.

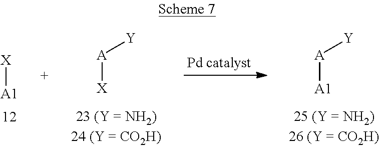

Within Scheme 7, it will be understood by those skilled in the art that there are additional synthetic equivalents for the Y-groups of 23 and 24 that can be used interchangeably with NH₂ and CO₂H with the addition of additional transforming steps. For example, the Y group of 23 might also be a protected amino group such as N-Boc or a surrogate amino group such as nitro that would give rise to compounds of formula 25 after acidic hydrolysis or reduction respectively. Similarly, it will be recognized that the Y group of 24 might also be an ester or nitrile which could be hydrolyzed to an acid of formula 26 by standard synthetic methods.

A non limiting example of Scheme 7 is illustrated by the preparation of compound 29, an example of general intermediate A-NH₂ 6, above. Thus, commercially available quinoline 6-boronic acid 27 can be combined with commercially available 5-fluoro-2-iodoaniline 28 in the presence of a palladium catalyst to provide compound 29, an example of general intermediate A-NH₂ 6, above.

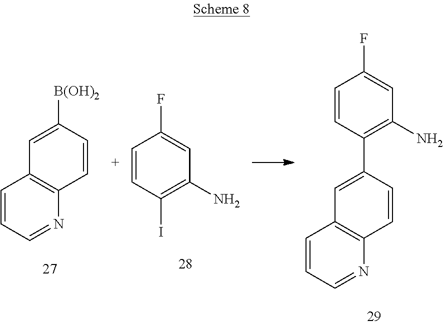

Amines 2 (Schemes 1 and 3, above) useful for the invention can be synthesized according to methods commonly known to those skilled in the art. Non-limiting examples are illustrated in the following schemes. A general preparation of aryl amine 32, an example of amine 2, above, is shown in Scheme 9. Thus, chloropyridines of formula 31 are reacted with phenols of formula 30 in the presence of base such as potassium tert-butoxide. Reactions are generally conducted at temperatures between 0° C. and 150° C. in solvents such as dimethylacetamide, dimethylformamide or dimethylsulfoxide. Some non-limiting examples of general synthetic Scheme 9 are shown in Schemes 10-12, below.

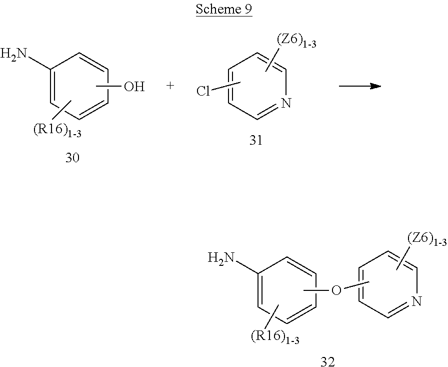

In Scheme 10, commercially available 3-fluoro-4-aminophenol is reacted with potassium tert-butoxide and chloropyridines 34 or 35 to provide amino ethers 36 and 37 respectively. The preferred solvent for this transformation is dimethylacetamide at a temperature between 80 and 100° C.

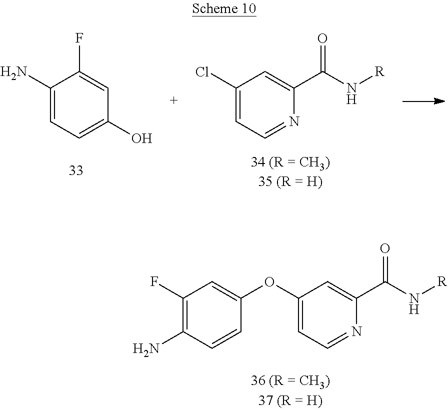

In a similar manner, commercially available 2-methyl-4-aminophenol 38 is combined with chloropyridines 34 and 35 to provide amino ethers 39 and 40, respectively (Scheme 11).

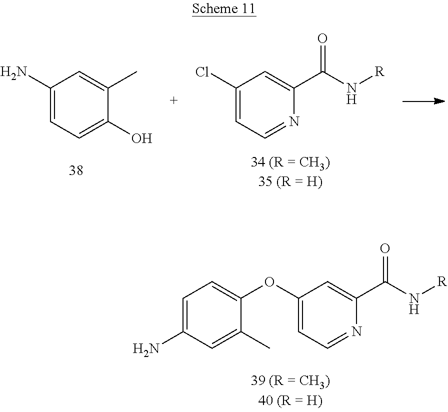

Scheme 12 illustrates the preparation of meta-substituted pyridyl ether amines 47 and 48, examples of general intermediate 2, above. As shown in Scheme 12, commercially available 2-chloro-4-fluorophenol 41 is treated with methyl chloroformate to provide carbonate 42. Nitration under standard conditions then provides adduct 43. Hydrolysis of the carbonate provides phenol 44. Concomitant reduction of both the nitro and chloro moieties provides aminophenol 45. Treatment of phenol 45 sequentially with potassium tert-butoxide and 3,5-dichloropyridine and heating in dimethylacetamide provides the compound 47. Removal of the chlorine atom of 47 by hydrogenation provides the amine of formula 48, an aspect of general amine 2.

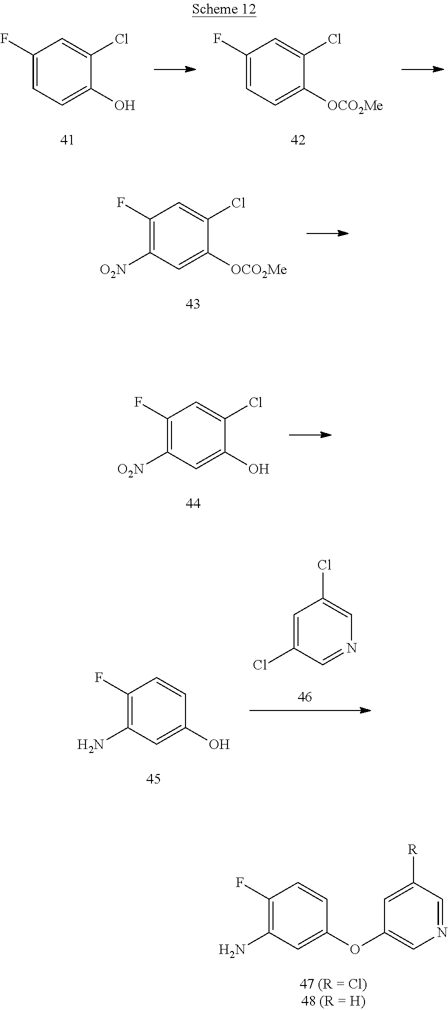

Amines of general formula 2 can also be prepared by the general route shown in Scheme 13. Thus, halo pyridine 49 (X is halogen) or halo pyrimidine 50 (X is halogen) can be converted to Z6-substituted pyridine 51 or Z6-substituted pyrimidine 52, respectively. There are several methods through which this can be accomplished, depending on the nature of the Z6. When the Z6 moiety is attached to the Q-containing ring through a Z6 nitrogen atom, preferred methods include heating compounds of formula 49 or 50 with an excess of the amine Z6-H either neat or in a solvent such as N-methylpyrrolidinone, DMF, DMSO or an alcoholic solvent at temperatures ranging from RT to 200° C. For the case of aryl and heteroaryl amines Z6-H, additional preferred methods include the heating of compounds 49 or 50 with an excess of the amine Z6-H and an acid catalyst (for example, TsOH, HCl, HOAc or the like) in a suitable solvent such as DMF, DMSO or an alcoholic solvent. Additional preferred methods for aryl and heteroarylamines Z6-H include combining Z6-H with compounds 49 or 50 in the presence of a transition metal catalyst such as a palladium catalyst in a suitable solvent like 1,4-dioxane or DMF with heating if necessary. When the Z6 moiety is attached to the Q-containing ring through a Z6 oxygen or sulfur atom, preferred methods include heating 49-50 with alcohol or thiol Z6-H in the presence of a strong base (for example, NaH or potassium tert-butoxide) either neat using Z6-H as the solvent, or in a polar solvent such as DMF or DMSO at temperatures ranging from RT to 200° C. When the Z6 moiety is attached to the Q-containing ring through a Z6 carbon atom, preferred methods include contacting compounds 49 or 50 with a species of formula Z6-M in the presence of a palladium catalyst, wherein M is a species that participates in transition-metal catalyzed cross-coupling reactions. Examples of suitable M groups include but are not limited to, boronic acids, boronic esters, zinc, trialkyltin, silicon, magnesium, lithium, and aluminum. Optionally, the transformations shown in Scheme 13 may be performed with microwave heating. It will be understood by those skilled in the art that the Z6 moieties introduced in Scheme 13 may contain optional protecting groups that will be removed in subsequent transformations (not shown). Some non-limiting examples of general Scheme 13 are shown in Schemes 14 and 15, below.

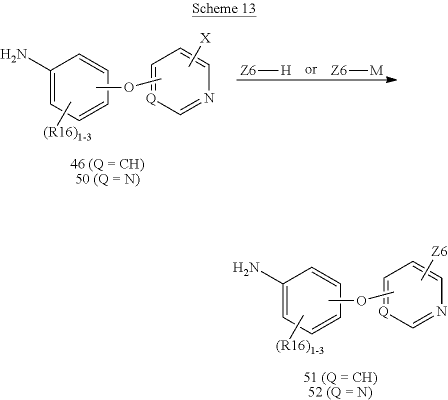

In Scheme 14, phenol 33 and 2,4-dichloropyridine (51) are combined using general Scheme 9 to provide the chloropyridine 52. Further reaction of chloropyridine 52 with the N-methylpyrazole boronate 53 in the presence of palladium tetrakis(triphenylphosphine) provides 54, an example of general amine 2.

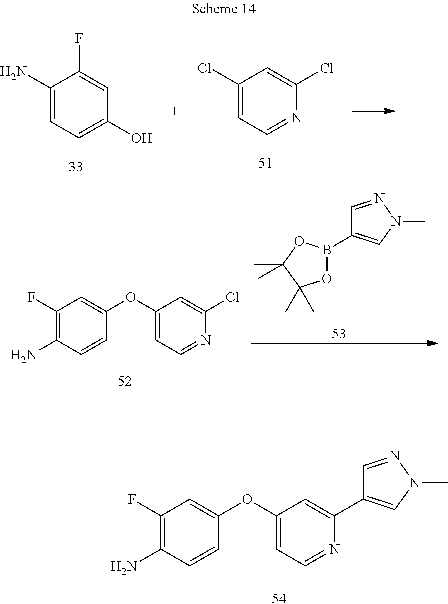

Scheme 15, shows the preparation of amino pyridine 55 from chloropyridine 52 by the general route of Scheme 13. Preferred conditions for this transformation include the contacting of chloropyridine 52 with isopropylamine in N-methylpyrrolidinone with microwave heating.

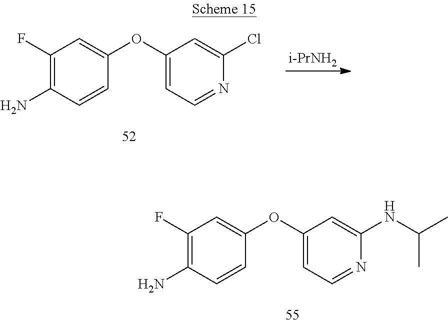

Scheme 16 illustrates an alternative preparation of compounds of general formula 1, represented by the preparation of urea 61. In the instance when general amine 2 is primary (R3=H), amine 2 can be converted to an isopropenyl carbamate 56, trichloroethyl carbamate 57, or 4-nitrophenyl carbamate 58 by reaction with isopropenyl chloroformate, trichloroethyl chloroformate or 4-nitrophenyl chloroformate, respectively. Alternatively, by analogy to Scheme 2, amine 2 (R3=H) can be converted to a discrete isocyanate 59. By analogy to Scheme 1, reaction of carbamates 56-58 or isocyanate 59 with R3-substituted amine 60 provides urea 61, an example of general formula 1.

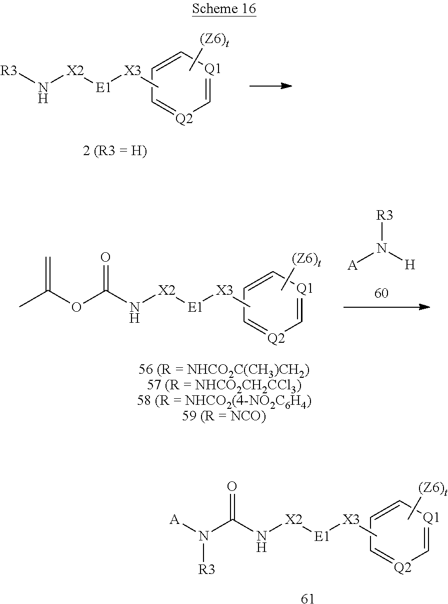

An additional subset of ureas of general formula 1 can be prepared as illustrated in Scheme 17. In the instances when R3 is not H, the mono-substituted ureas 1 or 61 can be optionally further transformed into bis-R3-substituted ureas 62 (Formula 1). Thus, in Scheme 17, exposure of 1 or 61 to alkyl halides or cycloalkyl halides in the presence of a base, for example potassium carbonate, sodium hydride or potassium tert-butoxide in a suitable solvent such as DMF provides ureas 62 wherein the newly incorporated R3 group is alkyl or cycloalkyl. Alternatively, exposure of ureas 1 or 61 to copper(II) acetate and Z3-substituted phenylboronic acids [See: Chan et. al, Tetrahedron Lett. 2003, 44, 3863-3865; Chan et. al, Tetrahedron Lett. 1998, 39, 2933-2936; Chan, D. M. T. Tetrahedron Lett. 1996, 37, 9013-9016] provides the analogous bis-R3-substituted ureas wherein the newly incorporated R3 is Z3-substituted phenyl.

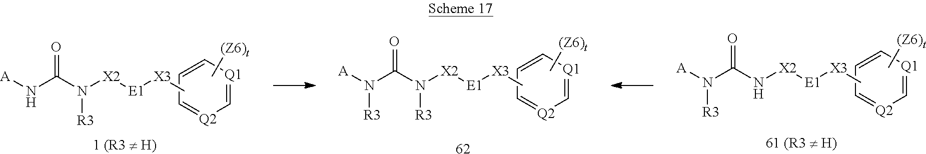

General amines A-NH₂ (6) wherein the A-ring is isoxazole can be prepared by the methods described in Scheme 18. Many examples of R2-substituted aminoisoxazoles 64 and 65 are commercially available. They can also be prepared from common keto nitrile intermediates 63 by condensation with hydroxylamine either under acidic or alkaline conditions as described in the literature (Takase, et al. Heterocycles, (1991), 32, pp 1153-1158). Bromination of isoxazoles 64 or 65 using standard conditions (see: Sircar, et. al. J. Org. Chem. (1985), 50, pp 5723-7; Carr, et. al. J. Med. Chem. (1977), 20, pp 934-9; Chan et al., U.S. Pat. No. 5,514,691) provides bromo isoxazoles 66 and 67 respectively. By analogy to Schemes 7 and 8, 66 and 67 can be converted to A1-containing amino isoxazoles 68 and 69, examples of general amine 6 and 25, through palladium-mediated couplings with reagents of formula A1-M (70), wherein the “M” moiety of A1-M is a moiety that participates in transition metal catalyzed cross coupling reactions, such as a boronic acid or ester, stannane, silane, organozinc or other organometallic moiety known by those skilled in the art to be a suitable substrate for such processes. Using the general methods of Schemes 1 and 2, amines 68 and 69 can be converted to ureas of general formula 1. It will be understood by those skilled in the art that the A1-moiety of 68-70 may contain protecting groups that may be removed prior to or after conversion to ureas of formula 1 by appropriate de-protection conditions. It will be further understood that the amino group of 64-69 may be optionally protected with a suitable protecting group (such as a tert-butylcarbamate) if desired to facilitate the bromination or palladium coupling steps.

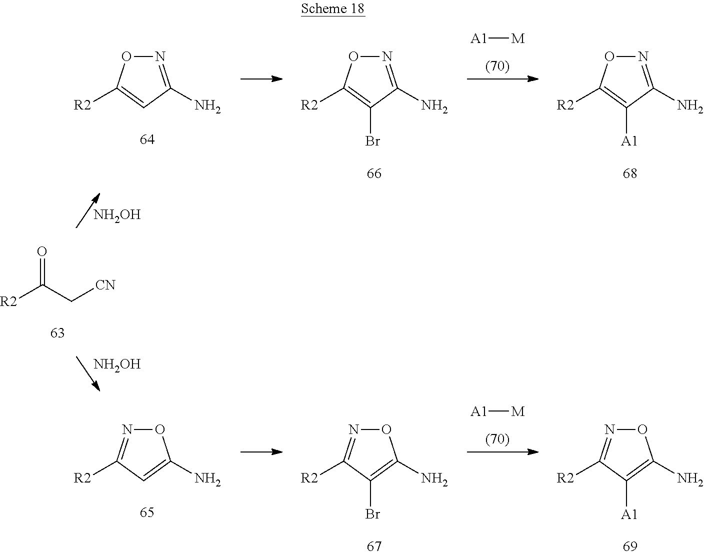

By analogy to Scheme 18, amines 73 and 74, examples of general amines A-NH₂ (6) wherein the A-ring is isothiazole, can be prepared as shown in Scheme 19 by the reaction of bromo isothiazoles 71 and 72 and A1-M (70). The requisite isothiazoles 71 and 72 are accessible by methods described in the literature (See; Vidyadher, H. B., WO 94/21647 (1994); Hackler, et. al. J. Heterocyclic Chem. (1989), 26, pp 1575-8). Using the general methods of Schemes 1 and 2, amines 73 and 74 can be converted to ureas of general formula 1.

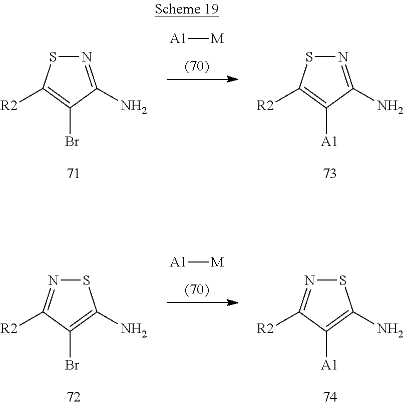

### 2.1 EXAMPLES

General Method A:

To a stirring solution of carboxylic acid (0.50 mmol, 1.00 eq) and DPPA (0.75 mmol, 1.50 eq) in 1,4-dioxane (5.0 ml) at RT was added Et₃N (1.5 mmol, 3.00 eq). After stirring for 30 min at RT, the appropriate amine (0.76 mmol, 1.50 eq) in dioxane was added and the mixture was heated at 95-100° C. After 2 h, the completed reaction was cooled to RT, diluted with brine and extracted with EtOAc (2×). The combined organics were washed with 3M HCl (1×), satd. NaHCO₃ (2×), and brine (1×), dried (MgSO₄), filtered and evaporated to give the crude product which was purified by flash column chromatography to afford the target urea.

### Example A1

4-Amino-2-fluorophenol (1.13 g, 8.9 mmol) and Example A22 (1.5 g, 8.9 mmol) were combined by the procedure of Example A2 to provide 4-(4-amino-2-fluorophenoxy)-N-methylpicolinamide (300 mg, 13% yield). ¹H-NMR (DMSO-d6) δ 8.78 (d, J=4.8 Hz, 1H), 8.47 (d, J=5.4 Hz, 1H), 7.32 (d, J=2.4 Hz, 1H), 7.11 (m, 1H), 7.01 (t, J=9.0 Hz, 1H), 6.51 (dd, J=13.2, 2.4 Hz, 1H), 6.42 (dd, J=8.4, 1.6 Hz, 1H), 5.51 (br s, 2H), 2.76 (d, J=4.8 Hz, 3H); MS (ESI) m/z: 262.1 (M+H⁺).

### Example A2

A solution of 4-amino-3-fluorophenol (2.00 g, 15.7 mmol) in anhydrous DMA (32 mL) was degassed by evacuation of the head space and backfilling with argon (repeated 3×). The solution was treated with potassium tert-butoxide (2.12 g, 18.9 mmol) and the resultant mixture was sonicated briefly to bring all solids into the solvent volume and was stirred at RT for 30 min. Example A22 (2.68 g, 15.7 mmol) was added. The reaction mixture was degassed a second time and the reaction mixture was heated to 100° C. overnight under argon. The reaction mixture was poured into ethyl acetate (400 mL) and washed with water (3×100 mL) and saturated brine (2×100 mL). The combined aqueous was extracted with EtOAc (100 mL). The combined organics were dried (MgSO₄), concentrated in vacuo to a brown oil and purified by silica gel chromatography to provide 4-(4-amino-3-fluorophenoxy)-N-methylpicolinamide (3.18 g, 77% yield). ¹H NMR (400 MHz, DMSO-d₆) δ 8.76 (m, 1H), 8.48 (d, J=5.7 Hz, 1H), 7.36 (d, J=2.6 Hz, 1H), 7.10 (dd, J=5.7, 2.6 Hz, 1H), 7.02 (dd, J=11.8, 2.6 Hz, 1H), 6.86 (t, J=9.8 Hz, 1H), 6.79 (dd, J=8.9, 2.5 Hz, 1H), 5.23 (s, 2H), 2.79 (d, J=4.9 Hz, 3H); MS (ESI) m/z: 262.0 (M+H⁺).

### Example A3

In NMP (15 mL) was placed 3-amino-4-chlorophenol (1.70 g, 11.8 mmol) and potassium t-butoxide (1.40 g, 12.4 mmol) and the mixture was stirred overnight at RT. The dark solution was treated with the 3,5-difluoropyridine (2.73 g, 23.7 mmol) and powdered potassium carbonate (818 mg, 5.92 mmol) and the mixture was then warmed to 80° C. and stirred for 24 h. The resulting black mixture was cooled to RT, diluted with brine (100 mL) and extracted with ethyl acetate (3×50 mL). The combined ethyl acetate extracts were washed with saturated sodium bicarbonate (50 mL), water (50 mL) and brine (50 mL), dried (Na₂SO₄), concentrated in vacuo and purified via column chromatography to yield 2-chloro-5-(5-fluoropyridin-3-yloxy)benzenamine as a thick oil which was used without further purification. ¹H-NMR (DMSO-d₆): δ 5.57 (br s, 2H), 6.26-6.30 (dd, 1H), 6.50 (s, 1H), 7.19-7.22 (m, 1H), 7.45-7.50 (m, 1H), 8.26 (s, 1H), 8.39 (s, 1H). MS (ESI) m/z: 239.0 (M+H⁺).

### Example A4

A mixture of Example A10 (4.6 g, 19.3 mmol) and 10% Pd(OH)₂/C (0.5 g, 0.35 mmol) in EtOH (50 mL) was stirred under a H₂ atmosphere at RT for 3 h. The mixture was filtered through Celite® and washed with EtOH. The filtrate was concentrated to give 2-fluoro-5-(pyridine-3-yloxy) aniline (3.5 g, 88% yield). ¹H NMR (300 MHz, DMSO-d₆) δ 8.53 (d, J=2.4 Hz, 1H), 8.48 (d, J=3.9 Hz, 1H), 7.80-7.69 (m, 2H), 7.05 (dd, J=11.1, 8.7 Hz, 1H), 6.53 (dd, J=7.5, 3.0 Hz, 1 H), 6.28 (dt, J=8.7, 3.3 Hz, 1H); MS (ESI) m/z: 205.3 (M+H⁺).

### Example A5

To a solution of 2,4-difluorophenol (2 g, 15.4 mmol) in CH₂Cl₂ (20 mL) was added triethyl amine (3.21 ml, 23 mmol) and ethyl chloroformate (1.77 ml, 18.4 mmol) at 0° C. After stirring the mixture for 1 h at RT, sat. NaHCO₃ solution (30 mL) was added, the organic layer was separated and the aqueous layer was extracted with CH₂Cl₂ (1×25 ml). The combined organic layers were washed with brine, dried (Na₂SO₄) and concentrated to afford 2,4-difluorophenyl ethyl carbonate (3.11 g, 100% yield) as a liquid.

To a solution of 2,4-difluorophenyl ethyl carbonate (3.1 g, 16 mmol) in sulphuric acid (10 mL) was added fuming HNO₃ (0.78 ml, 19 mmol) slowly, keeping the internal temperature around 0° C. After 15 min ice cold water (70 mL) was added, the product was extracted with ethyl acetate (2×50 mL), the combined organic layers were washed with brine, dried (Na₂SO₄) and concentrated to afford the nitro product as a thick syrup. This nitro product was dissolved in methanol (20 mL) and to this solution was added solid NaHCO₃ (4.0 g, 47 mmol) and the resultant mixture was stirred for 16 h at RT. The mixture was filtered and the filtrate was concentrated. The resulting solid was dissolved in water (20 ml) and acidified with 3M HCl solution to pH˜5. The product was extracted with CH₂Cl₂ (3×25 mL), the combined organic layers were washed with brine, dried (Na₂SO₄) and concentrated to afford 2,4-difluoro-5-nitrophenol (2.34 g, 84% yield). ¹H NMR (400 MHz, Acetone-d₆) δ 9.59 (s, 1H), 7.78 (t, J=7.2 Hz, 1H), 7.45 (t, J=10.4 Hz, 1H); MS (ESI) m/z: 176.0 (M+H⁺).

To a suspension of 2,4-difluoro-5-nitrophenol (1.01 g, 5.77 mmol) in EtOAc was added palladium hydroxide (0.08 g, 0.57 mmol) and the resulting slurry was stirred under a hydrogen atmosphere for 6 h. The mixture was filtered through a Celite® pad, washing with EtOAc (2×10 mL) and the filtrate was concentrated to afford 5-amino-2,4-difluorophenol (0.8 g, 96% yield) as a solid. ¹H NMR (400 MHz, DMSO-d₆) δ 9.28 (s, 1H), 6.91 (t, J=7.2 Hz, 1H), 6.35 (t, J=8.8 Hz, 1H), 4.84 (brs, 2H); MS (ESI) m/z: 146.0 (M+H⁺).

To a solution of 5-amino-2,4-difluorophenol (0.3 g, 2.07 mmol) in DMSO (2 mL) was added potassium t-butoxide (0.23 g, 2.07 mmol) at RT. After stirring for 1 h, 3,5-dichloropyridine (0.37 g, 2.5 mmol) and potassium carbonate (0.14 g, 1 mmol) were added and the mixture was heated to 190° C. for 1 h in microwave reactor. Water (30 mL) was added, and the product was extracted with EtOAc (2×35 mL) and the combined organic layers were washed with brine solution, dried (Na₂SO₄), concentrated in vacuo and purified by chromatography (EtOAc/hexane) to afford 5-(5-chloropyridin-3-yloxy)-2,4-difluorobenzenamine (0.35 g, 66% yield) as a solid. ¹H NMR (400 MHz, Acetone-d₆) δ 8.33-8.30 (m, 2H), 7.44 (t, J=2.4 Hz, 1H), 7.13 (t, J=10.8 Hz, 1H), 6.78 (t, J=8.4 Hz, 1H), 4.85 (brs, 2H); MS (ESI) m/z: 257.0 (M+H⁺).

To a solution of 5-(5-chloropyridin-3-yloxy)-2,4-difluorobenzenamine (0.35 g, 1.4 mmol) in 1M HCl solution (10 mL) was added Pd/C (0.015 g) and mixture was shaken on a Parr apparatus under a hydrogen atmosphere (40 psi) for 24 h. The mixture was filtered through Celite® and the filter pad was washed with water (2×5 mL) and the filtrate was concentrated on the lyophilizer to afford the hydrochloride salt. This compound was neutralized with sat aq NaHCO₃ solution, the free amine extracted into EtOAc (2×35 mL) and the combined organic layers were washed with brine, dried (Na₂SO₄) and concentrated to yield 2,4-difluoro-5-(pyridin-3-yloxy)benzenamine (0.19 g, 63% yield) as a solid. ¹H NMR (400 MHz, Acetone-d₆) δ 8.33-8.30 (m, 2H), 7.37-7.29 (m, 2H), 7.09 (t, J=10.4 Hz, 1H), 6.70 (t, J=8.4 Hz, 1H), 4.78 (brs, 2H); MS (ESI) m/z: 223.0 (M+H⁺).

### Example A6

A solution of 4-amino-o-cresol (0.301 g, 2.44 mmol) in anhydrous dimethylacetamide (6 mL) was de-gassed in vacuo and treated with potassium tert-butoxide (0.33 g, 2.93 mmol) under argon. The reaction mixture was sonicated briefly to suspend all solid matter in the liquid volume. The reaction was further stirred at RT for 30 min. Example A22 (0.417 g, 2.44 mmol) was added and the resultant mixture was heated to 100° C. overnight. The cooled reaction mixture was partitioned between ethyl acetate (50 mL) and water (20 mL). The organic layer was further washed with water (3×20 mL) and saturated brine (2×20 mL). The combined aqueous phases were extracted with ethyl acetate (2×20 mL). The combined organic phases were dried (MgSO₄), concentrated in vacuo, and purified by silica gel chromatography (EtOAc/hexanes) to provide 4-(4-amino-2-methylphenoxy)-N-methylpicolinamide (530 mg, 84% yield) as a yellow foam. ¹H NMR (400 MHz, DMSO-d₆) δ 8.75 (m, 1H), 8.45 (dd, J=4.6, 0.5 Hz, 1H), 7.27 (dd, J=2.6, 0.4 Hz, 1H), 7.04 (dd, J=5.5, 2.6 Hz, 1H), 6.78 (d, J=8.5 Hz, 1H), 6.53 (d, J=2.3 Hz, 1H), 6.48 (dd, J=8.6, 2.5 Hz, 1H), 5.10 (s, 2H), 2.78 (d, J=5.0 Hz, 3 H), 1.93 (s, 3H); MS (ESI) m/z: 258.0 (M+H⁺).

### Example A7

Using a procedure analogous to Example A2,4-amino-3-fluorophenol (14 g, 0.11 mmol) and Example A25 (16 g, 0.100 mmol) were combined to provide 4-(4-amino-3-fluorophenoxy)picolinamide (8.8 g, 36% yield). ¹H NMR (300 MHz, DMSO-d₆) δ 8.46 (d, J=5.7 Hz, 1H), 8.09 (br s, 1H), 7.68 (br s, 1H), 7.34 (d, J=2.4 Hz, 1H), 7.10 (dd, J=5.6, 2.6 Hz, 1H), 7.01 (dd, J=5.7, 2.4 Hz, 1 H), 6.84 (t, J=9.0 Hz, 1H), 6.77 (dd, J=5.7, 2.4 Hz, 1H), 5.22 (s, 2H); MS (ESI) m/z: 248.1 (M+H⁺).

### Example A8

A solution of Example A23 (2.0 g, 8.4 mmol) in 2-amino-ethanol (6.0 mL) was heated to 150° C. for 3 h. The solvent was removed under reduced pressure and the residue was purified by silica gel column chromatography to provide 2-(4-(4-amino-3-fluorophenoxy)-pyridin-2-ylamino)-ethanol (1.2 g, 54% yield). ¹H NMR (400 MHz, DMSO-d₆) δ 7.78 (d, J=5.6 Hz, 1H), 6.85 (dd, J=12.0, 2.4 Hz, 1H), 6.78 (t, J=8.8 Hz, 1H), 6.67 (dd, J=8.8, 2.0 Hz, 1H), 6.44 (t, J=5.2 Hz, 1H), 6.06 (dd, J=6.0, 2.4 Hz, 1H), 5.80 (d, J=2.0 Hz, 1H), 5.08 (s, 2H), 4.68 (br s, 1H), 3.43 (m, 2H), 3.25-3.20 (m, 2H); MS (ESI) m/z: (M+H⁺) 264.1

### Example A9

A solution of Example A23 (4.0 g, 16.8 mmol) and N,O-dimethylhydroxylamine HCl (3.3 g, 34 mmol) were combined in 1,4-dioxane (50 mL) and the reaction mixture was heated overnight at 110° C. The reaction mixture was concentrated in vacuo, neutralized with 3M NaOH and extracted with EtOAc (3×). The combined organic phases were washed with brine, dried (MgSO₄) and concentrated in vacuo to obtain 4-(4-amino-3-fluorophenoxy)-N-methoxy-N-methylpyridin-2-amine (4.4 g, 99% yield). ¹H NMR (DMSO-d₆) δ 8.06 (d, J=5.2 Hz, 1H), 6.95 (dd, J=12.4, 2.8 Hz, 1H), 6.83 (dd, J=8.8, 8.4 Hz, 1H), 6.75 (dd, J=8.4, 2.4 Hz, 1H), 6.43 (d, J=2.4 Hz, 1H), 6.37 (dd, J=5.6, 2.4 Hz, 1H), 5.16 (s, 2H), 3.61 (s, 3H), 3.14 (s, 3H); MS (ESI) m/z: 264.2 (M+H⁺).

A mixture of 2-fluoro-4-(2-(methoxy(methyl)amino)pyridine-4-yloxy)aniline (2.0 g, 7.6 mmol) and 10% Pd/C (200 mg, 0.18 mmol) in MeOH (15 mL) was stirred under a H₂ atmosphere (50 psi) at RT for 48 h. The mixture was filtered through Celite® and the cake was washed with MeOH. The filtrate was concentrated to afford 4-(4-amino-3-fluorophenoxy)-N-methylpyridin-2-amine (1.2 g, 68% yield). ¹H NMR (DMSO-d₆) δ 7.86 (d, J=6.3 Hz, 1H), 6.82-6.69 (m, 3H), 6.18 (dd, J=6.0, 2.1 Hz, 1H), 5.84 (d, J=2.1 Hz, 1H), 5.41 (br s, 1H), 3.62 (s, 2H), 2.84 (d, J=3.0 Hz, 3H); MS (ESI) m/z: 234.2 (M+H⁺).

### Example A10

A solution of Example A24 (0.95 g, 7.47 mmol) and potassium tert-butoxide (0.92 g, 8.2 mmol) in dimethylacetamide (2.0 mL) was degassed under vacuum and backfilled with N₂ (4×) and then stirred for 30 min. 3,5-Dichloropyridine was added and the resulting solution was heated to 80° C. overnight. The mixture was filtered and the filtrate was concentrated in vacuo and purified by silica gel chromatography to provide 5-(5-chloropyridin-3-yloxy)-2-fluoroaniline (0.5 g, 28% yield). ¹H NMR (400 MHz, DMSO-d₆) δ 8.37 (s, 1H), 8.29 (s, 1H), 7.51 (s, 1H), 7.00 (dd, J=10.8, 8.8 Hz, 1H), 6.46 (dd, J=7.6, 2.8 Hz, 1H), 6.22 (m, 1H), 5.38 (s, 2H); MS (ESI) m/z: 239.2 (M+H⁺).

### Example A11

### A mixture of Example A8 (0.263 g, 1.0 mmol), imidazole (0.0749 g, 1.1 mmol) and TBSCl (0.181 g, 1.2 mmol) in DMF (10 mL) was stirred at RT overnight. Solvent was removed under reduced pressure. The residue was quenched with H₂O (10 mL) and the pH was adjusted to ˜8 by using NaHCO₃. The aqueous solution was extracted with EtOAc (3×20 mL) and the combined organic layers were dried (MgSO₄), concentrated in vacuo and purified by chromatography to afford 4-(4-amino-3-fluorophenoxy)-N-(2-(tert-butyldimethylsilyloxy)ethyl)pyridin-2-amine (0.252 g, 67% yield) as a light yellow oil. MS (ESI) m/z: 378.3 (M+H⁺).

### Example A12

To a solution of Example A17 (7.5 g, 32.5 mmol) in EtOH (60 mL) was added 1.0 M aqueous NaOH (10 mL, 100 mmol). The resultant mixture was heated at 85° C. overnight. The majority of ethanol was removed in vacuo and the concentrate was diluted with water (50 mL) and washed with ethyl acetate. The aqueous layer was acidified to pH 1-2 by the addition of 3 M HCl. The acidic solution was extracted with EtOAc (3×200 mL) and the extracts were washed with brine, dried (MgSO₄) and concentrated in vacuo to give 5-(3-amino-4-fluorophenoxy)picolinic acid (6.2 g, 77%, yield). ¹H-NMR (300 MHz, DMSO-d₆) δ 8.40 (d, J=2.7 Hz, 1H), 8.01 (d, J=8.4 Hz, 1H), 7.38 (dd, J=8.7, 2.7 Hz, 1H), 7.03 (dd, J=11.4, 8.7 Hz, 1H), 6.50 (dd, J=7.5, 3.0 Hz, 1H), 6.26 (m, 1H), 5.39 (br, s, 2H); MS (ESI) m/z: 249.1 (M+H⁺).

5-(3-amino-4-fluorophenoxy)picolinic acid (0.14 g, 0.56 mmol) was dissolved in THF (3 mL) and stirred at 0° C. for 5 min. 1M Borane (3.4 mL) solution was added dropwise to the reaction mixture at 0° C. over a period of 30 min. The ice bath was removed and stirring continued at RT for 7 hours. The reaction mixture was cooled in an ice bath and treated with 3M HCl (5 mL). The solution was heated for 1 h at 50° C. The solution was washed with EtOAc (2×) and the aqueous layer was cooled in an ice bath and neutralized with 3M NaOH. The solution was extracted with EtOAc (3×), the combined organic layers were washed with brine, dried (Na₂SO₄) and concentrated in vacuo to obtain (5-(3-amino-4-fluorophenoxy)pyridin-2-yl)methanol (0.13 g, 98% yield). ¹H NMR (400 MHz, DMSO-d₆) δ 8.24 (d, J=2.8 Hz, 1H), 7.46 (d, J=8.8 Hz, 1H), 7.40 (dd, J=2.8, 8.4 Hz, 1H), 6.99 (dd, J=8.8, 11.2 Hz, 1H), 6.40 (dd, J=2.8, 7.6 Hz, 1H), 6.15 (dt, J=3.2, 8.8 Hz, 1H), 5.40 (t, J=5.6 Hz, 1H), 5.33 (s, 2H), 4.54 (d, J=6.0 Hz, 2H); MS (ESI) m/z: 235.0 (M+H⁺).

### Example A13

NaH (100 mg, 3.3 mmol) was slowly added to a solution of Example A12 (0.50 g, 2.1 mmol) in dry THF (50 mL) at 0° C. After 30 min, CS₂ (0.49 g, 6.4 mmol) was added and the reaction mixture was stirred at 0° C. for 1 hour. Methyl iodide (2.4 g, 17 mmol) was added at 0° C. and the reaction mixture was allowed to warm to RT overnight. The solvent was removed under reduced pressure to obtain the crude product. The crude, O-(5-(3-amino-4-fluorophenoxy)pyridin-2-yl)methyl S-methyl carbonodithioate (0.69 g, 2.1 mmol) was dissolved in toluene (5 mL) and tributyltin hydride (1 mL) and AIBN (50 mg) were added. The reaction mixture was heated under reflux for 3 hours. The solvent was removed under reduced pressure and the residue was filtered and washed with CH₂Cl₂. The filtrate was evaporated and the residue was purified by silica gel column chromatography to obtain 2-fluoro-5-(6-methylpyridin-3-yloxy)benzenamine (0.26 g, 56% yield). ¹H NMR (400 MHz, DMSO-d₆) δ 8.20 (d, J=2.8 Hz, 1H), 7.30 (dd, J=2.8, and 8.4 Hz, 1H), 7.25 (d, J=8.4 Hz, 1H), 6.97 (dd, J=8.8, 11.6 Hz, 1H), 6.38 (dd, J=3.2, 7.6 Hz, 1H), 6.13 (dt, J=3.2, 8.8 Hz, 1H), 5.31 (s, 1H), 2.44 (s, 3H); MS (ESI) m/z: 219.0 (M+H⁺).

### Example A14

A solution of 4-amino-3-fluorophenol (0.20 g, 1.6 mmol) in 4 mL of anhydrous DMA was treated with potassium tert-butoxide (0.24 g, 1.9 mmol). The resultant dark-red solution was stirred at RT for 1 hour in a capped vial. 4-Chloro-2-methoxypyridine (0.26 g, 1.6 mmol) was added and the reaction mixture was heated overnight at 100° C. Water (50 mL) was added and the solution was extracted with ethyl acetate (3×50 mL). The combined organic layers were washed with brine, dried (Na₂SO₄), concentrated in vacuo and purified by silica gel column chromatography to obtain 2-fluoro-4-(2-methoxypyridin-4-yloxy)benzenamine (0.20 g, 58% yield). ¹H NMR (400 MHz, DMSO-d₆) δ 8.02 (d, J=6.0 Hz, 1H), 6.95 (dd, J=2.8, 12.0 Hz, 1H), 6.82 (dd, J=8.4, 8.8 Hz, 1H), 6.73 (dd, J=2.0, 8.4 Hz, 1H), 6.54 (dd, J=2.4, 6.0 Hz, 1H), 6.10 (d, J=2.4 Hz, 1H), 5.17 (s, 1H), 3.81 (s, 3H); MS (ESI) m/z: 235.0 (M+H⁺).

### Example A15

A teflon capped vial was charged with 4-amino-3-fluorophenol (0.291 g, 2.29 mmol) and anhydrous DMF (2.3 mL). The resultant solution was de-gassed in vacuo and backfilled with argon (3×). The vial was treated with sodium tert-butoxide (0.27 g, 2.41 mmol) under argon and quickly capped. The reaction mixture was stirred at RT for 1 h. After addition of 4-chloropicolinonitrile (0.317 g, 2.29 mmol) and K₂CO₃ (0.174 g, 1.26 mmol), the vial was de-gassed again and heated in a 90° C. oil bath overnight. The reaction mixture was diluted with EtOAc (60 mL) and washed with brine (25 mL). The aqueous phase was back-extracted with EtOAc (50 mL). The combined organic layers were washed with brine (25 mL), dried (MgSO₄), concentrated in vacuo and purified by chromatography to afford 4-(4-amino-3-fluorophenoxy)picolinonitrile (0.162 g, 31% yield) as a colorless oil. ¹H NMR (DMSO-d₆) δ 8.56 (d, J=5.6 Hz, 1H), 7.62 (d, J=2.0 Hz, 1H), 7.14 (dd, J=6.0, 2.8 Hz, 1H), 7.03 (dd, J=11.6, 2.4 Hz, 1H), 6.88-6.77 (m, 2H), 5.25 (s, 2H); MS (ESI) m/z: 230.0 (M+H⁺).

### Example A16

A solution of 5-amino-2-chloro-4-fluorophenol (100 mg, 0.619 mmol) in degassed dimethylacetamide (2 mL) was treated with potassium t-butoxide (83 mg, 0.743 mmol) and 5-chloro-2-cyanopyridine (86 mg, 0.619 mmol). The resultant mixture was heated to 80° C. overnight, then cooled to RT and diluted with water (10 mL). The mixture was extracted with EtOAc (30 mL). The organic phase was washed with water (3×30 mL) and brine (30 mL) dried (Na₂SO₄) and concentrated in vacuo to provide 5-(5-amino-2-chloro-4-fluorophenoxy)picolinonitrile as a dark oil which was used without further purification. MS (ESI) m/z: 264.0 (M+H⁺).

### Example A17

A solution of 3-amino-4-fluoro-phenol (5.6 g, 44 mmol) in dimethylacetamide (60 mL) was degassed in vacuo and was treated with potassium tert-butoxide (5.3 g, 47 mmol). The resulting solution was stirred for 30 min. 5-Bromo-pyridine-2-carbonitrile (6.6 g, 36 mmol) was added in one-portion and the mixture was heated at 80° C. overnight. The solvent was removed in vacuo and the residue was purified by silica gel chromatography to provide 5-(3-amino-4-fluorophenoxy)picolinonitrile (3.5 g, 44% yield). ¹H-NMR (300 MHz, DMSO-d₆) δ 8.47 (d, J=3.0 Hz, 1H), 7.98 (d, J=8.4 Hz, 1H), 7.44 (dd, J=8.8, 2.7 Hz, 1H), 7.06 (t, J=9.2 Hz, 1H), 6.52 (d, J=7.6 Hz, 1H), 6.28 (m, 1H), 5.44 (br s, 2H); MS (ESI) m/z: 230.0 (M+H⁺).

### Example A18

In DMA (10 mL) was placed 3-amino-4-fluorophenol (500 mg, 3.93 mmol), potassium t-butoxide (441 mg, 3.93 mmol) and 4-chloro-2-(methylthio)pyrimidine (632 mg, 3.93 mmol). The mixture was warmed to 50° C. and stirred overnight. The mixture was cooled to RT and diluted with water (30 mL), extracted with ethyl acetate (2×25 mL) and the combined organic phases washed with brine, dried (Na₂SO₄) and concentrated to yield a dark oil. The oil was purified by column chromatography to yield 2-fluoro-5-(2-(methylthio)pyrimidin-4-yloxy)benzenamine (841 mg, 85% yield) as an oil which was used without further purification. MS (ESI) m/z: 252.0 (M+H⁺).

### Example A19

A solution of pyridine-3-boronic acid (0.68 g, 5.5 mmol) and 2-methyl-5-nitro phenol (0.85 g, 5.5 mmol) in DCM (10 mL) was treated with pyridine (1.00 mL, 12.4 mmol), copper acetate (1.5 g, 8.3 mmol) and powdered 4A molecular sieves (330 mg). The reaction mixture was stirred for 7 days at RT open to air. The mixture was poured into water (50 mL) and extracted with DCM (2×50 mL). The combined organic phases were washed with saturated aq NaHCO₃ (25 mL), water (25 mL), satd NH₄Cl (2×25 mL) and brine (25 mL), dried (Na₂SO₄), concentrated in vacuo and purified via chromatography on silica gel to provide 3-(2-methyl-5-nitrophenoxy)pyridine (81 mg, 6% yield). ¹H NMR (400 MHz, CDCl₃) δ 8.48 (dd, J=4.6, 1.0 Hz, 1H), 8.43 (d, J=2.4 Hz, 1H), 7.99 (dd, J=8.0, 2.0 Hz, 1 H), 7.70 (d, J=2.4 Hz, 1H), 7.46 (d, J=8.4 Hz, 1H), 7.39-7.30 (m, 2H), 2.42 (s, 3 H); MS (ESI) m/z: 231.0 (M+H⁺).

A solution of 3-(2-methyl-5-nitrophenoxy)pyridine (80 mg, 0.35 mmol) and 10% Pd/C (50% wet, 165 mg, 0.08 mmol) in methanol (4 mL) was treated with formic acid (89%, 1 mL, 35 mmol) and the resultant solution was stirred at RT. After 1 h, the reaction mixture was filtered through Celite®, and the filter cake was washed with methanol. The filtrates were concentrated in vacuo, diluted with 40 mL of a pH 12 aqueous solution and extracted with ethyl acetate (3×25 mL). The extracts were dried (Na₂SO₄) and concentrated in vacuo to provide 4-methyl-3-(pyridin-3-yloxy)benzenamine (58 mg, 83% yield). ¹H NMR (400 MHz, CDCl₃) δ 8.36 (m, 2 H), 8.32 (dd, J=4.6, 1.4 Hz, 1H), 7.26-7.18 (m, 3H), 7.05 (d, J=8.0 Hz, 1H), 6.49 (dd, J=8.8, 2.4 Hz, 1H), 6.29 (d, J=2.4 Hz, 1H), 2.11 (s, 3H); MS (ESI) m/z: 201.0 (M+H⁺).

### Example A20

In DMA (8 mL) was placed 3-amino-4-fluorophenol (281 mg, 2.21 mmol), potassium t-butoxide (248 mg, 2.21 mmol) and 5-bromo-2-(trifluoromethyl)pyridine (500 mg, 2.21 mmol). The mixture was warmed to 75° C. overnight, then cooled to RT and diluted with water (75 mL). The mixture was extracted with ethyl acetate (2×40 mL) and the combined organic phases washed with brine (40 mL), dried (Na₂SO₄), concentrated in vacuo and purified by column chromatography to yield 2-fluoro-5-(6-(trifluoromethyl)pyridin-3-yloxy)benzenamine (161 mg, 26% yield) as an oil which was used without further purification. MS (ESI) m/z: 273.0 (M+H⁺).

### Example A21

In DMF (5 mL) was placed 5-(3-amino-4-fluorophenoxy)picolinic acid from Example A12 (500 mg, 2.01 mmol), 2.0 M methylamine solution/THF (10 mL, 20.1 mmol) and HOBt (324 mg, 2.12 mmol). To this was added N1-((ethylimino)methylene)-N3,N3-dimethylpropane-1,3-diamine hydrochloride (772 mg, 4.03 mmol) and the solution stirred overnight at RT. The solution was treated with an additional equiv of N1-((ethylimino)methylene)-N3,N3-dimethylpropane-1,3-diamine hydrochloride (775 mg) and warmed to 40° C., then cooled to RT and stirred overnight. The solution was diluted with ethyl acetate (30 mL) and washed with water (30 mL), brine (30 mL), dried (Na₂SO₄) and concentrated in vacuo to yield 5-(3-amino-4-fluorophenoxy)-N-methylpicolinamide (530 mg, 101% yield) as a thick oil, which was used without further purification. MS (ESI) m/z: 262.0 (M+H⁺).

### Example A22

To stirring anhydrous DMF (25 mL) was slowly added SOCl₂ (125 mL) at such a rate that the reaction temperature was maintained at 40-50° C. Pyridine-2-carboxylic acid (25 g, 0.2 mol) was added in portions over 30 min and the resulting mixture was heated at reflux for 16 h during which time a yellow solid precipitated. After cooling to RT, the mixture was diluted with toluene (80 mL) and concentrated. This process was repeated three times. The resulting dry residue was washed with toluene and dried under reduced pressure to yield 4-chloro-pyridine-2-carbonyl chloride (27.6 g, 79% yield), which was used in the next step without purification.

To a solution of 4-chloro-pyridine-2-carbonyl chloride (27.6 g, 0.16 mol) in anhydrous THF (100 mL) at 0° C. was added dropwise a solution of MeNH₂ in EtOH. The resulting mixture was stirred at 3° C. for 4 h. The reaction mixture was concentrated under reduced pressure to yield a solid, which was suspended in EtOAc and filtered. The filtrate was washed with brine (2×100 mL), dried and concentrated to yield 4-chloro-N-methylpicolinamide (16.4 g, 60% yield) as a yellow solid. ¹H NMR (400 MHz, DMSO-d₆) δ 8.78 (br s, 1H), 8.55 (d, J=5.2 Hz, 1H), 7.97 (d, J=2.0 Hz, 1H), 7.66 (m, 1H), 2.82 (d, J=4.8 Hz, 3H); MS (ESI) m/z: 171.0 (M+H⁺).

### Example A23

Using a procedure analogous to Example A2, 2,4-dichloropyridine (8.0 g, 54 mmol) and 3-fluoro-4-aminophenol (8.0 g, 62.9 mmol) were combined to provide 4-(2-chloro-pyridin-4-yloxy)-2-fluorophenylamine (11 g, 86% yield). ¹H NMR (300 MHz, DMSO-d₆) δ 8.24 (d, J=5.7 Hz, 1H), 7.00 (dd, J=9.0, 2.7 Hz, 1H), 6.89-6.73 (m, 4H), 5.21 (br s, 2H); MS (ESI) m/z: 239.2 (M+H⁺).

### Example A24

Methyl chloroformate (77.3 g, 0.82 mol) was added dropwise to a −10° C. solution of 2-chloro-4-fluorophenol (100 g, 0.68 mol) and sodium hydroxide (32.8 g, 0.82 mol) in water (550 mL). After complete addition, the precipitated solid was collected by filtration and washed with water to give 2-chloro-4-fluorophenyl methyl carbonate (110 g, 79% yield). ¹H NMR (300 MHz, DMSO-d₆) δ 7.62 (dd, J=8.1, 2.7 Hz, 1H), 7.50 (dd, J=9.0, 5.4 Hz, 1H), 7.30 (td, J=8.1, 3.0 Hz, 1H), 3.86 (s, 3H); MS (ESI) m/z: 205.2 (M+H⁺).

To a suspension of 2-chloro-4-fluorophenyl methyl carbonate (110 g, 0.54 mol) in cone. H₂SO₄ (50 mL) was slowly added a mixture comprised of cone. H₂SO₄ (40 mL) and fuming HNO₃ (40.8 mL, 0.89 mol). The resultant mixture was stirred for 30 min at 0° C. The reaction mixture was poured into ice water and the precipitated solid was collected by filtration and washed with water to give 2-chloro-4-fluoro-5-nitrophenyl methyl carbonate (120 g, 90% yield). ¹H NMR (400 MHz, DMSO-d₆): δ 8.45 (d, J=7.2 Hz, 1H), 8.12 (d, J=10.8 Hz, 1H), 3.89 (s, 3H); MS (ESI) m/z: 250.1 (M+H⁺).

A mixture of 2-chloro-4-fluoro-5-nitrophenyl methyl carbonate (120 g 0.48 mol) and sodium hydroxide (22.7 g, 0.57 mol) in water (300 mL) was refluxed for 4 h. The insoluble solids were removed by filtration and the filtrate was acidified with dilute HCl. The precipitated solid was collected by filtration and washed with water to give 2-chloro-4-fluoro-5-nitrophenol (90 g, 98% yield). ¹H NMR (400 MHz, DMSO-d₆) δ 11.18 (s, 1H), 8.10 (d, J=10.4 Hz, 1H), 7.62 (d, J=7.2 Hz, 1H); MS (ESI) m/z: 192.1 (M+H⁺)

2-Chloro-4-fluoro-5-nitrophenol (85 g, 0.45 mol) and 10% Pd/C (25 g, 0.023 mol) were combined in EtOH and hydrogenated (50 psi) for 12 h. The reaction mixture was filtered, concentrated in vacuo and purified by silica gel chromatography to provide 3-amino-4-fluorophenol (40 g 70% yield). ¹H NMR (400 MHz, DMSO-d₆) δ 8.87 (s, 1H), 6.70 (dd, J=11.2, 8.8 Hz, 1H), 6.14 (dd, J=7.8, 2.4 Hz, 1H), 5.84 (m, 1H), 4.92 (s, 2H); MS (ESI) m/z: 128.2 (M+H⁺).

### Example A25

4-Chloropicolinamide was prepared using a procedure analogous to Example A22 by substituting NH₃ for MeNH₂. ¹H NMR (300 MHz, DMSO-d₆) δ 8.59 (d, J=5.2 Hz, 1H), 8.18 (br s, 1H), 8.00 (d, J=2.0 Hz, 1H), 7.79 (br s, 1H), 7.72 (dd, J=5.2, 2.0 Hz, 1H); MS (ESI) m/z: 157.0 (M+H⁺).

### Example A26

Using a procedure analogous to Example A2,2-fluoro-4-aminophenol (2.6 g, 24 mmol) and 2,4-dichloropyridine (2.88 g, 20 mol) were combined to provide 4-(2-chloropyridin-4-yloxy)-3-fluoro-phenylamine (3.2 g, 67% yield). ¹H NMR (400 MHz, DMSO-d₆) δ 8.25 (d, J=5.6 Hz, 1H), 6.99 (m, 1H), 6.90 (m, 2H), 6.50 (d, J=1.6 Hz, 1H), 6.41 (d, J=10.4 Hz, 1H), 5.51 (s, 2H); MS (ESI) m/z: 239.1 (M+H⁺).

A mixture of 4-(2-chloro-pyridin-4-yloxy)-3-fluoro-phenylamine (2.0 g, 8.4 mmol) and benzylmethylamine (20 mL) was heated to 200° C. overnight in a steel bomb. The reaction mixture was concentrated in vacuo and purified by silica gel chromatography to give 4-(4-amino-2-fluorophenoxy)-N-benzyl-N-methylpyridin-2-amine (1.0 g, 37% yield). MS (ESI) m/z: 324.2 (M+H⁺).

To a solution of 4-(4-amino-2-fluorophenoxy)-N-benzyl-N-methylpyridin-2-amine (1.0 g, 3.1 mmol) in MeOH (10 mL) was added 10% Pd/C (0.25 g, 0.23 mmol). The reaction was stirred under a H₂ atmosphere (50 psi) at 75° C. for 12 h. The reaction mixture was filtered, concentrated under reduced pressure and purified by reverse phase prep-HPLC to provide 4-(4-amino-2-fluorophenoxy)-N-methylpyridin-2-amine (560 mg, 78% yield). ¹H NMR (400 MHz, DMSO-d₆) δ 7.80 (d, J=5.6 Hz, 1H), 6.90 (t, J=9.0 Hz, 1H), 6.40-6.45 (m, 3H), 6.06 (dd, J=8.0, 2.8 Hz, 1H), 5.73 (d, J=2.8 Hz, 1H), 5.37 (s, 2H), 2.68 (d, J=4.8 Hz, 3H); MS (ESI) m/z: (M+H⁺): 234.2.

### Example A27

Example A23 (0.597 g, 2.5 mmol), 4-(4,4,5,5-tetramethyl-1,3,2-dioxaborolan-2-yl)-1H-pyrazole (0.728 g, 3.75 mmol), Cs₂CO₃ (3.10 g, 9.5 mmol) and Pd(PPh₃)₄ (0.289 g, 0.25 mmol) were combined in DMF/H₂O (20 mL). The reaction mixture was degassed, blanketed with N₂ and heated at 90° C. overnight. The completed reaction was diluted with H₂O (5 mL) and extracted with EtOAc (3×50 mL). The combined organics were washed with brine (20 mL), dried (MgSO₄), concentrated in vacuo and purified by chromatography to afford 4-(2-(1H-pyrazol-4-yl)pyridin-4-yloxy)-2-fluorobenzenamine (0.56 g, 83%) as a light yellow solid. ¹H NMR (400 Hz, DMSO-d₆) δ 13.01 (s, 1H), 8.38 (d, J=5.6 Hz, 1H), 8.35 (s, 1H), 8.06 (s, 1H), 7.29 (d, J=2.4 Hz, 1H), 7.03 (dd, J=11.6, 2.4 Hz, 1H), 6.89 (t, J=8.8 Hz, 1H), 6.84 (m, J=8.4 Hz, 1H), 6.60 (m, 1H), 5.20 (s, 2H); MS (ESI) m/z: 271.0 (M+H⁺).

### Example A28

A solution of Example A23 (3 g, 12.6 mmol), 1-methyl-3-(4,4,5,5-tetramethyl-[1,3,2]dioxaborolan-2-yl)-1H-pyrazole (5.2 g, 25.2 mmol), and Na₂CO₃ (2.7 g, 25.2 mmol) in DME (18 mL) and water (6 mL) was sparged with nitrogen for 20 min. Pd(PPh₃)₄ (729 mg, 0.63 mmol) was added and the resulting mixture was heated to 100° C. for 16 h. The solvent was removed under reduced pressure and the crude product was suspended in water and extracted with EtOAc. The organic layer was washed with brine, dried (Na₂SO₄), concentrated in vacuo and purified by silica gel chromatography to give 2-fluoro-4-(2-(1-methyl-1H-pyrazol-4-yl)pyridin-4-yloxy)aniline (2 g, 56% yield). ¹H NMR (300 MHz, DMSO-d₆) δ 8.31 (d, J=5.7 Hz, 1H), 8.21 (s, 1H), 7.92 (s, 1H), 7.12 (s, J=2.4 Hz, 1H), 6.96 (m, 1 H), 6.85-6.72 (m, 2H), 6.56 (m, 1H), 5.15 (s, 2H), 3.84 (s, 3H); MS (ESI) m/z: 285.0 (M+H⁺)

### Example A29

By analogy to Example A2,4-amino-3-fluorophenol (0.12 g, 0.53 mmol), potassium tert-butoxide (0.080 g, 0.71 mmol) and tert-butyl 4-chloropicolinate (159 mg, 0.53 mmol) were combined to provide tert-butyl 4-(4-amino-3-fluorophenoxy)picolinate (151 mg, 67% yield). MS (ESI) m/z: 305.0 (M+H⁺)

To a solution of LiAlH₄ (699 mg, 18.4 mmol) in THF (15 mL) was added tert-butyl 4-(4-amino-3-fluorophenoxy)picolinate (1.4 g, 4.6 mmol) at 0° C. under N₂. The mixture was stirred at 0° C. for 2 h. The reaction mixture was quenched with 10% aq NaOH solution (4 mL), the resultant suspension was filtered and the filtrate was extracted with EtOAc (3×30 mL) to give (4-(4-amino-3-fluorophenoxy)pyridin-2-yl)methanol (700 mg, 70% yield). MS (ESI) m/z: 235.1 (M+H⁺).

A solution of (4-(4-amino-3-fluorophenoxy)pyridin-2-yl)methanol (750 mg, 3.2 mmol) and Et₃N (821 mg, 8 mmol) in DMF (10 ml) at 0° C. was treated with tert-butyldimethylsilyl chloride (624 mg, 4.16 mmol). The resulting solution was stirred at RT for 4 hours. The solvent was removed in vacuo and the residue was purified by silica gel column chromatography to provide 4-(2-((tert-butyldimethylsilyloxy)methyl)pyridin-4-yl)pyridin-4-yloxy)-2-fluorobenzenamine (370 mg, 33% yield). ¹H NMR (400 MHz, DMSO-d₆) δ 8.32 (d, J=5.6 Hz, 1H), 7.02 (s, 1H), 6.67-6.82 (m, 4H), 4.76 (s, 2H), 3.71 (s, 2H), 0.89 (s, 9H), 0.07 (s, 6H); MS (ESI) m/z: 349.2 (M+H⁺).

### Example A30

Example A23 (1 g, 4.2 mmol) and ethyl(4-methoxy-benzyl)amine (10 mL) were combined and heated to 200° C. for 30 hours. The reaction solution was poured into HOAc/water (20%, V/V) and extracted with EtOAc (3×100 mL). The combined organics were washed with brine (3×50 mL) and saturated NaHCO₃ solution (2×100 mL), dried (NaSO₄), concentrated in vacuo and purified by silica gel chromatography to give [4-(4-amino-3-fluoro-phenoxy)-pyridin-2-yl]-ethyl-(4-methoxybenzyl)amine (1.2 g, 78% yield). ¹H NMR (400 MHz, DMSO-d₆) 67.90 (d, J=5.6 Hz, 1H), 7.07 (d, J=8.4 Hz, 2H), 6.82 (d, J=8.0 Hz, 2H), 6.74 (m, 2H), 6.63 (d, J=7.2 Hz, 1H), 6.02 (d, J=4.0 Hz, 1H), 5.90 (s, 1H), 5.09 (s, 2H), 4.53 (s, 2H), 3.67 (s, 3H), 3.44 (m, 2H), 1.00 (t, J=6.8, 3H); MS (ESI) m/z: 368.2 (M+H⁺).

Trifluoroacetic acid (10 mL) was added to a solution of [4-(4-amino-3-fluoro-phenoxy)-pyridin-2-yl]-ethyl-(4-methoxybenzyl)amine (1.2 g, 3.27 mmol) in CH₂Cl₂ (50 mL) and the resulting solution was heated to 40° C. overnight. The reaction mixture was concentrated under reduced pressure and the residue was treated with HCl (5 mL, 12M, 60 mmol) and water (50 mL). The solution was washed with EtOAc (4×50 mL). The aqueous layer was treated with NaHCO₃ until pH=8 and then extracted with EtOAc (3×50 mL). The combined extracts were washed with brine (3×50 mL), dried (NaSO₄) and concentrated in vacuo to give 4-(4-amino-3-fluorophenoxy)-N-ethylpyridin-2-amine (0.45 g, 67% yield). ¹H NMR (300 MHz, DMSO-d₆) δ7.79 (d, J=5.7, 1H), 6.85 (dd, J=11.7, 2.4 Hz, 1H), 6.78 (t, J=8.7 Hz, 1H), 6.67 (dd, J=8.7, 2.4 Hz, 1H), 6.39 (m, 1H), 6.05 (dd, J=5.7, 2.1 Hz, 1H), 5.72 (d, J=2.1 Hz, 1H), 5.09 (s, 2H), 3.15 (m, 2H), 1.03 (t, J=7.2, 3H); MS (ESI) m/z: 248.2 (M+H⁺).

### Example A31

To a solution of Example A23 (0.30 g, 1.3 mmol) in NMP (5 mL) was added isopropylamine (0.54 mL, 6.3 mmol) and it was heated under microwave at 200° C. for 6 hours. Water was added and the solution was extracted with ethyl acetate. The organic layer was washed with brine, dried (MgSO₄), concentrated in vacuo and purified by silica gel column chromatography (EtOAc/hexane:EtOAc:MeOH/CH₂Cl₂) to obtain 4-(4-amino-3-fluorophenoxy)-N-isopropylpyridin-2-amine (0.16 g, 49% yield). MS (ESI) m/z: 262.2 (M+H⁺).

### Example A32

A solution of 3,5-dinitro-benzonitrile (5 g, 25.9 mol), 5-chloro-pyridin-3-ol (3.35 g, 25.9 mol) and K₂CO₃ (7.2 g, 52 mol) in DMF (150 mL) was heated at 100° C. overnight. The mixture was concentrated in vacuo and the residue was poured into water. The aqueous layer was extracted with ethyl acetate (3×150 mL) and the combined organics were washed with brine, dried (Na₂SO₄), concentrated in vacuo and purified by silica gel chromatography to afford 3-(5-chloro-pyridin-3-yloxy)-5-nitro-benzonitrile (3.1 g, 44% yield). ¹H NMR (400 MHz, DMSO-d₆) δ 8.56 (s, 1H), 8.51 (s, 1H), 8.47 (s, 1H), 8.22 (s, 1H), 8.19 (s, 1H), 7.87 (s, 1H).

Iron powder (6.3 g, 112 mmol) was added to a mixture of 3-(5-chloro-pyridin-3-yloxy)-5-nitro-benzonitrile (3.1 g, 11.2 mol) in acetic acid (100 mL) and the reaction was stirred at RT for 6 h. Water (200 mL) was added and the mixture was neutralized to pH 7 with saturated Na₂CO₃ solution and was extracted with EtOAc (3×150 mL). The combined organics were washed with brine, dried (Na₂SO₄), concentrated in vacuo and purified on silica gel to give 3-amino-5-(5-chloropyridin-3-yloxy)benzonitrile (1.92 g, 71% yield). ¹H NMR (400 MHz, DMSO-d₆) δ 8.53 (d, J=1.6 Hz, 1H), 8.44 (d, J=2.4 Hz, 1H), 7.80 (t, J=2.4 Hz, 1H), 6.77 (s, 1H), 6.72 (d, J=1.6 Hz, 1H), 6.56 (d, J=2.0 Hz, 1H), 5.92 (s, 2H); MS (ESI) m/z: 246.2 [M+H]⁺.

### Example A33

3,5-dinitro-benzonitrile (3 g, 16 mmol), 6-methylpyridin-3-ol (1.7 g, 16 mmol), and K₂CO₃ (4.3 g, 31 mmol) were dissolved in DMF and heated to 110° C. overnight. The reaction mixture was poured into water and the mixture was extracted with EtOAc. The combined organics were washed with brine, dried (Na₂SO₄), concentrated in vacuo and purified by silica gel chromatography to provide 3-(6-methylpyridin-3-yloxy)-5-nitrobenzonitrile (3 g, 76% yield). ¹H NMR (400 MHz, DMSO) δ 8.50 (s, 1H), 8.38 (s, 1H), 8.08 (s, 1H), 8.01 (s, 1H), 7.59-7.56 (d, J=10 Hz, 1H), 7.38-7.36 (d, J=8.4 Hz, 1H), 1.98 (s, 3H); MS (ESI) m/z: 256.3 [M+H]⁺.

A mixture of 3-(6-methylpyridin-3-yloxy)-5-nitrobenzonitrile (3 g, 0.012 mol) and iron powder in acetic acid (200 mL) was stirred at RT for 6 h. H₂O was added and the mixture was adjusted to pH 7 with saturated Na₂CO₃ solution. The aqueous layer was extracted with EtOAc, and the combined organics were washed with brine, dried (MgSO₄), concentrated in vacuo and purified by silica gel chromatography to afford 3-amino-5-(6-methylpyridin-3yloxy)benzonitrile (2 g, 76% yield). ¹H NMR (400 MHz, DMSO) δ 8.25 (s, 1H), 7.42 (d, J=10 Hz, 1H), 7.30 (d, J=8.4 Hz, 1H), 6.62 (s, 1H), 6.51 (s, 1H), 6.38 (s, 1H), 5.78 (s, 2H), 2.49 (s, 3H); MS (ESI) m/z: 226.2 [M+H]⁺.

### Example A34

3,5-Dinitrobenzonitrile (1.50 g, 7.77 mmol) was added to a slurry of pyridin-3-ol (739 mg, 7.77 mmol) and potassium carbonate (10.7 g, 77.7 mmol) in DMF (15 mL), the mixture was warmed to 60° C. and stirred overnight. After cooling to RT the reaction was diluted with ethyl acetate (50 mL) and water (100 mL). The organic phase was separated, washed with saturated sodium bicarbonate (50 mL) and brine (50 mL), dried (Na₂SO₄), concentrated in vacuo and purified by chromatography (Si-40 column, ethyl acetate/hexanes) to give a light yellow solid identified as 3-nitro-5-(pyridin-3-yloxy)benzonitrile (1.31 g, 69% yield). MS (ESI) m/z: 242.0 (M+H⁺).

A solution of 3-nitro-5-(pyridin-3-yloxy)benzonitrile (1.31 g, 9.42 mmol) and tin(II)chloride dehydrate (6.13 g, 27.2 mmol) in ethanol (20 mL) was warmed to 70° C. for 2 hrs. After cooling to RT, the reaction was poured onto ice/water (100 mL). The aqueous mixture was made basic (pH˜=8) with sodium hydroxide, diluted with ethyl acetate (50 mL) and filtered through paper to remove most salts. This solution was extracted with ethyl acetate (2×75 mL) and the combined organics washed with brine, dried (Na2SO4) and concentrated in vacuo to give a light yellow solid identified as 3-amino-5-(pyridin-3-yloxy)benzonitrile (660 mg, 57% yield). MS (ESI) m/z: 212.0 (M+H⁺).

### Example A35

Using a procedure analogous to Example A3, 3-amino-4-fluorophenol (491 mg, 3.86 mmol) and 4-chloropyrimidin-2-amine (500 mg, 3.86 mmol) were combined to give 4-(3-amino-4-fluorophenoxy)pyrimidin-2-amine (509 mg, 59% yield). MS (ESI) m/z: 221.0 (M+H⁺).

### Example A36

A solution of 1,3-difluoro-2-methylbenzene (15 g, 0.12 mol) in H₂SO₄ (100 mL) was treated dropwise with HNO₃ (65%, 11.4 g, 0.12 mol) at −10° C. The resultant mixture was stirred for about 30 min. The mixture was poured into ice-water and extracted with EtOAc (3×200 mL). The combined organics were washed with brine, dried (NaSO₄) and concentrated in vacuo to give 1,3-difluoro-2-methyl-4-nitrobenzene (16 g, 78% yield). ¹H NMR (400 MHz, CDCl₃) δ 7.80 (m, 1 H), 6.8-7.1 (m, 1H), 2.30 (s, 3H).

1,3-difluoro-2-methyl-4-nitrobenzene (16 g, 0.092 mol), benzyl alcohol (10 g, 0.092 mol) and K₂CO₃ (25.3 g, 0.18 mol) were combined in DMF (250 mL) and heated to 100° C. overnight. The mixture was poured into water and extracted with EtOAc (3×200 mL). The combined organics were washed with brine, dried (Na₂SO₄), concentrated in vacuo and purified by column chromatography on silica gel to give 1-benzyloxy-3-fluoro-2-methyl-4-nitrobenzene (8 g, 33% yield). ¹H NMR (400 MHz, DMSO-d₆) δ 8.04 (t, J=8.8 Hz, 1H), 7.30-7.46 (m, 5H), 7.08 (d, J=9.2 Hz, 1H), 5.28 (s, 2H), 2.13 (s, 3H).

1-Benzyloxy-3-fluoro-2-methyl-4-nitrobenzene (8 g, 0.031 mol) and 10% Pd—C (1 g) were combined in methanol (100 mL) and the mixture was stirred under an H₂ atmosphere (1 atm) overnight. The reaction mixture was filtered and the filtrate was concentrated in vacuo to give 4-amino-3-fluoro-2-methylphenol (4.2 g, 96% yield). ¹H NMR (300 MHz, DMSO-d₆) δ 8.61 (s, 1H), 6.42 (t, J=8.4 Hz, 1H), 7.11 (d, J=8.4 Hz, 1H), 4.28 (s, 2H), 1.96 (s, 3H); MS (ESI) m/z: 142.1 [M+H]⁺.

Potassium tert-butoxide (3.5 g, 0.031 mol) was added to a solution of 4-amino-3-fluoro-2-methylphenol (4.2 g, 0.03 mol) in DMAc and the resultant mixture was stirred for 30 min at RT. To this mixture was added a solution of 2,4-dichloropyridine (4.38 g, 0.03 mol) in DMAc and the mixture was heated at 100° C. overnight. The reaction mixture was concentrated in vacuo and the residue was dissolved in ethyl acetate (200 mL) and filtered through silica gel, washing forward with EtOAc. The filtrate was concentrated and purified by silica gel chromatography to give 4-(2-chloropyridin-4-yloxy)-2-fluoro-3-methylbenzenamine (3.2 g, 42% yield). ¹H NMR (300 MHz, DMSO-d₆) δ 8.21 (d, J=6.0 Hz, 1H), 6.84 (s, 1H), 6.81 (dd, J=5.6, 2.4 Hz, 1H), 6.67 (m, 2H), 5.12 (s, 2H), 1.91 (s, 3H); MS (ESI) m/z 253.1 [M+H]⁺.

4-(2-Chloropyridin-4-yloxy)-2-fluoro-3-methylbenzenamine (1.0 g, 3.3 mmol), 1-methyl-4-(4,4,5,5-tetramethyl-[1,3,2]dioxaborolan-2-yl)-1H-pyrazole (1 g, 4.8 mmol), Na₂CO₃ (0.84 g, 6.6 mmol) and Pd(PPh₃)₄ (0.25 g, 0.2 mmol) were combined in DME (75 mL) and water (25 mL). The mixture was sparged with nitrogen for 15 min and was heated to reflux overnight. The reaction mixture was extracted with EtOAc (3×100 mL) and the combined organics were washed with brine, concentrated in vacuo and purified by silica gel chromatography to give 2-fluoro-3-methyl-4-(2-(1-methyl-1H-pyrazol-4-yl)pyridin-4-yloxy)aniline (0.74 g, 75% yield). ¹H NMR (300 MHz, DMSO-d₆) δ 8.27 (d, J=6.0 Hz, 1H), 8.18 (s, 1H), 7.90 (s, 1H), 7.07 (s, 1H), 6.63 (m, 2H), 6.45 (dd, J=5.6, 2.4 Hz, 1H), 5.06 (s, 2H), 3.82 (s, 3H), 1.95 (s, 3H); MS (ESI) m/z: 299.2 [M+H]⁺.

### Example A37

A solution of 1,2,3-trifluoro-4-nitro-benzene (30 g, 0.17 mol) and benzyl alcohol (18.4 g, 0.17 mol) in DMF (300 mL) was treated with K₂CO₃ (35 g, 0.25 mol) and the resulting mixture was stirred at RT for 8 h. Water (300 mL) was added, and the mixture was extracted with EtOAc (3×500 mL). The combined organics were washed with brine, dried (MgSO₄), concentrated in vacuo and chromatographed on silica gel to give 1-benzyloxy-2,3-difluoro-4-nitrobenzene (16 g, 36% yield). ¹H NMR (400 MHz, DMSO-d₆): δ 8.06 (m, 1H), 7.49-7.30 (m, 6H), 5.37 (s, 2H).

A mixture of 1-benzyloxy-2,3-difluoro-4-nitrobenzene (14 g, 52.8 mmol) and Pd/C (10%, 1.4 g) in MeOH (200 mL) was stirred under a hydrogen atmosphere (30 psi) for 2 h. The catalyst was removed by filtration and the filtrate was concentrated in vacuo to afford 4-amino-2,3difluoro-phenol (7 g, 92% yield). ¹H NMR (400 MHz, DMSO-d₆) δ 9.05 (s, 1H), 6.45 (t, J=8.8 Hz, 1H), 6.34 (t, J=9.2 Hz, 1H), 4.67 (s, 2H).

Using a procedure analogous to Example A2,4-amino-2,3-difluorophenol (6 g, 41.4 mmol), potassium tert-butoxide (4.9 g, 43.5 mmol) and 2,4-dichoropyridine (6.1 g, 41.4 mmol) were combined to afford 4-(2-chloro-pyridin-4-yloxy)-2,3-difluorophenylamine (7 g, 66% yield). ¹H NMR (400 MHz, DMSO-d₆) δ 8.27 (d, J=6.0 Hz, 1H), 7.05 (s, 1H), 6.95 (m, 1H), 6.92 (d, J=8.8 Hz, 1H), 6.62 (d, J=8.8 Hz, 1H), 5.60 (s, 2H).

### Example A38

A solution of Example A37 (2 g, 7.8 mmol), 1-methyl-4-(4,4,5,5-tetramethyl-[1,3,2]dioxaborolan-2-yl)-1H-pyrazole (1.6 g, 7.8 mmol) and Na₂CO₃ (1.65 mg, 15.6 mmol) in DME (12 mL) and H₂O (4 mL) was sparged with nitrogen for 20 min. Pd(PPh₃)₄ (450 mg, 0.4 mmol) was added and the resulting mixture was heated to 70° C. under nitrogen for 16 h. The solvent was removed under reduced pressure and the crude product was suspended in water and extracted with EtOAc (3×10 mL). The organic layer was washed with brine, dried (MgSO₄), concentrated in vacuo and purified by column chromatography on silica gel to give 2,3-difluoro-4-[2-(1-methyl-1H-pyrazol-4-yl)-pyridin-4-yloxy]phenylamine (1.3 g, 55% yield). ¹H NMR (400 MHz, DMSO-d₆) δ 8.40 (d, J=6.0 Hz, 1H), 8.32 (s, 1H), 8.02 (s, 1H), 7.26 (s, 1H), 6.96 (t, J=8.8 Hz, 1H), 6.70-6.67 (m, 2H), 5.62 (s, 2H), 3.92 (s, 3H); MS (ESI) m/z: 303.2[M+H]⁺.

### Example A39

Example A23 (2.0 g, 8.4 mmol) and 4-methoxybenzylamine (50 mL) were combined in a steel bomb and heated to 160° C. for 3 h. The reaction mixture was concentrated under reduced pressure and purified by reverse prep-HPLC to give N-(4-methoxybenzyl)-4-(4-amino-3-fluorophenoxy)pyridin-2-amine (1.0 g, 35% yield).

A solution of N-(4-methoxybenzyl)-4-(4-amino-3-fluorophenoxy)pyridin-2-amine (500 mg, 1.47 mmol) in CH₂Cl₂ (10 mL) was treated with ammonium cerium(IV) nitrate (1.64 g, 2.99 mmol) and the resultant mixture was stirred at RT overnight. The reaction mixture was washed with water, concentrated in vacuo and purified by silica gel chromatography to yield 4-(4-amino-3-fluorophenoxy)pyridin-2-amine (250 mg, 77% yield). ¹H NMR (300 MHz, DMSO-d₆) δ 7.73 (d, J=6.0 Hz, 1H), 6.88 (dd, J=9.0, 2.0 Hz, 1H), 6.80 (t, J=8.7 Hz, 1H), 6.68 (m, 1H), 6.06 (dd, J=4.5, 1.8 Hz, 1H), 5.84 (s, 2H), 5.75 (d, J=1.5 Hz, 1H), 5.08 (s, 2H); MS (ESI) m/z: 220.3 (M+H⁺).

### Example A40

A solution of 4-amino-2-methyl-phenol (4.25 g, 34.5 mmol) in dimethylacetamide (50 mL) was degassed in vacuo and blanketed with argon. Potassium tert-butoxide (5.0 g, 44.6 mmol) was added and the reaction mixture was de-gassed a second time and stirred at RT under argon for 30 min. 2,4-Dichloro-pyridine (4.6 g, 31.3 mmol) was added and the mixture was heated to 100° C. overnight. The solvent was removed under reduced pressure and the residue was purified by silica gel chromatography to give 4-(2-chloropyridin-4-yloxy)-3-methylbenzenamine (4.5 g, 56% yield). ¹H NMR (400 MHz, DMSO-d₆) δ 8.21 (d, J=5.2 Hz, 1H), 6.75-6.80 (m, 3H), 6.45-6.50 (m, 2H), 5.15 (s, 2H), 1.92 (s, 3H); MS (ESI) m/z: 235.1 (M+H⁺).

A solution of 4-(2-chloropyridin-4-yloxy)-3-methylbenzenamine (595 mg, 2.54 mmol), 1-methyl-4-(4,4,5,5-tetramethyl)-[1,3,2]dioxaborolan-2-yl)-4H-pyrazole (790 mg, 3.80 mmol) and Cs₂CO₃ (2.53 g, 7.77 mmol) in 10 mL of DMF (10 mL) and water (3 mL) was de-gassed under vacuum and blanketed with nitrogen. Pd(PPh₃)₄ (295 mg, 0.26 mmol) was added and the reaction mixture was heated to 90° C. overnight. The reaction mixture was diluted with EtOAc (30 mL) and washed with water (2×10 mL) and brine (2×10 mL). The aqueous portion was extracted with EtOAc (2×15 mL) and the combined organics were washed with brine (10 mL), concentrated in vacuo and purified on silica gel to provide 3-methyl-4-(2-(1-methyl-1H-pyrazol-4-yl)pyridin-4-yloxy)benzenamine as a pale yellow colored foam (627 mg, 88% yield). ¹H NMR (400 MHz, DMSO-d₆): δ 8.27 (d, J=6.0 Hz, 1H), 8.18 (s, 1H), 7.90 (d, J=0.7 Hz, 1H), 7.07 (d, J=2.2 Hz, 1H), 6.74 (d, J=8.6 Hz, 1H), 6.49 (d, J=2.5 Hz, 1H), 6.46-6.40 (m, 2H), 5.02 (s, 2H), 3.84 (s, 3H), 1.94 (s, 3 H); MS (ESI) m/z: 281.2 (M+H⁺).

### Example A41

4-Chloro-2-methylsulfanyl-pyrimidine (1.4 g, 8.8 mmol), 4-(4,4,5,5-tetramethyl-[1,3,2]dioxaborolan-2-yl)-1H-pyrazole (2.0 g, 10.3 mmol), Na₂CO₃ (2.8 g, 26.4) and Pd(PPh₃)₄ (500 mg, 0.43 mmol) were combined in a solvent comprised of toluene/EtOH/H₂O (4/4/1, 20 mL). The mixture was degassed by applying a vacuum and backfilling the headspace with argon. The reaction mixture was heated overnight at 100° C. The insoluble portion was filtered and the filtrate was concentrated and purified by silica gel chromatography to provide 2-(methylthio)-4-(1H-pyrazol-4-yl)pyrimidine (1.2 g, 71% yield). ¹H NMR (400 MHz, CDCl₃) δ 8.45 (d, J=6.4 Hz, 1H), 8.24 (s, 1H), 7.23 (s, 1H), 7.05 (d, J=6.4 Hz, 1 H), 2.51 (s, 3H).

To a solution of 2-(methylthio)-4-(1H-pyrazol-4-yl)pyrimidine (200 mg, 1 mmol) in dichloromethane (3 mL) and H₂O (1 mL) was added 4-methoxybenzylchloride (200 mg, 1.28 mmol) at 0° C. The mixture was stirred at RT overnight. The organic layer was separated, washed with brine and concentrated in vacuo to give crude 4-(1-(4-methoxybenzyl)-1H-pyrazol-4-yl)-2-(methylthio)pyrimidine. ¹H NMR (300 MHz, DMSO-d₆) δ 8.58 (s, 1H), 8.50, (d, J=5.4 Hz, 1H), 8.16 (s, 1H), 7.40 (d, J=5.4 Hz, 1H), 7.27 (d, J=8.4 Hz, 2H), 7.22 (d, J=8.4 Hz, 2H), 5.30 (s, 2H), 3.72 (s, 3H), 2.51 (s, 3H); MS (ESI) m/z: 313 (M+H⁺).

To a solution of 4-(1-(4-methoxybenzyl)-1H-pyrazol-4-yl)-2-(methylthio)pyrimidine (200 mg, 0.64 mmol) in dichloromethane was added m-CPBA (220 mg, 1.28 mmol). The reaction was stirred for 2 hour at RT. Water was added, the organic layer was separated and the aqueous layer was extracted with dichloromethane. The combined organics were washed with brine and concentrated in vacuo. The residue was combined with 3-amino-4-fluorophenol (165 mg, 1.28 mmol) and K₂CO₃ (176 mg, 1.28 mmol) in DMF (5 mL) and the resultant mixture was heated at 90° C. overnight. After filtration and concentration, the residue was purified by silica gel column chromatography to give 5-(4-(1-(4-methoxybenzyl)-1H-pyrazol-4-yl)pyrimidin-2-yloxy)-2-fluorobenzenamine (210 mg, 84% yield). ¹H NMR (300 MHz, DMSO-d₆) δ 8.50 (s, 1H), 8.44, (d, J=5.4 Hz, 1H), 8.10 (s, 1H), 7.42 (d, J=5.4 Hz, 1H), 7.25 (d, J=8.4 Hz, 2H), 6.98 (t, J=9.6 Hz, 1H), 6.91 (d, J=8.4 Hz, 2H), 6.52 (dd, J=2.7, 8.7 Hz, 1H), 6.28 (m, 1H), 5.30 (br s, 2H), 5.26 (s, 2H), 3.72 (s, 3H); MS (ESI) m/z: 392.2 (M+H⁺).

To a solution of 5-(4-(1-(4-methoxybenzyl)-1H-pyrazol-4-yl)pyrimidin-2-yloxy)-2-fluorobenzenamine (50 mg, 0.13 mmol) in dichloromethane (3 mL) was added TFA (0.3 mL) at 0° C. and the reaction stirred at RT for 12 h. The solvent was removed in vacuo, the residue was washed with ether and treated with saturated ammonia solution. The solid was collected via filtration and dried under vacuum to give 5-(4-(1H-pyrazol-4-yl)pyrimidin-2-yloxy)-2-fluorobenzenamine (15 mg, 43% yield). ¹H NMR (300 MHz, MeOD) δ 8.44 (d, J=5.1 Hz, 1H), 8.23 (br s, 2H), 7.40 (d, J=5.4, 1H), 7.02 (dd, J=10.8, 8.7 Hz, 1H), 6.73 (dd, J=2.7, 7.2 Hz, 1H), 6.50 (m, 1H); MS (ESI) m/z: 272.2 (M+H⁺).

### Example A42

Using a procedure analogous to Example A3,3-amino-4-fluorophenol (0.127 g, 1.0 mmol) and 5-bromo-2-nitropyridine (0.203 g, 1.0 mmol) were combined to afford 2-fluoro-5-(6-nitropyridin-3-yloxy)benzenamine (0.098 g, 39% yield) as a yellow solid. ¹H NMR (400 MHz, DMSO-d6) δ 8.36 (d, J=2.8 Hz, 1H), 8.30 (d, J=8.8 Hz, 1H), 7.56 (dd, J=8.8, 2.8 Hz, 1H), 7.07 (m, 1H), 6.53 (dd, J=7.6, 3.2 Hz, 1H), 6.31 (s, 1H), 5.48 (s, 2H); MS (ESI) m/z: 250.0 (M+H⁺).

### Example B1

To a stirring solution of benzyl 6-(4,4,5,5-tetramethyl-1,3,2-dioxaborolan-2-yl)-3,4-dihydroisoquinoline-2(1H)-carboxylate (0.991 g, 2.52 mmol, 1.00 eq) in THF (10 ml) and H₂O (2.5 ml) was added NaIO₄ (1.62 g, 7.56 mmol, 3.00 eq). The resulting suspension was stirred at 25° C. for 30 min and then treated with 3M HCl (1.68 ml, 5.04 mmol, 2.0 eq). The mixture was stirred for 2.5 h. The supernatant was decanted away from the solids, rinsing forward with THF. The combined organic phases were washed with brine (2×), dried (MgSO₄) and concentrated in vacuo to give crude 2-(benzyloxycarbonyl)-1,2,3,4-tetrahydroisoquinolin-6-ylboronic acid (0.640 g, 82% yield) as a foam which was used as is in the next reaction. ¹H NMR (400 MHz, DMSO-d₆) δ 7.68-7.58 (m, 2H), 7.45-7.29 (m, 6H), 7.17 (m, 1H), 5.13 (s, 2H), 4.62-4.56 (brm, 2H), 3.65 (brs, 2H), 2.86 (t, 2H, J=5.60 Hz); MS (ESI) m/z: 312.0 (M+H⁺).

To a stirring suspension of 2-(benzyloxycarbonyl)-1,2,3,4-tetrahydroisoquinolin-6-ylboronic acid (0.640 g, 2.06 mmol, 1.00 eq) and 4 Å MS (0.64 g) in CH₂Cl₂ (20 ml) was added pyridine (0.168 ml, 2.06 mmol, 1.00 eq) followed by ethyl 3-t-butyl-1H-pyrazole-5-carboxylate (0.404 g, 2.06 mmol, 1.00 eq) and Cu(OAc)₂ (0.374 g, 2.06 mmol, 1.00 eq). The resulting blue-green mixture was stirred at 25° C. After 40 h, the mixture was diluted with H₂O and decanted away from the molecular sieves. The layers were separated and the organic phase was washed with H₂O (2×). The combined aqueous phases were extracted with CH₂Cl₂ (1×). The combined organic phases were dried (MgSO₄), concentrated in vacuo and purified by flash chromatography (EtOAc/hexanes) to afford benzyl 6-(3-t-butyl-5-(ethoxycarbonyl)-1H-pyrazol-1-yl)-3,4-dihydroisoquinoline-2(1H)-carboxylate (0.46 g, 48% yield). ¹H NMR (400 MHz, DMSO-d₆) δ 7.41-7.28 (m, 5H), 7.24-7.20 (m, 3H), 6.96 (s, 1H), 5.15 (s, 2H), 4.67 (brm, 2H), 4.17 (q, 2H, J=7.2 Hz), 3.66 (brs, 2H), 2.86 (t, 2H, J=6.0 Hz), 1.29 (s, 9H), 1.18 (t, 3H, J=7.2 Hz); MS (ESI) m/z: 462.3 (M+H⁺).

To a stirring solution of benzyl 6-(3-t-butyl-5-(ethoxycarbonyl)-1H-pyrazol-1-yl)-3,4-dihydroisoquinoline-2(1H)-carboxylate (0.160 g, 0.347 mmol) in 1:1:1 THF/EtOH/H₂O (3 ml) at 22° C. was added LiOH.H₂O (0.0727 g, 1.73 mmol). After 3 h, the completed reaction was acidified (pH 2-3) with 1M HCl and extracted with EtOAc (3×). The combined organic phases were washed with brine (2×), dried (MgSO₄), filtered and evaporated to afford 1-(2-(benzyloxycarbonyl)-1,2,3,4-tetrahydroisoquinolin-6-yl)-3-t-butyl-1H-pyrazole-5-carboxylic acid (0.16 g, 106% yield) as an oil which was used without further purification. ¹H NMR (400 MHz, DMSO-d₆) δ 7.41-7.31 (m, 5H), 7.328-7.20 (m, 3H), 6.91 (s, 1H), 5.15 (s, 2H), 4.65 (brm, 2H), 3.66 (brs, 2H), 2.86 (t, 2H, J=6.0 Hz), 1.29 (s, 9H); MS (ESI) m/z: 434.2 (M+H⁺).

### Example B2

Ethyl 3-t-butyl-1-(2-(trifluoromethylsulfonyloxy)quinolin-6-yl)-1H-pyrazole-5-carboxylate (see WO 2006/071940A2, 0.380 g, 0.806 mmol), MeNH₂.HCl (0.109 g, 1.61 mmol) and Et₃N (0.449 ml, 3.22 mmol) were combined DMF (8 mL) and stirred at RT overnight. Additional portions of MeNH₂.HCl (0.109 g, 1.61 mmol) and Et₃N (0.449 ml, 3.22 mmol) were added and the reaction was stirred an additional 4 h at RT and 3 h at 60° C. The completed reaction was diluted with brine and extracted with EtOAc. The extracts were washed with brine, dried (Na₂SO₄), concentrated in vacuo and purified by silica gel chromatography to provide ethyl 3-tert-butyl-1-(2-(methylamino)quinolin-6-yl)-1H-pyrazole-5-carboxylate (240 mg, 85% yield). ¹H NMR (400 MHz, DMSO-d₆) δ 7.90 (d, J=9.2 Hz, 1H), 7.68 (d, J=2.8 Hz, 1H), 7.53 (d, J=9.2 Hz, 1H), 7.46 (dd, J=8.8, 2.0 Hz, 1H), 7.17 (q, J=4.8 Hz, 1H), 6.98 (s, 1H), 6.80 (d, J=8.8 Hz, 1H), 4.16 (q, J=7.2 Hz, 2H), 2.92 (d, J=4.8 Hz, 3H), 1.32 (s, 9H), 1.13 (t, J=7.2 Hz, 3H); MS (ESI) m/z: 353.2 (M+H⁺).

LiOH.H₂O (0.143 g, 3.40 mmol) was added to a solution of ethyl 3-tert-butyl-1-(2-(methylamino)quinolin-6-yl)-1H-pyrazole-5-carboxylate (0.240 g, 0.68 mmol) in a mixture of water/THF/EtOH (1:1:1, 9 mL). The reaction mixture was stirred overnight at RT, diluted with 3 M HCl and extracted with EtOAc and THF. The combined organics were washed with brine, dried (MgSO₄) and concentrated under vacuum to obtain 3-tert-butyl-1-(2-(methylamino)quinolin-6-yl)-1H-pyrazole-5-carboxylic acid (0.22 g, 100% yield). ¹H-NMR (DMSO-d₆) δ 7.90 (d, J=9.2 Hz, 1H), 7.66 (d, J=2.4 Hz, 1H), 7.52 (d, J=8.8 Hz, 1H), 7.46 (dd, J=9.2, 2.8 Hz, 1H), 7.14 (m, 1H), 6.88 (brs, 1H), 6.79 (d, J=9.2 Hz, 1H), 2.92 (d, J=4.8 Hz, 3H), 1.31 (s, 9H); MS (ESI) m/z: 325.2 (M+H⁺).

### Example B3

A solution of triflic anhydride (42.8 g, 0.15 mol) in CH₂Cl₂ (100 mL) was added dropwise to a 0° C. solution of 6-hydroxyquinoline (20.00 g, 0.138 mol) and pyridine (23 g, 0.277 mol) in CH₂Cl₂ (500 mL). The cooling bath was removed and the resulting solution was stirred at RT for 4 h. The reaction mixture was washed with water (3×300 mL) and the organic phase was dried (MgSO₄) and concentrated under vacuum to afford crude quinolin-6-yl trifluoromethanesulfonate (40 g, >100% yield) as an oil. ¹H-NMR (400 MHz, DMSO-d₆) δ 9.00 (d, 1H, J=2.8 Hz), 8.50 (d, 1H, J=8.0 Hz), 8.21 (d, J=2.8 Hz, 1H), 8.18 (d, J=9.2 Hz, 1H), 7.80 (m, 1H), 7.64 (m, 1H); MS (ESI) m/z: 277.9 (M+H⁺).

To a suspension of quinolin-6-yl trifluoromethanesulfonate (40 g, 0.14 mol), benzophenone hydrazone (35.6 g, 0.18 mol), cesium carbonate (74 g, 0.23 mol) and 1,1′-bis(diphenylphosphino)ferrocene (2.5 g, 4.5 mmol) in degassed toluene (1 L) was added palladium acetate (0.013 g, 0.058 mmol). The resultant mixture was heated to 90° C. under a nitrogen atmosphere. After 16 h, the mixture was concentrated in vacuo and the residue was purified via silica gel column chromatography (EtOAc/pet ether) to provide 1-(diphenylmethylene)-2-(quinolin-6-yl)hydrazine (32 g, 68.6% yield). ¹H-NMR (300 MHz, DMSO-d₆) δ 9.22 (s, 1H), 8.58 (t, J=1.8 Hz, 1H), 8.13 (d, J=3.6 Hz, 1H), 7.80 (d, J=3.6 Hz, 1H), 7.61 (d, J=3.9 Hz, 1H), 7.59-7.51 (m, 4H), 7.50 (d, J=3.6 Hz, 2H), 7.33-7.39 (m, 6H); MS (ESI) m/z: 324 (M+H⁺).

A solution of 1-(diphenylmethylene)-2-(quinolin-6-yl)hydrazine (32 g, 99 mmol) and 4,4-dimethyl-3-oxo-pentanenitrile (26 g, 0.15 mol) in ethanol (500 mL) was treated with conc HCl (80 ml, 12 N, 0.96 mol) and the mixture was heated to reflux overnight. The cooled reaction mixture was concentrated under vacuum and the residue was washed with Et₂O to remove the diphenylketone. The crude product was dissolved in EtOAc and neutralized (pH 8) with saturated Na₂CO₃ solution. The organic layer was dried (Na₂SO₄), concentrated in vacuo and purified by silica gel chromatography to give 5-tert-butyl-2-quinolin-6-yl-2H-pyrazol-3-ylamine (23 g, 87% yield). ¹H-NMR (300 MHz, DMSO-d₆) δ 8.86 (m, 1H), 8.39 (d, J=5.7 Hz, 1H), 8.11-8.02 (m, 3H), 7.54 (m, 1H), 5.46 (s, 1H), 5.42 (br s, 2H), 1.23 (s, 9H); MS (ESI) m/z: 267.2 (M+H⁺).

To a cold solution (−10° C.) of 5-tert-butyl-2-quinolin-6-yl-2H-pyrazol-3-ylamine (8.00 g, 30 mmol) in 100 ml of CH₂Cl₂ was added pyridine (8.0 ml, 99 mmol) and DMAP (100 mg), followed by a solution of trichloroethyl chloroformate (8.9 ml, 42 mmol) in 30 ml of CH₂Cl₂ over a period of 20 minutes. After stirring for 1 hour, water (100 ml) was added, stirring continued for 10 more minutes and the organic layer separated. The organic layer was washed with brine, dried and the dark brown residue obtained after removal of the solvent crystallized from acetonitrile to furnish 2,2,2-trichloroethyl 3-tert-butyl-1-(quinolin-6-yl)-1H-pyrazol-5-ylcarbamate as a white solid (8.23 g, 62% yield). ¹H NMR (DMSO-d₆) δ 10.15 (br s, 1H) 8.93 (m, 1H), 8.41 (d, J=8 Hz, 1H), 8.11 (m, 2H), 7.90 (dd, J=8, 2 Hz, 1H), 7.60 (dd, J=6.4, 4.2 Hz, 1H), 6.39 (s, 1H), 4.85 (s, 2H), 1.32 (s, 9H); MS (ESI) m/z: 442 (M+H⁺).

### Example B4

Quinolin-6-ylboronic acid (0.34 g, 2.0 mmol) was dissolved in CH₂Cl₂ (30 mL) and pyridine (1 mL) with MS (activated 4A) and stirred at RT for 6 hours. Ethyl 3-tert-butyl-1H-pyrazole-5-carboxylate (0.39 g, 2.0 mmol) and copper(II)acetate (0.36 g, 2.0 mmol) were added and the reaction was stirred at RT for 3 days open to air. The reaction mixture was filtered through a pad of Celite®, the filtrate was concentrated in vacuo and purified by silica gel chromatography to obtain ethyl 3-tert-butyl-1-(quinolin-6-yl)-1H-pyrazole-5-carboxylate (0.21 g, 33% yield). MS (ESI) m/z: 324.0 (M+H⁺).

Lithium hydroxide (62 mg, 2.6 mmol) was added to a solution of ethyl 3-tert-butyl-1-(quinolin-6-yl)-1H-pyrazole-5-carboxylate (0.21 g, 0.65 mmol) in dioxane-H₂O-EtOH (1:1:1, 6 mL). The reaction mixture was stirred overnight at RT. The solution was concentrated and the residue was dissolved in H₂O (2 mL). 3M HCl was added and the precipitate was collected by filtration and washed with water. The solid was dried under vacuum to obtain 3-tert-butyl-1-(quinolin-6-yl)-1H-pyrazole-5-carboxylic acid (0.18 g, 94% yield) as a white solid. ¹H NMR (400 MHz, DMSO-d₆) δ 8.96 (dd, J=2.0, 4.0 Hz, 1H), 8.47 (dd, J=1.2, 8.4 Hz, 1H), 8.09 (m, 1H), 8.06 (s, 1H), 7.82 (dd, J=2.8, 9.2 Hz, 1H), 7.61 (dd, J=4.8, 8.8 Hz, 1H), 7.01 (s, 1H), 1.33 (s, 9H); MS (ESI) m/z: 296.0 (M+H⁺).

### Example B5

[3-(5-amino-3-t-butyl-pyrazol-1-yl)naphthalen-1-yl]acetic acid ethyl ester hydrochloride (see WO 2006/071940, 1.60 g, 4.55 mmol) was treated with ammonia in methanol (7 M, 13 mL, 91 mmol) and the reaction mixture was heated in a sealed tube for 6 days. The solvent was removed in vacuo and the residue was chromatographed to provide 2-(3-(5-amino-3-tert-butyl-1H-pyrazol-1-yl)naphthalen-1-yl)acetamide (610 mg, 41% yield). MS (ESI) m/z: 323.3 (M+H⁺).

To a mixture of saturated sodium bicarbonate (20 mL), ethyl acetate (20 mL) and 2-(3-(5-amino-3-tert-butyl-1H-pyrazol-1-yl)naphthalen-1-yl)acetamide (300 mg, 0.931 mmol) was added Troc-Cl (296 mg, 1.40 mmol). The mixture was stirred vigorously overnight. The mixture was diluted with ethyl acetate (30 mL) and the organic phase was separated, washed with 5% citric acid (30 mL) and brine (30 mL), dried (Na₂SO₄) and concentrated in vacuo to give a solid which was triturated with ethyl acetate and filtered to provide 2,2,2-trichloroethyl 1-(4-(2-amino-2-oxoethyl)naphthalen-2-yl)-3-tert-butyl-1H-pyrazol-5-ylcarbamate (241 mg, 52% yield). MS (ESI) m/z: 499.0 (M+H⁺).

### Example B6

To a stirring suspension of tert-butyl 5-(5-amino-3-tert-butyl-1H-pyrazol-1-yl)-1H-indazole-1-carboxylate (see WO 2006/071940A2, 0.250 g, 0.70 mmol) and Troc-Cl (0.10 ml, 0.74 mmol) in EtOAc (7 ml) at RT was added sat'd. NaHCO₃ (2.9 ml, 2.1 mmol). After 3 h, the completed reaction was diluted with hexanes (35 ml) and filtered. The solid was rinsed well with hexanes and dried to afford tert-butyl 5-(3-tert-butyl-5-((2,2,2-trichloroethoxy)carbonyl)-1H-pyrazol-1-yl)-1H-indazole-1-carboxylate (0.36 g, 97% yield). MS (ESI) m/z: 532.0 (M+H⁺).

### Example B7

To a stirring solution of t-butyl 6-(5-amino-3-t-butyl-1H-pyrazol-1-yl)-3,4-dihydroisoquinoline-2(1H)-carboxylate (see WO 2006/071940A2, 0.075 g, 0.20 mmol) and Troc-Cl (0.028 ml, 0.21 mmol) in EtOAc (2 ml) was added sat'd. NaHCO₃ (0.82 ml, 0.61 mmol). The resulting biphasic solution was stirred at RT overnight. The layers were separated and the aqueous phase was extracted with EtOAc (2×). The combined organic phases were washed with brine (1×), dried (MgSO₄) and concentrated in vacuo to give crude t-butyl 6-(3-t-butyl-5-((2,2,2-trichloroethoxy)carbonyl)-1H-pyrazol-1-yl)-3,4-dihydroisoquinoline-2(1H)-carboxylate (0.110 g, 100% yield). ¹H NMR (DMSO-d₆) δ 9.93 (brs, 1H), 7.29-7.24 (m, 2H), 6.83-6.80 (m, 1H), 6.27 (s, 1H), 4.85 (s, 2H), 4.52 (brs, 2H), 3.57-3.53 (m, 2H), 2.82-2.79 (m, 2H), 1.44 (s, 9H), 1.27 (s, 9H); MS (ESI) m/z: 545.0 (M+H⁺).

### Example B8

A solution of tert-butyl 5-(5-amino-3-tert-butyl-1H-pyrazol-1-yl)-1H-indazole-1-carboxylate (see WO 2006/071940A2, 0.64 g, 1.80 mmol) in EtOAc (6 mL) was treated with 1M aq NaOH (2.7 mL). To the stirring biphasic reaction mixture at 0° C. was added isopropenyl chloroformate (0.26 mL) dropwise over 1 min. The reaction mixture was stirred for 4 h at RT. The reaction was diluted with EtOAc (20 ml). The organic layer was washed with H₂O (2×10 ml), brine (10 ml) dried (MgSO₄) and concentrated to afford tert-butyl 5-(3-tert-butyl-5-((prop-1-en-2-yloxy)carbonylamino)-1H-pyrazol-1-yl)-1H-indazole-1-carboxylate (0.69 g, 87% yield) as a light yellow foam. ¹H NMR (DMSO-d₆) δ 9.77 (s, 1H), 8.52 (s, 1H), 8.17 (d, J=9 Hz, 1H), 7.97 (d, J=2 Hz, 1H), 7.74 (dd, J=9, 2 Hz, 1H), 6.34 (s, 1H), 4.7 (m, 2H), 1.80 (s, 3H), 1.67 (s, 9H), 1.30 (s, 9H); MS (ESI) m/z: 440.2 (M+H⁺).

### Example B9

Using a procedure analogous to Example B3, 6-(2-(diphenylmethylene)hydrazinyl)quinoline (4.0 g, 12.3 mmol) and 4-methyl-3-oxo-pentanenitrile (1.5 g, 13.5 mmol) were combined to provide to 3-isopropyl-1-(quinolin-6-yl)-1H-pyrazol-5-amine. (1.1 g, 36% yield). ¹H NMR (400 MHz, CDCl₃) δ 8.93 (dd, J=4.4, 1.6 Hz, 1H), 8.21-8.18 (m, 2H), 8.05-8.02 (m, 2H), 7.44 (dd, J=8.4, 4.4 Hz, 1H), 5.56 (s, 1H), 3.85 (br s, 2H), 2.97 (m, 1H), 1.31 (d, J=6.8 Hz, 6 H); MS (ESI) m/z: 253.2 (M+H⁺).

Using a procedure analogous to Example B3 3-isopropyl-1-(quinolin-6-yl)-1H-pyrazol-5-amine (0.378 g, 1.5 mmol) was converted to 2,2,2-trichloroethyl 3-isopropyl-1-(quinolin-6-yl)-1H-pyrazol-5-ylcarbamate (0.391 g, 61% yield). MS (ESI) m/z: 427.0 (M+H⁺).

### Example B10

Using a procedure analogous to Example B3, 6-(2-(diphenylmethylene)hydrazinyl)quinoline (4.0 g, 12.3 mmol) and 3-oxo-pentanenitrile (1.3 g, 1.1 eq) were combined to yield 5-ethyl-2-quinolin-6-yl-2H-pyrazol-3-ylamine (2.5 g, 85% yield). ¹H NMR (300 MHz, DMSO-d₆) δ 8.87 (dd, J=7.8, 1.8 Hz, 1H), 8.39 (dd, J=8.4, 1.5 Hz, 1H), 8.12 (s, 1H), 8.06-8.03 (m, 2H), 7.54 (dd, J=8.4, 1.2 Hz, 1H), 5.46 (br s, 2H), 5.40 (s, 1H), 2.49 (q, J=7.5 Hz, 2H), 1.16 (t, J=7.5 Hz, 3H); MS (ESI) m/z: 239.2 (M+H⁺).

Using a procedure analogous to Example B3, 5-ethyl-2-quinolin-6-yl-2H-pyrazol-3-ylamine (0.378 g, 1.5 mmol) was converted to 2,2,2-trichloroethyl 3-ethyl-1-(quinolin-6-yl)-1H-pyrazol-5-ylcarbamate (0.287 g, 41% yield) as a white foam. MS (ESI) m/z: 413.0 (M+H⁺).

### Example B11

Using a procedure analogous to a procedure analogous to Example B3, 6-(2-(diphenylmethylene)hydrazinyl)quinoline (5.0 g, 15.5 mmol) and 4,4,4-trifluoro-3-oxo-butyronitrile (2.3 g, 16.8 mmol) were combined to yield 2-quinolin-6-yl-5-trifluoromethyl-2H-pyrazol-3-ylamine (2.3 g, 53% yield). ¹H NMR (300 MHz, DMSO-d₆) δ 8.95 (dd, J=1.5, 4.2 Hz, 1H), 8.47 (d, J=7.2 Hz, 1H), 8.22 (d, J=2.4 Hz, 1H), 8.14 (d, J=9.3 Hz, 1H), 7.97 (dd, J=2.4, 9.0 Hz, 1H), 7.60 (dd, J=7.2, 4.2 Hz, 1H), 5.96 (br s, 2H), 5.85 (s, 1H); MS (ESI) m/z: 279.2 (M+H⁺).

Using a procedure analogous to Example B3, 2-quinolin-6-yl-5-trifluoromethyl-2H-pyrazol-3-ylamine (0.47 g, 1.7 mmol) was converted to 2,2,2-trichloroethyl 1-(quinolin-6-yl)-3-(trifluoromethyl)-1H-pyrazol-5-ylcarbamate (0.333 g, 43% yield). MS (ESI) m/z: 453.0 (M+H⁺).

### Example B12

Using a procedure analogous to Example B3, 6-(2-(diphenylmethylene)hydrazinyl)quinoline (5.0 g, 15.5 mmol) and 3-cyclopentyl-3-oxopropanenitrile (3.0 g, 1.1 eq) were combined to yield 3-cyclopentyl-1-(quinolin-6-yl)-1H-pyrazol-5-amine (2.3 g, 53% yield). ¹H NMR (300 MHz, DMSO-d₆) δ 8.87 (m, 1H), 8.38 (dd, J=1.5, 8.4 Hz, 1H), 8.10 (s, 1H), 8.04-8.02 (m, 2H), 7.55 (dd, J=4.2, 8.1 Hz, 1H), 5.41 (br s, 2H), 5.38 (s, 1H), 2.90 (m, 1H), 1.85-1.96 (m, 2H), 1.53-1.70 (m, 6H); MS (ESI) m/z: 279.3 (M+H⁺).

Using a procedure analogous to Example B3, 3-cyclopentyl-1-(quinolin-6-yl)-1H-pyrazol-5-amine (0.418 g, 1.5 mmol) was converted to 2,2,2-trichloroethyl 3-cyclopentyl-1-(quinolin-6-yl)-1H-pyrazol-5-ylcarbamate (0.394 g, 58% yield). MS (ESI) m/z: 453.0 (M+H⁺).

### Example B13

Using a procedure analogous to Example B3, 6-(2-(diphenylmethylene)hydrazinyl)quinoline (4.0 g, 12.3 mmol) and 3-cyclobutyl-3-oxo-propionitrile (1.7 g, 1.1 eq) were combined to provide 5-cyclobutyl-2-quinolin-6-yl-2H-pyrazol-3-ylamine (1.3 g, 40% yield). ¹H NMR (300 MHz, CDCl₃) δ 8.92 (dd, J=4.5, 1.2 Hz, 1H), 8.16-8.20 (m, 2H), 8.00-8.04 (m, 2H), 7.43 (dd, J=8.4, 1.2 Hz, 1H), 5.64 (s, 1H), 3.83 (br s, 2H), 3.53 (m, 1H), 2.40-2.20 (m, 4H), 2.08-1.92 (m, 2 H); MS (ESI) m/z: 265.1 (M+H⁺).

Using a procedure analogous to Example B3, 5-cyclobutyl-2-quinolin-6-yl-2H-pyrazol-3-ylamine (0.396 g, 1.5 mmol) was converted to 2,2,2-trichloroethyl 3-cyclobutyl-1-(quinolin-6-yl)-1H-pyrazol-5-ylcarbamate (0.412 g, 63% yield). MS (ESI) m/z: 439.0 (M+H⁺).

### Example B14

A degassed mixture of ethyl 5-chloro-2-iodobenzoate (0.621 g, 2.00 mmol), Pd(PPh₃)₄ (0.116 mg, 0.1 mmol), quinolin-6-ylboronic acid (0.381 g, 2.2 mmol), K₂CO₃ (0.553 g, 4.0 mmol), dimethoxyethane (20 mL), and water (5 mL) was heated under reflux overnight. Solvents were removed under reduced pressure. The residue was diluted with sat'd NH₄Cl (15 mL) and extracted with EtOAc (3×30 mL). The combined organic layers were dried (MgSO₄), concentrated in vacuo and purified by chromatography to afford ethyl 5-chloro-2-(quinolin-6-yl)benzoate (0.244 g, 39% yield) as a colorless oil. MS (ESI) m/z: 312.0 (M+H⁺).

To a stirring solution of ethyl 5-chloro-2-(quinolin-6-yl)benzoate (0.244 g, 0.78 mmol) in 1:1:1 THF/EtOH/H₂O (21 ml) at RT was added LiOH—H₂O (0.164 g, 3.91 mmol). The resulting reaction mixture was stirred at RT overnight. Solvent was removed under reduced pressure and the residue was diluted with H₂O (10 mL). The aqueous solution was acidified to pH˜4 with 3M HCl and extracted with EtOAc (3×30 mL). The combined organic layers were washed with brine (20 mL), dried (MgSO₄) and concentrated to afford 5-chloro-2-(quinolin-6-yl)benzoic acid (0.201 g, 91% yield) as a white solid. MS (ESI) m/z: 284.0 (M+H⁺).

To a stirring solution of 5-chloro-2-(quinolin-6-yl)benzoic acid (0.201 g, 0.708 mmol) and TEA (0.148 ml, 1.06 mmol) in 1,4-dioxane (10 ml) at RT, was added DPPA (0.191 ml, 0.244 mmol). After stirring for 30 min at RT, 2,2,2-trichloroethanol (0.680 ml, 7.08 mmol) was added and the reaction was stirred with heating at 100° C. for 2 h. The completed reaction was diluted with brine (10 ml) and extracted with EtOAc (3×25 ml). The combined organics were washed with 5% citric acid (10 ml), sat'd. NaHCO₃ (10 ml) and brine (10 ml), dried (MgSO₄), concentrated in vacuo and purified by chromatography to afford 2,2,2-trichloroethyl 5-chloro-2-(quinolin-6-yl)phenylcarbamate (0.25 g, 82% yield) as a white solid. MS (ESI) m/z: 431.0 (M+H⁺).

### Example B15

2,2,2-Trichloroethyl 4-chloro-2-(quinolin-6-yl)phenylcarbamate was prepared from ethyl 4-chloro-2-iodobenzoate using a procedure analogous to Example B14. MS (ESI) m/z: 431.0 (M+H⁺).

### Example B16

A mixture of 5-nitro-1H-indazole (50 g, 0.31 mol) and 10% Pd/C (5.0 g) in MeOH (400 mL) was heated under H₂ (30 psi) atmosphere overnight. After the mixture was filtered, the filtrate was concentrated to give 1H-indazol-5-ylamine as a yellow solid (40 g, 97% yield). ¹H NMR (300 MHz, DMSO-d₆) δ 12.50 (br s, 1H), 7.70 (s, 1H), 7.22 (d, J=6.6 Hz, 1H), 6.77 (d, J=6.6 Hz, 1 H), 6.74 (s, 1H), 4.72 (br s, 1H); MS (ESI) m/z: 134.2 (M+H⁺).

To a solution of 1H-indazol-5-ylamine (8.0 g, 60.1 mmol) in concentrated HCl (20 mL, 240 mmol) was added an aqueous solution (50 mL) of NaNO₂ (4.2 g, 60.1 mmol) at 0° C. and the resulting mixture was stirred for 1 h. A solution of SnCl₂.2H₂O (27 g, 120 mmol) in conc HCl (30 mL) was then added at 0° C. The reaction was stirred for an additional 2 h at RT. A solution of 4-methyl-3-oxo-pentanenitrile (8.0 g, 1.1 eq) in ethanol (50 mL) was added and the resultant mixture was heated to reflux overnight. The reaction mixture was concentrated under reduced pressure and was purified by silica gel chromatography to provide 2-(1H-indazol-5-yl)-5-isopropyl-2H-pyrazol-3-ylamine (8.5 g, 59% yield, two steps). ¹H NMR (300 MHz, DMSO-d₆) 8.09 (s, 1H), 7.82 (s, 1H), 7.57 (d, J=6.6 Hz, 1H), 7.51 (d, J=6.6 Hz, 1H), 5.31 (s, 1H), 5.12 (s, 2H), 2.74 (m, 1H), 1.15 (d, J=5.1 Hz, 6H); MS (ESI) m/z: 242.3 (M+H⁺).

A stirring solution of 2-(1H-indazol-5-yl)-5-isopropyl-2H-pyrazol-3-ylamine (8.0 g, 33 mmol) in dioxane (80 mL)/10% NaOH (30 mL) was treated with (Boc)₂O (8.6 g, 39.4 mmol). The resultant mixture was stirred for 3 h and was then extracted with DCM (3×100 mL). The organic layer was concentrated in vacuo and the residue was purified by silica gel chromatography to give 5-(5-amino-3-isopropyl-pyrazol-1-yl)-indazole-1-carboxylic acid tert-butyl ester (6.8 g, 47%) as a white solid. ¹H NMR (300 MHz, DMSO-d₆) δ 8.43 (s, 1H), 8.10 (d, J=9.3 Hz, 1H), 8.00 (br s, 1 H), 7.82 (d, J=9.3 Hz, 1H), 5.36 (s, 1H), 5.29 (br s, 2H), 2.76 (m, 1H), 1.64 (s, 9 H), 1.16 (d, J=7.2 Hz, 6H). MS (ESI) m/z: 442.2 (M+H⁺).

A solution of tert-butyl 5-(5-amino-3-isopropyl-1H-pyrazol-1-yl)-1H-indazole-1-carboxylate (1.50 g) in EtOAc (15 mL) was treated with 1M aq NaOH (6.8 mL). To the stirred biphasic reaction mixture at 0° C. was added isopropenyl chloroformate (0.64 mL) drop-wise over 1 min. The reaction mixture was stirred at RT overnight. The reaction mixture was diluted with EtOAc (100 mL), washed with H₂O (2×30 mL), brine (30 mL), dried (MgSO₄) and concentrated to afford tert-butyl 5-(3-isopropyl-5-((prop-1-en-2-yloxy)carbonylamino)-1H-pyrazol-1-yl)-1H-indazole-1-carboxylate (1.90 g, 99% yield) as a white foam. MS (ESI) m/z: 425.8 (M+H⁺).

### Example B17

Using a procedure analogous to Example B16, 1H-indazol-5-ylamine (5.0 g, 37.5 mmol) and 3-oxo-pentanenitrile (4.0 g, 1.1 eq) were combined and purified by silica gel chromatography to give 5-ethyl-2-(1H-indazol-5-yl)-2H-pyrazol-3-ylamine (5.2 g, 61% yield, two steps). ¹H NMR (300 MHz, DMSO-d₆) δ 8.04 (s, 1H), 7.58 (s, 1H), 7.57 (d, J=6.6 Hz, 1H), 7.50 (d, J=6.6 Hz, 1H), 5.30 (s, 1H), 5.13 (br s, 2H), 2.47 (q, J=6.9 Hz, 2H), 1.14 (t, J=6.9 Hz, 3H); MS (ESI) m/z: 228.3 (M+H⁺).

Using a procedure analogous to Example B16, 5-ethyl-2-(1H-indazol-5-yl)-2H-pyrazol-3-ylamine (5.0 g, 22 mmol) was converted to 5-(5-amino-3-ethyl-pyrazol-1-yl)-indazole-1-carboxylic acid tert-butyl ester (3.0 g, 42% yield) as a white solid. ¹H NMR (300 MHz, DMSO-d₆): δ 8.42 (s, 1H), 8.09 (d, J=6.6 Hz, 1H), 7.98 (s, 1H), 7.81 (d, J=6.6 Hz, 1H), 5.35 (s, 1H), 5.29 (br s, 2H), 2.44.

tert-Butyl 5-(5-amino-3-ethyl-1H-pyrazol-1-yl)-1H-indazole-1-carboxylate (0.50 g) was converted to tert-butyl 5-(3-ethyl-5-((prop-1-en-2-yloxy)carbonylamino)-1H-pyrazol-1-yl)-1H-indazole-1-carboxylate (0.55 g, 88% yield) using a procedure analogous to Example 16. MS (ESI) m/z: 412.3 (M+H⁺).

### Example B18

A solution of N-benzhydrylidene-N′-quinolin-6-yl-hydrazine (32 g, 0.099 mol) in EtOH (500 mL) was treated with cone. HCl (80 ml, 0.96 mmol). After stirring for 10 min, 5,5-dimethyl-2,4-dioxo-hexanoic acid ethyl ester (26 g, 0.15 mol) was added, and the mixture was heated to 80° C. overnight. The reaction was concentrated in vacuo to give a residue which was washed with Et₂O to afford ethyl 5-tert-butyl-1-(quinolin-6-yl)-1H-pyrazole-3-carboxylate hydrochloride (40 g, 0.11 mol, 112% yield). MS (ESI) m/z: 324.1 (M+H⁺).

A suspension of ethyl 5-tert-butyl-1-(quinolin-6-yl)-1H-pyrazole-3-carboxylate hydrochloride (32 g, 0.089 mol) in THF (300 mL) was treated with aqueous LiOH (2 N, 100 mL, 0.20 mmol) and the resultant mixture was heated to 40° C. for 3 hours. The reaction was concentrated under reduced pressure and the remaining aqueous layer was washed with EtOAc. The aqueous phase was acidified to pH 3 and the resultant precipitate was collected by filtration, washed with cold ether and dried in vacuo to provide 5-tert-butyl-1-(quinolin-6-yl)-1H-pyrazole-3-carboxylic acid (21 g, 71% yield). ¹H-NMR (400 MHz, DMSO-d₆) δ 9.03 (m, 1H), 8.50 (d, J=8.7 Hz, 1H), 8.20 (d, J=2.4 Hz, 1H), 8.15 (d, J=8.8 Hz, 1H), 7.79 (dd, J=8.7 Hz, 2.4 Hz, 1H), 7.67 (dd, J=8.4, 4.4 Hz, 1H), 6.68 (s, 1H), 1.17 (s, 9H); MS (ESI) m/z: 296.3 (M+H⁺).

### Example B19

A solution of sodium nitrite (502 mg, 7.27 mmol) in H₂O (8 ml) was added dropwise to a well-stirred 0° C. mixture of 2-methylquinolin-6-amine (1.00 g, 6.32 mmol) in cone. HCl (10 ml). The resulting mixture was stirred at 0° C. for 1 h. Tin(II)chloride dihydrate (6.13 g, 27.2 mmol) in cone. HCl (8 ml) was added and stirring was continued at 0° C. for 1 h and then RT for 2 h. Ethanol (60 ml) and 4,4-dimethyl-3-oxopentanenitrile (1.03 g, 8.22 mmol) were added and the mixture was heated at reflux overnight. The completed reaction mixture was concentrated in vacuo and diluted with ethyl acetate (100 mL). The mixture was cooled in an ice/water bath and made basic (pH˜8) with solid sodium hydroxide. The solution was filtered through Celite, and the filter cake was washed with water (50 mL) and ethyl acetate (100 mL). The organic phase was separated, washed with brine, dried (Na₂SO₄), and concentrated to yield a foam. The foam was stirred in ether (50 mL) and allowed to stand for several hours. The resultant solid was collected by filtration and dried in vacuo to yield 3-tert-butyl-1-(2-methylquinolin-6-yl)-1H-pyrazol-5-amine (428 mg, 24% yield). MS (ESI) m/z: 281.2 (M+H⁺).

A solution of 3-tert-butyl-1-(2-methylquinolin-6-yl)-1H-pyrazol-5-amine (420 mg, 1.50 mmol) in CH₂Cl₂ (15 mL) was treated with pyridine (592 mg, 7.49 mmol) and TROC-Cl (333 mg, 1.57 mmol). The mixture was stirred at RT for 16 h, then washed with 5% citric acid (2×20 mL), saturated aq NaHCO₃ (20 mL) and brine (20 mL). The organic phase was dried (Na₂SO₄) and concentrated to provide a mixture of 2,2,2-trichloroethyl 3-tert-butyl-1-(2-methylquinolin-6-yl)-1H-pyrazol-5-ylcarbamate (73% yield) contaminated with 16% of the bis-Troc aduct. The mixture was used without further purification. MS (ESI) m/z: 456.5 (M+H⁺).

### Example B20

Using a procedure analogous to Example B4, imidazo[1,2-a]pyridin-6-ylboronic acid (0.200 g, 1.23 mmol) and ethyl 3-tert-butyl-1H-pyrazole-5-carboxylate (0.267 g, 1.36 mmol) were combined to afford ethyl 3-tert-butyl-1-(imidazo[1,2-a]pyridin-6-yl)-1H-pyrazole-5-carboxylate (0.0355 g, 9% yield) as a colorless oil. MS (ESI) m/z: 313.2 (M+H⁺).

Using a procedure analogous to Example B4, ethyl 3-tert-butyl-1-(imidazo[1,2-a]pyridin-6-yl)-1H-pyrazole-5-carboxylate (0.071 g, 0.23 mmol) was converted to 3-tert-butyl-1-(imidazo[1,2-a]pyridin-6-yl)-1H-pyrazole-5-carboxylic acid (0.0643 g, 99% yield) as a white solid. MS (ESI) m/z: 285.0 (M+H⁺).

### Example B21

Using a procedure analogous to Example B4, imidazo[1,2-a]pyridin-6-ylboronic acid (0.500 g, 3.09 mmol) and ethyl 3-isopropyl-1H-pyrazole-5-carboxylate (0.619 g, 3.40 mmol) were combined to afford ethyl 3-isopropyl-1-(imidazo[1,2-a]pyridin-6-yl)-1H-pyrazole-5-carboxylate (0.098 g, 11% yield) as a colorless oil. MS (ESI) m/z: 299.3 (M+H⁺).

Using a procedure analogous to Example B4, ethyl 3-isopropyl-1-(imidazo[1,2-a]pyridin-6-yl)-1H-pyrazole-5-carboxylate (0.098 g, 0.33 mmol) was converted to 3-isopropyl-1-(imidazo[1,2-a]pyridin-6-yl)-1H-pyrazole-5-carboxylic acid (0.087 g, 98% yield) as a white solid. MS (ESI) m/z: 271.0 (M+H⁺).

### Example B22

To a stirring suspension of 6-aminobenzothiazole (0.500 g, 3.33 mmol) in conc. HCl (5 ml) at 0-5° C. was added a solution of NaNO₂ (0.276 g, 3.99 mmol) in H₂O (5 ml). The mixture was stirred at 0-5° C. for 75 min until a clear yellow solution was obtained. To this was then added a solution of SnCl₂.2H₂O (2.76 g, 13.3 mmol) in conc. HCl (5 ml). After completing the addition, the suspension was stirred at RT for 2 h. 4-Methyl-3-oxopentanenitrile (0.444 g, 3.99 mmol) and EtOH (50 ml) were added and the reaction was stirred with heating at 75° C. After 18 h, the completed reaction was cooled to RT and concentrated to an aqueous residue. This was chilled thoroughly in ice and made strongly basic (pH 12-13) by the addition of 6M NaOH. While still cold the mixture was extracted with EtOAc (2×). The combined organics were washed with H₂O (2×), brine (1×), dried (MgSO₄), filtered and evaporated to afford crude 1-(benzo[d]thiazol-6-yl)-3-isopropyl-1H-pyrazol-5-amine (0.8 g, 93% yield) as an oil which was used as is in the next reaction. ¹H NMR (400 MHz, DMSO-d₆) δ 9.36 (s, 1H), 8.30 (d, J=2.4 Hz, 1H); 8.10 (d, J=8.8 Hz, 1H), 7.74 (dd, J=2.4 and 8.8 Hz, 1H), 5.36 (s, 1H), 5.33 (brs, 2H), 2.76 (septet, J=6.8 Hz, 1H), 1.17 (d, J=6.8 Hz, 6H); MS (ESI) m/z: 259.0 (M+H⁺).

To a stirring solution of 1-(benzo[d]thiazol-6-yl)-3-isopropyl-1H-pyrazol-5-amine (0.80 g, 3.1 mmol) and pyridine (0.51 ml, 6.2 mmol) in CH₂Cl₂ (30 ml) at RT was added Troc-Cl (0.51 ml, 3.7 mmol). After 2 h, the completed reaction was washed with 10% CuSO₄ (2×), H₂O (1×), brine (1×), dried (MgSO₄), evaporated and purified by flash column chromatography (EtOAc/hexanes) to afford 2,2,2-trichloroethyl 1-(benzo[d]thiazol-6-yl)-3-isopropyl-1H-pyrazol-5-ylcarbamat (0.31 g, 23% yield) as an oil. MS (ESI) m/z: 433.0 (M+H⁺), 435.0 (M+2+H⁺).

### Example B23

1-Methyl-5-nitro-1H-benzo[d]imidazole (prepared as described in WO 2005/092899; 1.14 g, 6.43 mmol) in EtOH (50 ml) was stirred under H₂ (1 atm) at RT in the presence of 10% Pd/C (50 wt % H₂O, 1.37 g, 0.643 mmol). After 18 h, the completed reaction was filtered on Celite, rinsing forward with EtOH. The combined filtrates were concentrated to afford crude 1-methyl-1H-benzo[d]imidazol-5-amine (1.02 g, 108% yield) as a dark orange oil which was used as is in the next reaction. ¹H NMR (400 MHz, DMSO-d₆) δ 7.87 (s, 1H), 7.17 (d, J=8.4 Hz, 1H), 6.75 (d, J=2.0 Hz, 1H), 6.59 (dd, J=2.0 and 8.4 Hz, 1H), 4.73 (brs, 2H), 3.69 (s, 3H); MS (ESI) m/z: 148.0 (M+H⁺).

Using a procedure analogous to Example B22, 1-methyl-1H-benzo[d]imidazol-5-amine (0.50 g, 3.4 mmol), NaNO₂ (0.28 g, 4.1 mmol), SnCl₂.2H₂O (2.8 g, 14 mmol) and 4-methyl-3-oxopentanenitrile (0.45 g, 4.1 mmol) were combined to afford crude 3-isopropyl-1-(1-methyl-1H-benzo[d]imidazol-5-yl)-1H-pyrazol-5-amine (0.63 g, 73% yield) as a foam which was used as is in the next reaction. ¹H NMR (400 MHz, DMSO-d₆): δ 8.22 (s, 1H), 7.72 (dd, J=0.40 and 1.2 Hz, 1H), 7.60 (dd, J=0.40 and 8.4 Hz, 1H), 7.42 (dd, J=2.0 and 8.4 Hz, 1H), 5.32 (s, 1H), 5.08 (brs, 2H), 3.85 (s, 3H), 2.75 (septet, J=6.8 Hz, 1H), 1.16 (d, J=6.8 Hz, 6H); MS (ESI) m/z: 250.0 (M+H⁺).

Using a procedure analogous to Example B22, 3-isopropyl-1-(1-methyl-1H-benzo[d]imidazol-5-yl)-1H-pyrazol-5-amine (0.63 g, 2.5 mmol) was converted to 2,2,2-trichloroethyl 3-isopropyl-1-(1-methyl-1H-benzo[d]imidazol-5-yl)-1H-pyrazol-5-ylcarbamate (0.5 g, 47% yield) and isolated as an oil. ¹H NMR (400 MHz, DMSO-d₆) δ 9.86 (brs, 1H), 8.24 (s, 1H), 7.67 (brs, 1H), 7.62 (d, J=8.4 Hz, 1H), 7.36 (dd, J=2.0 and 8.4 Hz, 1H), 6.23 (s, 1H), 4.81 (s, 2H), 3.85 (s, 3H), 2.90 (septet, J=6.8 Hz, 1H), 1.22 (d, J=6.8 Hz, 6H); MS (ESI) m/z: 430.0 (M+H⁺), 432.0 (M+2+H⁺).

### Example B24

To a stirring solution of 1-(2-(benzyloxycarbonyl)-1,2,3,4-tetrahydroisoquinolin-6-yl)-3-tert-butyl-1H-pyrazole-5-carboxylic acid from Example B1 (0.320 g, 0.738 mmol, 1.0 eq) and TEA (0.118 ml, 0.849 mmol, 1.15 eq) in 1,4-dioxane (7.5 ml) at 20° C. was added DPPA (0.183 ml, 0.849 mmol, 1.15 eq). After 30 min, 2,2,2-trichloroethanol (1.0 ml, 10.4 mmol, 14 eq) was added and the reaction was stirred with heating at 100° C. After 4 h, the completed reaction was diluted with brine and extracted with EtOAc (2×). The combined organics were washed with 5% citric acid (1×), satd. NaHCO₃ (1×) and brine (1×), dried (MgSO₄), concentrated in vacuo and purified by silica gel chromatography to afford benzyl 6-(3-tert-butyl-5-((2,2,2-trichloroethoxy)carbonyl)amino-1H-pyrazol-1-yl)-3,4-dihydroisoquinoline-2(1H)-carboxylate (0.260 g, 61% yield) as an oil. MS (ESI) m/z: 579.0 (M+H⁺), 581.0 (M+2+H⁺).

### Example B25

Using the procedure of Example B26, 3-isopropyl-1-(quinolin-6-yl)-1H-pyrazol-5-amine from Example B9 (1.00 g, 4.0 mmol), lithium bis(trimethylsilyl)amide (1.0 M in THF, 7.9 mL, 7.9 mmol) and isopropenyl chloroformate (0.48 mL, 4.4 mmol) were combined to provide prop-1-en-2-yl 3-isopropyl-1-(quinolin-6-yl)-1H-pyrazol-5-ylcarbamate (0.85 g, 65% yield). MS (ESI) m/z: 337.2 (M+H⁺).

### Example B26

A solution of 5-tert-butyl-2-quinolin-6-yl-2H-pyrazol-3-ylamine from Example B3 (1.00 g, 3.8 mmol) in THF (20 mL) was cooled to −78° C. and treated with lithium bis(trimethylsilyl)amide (1.0 M in THF, 7.5 mL, 7.5 mmol). The resultant mixture was stirred at −78° C. for 30 min. Isopropenyl chloroformate (0.45 mL, 0.41 mmol) was added and stirring was continued at −78° C. for 30 min. The reaction mixture was quenched at −78° C. with aq HCl (2 N, 4 mL, 8 mmol), was warmed to RT and partitioned between water (200 mL) and EtOAc (200 mL). The organic layer was separated, washed with brine, dried (MgSO₄), concentrated in vacuo and purified by silica gel chromatography to provide prop-1-en-2-yl 3-tert-butyl-1-(quinolin-6-yl)-1H-pyrazol-5-ylcarbamate (0.5 g, 38% yield). MS (ESI) m/z: 351.2 (M+H⁺).

### Example B27

4-Fluoro-3-nitrophenylboronic acid (0.9 g, 4.9 mmol) was dissolved in CH₂Cl₂ (10 mL) and pyridine (1 mL) with MS (activated 4A) and dried for 6 hours. A mixture of 4-fluoro-3-nitrophenylboronic acid, tert-butyl 3-isopropyl-1H-pyrazole-5-carboxylate (1.0 g, 4.9 mmol), copper(II) acetate (0.88 g, 4.9 mmol) and molecular sieves (4A activated, powder) was stirred at RT for 7 days open to the air. The reaction mixture was filtered through a pad of Celite. The filtrate was concentrated in vacuo and purified by silica gel column chromatography (EtOAc/hexane) to obtain tert-butyl 1-(4-fluoro-3-nitrophenyl)-3-isopropyl-1H-pyrazole-5-carboxylate (0.74 g, 44% yield). MS (ESI) m/z: 350.3 (M+H⁺).

To a solution of tert-butyl 1-(4-fluoro-3-nitrophenyl)-3-isopropyl-1H-pyrazole-5-carboxylate (0.74 g, 2.1 mmol) in THF/water (12 mL) was added LiOH (300 mg, 13 mmol) and H₂O₂ (30% wt, 0.96 mL). The reaction mixture was heated overnight at 60° C. Na₂S₂O₃ solution was added until the peroxide test (starch-iodide paper) was negative. Acetic acid was added until the pH was 4-5. The solution was extracted with EtOAc and the organic layer was washed with brine, dried (MgSO₄), concentrated in vacuo and purified by silica gel column chromatography (EtOAc/hexanes) to obtain tert-butyl 1-(4-hydroxy-3-nitrophenyl)-3-isopropyl-1H-pyrazole-5-carboxylate (0.27 g, 37% yield). MS (ESI) m/z: 348.3 (M+H⁺).

To a solution of tert-butyl 1-(4-hydroxy-3-nitrophenyl)-3-isopropyl-1H-pyrazole-5-carboxylate (0.27 g, 0.78 mmol) in ethyl acetate/methanol (1:1, 10 mL) was added palladium on carbon (30 mg) and the mixture was hydrogenated (50 psi) overnight under Parr. The solution was filtered and washed with methanol. The combined filtrate was concentrated to afford tert-butyl 1-(3-amino-4-hydroxyphenyl)-3-isopropyl-1H-pyrazole-5-carboxylate. The crude tert-butyl 1-(3-amino-4-hydroxyphenyl)-3-isopropyl-1H-pyrazole-5-carboxylate was treated with 25% TFA in CH₂Cl₂ (2 mL) and stirred overnight at RT. The solvent was evaporated to obtain 1-(benzo[d]oxazol-5-yl)-3-tert-butyl-1H-pyrazole-5-carboxylic acid. To a solution of 1-(benzo[d]oxazol-5-yl)-3-tert-butyl-1H-pyrazole-5-carboxylic acid in xylenes (3 mL) was added triethyl orthoformate (0.16 mL, 0.96 mmol) and a catalytic amount of PPTS. The reaction mixture was heated at 140° C. for 4 hours. The solvent was evaporated and the residue was treated with methylene chloride with stirring for 1 hour. The resulting solid was filtered and washed with methylene chloride to obtain 1-(benzo[d]oxazol-5-yl)-3-isopropyl-1H-pyrazole-5-carboxylic acid (0.1 g, 45% yield: for three steps). MS (ESI) m/z: 272.0 (M+H⁺).

### Example B28

In toluene (8 mL) was placed 1-(diphenylmethylene)hydrazine (1.00 g, 5.10 mmol), palladium acetate (10.4 mg, 0.0464 mmol) and 2-(diphenylphosphino)-1-(2-(diphenylphosphino)naphthalen-1-yl)naphthalene (44 mg, 0.0696 mmol) and the reaction was stirred at 100° C. under Ar for 5 min and then cooled to RT. To this dark purple solution was added 6-bromoquinoxaline (970 mg, 4.64 mmol), sodium t-butoxide (624 mg, 6.50 mmol) and toluene (2 mL). The reaction was placed under Ar and warmed to 100° C. for 5 hrs, cooled to RT and stirred overnight. The reaction was diluted with ether (50 mL) and water (30 mL) and filtered through a Celite pad. The pad was washed with ether (20 mL) and water (20 mL). The combined organic layers were washed with brine (50 mL), dried (Na₂SO₄), concentrated in vacuo and purified by chromatography (ethyl acetate/hexanes) to give 1-(diphenylmethylene)-2-(quinoxalin-6-yl)hydrazine (305 mg, 20% yield) as a bright yellow foam. ¹H NMR (300 MHz, DMSO-d₆) δ 7.35-7.41 (m, 5H), 7.51-7.53 (m, 2H), 7.58-7.65 (m, 3H), 7.75 (s, 1H), 7.89 (s, 2H), 8.61 (s, 1H), 8.74 (s, 1H), 9.60 (s, 1H); MS (ESI) m/z: 325.0 (M+H⁺).

In ethanol (10 mL) was placed 1-(diphenylmethylene)-2-(quinoxalin-6-yl)hydrazine (300 mg, 0.925 mmol), pivaloylacetonitrile (156 mg, 1.25 mmol) and p-toluenesulfonic acid hydrate (704 mg, 3.70 mmol). The reaction was brought to reflux and stirred overnight. The reaction was cooled to RT, diluted with ethyl acetate (50 mL) and saturated sodium bicarbonate (50 mL). The organic phase was separated, washed with 1N NaOH (30 mL) and brine (30 mL), dried (Na₂SO₄), concentrated in vacuo and purified by chromatography (Si-25 column, ethyl acetate/hexanes) to give a tan foam, identified as 3-tert-butyl-1-(quinoxalin-6-yl)-1H-pyrazol-5-amine (57 mg, 23% yield). MS (ESI) m/z: 268.2 (M+H⁺).

### Example B29

To a solution of phenethylamine (60.5 g, 0.5 mol) and Na₂CO₃ (63.6 g, 0.6 mol) in EtOAc/H O (800 mL, 4:1) was added ethyl chloroformate, dropwise, (65.1 g, 0.6 mol) at 0° C. during a period of 1 h. The mixture was warmed to RT and stirred for an additional 1 h. The organic phase was separated and the aqueous layer was extracted with EtOAc. The combined organic phases were washed with H₂O and brine, dried (Na₂SO₄), concentrated in vacuo and purified by flash chromatography to afford ethyl phenethylcarbamate (90.2 g). ¹H NMR (400 MHz, CDCl₃) δ 7.32-7.18 (m, 5H), 4.73 (brs, 1H), 4.14-4.08 (q, J=6.8 Hz, 2H), 3.44-3.43 (m, 2H), 2.83-2.79 (t, J=6.8 Hz, 2H), 1.26-1.21 (t, J=6.8 Hz, 3H).

A suspension of ethyl phenethylcarbamate (77.2 g, 40 mmol) in polyphosphoric acid (300 mL) was heated to 140-160° C. and stirred for 2.5 h. The reaction mixture was cooled to RT, carefully poured into ice-H₂O and stirred for 1 h. The aqueous solution was extracted with EtOAc (3×300 mL). The combined organic phases were washed with H₂O, 5% K₂CO₃ and brine, dried (Na₂SO₄), concentrated in vacuo and purified by flash chromatography to afford 3,4-dihydro-2H-isoquinolin-1-one (24 g). ¹H NMR (400 MHz, DMSO-d₆) δ 7.91 (brs, 1H), 7.83 (d, J=7.5 Hz, 1H,), 7.43 (t, J=7.5 Hz, 1H), 7.33-7.25 (m, 2H), 3.37-3.32 (m, 2H), 2.87 (t, J=6.6 Hz, 2H).

To an ice-salt bath cooled mixture of cone. HNO₃ and cone. H₂SO₄ (200 mL, 1:1) was added 4-dihydro-2H-isoquinolin-1-one (15 g, 0.102 mol) dropwise over min. After stirring for 2 h, the resulting mixture was poured into ice-H₂O and stirred for 30 min. The precipitate was filtered, washed with H₂O, and dried in air to afford 7-nitro-3,4-dihydro-2H-isoquinolin-1-one (13 g). ¹H NMR (300 MHz, DMSO-d₆) δ 8.53 (d, J=2.4 Hz, 1H,), 8.31 (d, J=2.4 Hz, 1H), 8.29 (d, J=2.4 Hz, 1H), 7.62 (d, J=8.4 Hz, 1H), 3.44-3.39 (m, 2H), 3.04 (t, J=6.6 Hz, 2H).

A suspension of 7-nitro-3,4-dihydro-2H-isoquinolin-1-one (11.6 g, 60 mmol) and 10% Pd/C (1.2 g,) in MeOH was stirred overnight at RT under H₂ (40 psi). The mixture was filtered through Celite® and washed with MeOH. The filtrate was evaporated under vacuum to afford 8.2 g of 7-amino-3,4-dihydro-2H-isoquinolin-1-one, which was used without further purification.

To a suspension of 7-amino-3,4-dihydro-2H-isoquinolin-1-one (8.1 g, 50 mmol) in cone. HCl (100 mL) in an ice-H₂O bath was added a solution of NaNO₂ (3.45 g, 50 mmol) in H₂O dropwise at such a rate that the reaction mixture never rose above 5° C. A solution of SnCl₂.2H₂O (22.5 g, 0.1 mol) in cone. HCl (150 mL) was added dropwise after 30 min. The resulting mixture was stirred for another 2 h at 0° C. The precipitate was collected by suction, washed with ether to afford 7-hydrazino-3,4-dihydro-2H-isoquinolin-1-one (8.3 g), which was used for the next reaction without further purification.

A mixture of 7-hydrazino-3,4-dihydro-2H-isoquinolin-1-one (8.0 g, 37.6 mmol) and 4,4-dimethyl-3-oxo-pentanenitrile (5.64 g, 45 mmol) in EtOH (100 mL) and conc. HCl (10 mL) was heated at reflux overnight. After removal of the solvent, the residue was washed with ether to afford 7-(5-amino-3-t-butyl-pyrazol-1-yl)-3,4-dihydro-2H-isoquinolin-1-one hydrochloride as a yellow solid (11.5 g, 96% yield), which was used without further purification.

To a solution of 7-(5-amino-3-t-butyl-pyrazol-1-yl)-3,4-dihydro-2H-isoquinolin-1-one hydrochloride (0.5 g, 1.76 mmol) in CH₂Cl₂ (25 mL) were added pyridine (0.22 mL) and trichloroethyl chloroformate (0.27 mL) at 0° C. and the mixture was stirred overnight at RT. LCMS showed the reaction was incomplete. Pyridine (0.25 mL) and TROC-Cl (0.25 mL) were added and then the mixture stirred at RT for 2 hours. The reaction mixture was diluted with CH₂Cl₂, the organic layer was washed with 3M HCl and brine, dried (Na₂SO₄) and concentrated in vacuo. The residue was dissolved in EtOAc and hexane was added. The solid was filtered to obtain 2,2,2-trichloroethyl 3-tert-butyl-1-(1-oxo-1,2,3,4-tetrahydroisoquinolin-7-yl)-1H-pyrazol-5-ylcarbamate (0.46 g, 57% yield). MS (ESI) m/z: 458.0 (M+H⁺).

### Example B30

To a solution of 7-(5-amino-3-t-butyl-pyrazol-1-yl)-3,4-dihydro-2H-isoquinolin-1-one hydrochloride from Example B29 (20 g, 0.070 mol) in THF (400 mL) was added LAH (15 g, 0.395 mol) in portions at 0-5° C. The resulting mixture was heated at reflux overnight, followed by the addition of 10% NaOH solution. After stirring for 1 h at RT, Boc₂O (23 g, 0.106 mol) was added and the solution stirred overnight. After filtration, the filtrate was concentrated to afford the crude product, which was purified by reverse phase chromatography to give 7-(5-amino-3-t-butyl-pyrazol-1-yl)-3,4-dihydro-1H-isoquinoline-2-carboxylic acid t-butyl ester (12 g, 75% yield). ¹H NMR (300 MHz, DMSO-d₆) δ 7.32 (s, 1H), 7.29 (d, J=2.7 Hz, 1H), 7.18 (d, J=8.4 Hz, 1H), 5.32 (s, 1H), 5.15 (s, 1H), 4.51 (s, 2H), 3.52 (t, J=5.6 Hz, 2H), 2.75 (t, J=5.6 Hz, 2H), 1.40 (s, 9H), 1.17 (s, 9H); MS (ESI) m/z: 371(M+H⁺).

To a stirring solution of tert-butyl 7-(5-amino-3-tert-butyl-1H-pyrazol-1-yl)-3,4-dihydroisoquinoline-2(1H)-carboxylate (0.50 g, 1.35 mmol) and Troc-Cl (0.19 ml, 1.38 mmol) in EtOAc (15 mL) was added satd. NaHCO₃ (2.75 ml, 2.02 mmol). The resulting biphasic mixture was stirred at RT for 5 h. The layers were separated and the organic washed with sat'd. NaHCO₃ (1×) and brine (1×), dried (Na₂SO₄) and concentrated in vacuo to obtain tert-butyl 7-(3-tert-butyl-5-((2,2,2-trichloroethoxy)carbonyl)-1H-pyrazol-1-yl)-3,4-dihydroisoquinoline-2(1H)-carboxylate (0.69 g, 94% yield). MS (ESI) m/z: 545.0 (M+H⁺).

### Example 1

A solution of Example B3 (7.0 g, 15.8 mmol), Example A2 (4.14 g, 15.8 mmol) and DIEA (4.5 g, 34.9 mmol) in DMSO (70 ml) was heated in an oil-bath at 70° C. for 8 hrs. The reaction mixture was poured into water (500 ml), stirred overnight and the solids were separated by filtration. Successive crystallization of the crude product from toluene and acetone provided 1-(3-tert-butyl-1-(quinolin-6-yl)-1H-pyrazol-5-yl)-3-(2-fluoro-4-(2-(methylcarbamoyl)pyridin-4-yloxy)phenyl)urea as a white crystalline solid (4.06 g, 46% yield). ¹H NMR (DMSO-d₆) δ 8.90 (m, 2H), 8.79 (m, 1H), 8.52 (m, 2H), 8.2 (m, 3H), 7.96 (dd, J=9, 2 Hz, 1H), 7.63 (dd, J=8, 4 Hz, 1H), 7.40 (br s, 1H), 7.30 (dd, J=3, 12 Hz, 1H), 7.17 (m, 1H), 7.05 (d, J=9 Hz, 1H), 6.50 (s, 1H), 2.80 (d, J=5 Hz), 1.32 (s, 9H); MS (ESI) m/z: 554 (M+H⁺). The free base was treated with 0.1 M HCl to provide 1-(3-tert-butyl-1-(quinolin-6-yl)-1H-pyrazol-5-yl)-3-(2-fluoro-4-(2-(methylcarbamoyl)pyridin-4-yloxy)phenyl)urea bis hydrochloride salt as a pale yellow fluffy solid (2.40 g). ¹H NMR (DMSO-d₆) δ 9.56 (s, 1H), 9.26 (m, 2H), 9.10 (d, J=8 Hz, 1H), 8.85 (m, 1H), 8.55 (m, 2H), 8.46 (d, J=9 Hz, 1H), 8.33 (dd, J=9, 2 Hz, 1H), 8.11 (t, J=9 Hz, 1H), 8.03 (dd, dd, J=9, 2 Hz, 1H), 7.46 (d, J=3 Hz, 1H), 7.30 (dd, J=3, 12 Hz, 1H), 7.20 (dd, J=3, 6 Hz, 1H), 7.04 (brd, J=7 Hz, 1H), 6.49 (s, 1H), 2.80 (d, J=4.5 Hz), 1.33 (s, 9H).

### Example 2

Example B1 (142 mg, 0.33 mmol) and Et₃N (0.15 mL, 0.72 mmol) were combined in dioxane (3 mL). DPPA (0.13 mL, 0.59 mmol) was added and the reaction mixture was stirred at RT for 90 min. Example A2 (94 mg, 0.36 mmol) was added and the resultant mixture was heated to 95° C. for 4 h. The reaction mixture was concentrated in vacuo and purified by silica gel chromatography to provide benzyl 6-(3-tert-butyl-5-(3-(2-fluoro-4-(2-(methylcarbamoyl)pyridin-4-yloxy)phenyl)ureido)-1H-pyrazol-1-yl)-3,4-dihydroisoquinoline-2(1H)-carboxylate (95 mg, 42% yield). ¹H NMR (400 MHz, DMSO-d₆) δ 9.00 (br s, 1H), 8.84 (s, 1H), 8.79 (q, J=4.8 Hz, 1H), 8.52 (d, J=5.6 Hz, 1H), 8.20 (t, J=9.2 Hz, 1H), 7.40-7.28 (m, 10H), 7.17 (dd, J=5.6, 2.8 Hz, 1H), 7.05 (m, 1H), 6.40 (s, 1H), 5.14 (s, 2H), 4.66 (m, 2H), 3.68 (m, 2H), 2.91 (t, J=5.6 Hz, 2H), 2.79 (d, J=4.8 Hz, 3H), 1.27 (s, 9H); MS (ESI) m/z: 692.2 (M+H⁺).

A solution of benzyl 6-(3-tert-butyl-5-(3-(2-fluoro-4-(2-(methylcarbamoyl)pyridin-4-yloxy)phenyl)ureido)-1H-pyrazol-1-yl)-3,4-dihydroisoquinoline-2(1H)-carboxylate (93 mg, 0.13 mmol) in methanol (3 mL) was treated with 10% Pd/C (50% wet, 74 mg, 0.03 mmol) and formic acid (88%, 0.60 mL, 14 mmol). The resultant reaction mixture was stirred for 90 min and filtered through Celite, washing forward with methanol. The filtrate was concentrated in vacuo and purified on silica gel to provide 1-(3-tert-butyl-1-(1,2,3,4-tetrahydroisoquinolin-6-yl)-1H-pyrazol-5-yl)-3-(2-fluoro-4-(2-(methylcarbamoyl)pyridin-4-yloxy)phenyl)urea (42 mg, 56% yield). The product was treated with aqueous HCl (0.1 M, 0.75 mL) to provide 1-(3-tert-butyl-1-(1,2,3,4-tetrahydroisoquinolin-6-yl)-1H-pyrazol-5-yl)-3-(2-fluoro-4-(2-(methylcarbamoyl)pyridin-4-yloxy)phenyl)urea hydrochloride. ¹H NMR (400 MHz, DMSO-d₆) δ 9.38 (br s, 2H), 9.10 (d, J=1.8 Hz, 1H), 9.05 (s, 1H), 8.80 (m, 1H), 8.53 (d, J=5.4 Hz, 1H), 8.15 (t, J=9.1 Hz, 1H), 7.46-7.34 (m, 4H), 7.32 (dd, J=11.6, 2.8 Hz, 1H), 7.18 (m, 1H), 7.05 (m, 1H), 6.39 (s, 1H), 4.33 (br s, 2 H), 3.40 (2H obscured by H₂O), 3.09 (t, J=6.0 Hz, 2H), 2.79 (d, J=5.0 Hz, 3H), 1.28 (s, 9H); MS (ESI) m/z: 558.3 (M+H⁺).

### Example 3

Using general method A, Example B4 (80 mg, 0.27 mmol), Example A1 (0.18 g, 0.68 mmol), triethyl amine (30 mg, 0.30 mmol), and DPPA (82 mg, 0.30 mmol) were combined to yield 1-(3-tert-butyl-1-(quinolin-6-yl)-1H-pyrazol-5-yl)-3-(3-fluoro-4-(2-(methylcarbamoyl)pyridin-4-yloxy)phenyl)urea which was treated with 3M HCl/EtOAc to obtain its HCl salt (125 mg, 78% yield). ¹H NMR (400 MHz, DMSO-d₆): δ 9.79 (brm, 1H), 9.16 (brm, 1H), 9.05 (brm, 1H), 8.93 (brm, 1H), 8.79 (brm, 1H), 8.53 (d, J=5.6 Hz, 1H), 8.42 (brm, 1H), 8.33 (brm, 1H), 8.22 (brm, 1H), 7.91 (brm, 1H), 7.68 (dd, J=2.4, and 14.4 Hz, 1H), 7.37 (d, J=2.4 Hz, 1H), 7.34 (t, J=9.2 Hz, 1H), 7.19 (brm, 1H), 6.49 (s, 1H), 2.79 (d, J=5.2 Hz, 3H), 1.31 (s, 9H); MS (ESI) m/z: 554.2 (M+H⁺).

### Example 4

To a solution of Example B8 (0.132 g, 0.30 mmol) in THF (1.0 ml) were added Example A2 (0.083 g, 0.315 mmol) and 1-methylpyrrolidine (2.6 mg, 0.03 mmol). The mixture was heated at 55° C. overnight. Solvent was removed and the residue was dissolved in MeOH (4.5 ml), to which 3M HCl/EtOAc (1.3 ml, 3.8 mmol) was added. The resulting mixture was stirred at RT overnight, followed by heating at 55° C. for 3 h. The reaction mixture was concentrated to dryness, diluted with sat'd. NaHCO₃ (7 ml) and extracted with EtOAc (3×20 ml). The combined organic layers was washed with sat'd. NaHCO₃ (7 ml), H₂O (7 ml) and brine (7 ml), dried (MgSO₄), concentrated in vacuo and purified by chromatography to afford 1-(3-tert-butyl-1-(1H-indazol-5-yl)-1H-pyrazol-5-yl)-3-(2-fluoro-4-(2-(methylcarbamoyl)pyridin-4-yloxy)phenyl)urea (80 mg, 49% yield) as a white solid. This was converted to corresponding HCl salt by reacting with HCl (4.0 M in dioxane, 1.0 eq.). ¹H NMR (DMSO-d₆) δ 9.17 (s, 1H), 9.13 (s, 1H), 8.99 (m, 1H), 8.56 (d, J=5.6 Hz, 1H), 8.23-8.18 (m, 2H), 7.96 (d, J=2.0 Hz, 1H), 7.72 (d, J=8.8 Hz, 1H), 7.58 (d, J=2.4 Hz, 1H), 7.49 (dd, J=8.8, 1.6 Hz, 1H), 7.32 (dd, J=11.6, 2.8 Hz, 1H), 7.24 (dd, J=6.0, 3.0 Hz, 1H), 7.07 (dd, J=8.8, 1.6 Hz, 1H), 6.47 (s, 1H), 2.81 (d, J=4.8 Hz, 3H), 1.30 (s, 9H); MS (ESI) m/z: 543.2 (M+H⁺).

### Example 5

Using general method A, Example B4 (80 mg, 0.27 mmol) and Example A6 (99 mg, 0.38 mmol) were combined to provide 1-(3-tert-butyl-1-(quinolin-6-yl)-1H-pyrazol-5-yl)-3-(3-methyl-4-(2-(methylcarbamoyl)pyridin-4-yloxy)phenyl)urea (149 mg, 99% yield). ¹H NMR (400 MHz, DMSO-d₆) δ 9.08 (s, 1 H), 8.97 (dd, J=4.1, 1.2 Hz, 1H), 8.77 (q, J=4.6 Hz, 1H), 8.62 (s, 1H), 8.51-8.48 (m, 2H), 8.20-8.16 (m, 2H), 7.97 (d, J=8.9, 2.0 Hz, 1H), 7.63 (dd, J=8.5, 4.2 Hz, 1H), 7.46 (d, J=2.4 Hz, 1H), 7.32 (dd, J=8.7, 2.5 Hz, 1H), 7.27 (d, J=2.6 Hz, 1 H), 7.08 (m, 1H), 7.06 (d, J=8.7 Hz, 1H), 6.47 (s, 1H), 2.78 (d, J=4.6 Hz, 3H), 2.04 (s, 3H), 1.33 (s, 9H); MS (ESI) m/z: 550.2 (M+H⁺).

### Example 6

Using a procedure analogous to Example 1, Example B3 (0.19 g, 0.43 mmol) and Example A7 (0.11 g, 0.43 mmol) were combined to provide 1-(3-tert-butyl-1-(quinolin-6-yl)-1H-pyrazol-5-yl)-3-(4-(2-carbamoylpyridin-4-yloxy)-2-fluorophenyl)urea hydrochloride (0.160 g, 64% yield). ¹H NMR (DMSO-d₆) δ 9.55 (s, 1H), 9.27-9.24 (m, 2H), 9.10 (d, J=8.8 Hz, 1H), 8.56-8.54 (m, 2H), 8.46 (d, J=9.2 Hz, 1H), 8.32 (dd, J=9.6, 2.4 Hz, 1H), 8.27 (s, 1H), 8.13 (t, J=9.2 Hz, 1H), 8.04 (dd, J=8.4, 5.2 Hz, 1H), 7.85 (s, 1H), 7.52 (d, J=2.4 Hz, 1H), 7.32 (dd, J=11.6, 2.4 Hz, 1H), 7.24 (dd, J=6.0, 2.8 Hz, 1H), 7.05 (dq, J=8.8, 1.2 Hz, 1H), 6.50 (s, 1H), 1.33 (s, 9H); MS (ESI) m/z: 540.3 (M+H⁺).

### Example 7

### Example B3 (0.12 g, 0.27 mmol), Example A9 (63 mg, 0.27 mmol) and DIEA (77 mg, 0.60 mmol) were combined in DMSO (1 mL) and heated overnight at 50-55° C. Water was added (50 mL) and the mixture was extracted with EtOAc (3×100 mL), dried (MgSO₄), concentrated in vacuo and purified by silica gel column chromatography (EtOAc/hexane) to obtain 1-(3-tert-butyl-1-(quinolin-6-yl)-1H-pyrazol-5-yl)-3-(2-fluoro-4-(2-(methylamino)pyridin-4-yloxy)phenyl)urea. The solid was treated with 0.100M HCl (2 equiv.) to obtain and 1-(3-tert-butyl-1-(quinolin-6-yl)-1H-pyrazol-5-yl)-3-(2-fluoro-4-(2-(methylamino)pyridin-4-yloxy)phenyl)urea hydrochloride (52 mg, 32% yield). ¹H NMR (400 MHz, DMSO-d₆) δ 9.23 (brs, 1H), 9.17 (brs, 1H), 9.06 (brm, 1H), 8.66 (brm, 1H), 8.53 (brs, 1H), 8.0-8.3 (m, 4H), 7.92 (d, J=6.8 Hz, 1H), 7.74 (m, 1H), 7.35 (dd, J=2.8, and 11.6 Hz, 1H), 7.07 (m, 1H), 6.62 (d, J=6.4 Hz, 1H), 6.48 (s, 1H), 6.18 (brs, 1H), 2.88 (d, J=4.8 Hz, 2H), 1.32 (s, 9H); LC-MS (EI) m/z: 526.2 (M+H⁺).

### Example 8

Using a procedure analogous to Example 1, Example B6 (0.178 g, 0.335 mmol), Example A10 (0.0840 g, 0.352 mmol) and DIEA (0.0701 ml, 0.402 mmol) were combined, purified by flash column chromatography (EtOAc/hexanes) and purified a second time by flash column chromatography (EtOAc/CH₂Cl₂) to afford t-butyl 5-(3-t-butyl-5-(3-(5-(5-chloropyridin-3-yloxy)-2-fluorophenyl)ureido)-1H-pyrazol-1-yl)-1H-indazole-1-carboxylate (0.0486 g, 23% yield) as a solid. ¹H NMR (400 MHz, acetone-d₆) δ 8.52 (brd, 1H, J=2.8 Hz), 8.46 (s, 1H), 8.37 (d, 1H, J=2.0 Hz), 8.35-8.32 (m, 2H), 8.24 (dt, 1H, J=0.8 and 8.8 Hz), 8.818 (dd, 1H, J=2.8 and 6.8 Hz), 7.22 (dd, 1H, J=8.8 and 10.8 Hz), 6.81 (ddd, 1H, J=3.2, 4.0 and 8.8 Hz), 1.73 (s, 9H), 1.34 (s, 9H); MS (ESI) m/z: 620.2 (M+H⁺).

The material from the previous step (0.0486 g, 0.078 mmol) and 4M HCl in dioxane (5.0 ml) were combined at RT. A little MeOH was added to give a homogeneous solution. The mixture was heated overnight at 40° C. The completed reaction was concentrated in vacuo, dissolved in MeCN/H₂O, frozen and lyophilized to afford 1-(3-t-butyl-1-(1H-indazol-5-yl)-1H-pyrazol-5-yl)-3-(5-(5-chloropyridin-3-yloxy)-2-fluorophenyl)urea (0.0475 g, 103% yield) as the bis-HCl salt. ¹H NMR (400 MHz, DMSO-d₆) δ 9.14 (s, 1H), 8.95 (s, 1H), 8.43-8.42 (m, 1H), 8.34-8.33 (m, 1H), 8.20 (s, 1H), 8.00-7.97 (m, 1H), 7.88-7.87 (m, 1H), 7.70-7.67 (m, 1H), 7.60-7.59 (m, 1H), 7.45-7.42 (m, 1H), 7.32-7.27 (m, 1H), 6.81-6.77 (m, 1H), 6.38 (s, 1H), 1.27 (s, 9H); MS (ESI) m/z: 520.2 (M+H⁺).

### Example 9

Using a procedure analogous to Example 1, Example B7 (0.300 g, 0.550 mmol), Example A10 (0.138 g, 0.577 mmol) and DIEA (0.115 ml, 0.659 mmol) were combined and purified by flash column chromatography (EtOAc/hexanes) to afford tert-butyl 6-(3-tert-butyl-5-(3-(5-(5-chloropyridin-3-yloxy)-2-fluorophenyl)ureido)-1H-pyrazol-1-yl)-3,4-dihydroisoquinoline-2(1H)-carboxylate (0.090 g, 26% yield) as a film. ¹H NMR (400 MHz, acetone-d₆) δ 8.50 (brs, 1H), 8.36 (s, 1H), 8.35-8.32 (m, 2H), 8.19-8.16 (m, 1H), 7.47-7.46 (m, 1H), 7.38-7.36 (m, 2H), 7.31-7.29 (m, 1H), 7.27-7.22 (m, 1H), 6.83-6.79 (m, 1H), 6.46 (s, 1H), 4.63 (brs, 2H), 3.68-3.65 (m, 2H), 2.89-2.86 (m, 2H), 1.50 (s, 9H), 1.32 (s, 9H); MS (ESI) m/z: 635.2 (M+H⁺).

The material from the previous reaction (0.090 g, 0.14 mmol, 1.00 eq) and 4M HCl in dioxane (5.00 ml) were combined at 22° C. A little MeOH was added to make the mixture homogeneous. After 2.5 h, the completed reaction was concentrated in vacuo, dissolved in MeCN/H₂O, frozen and lyophilized to afford 1-(3-tert-butyl-1-(1,2,3,4-tetrahydroisoquinolin-6-yl)-1H-pyrazol-5-yl)-3-(5-(5-chloropyridin-3-yloxy)-2-fluorophenyl)urea (76 mg, 89% yield) as the bis-HCl salt. ¹H NMR (400 MHz, DMSO-d₆) δ 9.51 (brs, 2H), 9.26 (brs, 1H), 9.22 (s, 1H), 8.42-8.41 (m, 1H), 8.33-8.32 (m, 1H), 7.95-7.92 (m, 1H), 7.60-7.59 (m, 1H), 7.42-7.29 (m, 4H), 6.82-6.78 (m, 1H), 6.34 (s, 1H), 4.32-4.30 (m, 2H), 3.39-3.35 (m, 2H), 3.10-3.06 (m, 2H), 1.26 (s, 9H); MS (ESI) m/z: 535.2 (M+H⁺).

### Example 10

Using a procedure analogous to Example 1, Example B9 (0.150 g, 0.351 mmol) and Example A2 (0.101 g, 0.386 mmol) were combined to provide 1-(2-fluoro-4-(2-(methylcarbamoyl)pyridin-4-yloxy)phenyl)-3-(3-isopropyl-1-(quinolin-6-yl)-1H-pyrazol-5-yl)urea hydrochloride (0.126 g, 62% yield). ¹H NMR (DMSO-d₆) δ 9.36 (s, 1H), 9.18-9.15 (m, 2H), 8.92 (d, J=8.4 Hz, 1H), 8.85-8.80 (m, 1H), 8.53 (d, J=5.6 Hz, 1H), 8.44 (d, J=2.4 Hz, 1H), 8.36 (d, J=9.2 Hz, 1H), 8.22 (dd, J=9.2, 2.4 Hz, 1H), 8.14 (t, J=9.2 Hz, 1H), 7.92 (dd, J=8.4, 4.8 Hz, 1H), 7.42 (d, J=2.4 Hz, 1H), 7.31 (dd, J=11.6, 2.8 Hz, 1H), 7.19 (dd, J=5.6, 2.8 Hz, 1H), 7.04 (dd, J=8.8, 2.0 Hz, 1H), 6.45 (s, 1H), 2.96 (m, 1H), 2.79 (d, J=4.8 Hz, 3H), 1.28 (d, J=6.8 Hz, 6H); MS (ESI) m/z: 540.3 (M+H⁺).

### Example 11

Using a procedure analogous to Example 1, Example B10 (0.15 g, 0.363 mmol) and Example A2 (0.100 g, 0.38 mmol) were combined to provide 1-(3-ethyl-1-(quinolin-6-yl)-1H-pyrazol-5-yl)-3-(2-fluoro-4-(2-(methylcarbamoyl)pyridin-4-yloxy)phenyl)urea hydrochloride (0.120 g, 58% yield). ¹H NMR (DMSO-d₆) δ 9.42 (s, 1H), 9.21-9.18 (m, 2H), 8.96 (d, J=8.4 Hz, 1H), 8.87-8.82 (m, 1H), 8.53 (d, J=5.6 Hz, 1H), 8.48 (d, J=1.6 Hz, 1H), 8.38 (d, J=9.2 Hz, 1H), 8.25 (dd, J=9.2, 1.6 Hz, 1H), 8.14 (t, J=8.8 Hz, 1H), 7.95 (dd, J=8.0, 4.8 Hz, 1H), 7.43 (d, J=2.0 Hz, 1H), 7.31 (dd, J=12.0, 2.4 Hz, 1H), 7.19 (dd, J=5.2, 2.0 Hz, 1H), 7.05 (dt, J=8.8, 1.6 Hz, 1H), 6.44 (s, 1H), 2.79 (d, J=4.8 Hz, 3H), 2.64 (q, J=7.6 Hz, 2H), 1.25 (t, J=7.6 Hz, 3H); MS (ESI) m/z: 526.2 (M+H⁺).

### Example 12

Using a procedure analogous to Example 1, Example B3 (0.195 g, 0.441 mmol), Example A10 (0.111 g, 0.464 mmol) and DIEA (0.0923 ml, 0.530 mmol) were combined and purified first by flash column chromatography (EtOAc/hexanes) and then by reverse phase chromatography (MeCN (w/0.1% TFA)/H₂O (w/0.1% TFA)) to provide an aqueous solution of the TFA salt of the desired product. The aqueous residue was treated with satd. NaHCO₃ (pH 8) and extracted with EtOAc (3×). The combined organics were washed with brine (1×), dried (MgSO₄), and evaporated to afford product (0.0258 g, 11% yield) as the free base. The free base was treated with certified 0.1N HCl (0.97 ml, 2.0 eq) to afford 1-(3-t-butyl-1-(quinolin-6-yl)-1H-pyrazol-5-yl)-3-(5-(5-chloropyridin-3-yloxy)-2-fluorophenyl)urea (0.0262 g, 10% yield) as the bis-HCl salt. ¹H NMR (400 MHz, DMSO-d₆) δ 9.33 (s, 1H), 9.22-9.21 (m, 1H), 9.14-9.13 (m, 1H), 8.83-8.81 (m, 1H), 8.42-8.41 (m, 1H), 8.36 (brs, 1H), 8.33-8.29 (m, 2H), 8.15-8.12 (m, 1H), 7.94-7.91 (m, 1H), 7.88-7.84 (m, 1H), 7.59-7.57 (m, 1H), 7.34-7.28 (m, 1H), 6.82-6.78 (m, 1H), 6.46 (s, 1H), 1.30 (s, 9H); MS (ESI) m/z: 531.0 (M+H⁺).

### Example 13

Using a procedure analogous to Example 1, Example B3 (100 mg, 0.226 mmol), DIEA (73 mg, 0.566 mmol) and Example A18 (63 mg, 0.25 mmol) were combined to yield 1-(3-tert-butyl-1-(quinolin-6-yl)-1H-pyrazol-5-yl)-3-(2-fluoro-5-(2-(methylthio)pyrimidin-4-yloxy)phenyl)urea hydrochloride (61 mg, 50% yield). ¹H-NMR (DMSO-d₆) δ 1.30 (s, 9H), 2.50 (s, 3H), 6.47 (s, 1H), 6.76 (d, 1H), 6.86-6.90 (m, 1H), 7.29-7.34 (m, 1H), 7.92-7.98 (m, 2H), 8.20-8.23 (m, 1H), 8.37 (d, 1H), 8.44 (s, 1H), 8.50 (d, 1H), 8.95 (d, 1H), 9.19-9.20 (m, 1H), 9.28 (s, 1H), 9.46 (s, 1H); MS (ESI) m/z: 544.2 (M+H⁺).

### Example 14

Using a procedure analogous to Example 1, Example B3 (0.10 g, 0.23 mmol), Example A12 (53 mg, 0.23 mmol) and DIEA (64 mg, 0.50 mmol) were combined and purified by reverse phase column chromatography to obtain 1-(3-tert-butyl-1-(quinolin-6-yl)-1H-pyrazol-5-yl)-3-(2-fluoro-5-(6-(hydroxymethyl)pyridin-3-yloxy)phenyl)urea TFA salt. The residue was dissolved in 3M HCl and co-evaporated with isopropyl alcohol (3×). EtOAc was added to the residue and the solid was filtered, washed with EtOAc, and dried under vacuum to obtain 1-(3-tert-butyl-1-(quinolin-6-yl)-1H-pyrazol-5-yl)-3-(2-fluoro-5-(6-(hydroxymethyl)pyridin-3-yloxy)phenyl)urea HCl salt (40 mg, 34% yield). ¹H NMR (400 MHz, DMSO-d₆) δ 9.15 (brm, 1H), 9.05 (brm, 1H), 8.63 (brm, 1H), 8.32 (brm, 1H), 8.23 (brm, 2H), 8.03 (m, 1H), 7.90 (m, 1H), 7.73 (brm, 1H), 7.56 (m, 2H), 7.28 (dd, J=9.2, 12.4 Hz, 1H), 6.74 (m, 1H), 6.44 (s, 1H), 4.60 (m, 2H), 1.30 (s, 9H); MS (ESI) m/z: 527.2 (M+H⁺).

### Example 15

Using a procedure analogous to Example 1, Example B9 (0.120 g, 0.281 mmol) and Example A7 (0.0763 g, 0.309 mmol) were combined to provide 1-(4-(2-carbamoylpyridin-4-yloxy)-2-fluorophenyl)-3-(3-isopropyl-1-(quinolin-6-yl)-1H-pyrazol-5-yl)urea hydrochloride (0.101 g, 65% yield). ¹H NMR (DMSO-d₆) δ 9.23 (s, 1H), 9.11-9.08 (m, 2H), 8.77 (d, J=4.8 Hz, 1H), 8.53 (d, J=6.0 Hz, 1H), 8.35 (d, J=2.0 Hz, 1H), 8.29 (d, J=8.8 Hz, 1H), 8.18-8.11 (m, 3H), 7.84-7.80 (m, 1H), 7.75 (s, 1H), 7.43 (d, J=2.4 Hz, 1H), 7.31 (dd, J=11.6, 2.4 Hz, 1H), 7.20 (dd, J=6.0, 2.4 Hz, 1H), 7.05 (dd, J=9.6, 2.8 Hz, 1H), 6.45 (s, 1H); MS (ESI) m/z: 526.2 (M+H⁺).

### Example 16

Using a procedure analogous to Example 1, Example B3 (85 mg, 0.19 mmol), Example A13 (42 mg, 0.19 mmol) and DIEA (55 mg, 0.42 mmol) were combined in DMSO (1 mL) and heated overnight at 50-55° C. Water was added (50 mL) and the mixture was extracted with EtOAc (3×100 mL), dried (MgSO₄), concentrated in vacuo and purified by silica gel column chromatography to obtain 1-(3-tert-butyl-1-(quinolin-6-yl)-1H-pyrazol-5-yl)-3-(2-fluoro-5-(6-methylpyridin-3-yloxy)phenyl)urea. The product treated with 0.10M aq HCl solution to obtain 1-(3-tert-butyl-1-(quinolin-6-yl)-1H-pyrazol-5-yl)-3-(2-fluoro-5-(6-methylpyridin-3-yloxy)phenyl)urea salt HCl salt (56 mg, 52% yield). ¹H NMR (400 MHz, DMSO-d₆) δ 9.38 (brs, 1H), 9.27 (d, J=2.4 Hz, 1H), 9.11 (dd, J=1.6, and 4.8 Hz, 1H), 8.77 (d, J=8.0 Hz, 1H), 8.50 (d, J=3.2 Hz, 1H), 8.34 (d, J=2.4 Hz, 1H), 8.29 (d, J=9.2 Hz, 1H), 8.11 (dd, J=2.4, and 9.2 Hz, 1H), 7.94 (dd, J=3.2, and 6.8 Hz, 1H), 7.83 (m, 2H), 7.68 (d, J=8.8 Hz, 1H), 7.32 (dd, J=9.2, 10.8 Hz, 1H), 6.79 (m, 1H), 6.44 (s, 1H), 2.61 (s, 3H), 1.30 (s, 9H); MS (ESI) m/z: 511.2 (M+H⁺).

### Example 17

Using a procedure analogous to Example 1, Example B9 (213 mg, 0.50 mmol), Example A6 (145 mg, 0.56 mmol) and DIEA (0.09 mL, 0.517 mmol) were combined in DMF (2 mL) to provide 1-(3-isopropyl-1-(quinolin-6-yl)-1H-pyrazol-5-yl)-3-(3-methyl-4-(2-(methylcarbamoyl)pyridin-4-yloxy)phenyl)urea (194 mg, 73% yield). ¹H NMR (400 MHz, DMSO-d₆): δ 9.07 (s, 1H), 8.97 (dd, J=4.2, 1.8 Hz, 1H), 8.76 (q, J=4.9 Hz, 1H), 8.64 (s, 1H), 8.51-8.48 (m, 2H), 8.19-8.16 (m, 2H), 7.97 (dd, J=9.0, 2.4 Hz, 1H), 7.63 (dd, J=8.3, 4.2 Hz, 1H), 7.45 (d, J=2.4 Hz, 1H), 7.33 (dd, J=8.9, 2.6 Hz, 1H), 7.28 (d, J=2.6 Hz, 1H), 7.10-7.04 (m, 2H), 6.43 (s, 1H), 2.95 (m, 1H), 2.78 (d, J=4.9 Hz, 3H), 2.04 (s, 3H), 1.28 (d, J=6.7 Hz, 6H); MS (ESI) m/z: 536.2 (M+H⁺).

### Example 18

mCPBA (1.07 g of −70%, 4.34 mmol) was added to a solution of Example A18 (545 mg, 2.17 mmol) in CH₂Cl₂ (15 mL) and the solution was stirred at RT. The mixture was washed with saturated sodium bicarbonate (3×mL) and brine (30 mL), dried (Na₂SO₄) and concentrated in vacuo to yield 0.65 g of a tan foam, which proved to be a mixture of the sulfoxide and sulfone, and which was used as is. In 2.0N methylamine/THF (22 mL) was placed the crude sulfoxide/sulfone mixture (0.61 g, 2.2 mmol) with stirring overnight at 40° C. The mixture was cooled to RT, diluted with ethyl acetate (25 mL), washed with 5% citric acid (25 mL), saturated sodium bicarbonate (25 mL) and brine (25 mL), dried (Na₂SO₄), concentrated in vacuo and purified by reverse phase chromatography to yield 4-(3-amino-4-fluorophenoxy)-N-methylpyrimidin-2-amine trifluoroacetic acid salt (301 mg, 60% yield). MS (ESI) m/z: 235.0 (M+H⁺).

In DMSO (2 mL) was placed Example B3 (159 mg, 0.359 mmol), DIEA (139 mg, 1.08 mmol) and 4-(3-amino-4-fluorophenoxy)-N-methylpyrimidin-2-amine trifluoroacetic acid salt (150 mg, 0.431 mmol). The mixture was warmed to 50° C. overnight, then diluted with ethyl acetate (25 mL), washed with 5% citric acid (50 mL), saturated sodium bicarbonate (50 mL) and brine (50 mL), dried (Na₂SO₄), concentrated in vacuo and purified by column chromatography to yield 1-(3-tert-butyl-1-(quinolin-6-yl)-1H-pyrazol-5-yl)-3-(2-fluoro-5-(2-(methylamino)pyrimidin-4-yloxy)phenyl)urea (93 mg, 49% yield). ¹H-NMR (DMSO-d₆) 1.31 (s, 9H), 2.54-2.86 (br d, 3H), 6.46 (s, 1H), 6.57-6.61 (br m, 1H), 6.91-6.93 (br m, 1H), 7.32-7.37 (m, 1H), 7.94-8.05 (m, 2H), 8.23-8.33 (m, 2H), 8.40 (d, 1H), 8.48 (s, 1H), 8.98 (d, 1H), 9.19-9.21 (m, 1H), 9.43-9.47 (br m, 1H), 9.68-9.73 (br m, 1H); MS (ESI) m/z: 527.2 (M+H⁺).

### Example 19

Using a procedure analogous to Example 1, Example B9 (85 mg, 0.20 mmol), Example A9 (46 mg, 0.20 mmol) and DIEA (57 mg, 0.44 mmol) were combined in DMSO (1 mL) to obtain 1-(2-fluoro-4-(2-(methylamino)pyridin-4-yloxy)phenyl)-3-(3-isopropyl-1-(quinolin-6-yl)-1H-pyrazol-5-yl)urea. The product was treated with 0.100M aq HCl solution to obtain 1-(2-fluoro-4-(2-(methylamino)pyridin-4-yloxy)phenyl)-3-(3-isopropyl-1-(quinolin-6-yl)-1H-pyrazol-5-yl)urea HCl salt (52 mg, 48% yield). ¹H NMR (400 MHz, DMSO-d₆) δ 9.17 (s, 1H), 9.14 (brs, 1H), 8.98 (dd, J=1.2, and 4.0 Hz, 1H), 8.50 (d, J=8.4 Hz, 1H), 8.42 (brs, 1H), 8.20 (d, J=2.8 Hz, 1H), 8.17 (d, J=9.2 Hz, 1H), 7.97 (dd, J=2.4, and 9.2 Hz, 1H), 7.91 (d, J=7.2 Hz, 1H), 7.64 (dd, J=4.0, and 8.4 Hz, 1H), 7.34 (dd, J=2.4, and 11.6 Hz, 1H), 7.07 (dd, J=1.2, and 8.8 Hz, 1H), 6.60 (d, J=6.4 Hz, 1H), 6.43 (s, 1H), 6.17 (brs, 1H), 2.95 (m, 1H), 2.87 (d, J=4.4 Hz, 3H), 1.27 (d, J=6.8 Hz, 6H); MS (ESI) m/z: 512.3 (M+H⁺).

### Example 20

Using a procedure analogous to Example 1, Example B10 (0.13 g, 0.314 mmol), Example A7 (0.086 g, 0.346 mmol) and DIEA (0.12 mL, 0.69 mmol) were dissolved in DMSO (1.5 mL) and the mixture was heated at 55° C. overnight to afford 1-(4-(2-carbamoylpyridin-4-yloxy)-2-fluorophenyl)-3-(3-ethyl-1-(quinolin-6-yl)-1H-pyrazol-5-yl)urea (0.088 g, 55% yield). This was converted to corresponding HCl salt by reacting with HCl (4.0 M HCl/dioxane, 1.0 eq.). ¹H NMR (DMSO-d₆) δ 9.37 (s, 1H), 9.18-9.15 (m, 2H), 8.90 (d, J=8.0 Hz, 1H), 8.54 (d, J=5.6 Hz, 1H), 8.43 (s, 1H), 8.21 (d, J=8.8 Hz, 1H), 8.22-8.12 (m, 3H), 7.91 (m, 1H), 7.78 (s, 1H), 7.45 (d, J=1.6 Hz, 1H), 7.31 (dd, J=12, 2.0 Hz, 1H), 7.21 (dd, J=5.2, 1.4 Hz, 1H), 7.05 (d, J=9.2 Hz, 1H), 6.44 (s, 1H), 2.64 (q, J=7.6 Hz, 2H), 1.25 (t, J=7.2 Hz, 3H); MS (ESI) m/z: 512.3 (M+H⁺).

### Example 21

Using a procedure analogous to Example 1, Example B3 (198 mg, 373 mmol), DIEA (121 mg, 0.933 mmol) and Example A21 (117 mg, 0.448 mmol) were combined to yield 1-(3-tert-butyl-1-(quinolin-6-yl)-1H-pyrazol-5-yl)-3-(2-fluoro-5-(6-(methylcarbamoyl)pyridin-3-yloxy)phenyl)urea (140 mg, 67% yield) as the hydrochloride salt. ¹H-NMR (DMSO-d₆) δ 1.30 (s, 9H), 2.81 (d, 3H), 6.45 (s, 1H), 6.81-6.83 (m, 1H), 7.30-7.35 (m, 1H), 7.43-7.46 (m, 1H), 7.91-8.02 (m, 3H), 8.19-8.21 (m, 1H), 8.34-8.43 (m, 3H), 8.65-8.66 (m, 1H), 8.91 (d, 1H), 9.17-9.19 (m, 1H), 9.28 (br s, 1H), 9.44 (s, 1H); MS (ESI) m/z: 554.2 (M+H⁺).

### Example 22

Using a procedure analogous to Example 1, Example B14 (0.125 g, 0.291 mmol) and Example A7 (0.079 g, 0.320 mmol) were combined to provide 1-(4-(2-carbamoylpyridin-4-yloxy)-2-fluorophenyl)-3-(5-chloro-2-(quinolin-6-yl)phenyl)urea hydrochloride (0.070 g, 43% yield). ¹H NMR (DMSO-d₆) δ 9.20 (d, J=3.6 Hz, 1H), 9.04 (d, J=1.6 Hz, 1H), 8.92 (d, J=8.0 Hz, 1H), 8.54-8.52 (m, 2H), 8.36 (d, J=9.2 Hz, 1H), 8.32 (d, J=1.6 Hz, 1H), 8.23 (t, J=8.8 Hz, 1H), 8.18-8.17 (m, 2H), 8.02 (dd, J=8.4, 1.6 Hz, 1H), 7.93-7.90 (m, 1H), 7.76 (s, 1H), 7.43-7.39 (m, 2H), 7.31-7.26 (m, 2H), 7.20 (dd, J=5.6, 2.4 Hz, 1H), 7.06 (dd, J=8.8, 1.2 Hz, 1H); MS (ESI) m/z: 528.0 (M+H⁺).

### Example 23

Using a procedure analogous to Example 1, Example B9 (35 mg, 0.02 mmol), Example A14 (47 mg, 0.20 mmol) and DIEA were combined in DMSO and heated overnight at 60° C. to obtain 1-(2-fluoro-4-(2-methoxypyridin-4-yloxy)phenyl)-3-(3-isopropyl-1-(quinolin-6-yl)-1H-pyrazol-5-yl)urea HCl salt (54 mg, 49% yield). ¹H NMR (400 MHz, DMSO-d₆) δ 9.35 (brs, 1H), 9.13 (brs, 1H), 8.85 (d, J=2.0 Hz, 1H), 8.74 (s, 1H), 8.35 (dd, J=1.6, and 8.4 Hz, 1H), 8.25 (m, 1H), 7.90 (s, 1H), 7.74 (d, J=8.4 Hz, 1H), 7.71 (brs, 1H), 7.29 (m, 2H), 6.46 (s, 1H), 4.31 (q, J=7.2 Hz, 2H), 2.66 (s, 3H), 1.29 (s, 9H), 1.22 (t, J=7.2 Hz, 3H); MS (ESI) m/z: 556.3 (M+H⁺).

### Example 24

Using a procedure analogous to Example 1, Example B19 (150 mg, 0.329 mmol) and Example A2 (94 mg, 0.362 mmol) were combined to provide 1-(3-tert-butyl-1-(2-methylquinolin-6-yl)-1H-pyrazol-5-yl)-3-(2-fluoro-4-(2-(methylcarbamoyl)pyridin-4-yloxy)phenyl)urea hydrochloride (113 mg, 60% yield). ¹H-NMR (DMSO-d₆) δ 1.33 (s, 9H), 2.79 (d, 3H), 3.00 (s, 3H), 6.49 (s, 1H), 7.02-7.04 (m, 1H), 7.19-7.20 (m, 1H), 7.30 (d, 1H), 7.45 (s, 1H), 8.01 (d, 1H), 8.07-8.09 (m, 1H), 8.34-8.37 (m, 1H), 8.50-8.57 (m, 3H), 8.85-8.87 (m, 1H), 9.10 (d, 1H), 9.29 (s, 1H), 9.61 (s, 1H); MS (ESI) m/z: 568.2 (M+H⁺).

### Example 25

Using a procedure analogous to Example 1, Example B9 (120 mg, 0.28 mmol), Example A20 (80 mg, 0.29 mmol), and DIEA (110 mg, 0.84 mmol) were combined to yield 1-(2-fluoro-5-(6-(trifluoromethyl)pyridin-3-yloxy)phenyl)-3-(3-isopropyl-1-(quinolin-6-yl)-1H-pyrazol-5-yl)urea hydrochloride (62 mg, 40% yield). ¹H-NMR (DMSO-d₆) δ 1.25 (d, 6H), 2.93 (pen, 1H), 6.41 (s, 1H), 6.85-6.88 (m, 1H), 7.32-7.37 (m, 1H), 7.51-7.54 (m, 1H), 7.87-7.90 (m, 2H), 7.96-7.98 (m, 1H), 8.16-8.18 (m, 1H), 8.33 (d, 1H), 8.40 (s, 1H), 8.52 (s, 1H), 8.87 (d, 1H), 9.15-9.16 (m, 1H), 9.28 (s, 1H), 9.42 (s, 1H); MS (ESI) m/z: 551.2 (M+H⁺).

### Example 26

Using a procedure analogous to Example 1, Example B9 (0.200 g, 0.468 mmol) and Example A15 (0.113 g, 0.491 mmol) were combined to provide 1-(4-(2-cyanopyridin-4-yloxy)-2-fluorophenyl)-3-(3-isopropyl-1-(quinolin-6-yl)-1H-pyrazol-5-yl)urea (0.238 g, 100%). MS (ESI) m/z: 508.3 (M+H⁺)

1-(4-(2-Cyanopyridin-4-yloxy)-2-fluorophenyl)-3-(3-isopropyl-1-(quinolin-6-yl)-1H-pyrazol-5-yl)urea (0.108 g, 0.221 mmol) and N-acetylcysteine (0.072 g, 0.441 mmol) were dissolved in MeOH (0.3 mL). Ammonium acetate (0.041 g, 0.0.529 mmol) was added and the reaction mixture was heated at 60° C. under N₂ overnight. The completed reaction was diluted with H₂O (10 ml), basified by K₂CO₃, extracted with EtOAc (2×30 mL) and THF (20 mL). The combined organic layers were washed with brine (20 mL), dried (MgSO₄), concentrated in vacuo and purified by chromatography to afford 1-(4-(2-carbamimidoylpyridin-4-yloxy)-2-fluorophenyl)-3-(3-isopropyl-1-(quinolin-6-yl)-1H-pyrazol-5-yl)urea (0.019 g, 17% yield) as a white solid. This was converted to corresponding HCl salt by reacting with HCl (4.0 M HCl/dioxane, 1.0 eq.). ¹H NMR (DMSO-d₆) δ 9.57 (s, 2H), 9.36-9.34 (m, 2H), 9.20 (d, J=1.2 Hz, 1H), 9.09 (dd, J=4.4, 1.2 Hz, 1H), 8.74 (d, J=8.0 Hz, 1H), 8.68 (d, J=5.2 Hz, 1H), 8.35 (d, J=2.0 Hz, 1H), 8.28 (d, J=9.2 Hz, 1H), 8.18-8.10 (m, 2H), 7.92 (d, J=2.4 Hz, 1H), 7.80 (dd, J=8.4, 4.8 Hz, 1H), 7.32-7.26 (m, 2H), 7.05 (dd, J=8.8, 1.2 Hz, 1H), 6.44 (s, 1H), 2.97-2.93 (m, 1H), 1.28 (d, J=6.8 Hz, 6H); MS (ESI) m/z: 525.3 (M+H⁺).

### Example 27

Using a procedure analogous to Example 1, Example B7 (159 mg, 0.291 mmol), DIEA (45 mg, 0.35 mmol) and Example A34 (74 mg, 0.35 mmol) were combined to give tert-butyl 6-(3-tert-butyl-5-(3-(3-cyano-5-(pyridin-3-yloxy)phenyl)ureido)-1H-pyrazol-1-yl)-3,4-dihydroisoquinoline-2(1H)-carboxylate (83 mg, 47% yield). MS (ESI) m/z: 608.3 (M+H⁺).

In CH₂Cl₂ (8 mL) was placed tert-butyl 6-(3-tert-butyl-5-(3-(3-cyano-5-(pyridin-3-yloxy)phenyl)ureido)-1H-pyrazol-1-yl)-3,4-dihydroisoquinoline-2(1H)-carboxylate (83 mg, 0.14 mmol). HCl (g) was bubbled into reaction mixture until the solution was saturated and the solution was then stirred at RT for 4 hrs. Concentration in vacuo gave a solid which was triturated with ether (10 mL). The solid was collected by filtration, washed with ether (2 mL) and dried to afford 1-(3-tert-butyl-1-(1,2,3,4-tetrahydroisoquinolin-6-yl)-1H-pyrazol-5-yl)-3-(3-cyano-5-(pyridin-3-yloxy)phenyl)urea hydrochloric acid salt (69 mg, 93% yield). ¹H NMR (300 MHz, DMSO-d₆) δ 1.26 (s, 9H), 3.06-3.09 (m, 2H), 3.35-3.40 (m, 2H), 4.28-4.30 (m, 2H), 6.33 (s, 1H), 7.23-7.24 (m, 1H), 7.31-7.34 (m, 1H), 7.39-7.47 (m, 4 H), 7.63-7.67 (m, 2H), 7.77-7.78 (m, 1H), 8.52-8.54 (m, 1H), 8.59 (m, 1H), 8.93 (s, 1H), 9.42-9.43 (m, 2H), 10.16 (s, 1H); MS (ESI) m/z: 527.2 (M+H⁺).

### Example 28

Using a procedure analogous to Example 1, Example A35 (95 mg, 0.428 mmol), DIEA (158 mg, 1.22 mmol) and Example B3 (180 mg, 0.407 mmol) were combined to give 1-(5-(2-aminopyrimidin-4-yloxy)-2-fluorophenyl)-3-(3-tert-butyl-1-(quinolin-6-yl)-1H-pyrazol-5-yl)urea hydrochloride salt (102 mg, 48% yield). ¹H NMR (300 MHz, DMSO-d₆) δ 1.31 (s, 9H), 6.46 (s, 1H), 6.65 (d, J=6.8 Hz, 1H), 6.91-6.94 (m, 1H), 7.32-7.37 (m, 1H), 7.91-7.94 (m, 1H), 7.97-8.00 (m, 1 H), 8.20-8.23 (m, 1H), 8.31-8.33 (m, 1H), 8.36-8.39 (m, 1H), 8.45-8.46 (m, 1H), 8.92-8.94 (m, 1H), 9.18 (m, 1H), 9.45 (m, 1H), 9.66 (s, 1H), NH2 missing; MS (ESI) m/z: 513.3 (M+H⁺).

### Example 29

Using a procedure analogous to Example 1, Example B9 (0.200 g, 0.468 mmol) and Example A15 (0.113 g, 0.491 mmol) in presence of DIEA (0.179 mL, 0.1.03 mmol) were combined to afford 1-(4-(2-cyanopyridin-4-yloxy)-2-fluorophenyl)-3-(3-isopropyl-1-(quinolin-6-yl)-1H-pyrazol-5-yl)urea (0.238 g, 100%) as a colorless oil. It was converted to corresponding HCl salt by reacting with HCl (4.0 M in dioxane, 1.0 eq.). ¹H NMR (400 MHz, DMSO-d₆) δ 9.19 (s, 1H), 9.09-9.08 (m, 2H), 8.73 (d, J=8.0 Hz, 1H), 8.60 (d, J=6.0 Hz, 1H), 8.32 (d, J=2.4 Hz, 1H), 8.27 (d, J=8.8 Hz, 1H), 8.16 (t, J=9.2 Hz, 1H), 8.10 (dd, J=9.2, 2.4 Hz, 1H), 7.80 (dd, J=8.0, 4.4 Hz, 1H), 7.72 (d, J=2.8 Hz, 1H), 7.31 (dd, J=11.6, 2.8 Hz, 1H), 7.23 (dd, J=5.6, 2.8 Hz, 1H), 7.05 (dd, J=9.2, 2.8 Hz, 1H), 6.45 (s, 1H), 2.95 (m, 1H), 1.27 (d, J=7.2 Hz, 6H); MS (ESI) m/z: 508.3 (M+H⁺).

### Example 30

Using a procedure analogous to Example 1, Example B3 (0.2 g, 0.453 mmol) and Example A29 (0.158 g, 0.453 mmol) were combined in DMSO (4 mL) at 70° C. in presence of DIEA (0.176 g, 1.36 mmol) to provide 1-(3-tert-butyl-1-(quinolin-6-yl)-1H-pyrazol-5-yl)-3-(4-(2-((tert-butyldimethylsilyloxy)methyl)pyridin-4-yloxy)-2-fluorophenyl)urea (0.12 g, 43% yield). ¹H NMR (400 MHz, CDCl₃) δ 9.02 (brs, 1H), 8.86 (d, J=8.5 Hz, 1H), 7.65 (m, 3H), 7.27 (dd, J=8, 4.4 Hz, 1H), 6.99 (s, 1H), 6.89 (brd, J=9.0 Hz, 1H), 6.73 (dd, J=12, 2.5 Hz, 1H), 6.65 (s, 1H), 6.60 (m, 1H), 4.71 (s, 2H), 1.36 (s, 9H), 0.85 (s, 9H), 0.05 (s, 6H); MS (ESI) m/z: 641.3 (M+H⁺).

A solution of 1-(3-tert-butyl-1-(quinolin-6-yl)-1H-pyrazol-5-yl)-3-(4-(2-((tert-butyldimethylsilyloxy)methyl)pyridin-4-yloxy)-2-fluorophenyl)urea (0.12 g, 0.19 mmol) in THF (2 ml) was treated with TBAF (1.0 ml, 1.0 M solution in THF) at RT for 1 hour. Water (10 ml) was added and the separated solid was filtered, washed with water and dried to give desilylated product 1-(3-tert-butyl-1-(quinolin-6-yl)-1H-pyrazol-5-yl)-3-(2-fluoro-4-(2-(hydroxymethyl)pyridin-4-yloxy)phenyl)urea as a white solid (0.090 g, 91% yield). ¹H NMR (400 MHz, DMSO-d₆) δ 9.01 (brs, 1H), 8.97 (dd, J=4.2, 1.6 Hz, 2H), 8.50 (brd, J=8.3 Hz, 1H), 8.36 (d, J=5.5 Hz, 2H), 8.18 (m, 2H), 7.97 (dd, J=9, 2 Hz, 1H), 7.63 (dd, J=9, 4.4 Hz, 1H), 7.22 (dd, J=12, 2.5 Hz, 1H), 6.99 (m, 1H), 6.93 (d, J=2.5 Hz, 1H), 6.82 (dd, J=5.7, 2.5 Hz, 1H), 6.48 (s, 1H), 5.40 (t, J=6 Hz, 1H), 4.50 (d, J=8 Hz, 2H), 1.32 (s, 9H); MS (ESI) m/z: 527.2 (M+H⁺). The free base was converted to hydrochloride salt. ¹H NMR (400 MHz, DMSO-d₆) δ 9.31 (brs, 1H), 9.23 (m, 1H), 9.07 (dd, J=4.2, 1.6 Hz, 1H), 8.70 (brd, J=8.3 Hz, 1H), 8.65 (d, J=6.8 Hz, 2H), 8.32 (d, J=2 Hz, 1H), 8.27 (d, J=9 Hz, 1H), 8.22 (d, J=9 Hz, 1H), 8.09 (dd, J=9, 2.3 Hz, 1H), 7.75 (dd, J=8, 4.5 Hz, 1H), 7.43-7.37 (m, 2H), 7.34 (d, 2.8 Hz, 1H), 7.12 (m, 1H), 6.48 (s, 1H), 4.77 (s, 2H), 1.32 (s, 9H); MS (ESI) m/z: 527.2 (M+H⁺).

### Example 31

Using a procedure analogous to Example 4, Example B25 (0.30 g, 0.89 mmol) and Example A31 (0.26 g, 0.98 mmol) in presence of N-methyl pyrrolidine (catalytic amount) were combined to afford 1-(2-fluoro-4-(2-(isopropylamino)pyridin-4-yloxy)phenyl)-3-(3-isopropyl-1-(quinolin-6-yl)-1H-pyrazol-5-yl)urea (0.26 g, 54% yield). The product was treated with methanesulfonic acid to afford 1-(2-fluoro-4-(2-(isopropylamino)pyridin-4-yloxy)phenyl)-3-(3-isopropyl-1-(quinolin-6-yl)-1H-pyrazol-5-yl)urea mesylate salt (260 mg, 88% yield). ¹H NMR (400 MHz, DMSO-d₆) δ 9.03 (m, 1H), 9.01 (s, 1H), 8.96 (dd, J=1.6, and 4.0 Hz, 1H), 8.49 (brd, J=8.4 Hz, 1H), 8.33 (brm, 1H), 8.17 (m, 2H), 7.95 (dd, J=2.8, and 9.2 Hz, 1H), 7.87 (d, J=7.6 Hz, 1H), 7.63 (d, J=4.4, and 8.4 Hz, 1H), 7.33 (dd, J=2.8, and 11.6 Hz, 1H), 7.06 (m, 1H), 6.61 (dd, J=2.4, and 7.2 Hz, 1H), 6.41 (s, 1H), 6.09 (brs, 1H), 3.81 (m, 1H), 2.91 (m, 1H), 2.30 (s, 3H), 1.25 (d, J=6.8 Hz, 6H), 1.13 (d, J=6.0 Hz, 6H); MS (ESI) m/z: 540.3 (M+H⁺).

### Example 32

Using general method A, Example B20 (0.0643 g, 0.226 mmol) and Example A7 (0.168 g, 0.678 mmol) were combined to afford 1-(3-tert-butyl-1-(imidazo[1,2-a]pyridin-6-yl)-1H-pyrazol-5-yl)-3-(4-(2-carbamoylpyridin-4-yloxy)-2-fluorophenyl)urea (0.071 g, 59%) as a white solid. It was converted to corresponding HCl salt by reacting with HCl (4.0 M in dioxane, 1.0 eq.). ¹H NMR (400 MHz, DMSO-d₆) δ 9.48 (s, 1H), 9.33 (d, J=0.8 Hz, 1H), 9.13 (d, J=1.6 Hz, 1H), 8.53 (d, J=5.2 Hz, 1H), 8.41 (d, J=2.4 Hz, 1H), 8.26 (d, J=2.0 Hz, 1H), 8.17-8.09 (m, 4H), 7.72 (s, 1H), 7.39 (d, J=2.4 Hz, 1H), 7.32 (dd, J=12.0, 2.8 Hz, 1H), 7.20 (dd, J=5.6, 2.8 Hz, 1H), 7.05 (dd, J=9.2, 1.6 Hz, 1H), 6.49 (s, 1H), 1.32 (s, 9H); MS (ESI) m/z: 529.3 (M+H⁺).

### Example 33

Using a procedure analogous to Example 1, Example B9 (100 mg, 0.23 mmol) and Example A12 (55 mg, 0.23 mmol) in presence of DIEA (90 μL, 0.51 mmol) were combined to afford 1-(2-fluoro-5-(6-(hydroxymethyl)pyridin-3-yloxy)phenyl)-3-(3-isopropyl-1-(quinolin-6-yl)-1H-pyrazol-5-yl)urea (30 mg, 25% yield). The product was treated with methanesulfonic acid to afford 1-(2-fluoro-5-(6-(hydroxymethyl)pyridin-3-yloxy)phenyl)-3-(3-isopropyl-1-(quinolin-6-yl)-1H-pyrazol-5-yl)urea mesylate salt (23 mg, 65% yield). ¹H NMR (400 MHz, DMSO-d₆) δ 9.11 (brs, 1H), 9.10 (m, 1H), 9.06 (m, 1H), 8.65 (d, J=8.4 Hz, 1H), 8.34 (s, 1H), 8.25 (d, J=1.6 Hz, 1H), 8.21 (d, J=9.2 Hz, 1H), 8.03 (dd, J=2.4, and 9.2 Hz, 1H), 7.91 (dd, J=2.8, and 6.4 Hz, 1H), 7.75 (dd, J=4.8, and 8.4 Hz, 1H), 7.58 (s, 1H), 7.30 (m, 1H), 6.75 (m, 1H), 6.40 (s, 1H), 4.61 (s, 2H), 2.92 (m, 1H), 2.32 (s, 3H), 1.25 (d, J=6.8 Hz, 6H); MS (ESI) m/z: 513.3 (M+H⁺).

### Example 34

Using a procedure analogous to Example B19 step 2, Example A2 (1.00 g, 3.83 mmol) and 2,2,2-trichloroethyl carbonochloridate (1.30 g, 6.12 mmol) were combined to give 2,2,2-trichloroethyl 2-fluoro-4-(2-(methylcarbamoyl)pyridin-4-yloxy)phenylcarbamate. MS (ESI) m/z: 436.0, 438.0 (M+H).

A solution of Example B28 (57 mg, 0.213 mmol), 2,2,2-trichloroethyl 2-fluoro-4-(2-(methylcarbamoyl)pyridin-4-yloxy)phenylcarbamate (102 mg, 0.235 mmol) and DIEA (110 mg, 0.853 mmol) in DMSO (1.5 mL) was placed was warmed to 60° C. overnight. It was then treated with additional 2,2,2-trichloroethyl 2-fluoro-4-(2-(methylcarbamoyl)pyridin-4-yloxy)phenylcarbamate (˜200 mg), warmed to 60° C. overnight. The reaction was diluted with ethyl acetate (25 mL) and 5% citric acid (20 mL). The organic phase was separated, washed with saturated sodium bicarbonate (20 mL) and brine (20 mL), dried (Na₂SO₄), concentrated in vacuo and purified by chromatography (Si-25 column, MeOH/EtOAc) to afford impure product. Repurification via reverse phase chromatography (C18-25 column, CH₃CN/H₂O) gave a residue which was treated with 1N sodium hydroxide (3 mL) and extracted with ethyl acetate (2×20 mL). The combined organic phases were dried (Na₂SO₄), concentrated in vacuo and treated with 4N HCl/dioxane (0.1 mL) to afford 1-(3-tert-butyl-1-(quinoxalin-6-yl)-1H-pyrazol-5-yl)-3-(2-fluoro-4-(2-(methylcarbamoyl)pyridin-4-yloxy)phenyl)urea hydrochloric acid salt (14 mg, 12% yield). ¹H NMR (300 MHz, DMSO-d₆) δ 1.31 (s, 9H), 2.77 (d, 3H), 6.47 (s, 1H), 7.00-7.05 (m, 1H), 7.15-7.18 (m, 1H), 7.26-7.28 (m, 1H), 7.39 (m, 1H), 7.65 (m, 1 H), 8.08-8.13 (m, 2H), 8.21-8.25 (m, 2H), 8.50 (m, 1H), 8.78 (m, 1H), 8.97-9.03 (m, 3H), 9.13 (s, 1H); MS (ESI) m/z: 555.2 (M+H⁺).

### Example 35

Using a procedure analogous to Example 1, Example B9 (0.145 g, 0.339 mmol) and Example A27 (0.087 g, 0.323 mmol) in presence of DIEA (0.124 mL, 0.710 mmol) were combined to afford 1-(4-(2-(1H-pyrazol-4-yl)pyridin-4-yloxy)-2-fluorophenyl)-3-(3-isopropyl-1-(quinolin-6-yl)-1H-pyrazol-5-yl)urea (0.112 g, 63%) as a white foam. It was converted to corresponding mesylate salt by reacting with MsOH (1.0 eq.). ¹H NMR (400 MHz, DMSO-d₆) δ 9.10-9.03 (m, 3H), 8.63-8.52 (m, 4H), 8.26-8.20 (m, 2H), 8.03 (d, J=3.6 Hz, 1H), 7.78-7.70 (m, 2H), 7.40 (d, J=10.8 Hz, 1H), 7.14-7.09 (m, 2H), 6.44 (s, 1H), 2.95 (m, 1H), 2.33 (s, 3H), 1.27 (d, J=7.2 Hz, 6H); MS (ESI) m/z: 549.3 (M+H⁺).

### Example 36

Example B22 (0.310 g, 0.715 mmol), Example A2 (0.187 g, 0.715 mmol) and DIEA (0.274 ml, 1.57 mmol) were combined in DMSO (3 ml) and stirred at 70° C. After 18 h, the completed reaction was cooled to RT, diluted with brine and extracted with EtOAc (3×). The combined organics were washed with brine (2×), dried (MgSO₄), evaporated and purified by flash column chromatography (EtOAc/hexanes) to afford the free base (84.1 mg, 22% yield). The free base thus obtained was treated with certified 0.1N HCl (3.1 ml, 2.0 eq) to afford 1-(1-(benzo[d]thiazol-6-yl)-3-isopropyl-1H-pyrazol-5-yl)-3-(2-fluoro-4-(2-(methylcarbamoyl)pyridin-4-yloxy)phenyl)urea (45 mg) as the bis-HCl salt. ¹H NMR (400 MHz, DMSO-d₆) δ 9.49 (s, 1H), 9.00 (s, 2H), 8.81 (q, J=4.8 Hz, 1H), 8.52 (d, J=5.6 Hz, 1H), 8.39 (d, J=1.6 Hz, 1H), 8.24 (d, J=8.80 Hz, 1H), 8.19 (t, J=9.2 Hz, 1H), 7.70 (dd, J=2.4 and 8.8 Hz, 1H), 7.42 (d, J=2.4 Hz), 7.31 (dd, J=3.2 and 12.0 Hz, 1H), 7.18 (dd, J=2.8 and 6.0 Hz, 1H), 7.06 (ddd, J=1.2, 2.8 and 8.8 Hz, 1H), 6.42 (s, 1H), 2.92 (septet, J=7.2 Hz, 1H), 2.79 (d, J=4.8 Hz, 3H), 1.26 (d, J=7.2 Hz, 6H); MS (ESI) m/z: 546.3 (M+H⁺).

### Example 37

Example B23 (0.200 g, 0.464 mmol), Example A2 (0.121 g, 0.464 mmol) and i-Pr₂NEt (0.178 ml, 1.02 mmol) were combined in DMSO (2 ml) and stirred with heating at 70° C. After 18 h, the completed reaction was cooled to RT, diluted with brine and extracted with EtOAc (3×). The combined organics were washed with brine (2×), dried (MgSO₄), concentrated in vacuo and purified by flash column chromatography (EtOAc/hexanes to EtOAc to THF) to afford impure product. This was purified a second time by reverse phase chromatography (MeCN (w/0.1% TFA)/H₂O (w/0.1% TFA)) to afford desired product (110 mg, 36% yield) as the TFA salt following lyophilization. The TFA salt thus obtained was dissolved in THF and shaken orbitally with MP-carbonate resin (110 mg) for 2 h. The supernatant was decanted away and the beads washed with THF (2×). The combined decants were concentrated, diluted with MeCN/H₂O and then treated with certified 0.1N HCl (3.3 ml, 2.0 eq) to afford 1-(2-fluoro-4-(2-(methylcarbamoyl)pyridin-4-yloxy)phenyl)-3-(3-isopropyl-1-(1-methyl-1H-benzo[d]imidazol-5-yl)-1H-pyrazol-5-yl)urea (31 mg) as the bis-HCl salt. ¹H NMR (400 MHz, DMSO-d₆) δ 9.46 (brs, 1H), 9.11 (s, 1H), 9.07 (s, 1H), 8.76 (brq, J=4.8 Hz, 1H), 8.50 (d, J=5.6 Hz, 1H), 8.11 (t, J=9.2 Hz, 1H), 8.06 (d, J=8.8 Hz), 7.98 (d, J=2.0 Hz, 1H), 7.78 (m, 1H), 7.37 (d, J=2.8 Hz, 1H), 7.28 (dd, J=2.4 and 11.2 Hz, 1H), 7.16 (dd, J=2.4 and 5.6 Hz, 1H), 7.02 (ddd, J=1.2. 2.8 and 8.8 Hz, 1H), 6.38 (s, 1H), 4.08 (s, 3H), 2.92 (septet, J=6.8 Hz, 1H), 2.76 (d, J=4.8 Hz, 3H), 1.24 (d, J=6.8 Hz, 6H); MS (ESI) m/z: 543.2 (M+H⁺).

### Example 38

Using general method A, Example B21 (0.0.054 g, 0.20 mmol) and Example A2 (0.16 g, 0.60 mmol) were combined to afford 1-(1-(imidazo[1,2-a]pyridin-6-yl)-3-isopropyl-1H-pyrazol-5-yl)-3-(2-fluoro-4-(2-(methylcarbamoyl)pyridin-4-yloxy)phenyl)urea (0.045 g, 43% yield) as a white solid. It was converted to corresponding mesylate salt by reacting with MsOH (1.0 eq.). ¹H NMR (400 MHz, DMSO-d₆) δ 9.19 (m, 1H), 8.49 (d, J=6.0 Hz, 1H), 8.33 (d, J=2.0 Hz, 1H), 8.24 (dd, J=9.6, 3.0 Hz, 1H), 7.15 (d, J=2.0 Hz, 1H), 8.08 (d, J=10.0 Hz, 1H), 8.01 (t, J=8.8 Hz, 1H), 7.53 (d, J=3.5 Hz, 1H), 7.12 (dd, J=6.0, 3.0 Hz, 1H), 7.06 (dd, J=11.6, 2.8 Hz, 1H), 6.96 (m, 1H), 6.45 (s, 1H), 3.01 (m, 1H), 2.94 (s, 3H), 2.70 (s, 3H), 1.33 (d, J=6.4 Hz, 6H); MS (ESI) m/z: 529.3 (M+H⁺).

### Example 39

Using general method A, Example B21 (0.030 g, 0.11 mmol) and Example A7 (0.082 g, 0.33 mmol) were combined to afford 1-(1-(imidazo[1,2-a]pyridin-6-yl)-3-isopropyl-1H-pyrazol-5-yl)-3-(4-(2-carbamoylpyridin-4-yloxy)-2-fluorophenyl)urea (0.0245 g, 43% yield) as a white solid. It was converted to corresponding HCl salt by reacting with HCl (4.0 M in dioxane, 1.0 eq.). ¹H NMR (400 MHz, DMSO-d₆) δ 9.26 (d, J=0.8 Hz, 1H), 8.69 (d, J=6.4 Hz, 1H), 8.38 (d, J=1.6 Hz, 1H), 8.26 (dd, J=9.6, 1.2 Hz, 1H), 8.20-8.11 (m, 3H), 7.96 (s, 1H), 7.48 (d, J=5.6 Hz, 1H), 7.23 (dd, J=11.6, 2.8 Hz, 1H), 7.10 (d, J=9.2 Hz, 1H), 6.51 (s, 1H), 3.03 (m, 1H), 1.37 (d, J=6.8 Hz, 6H); MS (ESI) m/z: 515.2 (M+H⁺).

### Example 40

Using a procedure analogous to Example 1, Example A39 (63 mg, 0.29 mmol) and Example B9 (122 mg, 0.29 mmol) were combined to provide 1-(4-(2-aminopyridin-4-yloxy)-2-fluorophenyl)-3-(3-isopropyl-1-(quinolin-6-yl)-1H-pyrazol-5-yl)urea contaminated with 2,2,2-trichloroethanol (56 mg, 28% yield). ¹H NMR (400 MHz, DMSO-d₆) δ 8.99-8.96 (m, 2H), 8.93 (d, J=1.5 Hz, 1 H), 8.49 (m, 1H), 8.19-8.16 (m, 2H), 8.10 (t, J=9.2 Hz, 1H), 7.95 (dd, J=9.1, 2.3 Hz, 1H), 7.80 (d, J=5.8 Hz, 1H), 7.63 (dd, J=8.3, 4.0 Hz, 1H), 7.15 (dd, J=11.8, 2.8 Hz, 1H), 6.95 (m, 1H), 6.44 (s, 1H), 6.13 (dd, J=5.9, 2.2 Hz, 1H), 5.94 (s, 2 H), 5.82 (d, J=2.0 Hz, 1H), 2.94 (m, 1H), 1.27 (d, J=6.8 Hz, 6H); MS (ESI) m/z: 498.2 (M+H⁺).

A solution of the above 1-(4-(2-aminopyridin-4-yloxy)-2-fluorophenyl)-3-(3-isopropyl-1-(quinolin-6-yl)-1H-pyrazol-5-yl)urea (44 mg, 0.061 mmol theory) and pyridine (0.30 mL, 3.7 mmol) in CH₂Cl₂ (1 mL) was treated with acetic anhydride (0.040 mL, 0.39 mmol). The reaction was stirred for 60 h and then partitioned between EtOAc and 2 M aq Na₂CO₃. The organic layer was washed with water and brine. The aqueous phases were back extracted with EtOAc. The combined organic phases were dried (Na₂SO₄), concentrated in vacuo and purified by reverse-phase chromatography to provide 1-(4-(2-acetamidopyridin-4-yloxy)-2-fluorophenyl)-3-(3-isopropyl-1-(quinolin-6-yl)-1H-pyrazol-5-yl)urea (25 mg, 76% yield). ¹H NMR (400 MHz, DMSO-d₆) δ 10.53 (s, 1H), 9.01 (s, 1H), 8.96-8.94 (m, 2H), 8.49 (m, 1H), 8.18-8.11 (m, 4H), 7.95 (dd, J=8.8, 2.4 Hz, 1H), 7.64-7.59 (m, 2H), 7.21 (dd, J=11.8, 2.7 Hz, 1H), 6.98 (m, 1H), 6.65 (dd, J=5.8, 2.4 Hz, 1H), 6.43 (s, 1H), 2.93 (m, 1H), 2.03 (s, 3H), 1.26 (d, J=6.8 Hz, 6H); MS (ESI) m/z: 540.3 (M+H⁺).

### Example 41

Using as procedure analogous to Example 4, Example B25 (100 mg, 0.30 mmol) and Example A30 (74 mg, 0.30 mmol) in presence of N-methyl pyrrolidine (catalytic amount) were combined to afford 1-(4-(2-(ethylamino)pyridin-4-yloxy)-2-fluorophenyl)-3-(3-isopropyl-1-(quinolin-6-yl)-1H-pyrazol-5-yl)urea (70 mg, 45% yield).

The product was treated with methanesulfonic acid to afford 1-(4-(2-(ethylamino)pyridin-4-yloxy)-2-fluorophenyl)-3-(3-isopropyl-1-(quinolin-6-yl)-1H-pyrazol-5-yl)urea mesylate salt (71 mg, 87% yield). ¹H NMR (400 MHz, DMSO-d₆) δ 9.02 (m, 1H), 9.01 (s, 1H), 8.97 (dd, J=1.6, and 4.0 Hz, 1H), 8.49 (brd, J=8.4 Hz, 1H), 8.37 (brs, 1H), 8.17 (m, 2H), 7.95 (dd, J=2.4, and 8.8 Hz, 1H), 7.88 (d, J=7.2 Hz, 1H), 7.63 (d, J=4.4, and 8.4 Hz, 1H), 7.33 (dd, J=2.8, and 11.6 Hz, 1H), 7.06 (m, 1H), 6.61 (dd, J=2.0, and 7.2 Hz, 1H), 6.41 (s, 1H), 6.13 (brs, 1H), 3.23 (m, 2H), 2.92 (m, 1H), 2.28 (s, 3H), 1.25 (d, J=6.8 Hz, 6H), 1.13 (t, J=7.2 Hz, 3H); MS (ESI) m/z: 526.2 (M+H⁺).

### Example 42

Using a procedure analogous to Example 1, Example B9 (295 mg, 0.69 mmol) and Example A40 (214 mg, 0.763 mmol) were combined in DMF (3 mL) to provide 1-(3-isopropyl-1-(quinolin-6-yl)-1H-pyrazol-5-yl)-3-(3-methyl-4-(2-(1-methyl-1H-pyrazol-4-yl)pyridin-4-yloxy)phenyl)urea (278 mg, 72% yield). ¹H NMR (400 MHz, DMSO-d₆) δ 9.00 (s, 1H), 8.94 (dd, J=4.2, 1.6 Hz, 1H), 8.59 (s, 1H), 8.45 (dd, J=8.6, 1.0 Hz, 1H), 8.29 (d, J=6.0 Hz, 1H), 8.20 (s, 1 H), 8.15-8.13 (m, 2H), 7.94 (dd, J=9.1, 2.4 Hz, 1H), 7.91 (s, 1H), 7.60 (dd, J=8.5, 4.1 Hz, 1H), 7.40 (d, J=2.3 Hz, 1H), 7.27 (dd, J=8.6, 2.4 Hz, 1H), 7.11 (d, J=2.2 Hz, 1H), 6.99 (d, J=8.8 Hz, 1H), 6.45 (dd, J=5.7, 2.4 Hz, 1H), 6.39 (s, 1H), 3.83 (s, 3H), 2.92 (m, 1H), 2.05 (s, 3H), 1.25 (d, J=6.9 Hz, 6H); MS (ESI) m/z: 559.2 (M+H⁺).

### Example 43

Using a procedure analogous to Example 1, Example B9 (0.711 g, 1.66 mmol) and Example A28 (0.450 g, 1.58 mmol) in presence of DIEA (0.61 mL, 3.48 mmol) were combined to afford 1-(2-fluoro-4-(2-(1-methyl-1H-pyrazol-4-yl)pyridin-4-yloxy)phenyl)-3-(3-isopropyl-1-(quinolin-6-yl)-1H-pyrazol-5-yl)urea (0.431 g, 48% yield) as a white solid. It was converted to corresponding mesylate salt by reacting with MsOH (1.0 eq.). ¹H NMR (400 MHz, DMSO-d₆) δ 9.08-9.04 (m, 3H), 8.66 (d, J=8.8 Hz, 1H), 8.57-8.54 (m, 2H), 8.26-8.16 (m, 4H), 8.05 (dd, J=9.2, 2.4 Hz, 1H), 7.75 (q, J=4.4 Hz, 1H), 7.64 (s, 1H), 7.37 (dd, J=11.6, 2.0 Hz, 1H), 7.12-7.08 (m, 2H), 6.41 (s, 1H), 3.90 (s, 3H), 2.92 (m, 1H), 2.33 (s, 3H), 1.24 (d, J=7.2 Hz, 6H); MS (ESI) m/z: 563.3 (M+H⁺).

### Example 44

Using a procedure analogous to Example 4, Example B26 (100 mg, 0.29 mmol) and Example A31 (75 mg, 0.29 mmol) in presence of N-methyl pyrrolidine (catalytic amount) were combined to afford 1-(3-tert-butyl-1-(quinolin-6-yl)-1H-pyrazol-5-yl)-3-(2-fluoro-4-(2-(isopropylamino)pyridin-4-yloxy)phenyl)urea (59 mg, 32% yield).

The product was treated with methanesulfonic acid to afford 1-(3-tert-butyl-1-(quinolin-6-yl)-1H-pyrazol-5-yl)-3-(2-fluoro-4-(2-(isopropylamino)pyridin-4-yloxy)phenyl)urea mesylate salt (63 mg, 93% yield). ¹H NMR (400 MHz, DMSO-d₆) δ 9.03 (m, 1H), 9.00 (s, 1H), 8.98 (m, 1H), 8.54 (brd, J=8.4 Hz, 1H), 8.35 (brm, 1H), 8.17 (m, 2H), 7.97 (dd, J=2.4, and 9.2 Hz, 1H), 7.86 (d, J=7.2 Hz, 1H), 7.66 (d, J=4.4, and 8.4 Hz, 1H), 7.33 (dd, J=2.8, and 11.6 Hz, 1H), 7.05 (m, 1H), 6.61 (dd, J=2.4, and 6.8 Hz, 1H), 6.45 (s, 1H), 6.08 (brs, 1H), 3.81 (m, 1H), 2.29 (s, 3H), 1.29 (s, 9H), 1.13 (d, J=6.0 Hz, 6H); MS (ESI) m/z: 554.2 (M+H⁺).

### Example 45

Using a procedure analogous to Example 1, Example B10 (0.060 g, 0.15 mmol) and Example A28 (0.041 g, 0.15 mmol) in presence of DIEA (0.056 mL, 0.32 mmol) were combined to afford 1-(3-ethyl-1-(quinolin-6-yl)-1H-pyrazol-5-yl)-3-(2-fluoro-4-(2-(1-methyl-1H-pyrazol-4-yl)pyridin-4-yloxy)phenyl)urea (47.6 mg, 60% yield) as a white foam. It was converted to corresponding mesylate salt by reacting with MsOH (1.0 eq.). ¹H NMR (400 MHz, DMSO-d₆) δ 9.03-8.95 (m, 3H), 8.55-8.48 (m, 3H), 8.19-8.13 (m, 3H), 7.95 (dd, J=9.2, 2.4 Hz, 1H), 7.64 (dd, J=8.4, 4.4 Hz, 1H), 7.55 (s, 1H), 7.32 (dd, J=12.0, 2.8 Hz, 1H), 7.07-7.01 (m, 2H), 6.36 (s, 1H), 3.86 (s, 3H), 2.56 (q, J=7.2 Hz, 2H), 2.25 (s, 3H), 1.18 (t, J=7.6 Hz, 3H); MS (ESI) m/z: 549.3 (M+H⁺).

### Example 46

Using general method A, Example B27 (77 mg, 0.28 mmol) and Example A2 (150 mg, 0.57 mmol) in presence of DPPA (67 μL, 0.31 mmol) and Et₃N (44 μL, 0.31 mmol) were combined to afford 1-(1-(benzo[d]oxazol-5-yl)-3-isopropyl-1H-pyrazol-5-yl)-3-(2-fluoro-4-(2-(methylcarbamoyl)pyridin-4-yloxy)phenyl)urea (105 mg, 70% yield). ¹H NMR (400 MHz, DMSO-d₆) δ 8.96 (d, J=2.0 Hz, 1H), 8.88 (s, 1H), 8.86 (s, 1H), 8.77 (q, J=4.8 Hz, 1H), 8.49 (d, J=6.0 Hz, 1H), 8.16 (t, J=9.2 Hz, 1H), 7.94 (dd, J=3.2 and 5.2 Hz, 1H), 7.57 (dd, J=2., and 8.8 Hz, 1H), 7.38 (d, J=2.8 Hz, 1H), 7.28 (dd, J=2.4 and 11.6 Hz, 1H), 7.14 (dd, J=2.8 and 5.6 Hz, 1H), 7.03 (m, 1H), 6.37 (s, 1H), 2.76 (d, J=4.8 Hz, 3H), 1.23 (d, J=6.8 Hz, 6H); MS (ESI) m/z: 530.2 (M+H⁺).

### Example 47

To a suspension of 5-amino-2-fluorobenzonitrile (1.00 g, 7.38 mmol) in cone HCl (15 mL) at 0° C. was added a solution of NaNO₂ (0.64 g, 9.28 mmol) in water (15 mL) slowly over 15 min. The resultant mixture was stirred for 90 min at 0° C. A solution comprised of SnCl₂.2H₂O (3.37 g, 14.9 mmol), cone HCl (5 mL) and water (5 mL) was added drop wise over 20 min. The mixture was stirred for 2 h at 0° C., and was extracted with EtOAc (4×25 mL). The aqueous portion was cooled with an ice bath and cautiously treated with 70 mL of 3 M NaOH (70 mL) to a final pH of 5. The aqueous was extracted with EtOAc (2×50 mL). All organics were combined and concentrated in vacuo to afford a brown oil (2.58 g), which was combined with pivaloylacetonitrile (1.00 g, 8.0 mmol) in isopropanol (15 mL). The resultant solution was heated to reflux for 28 h. The reaction mixture was concentrated in vacuo, diluted with EtOAc (30 mL) and washed with water (20 mL), satd aq NaHCO₃ (20 mL), water (20 mL) and brine (20 mL). The aqueous was further extracted with EtOAc (2×20 mL). The combined organics were dried (MgSO₄), concentrated in vacuo and purified by chromatography on silica gel to provide 5-(5-amino-3-tert-butyl-1H-pyrazol-1-yl)-2-fluorobenzonitrile (1.24 g, 65% yield). ¹H NMR (400 MHz, DMSO-d₆) δ 8.05 (m, 1H), 7.97 (m, 1H), 7.61 (t, J=9.0 Hz, 1H), 5.43 (s, 1H), 5.42 (s, 2H); MS (ESI) m/z: 259.3 (M+H⁺).

A solution 5-(5-amino-3-tert-butyl-1H-pyrazol-1-yl)-2-fluorobenzonitrile (86 mg, 0.33 mmol) and acetone oxime (37 mg, 0.50 mmol) in DMAc (1 mL) was treated with potassium tert-butoxide (56 mg, 0.50 mmol). The reaction mixture was stirred 45 min at RT. The mixture was diluted with EtOAc (30 mL), washed with water (10 mL) and brine (2×10 mL), dried (Na₂SO₄), concentrated in vacuo and purified via silica gel chromatography to provide propan-2-one O-2-cyano-4-(5-amino-3-tert-butyl-1H-pyrazol-1-yl)phenyl oxime (47 mg, 45% yield). ¹H NMR (400 MHz, Acetone-d₆) δ 7.93-7.89 (m, 2H), 7.63 (dd, J=8.8, 0.8 Hz, 1H), 5.52 (s, 1H), 4.87 (s, 2H), 2.17 (s, 3H), 2.08)s, 3H), 1.26 (s, 9H); MS (ESI) m/z: 312.3 (M+H⁺).

A solution of propan-2-one O-2-cyano-4-(5-amino-3-tert-butyl-1H-pyrazol-1-yl)phenyl oxime (47 mg, 0.15 mmol) in ethyl acetate (5 mL) was treated with 2 M aq Na₂CO₃ (0.67 mL) and isopropenyl chloroformate (0.050 mL, 0.46 mmol). The reaction was stirred at RT. After 2 h, additional isopropenyl chloroformate (0.1 mL, 0.92 mmol) was added. After 1 h, additional isopropenyl chloroformate (0.1 mL, 0.92 mmol) and 2 M aq Na₂CO₃ (0.5 mL, 1 mmol) were added. After another hour, the reaction was diluted with EtOAc (10 mL), washed with water (10 mL) and brine (10 mL), dried (MgSO₄) and concentrated in vacuo to provide the isopropenyl carbamate of propan-2-one O-2-cyano-4-(5-amino-3-tert-butyl-1H-pyrazol-1-yl)phenyl oxime (62 mg, 58% yield) that was used without further purification. MS (ESI) m/z: 396.2 (M+H⁺).

The isopropenyl carbamate from the previous step (60 mg, 0.15 mmol), Example A2 (40 mg, 0.15 mmol) and N-methylpyrrolidine (1 mg, 0.015 mmol) were combined in THF (1 mL) and heated to 55° C. overnight. The reaction was concentrated and chromatographed to provide the corresponding urea (97 mg, >100% yield) as a dark foam. MS (ESI) m/z: 599.2 (M+H⁺).

The above urea was dissolved in ethanol and treated with 3 M aq HCl (0.5 mL). After 24 h, another 0.5 mL of 3 M aq HCL was added and the stirring was continued for 3 days. The reaction mixture was partitioned aqueous 2 M Na₂CO₃ and EtOAc. The organic layer was washed with sat aq NaHCO3, water, and brine, dried (Na₂SO₄), concentrated in vacuo and purified by silica gel chromatography and recrystallization from acetone to provide 1-(1-(3-aminobenzo[d]isoxazol-5-yl)-3-tert-butyl-1H-pyrazol-5-yl)-3-(2-fluoro-4-(2-(methylcarbamoyl)pyridin-4-yloxy)phenyl)urea (33 mg, 39% yield over 2 steps). ¹H NMR (400 MHz, DMSO-d₆) δ 8.93 (d, J=2.2 Hz, 1H), 8.86 (s, 1H), 7.77 (q, J=4.8 Hz, 1H), 8.50 (d, J=5.4 Hz, 1H), 8.20 (t, J=9.3 Hz, 1H), 7.99 (d, J=1.2 Hz, 1H), 7.64-7.59 (m, 2H), 7.37 (d, J=2.4 Hz, 1H), 7.29 (dd, J=11.9, 2.6 Hz, 1H), 7.15 (dd, J=5.6, 2.6 Hz, 1H), 7.03 (m, 1H), 6.55 (s, 2H), 6.41 (s, 1H), 2.77 (d, J=4.7 Hz, 3H), 1.27 (s, 9H); MS (ESI) m/z: 559.2 (M+H⁺).

### Example 48

Using a procedure analogous to Example 1, Example B9 (0.175 g, 0.41 mmol) and Example A42 (0.097 g, 0.389 mmol) were combined to afford 1-(2-fluoro-5-(6-nitropyridin-3-yloxy)phenyl)-3-(3-isopropyl-1-(quinolin-6-yl)-1H-pyrazol-5-yl)urea (0.129 g, 63% yield) as a light yellow oil. ¹H NMR (400 MHz, DMSO-d6) δ 8.94 (dd, J=4.4, 2.0 Hz, 1H), 8.48 (d, J=8.4 Hz, 1H), 8.31 (d, J=8.8 Hz, 1H), 8.26 (d, J=2.8 Hz, 1H), 8.20 (d, J=8.8 Hz, 1H), 8.11 (d, J=2.4 Hz, 1H), 8.00 (m, 1H), 7.91 (dd, J=9.2, 2.4 Hz, 1H), 7.63 (m, 1H), 7.58 (dd, J=8.8, 2.8 Hz, 1H), 7.22 (m, 1H), 6.84 (m, 1H), 6.46 (s, 1H), 2.98 (m, 1H), 1.30 (d, J=7.2 Hz, 6H); MS (ESI) m/z: 528.3 (M+H⁺).

1-(2-fluoro-5-(6-nitropyridin-3-yloxy)phenyl)-3-(3-isopropyl-1-(quinolin-6-yl)-1H-pyrazol-5-yl)urea (0.129 g, 0.245 mmol) was dissolved in MeOH (2.0 mL), to which NH₄C1 (0.131 g, 2.45 mmol) and zinc power (0.160 g, 2.45 mmol) were added and the reaction mixture was stirred at RT for 4 h. The reaction mixture was filtered through Celite and washed with methanol (30 mL) and EtOAc (50 mL). The filtrate was concentrated in vacuum, partitioned between EtOAc (30 mL) and water (20 mL). The separated organic phase was washed with brine (10 mL), dried (MgSO₄) and concentrated to afford 1-(5-(6-aminopyridin-3-yloxy)-2-fluorophenyl)-3-(3-isopropyl-1-(quinolin-6-yl)-1H-pyrazol-5-yl)urea (0.0495 g, 41% yield) as a white foam. MS (ESI) m/z: 498.2 (M+H⁺).

1-(5-(6-aminopyridin-3-yloxy)-2-fluorophenyl)-3-(3-isopropyl-1-(quinolin-6-yl)-1H-pyrazol-5-yl)urea (0.0495 g, 0.099 mmol) was dissolved in DCM (1.0 mL), to which pyridine (0.49 mL, 6.0 mmol) and acetic anhydride (0.066 mL, 0.65 mmol) were added. The reaction mixture was stirred at RT for 12 h. The completed reaction was quenched with 2M NaHCO₃ (12 mL) and extracted with EtOAc (25 mL). The organic layer was washed with H₂O (15 mL) and brine (10 mL), dried (MgSO₄), concentrated in vacuo and purified by chromatography to afford 1-(5-(6-acetamidopyridin-3-yloxy)-2-fluorophenyl)-3-(3-isopropyl-1-(quinolin-6-yl)-1H-pyrazol-5-yl)urea (0.0234 g, 44% yield) as a yellow foam. It was converted to corresponding mesylate salt by reacting with MsOH (1.0 eq.). ¹H NMR (400 MHz, DMSO-d6) δ 10.54 (s, 1H), 9.09 (s, 1H), 9.07-9.04 (m, 2H), 8.65 (d, J=8.0 Hz, 1H), 8.25 (d, J=2.0 Hz, 1H), 8.21 (d, J=8.8 Hz, 1H), 8.11-8.07 (m, 2H), 8.02 (dd, J=8.8, 2.4 Hz, 1H), 7.85 (m, 1H), 7.75 (m, 1H), 4.48 (dd, J=8.8, 3.2 Hz, 1H), 7.24 (m, 1H), 6.67 (m, 1H), 6.40 (s, 1H), 2.92 (m, 1H), 2.31 (s, 3H), 2.08 (s, 3H), 1.24 (d, J=7.2 Hz, 6H); MS (ESI) m/z: 540.0 (M+H⁺).

### Example 49

Using a procedure analogous to Example 1, Example B24 (150 mg, 0.26 mmol) and Example A28 (74 mg, 0.26 mmol) in presence of DIEA (90 μL, 0.52 mmol) were combined to afford benzyl 6-(3-tert-butyl-5-(3-(2-fluoro-4-(2-(1-methyl-1H-pyrazol-4-yl)pyridin-4-yloxy)phenyl)ureido)-1H-pyrazol-1-yl)-3,4-dihydroisoquinoline-2(1H)-carboxylate (100 mg, 56% yield).

To a solution of benzyl 6-(3-tert-butyl-5-(3-(2-fluoro-4-(2-(1-methyl-1H-pyrazol-4-yl)pyridin-4-yloxy)phenyl)ureido)-1H-pyrazol-1-yl)-3,4-dihydroisoquinoline-2(1H)-carboxylate (100 mg, 0.14 mmol) in methanol/EtOAc (1:1, 10 mL) was added 10% Pd/C. The solution was stirred overnight under H₂ (1 atm) at RT. The solution was filtered and concentrated in vacuo to obtain 1-(3-tert-butyl-1-(1,2,3,4-tetrahydroisoquinolin-6-yl)-1H-pyrazol-5-yl)-3-(2-fluoro-4-(2-(1-methyl-1H-pyrazol-4-yl)pyridin-4-yloxy)phenyl)urea (73 mg, 90% yield) ¹H NMR (400 MHz, DMSO-d₆) δ 9.00 (brs, 1H), 8.02 (m, 1H), 8.35 (d, J=5.6 Hz, 1H), 8.25 (s, 1H), 8.15 (dt, J=2.4, and 8.8, 1H), 7.95 (s, 1H), 7.1-7.3 (m, 3H), 7.99 (m, 1H), 6.65 (m, 1H), 6.36 (d, J=2.8 Hz, 1H), 3.95 (m, 1H), 3.84 (s, 3H), 3.53 (m, 1H), 3.01 (m, 1H), 2.88 (m, 1H), 2.79 (m, 1H), 2.60 (m, 1H), 1.25 (s, 9H); MS (ESI) m/z: 581.3 (M+H⁺).

### Example 50

Using a procedure analogous to Example 1, Example B29 (0.20 g, 0.43 mmol) and Example A27 (118 mg, 0.43 mmol) were combined to afford 1-(4-(2-(1H-pyrazol-4-yl)pyridin-4-yloxy)-2-fluorophenyl)-3-(3-tert-butyl-1-(1-oxo-1,2,3,4-tetrahydroisoquinolin-7-yl)-1H-pyrazol-5-yl)urea (123 mg, 47% yield). ¹H NMR (400 MHz, DMSO-d₆) δ 8.88 (brs, 1H), 8.83 (s, 1H), 8.33 (d, J=5.6 Hz, 1H), 8.10 (d, J=8.8 Hz, 1H), 8.07 (m, 2H), 7.85 (d, J=2.0 Hz, 1H), 7.57 (dd, J=2.4, and 8.0 Hz, 1H), 7.42 (d, J=8.0 Hz, 1H), 7.31 (brs, 1H), 7.18 (dd, J=2.4, and 12.0 Hz, 1H), 6.95 (m, 1H), 6.65 (m, 1H), 6.33 (s, 1H), 3.35 (m, 2H), 2.91 (m, 2H), 1.22 (s, 9H); MS (ESI) m/z: 581.3 (M+H⁺).

### Example 51

Using a procedure analogous to Example 1, Example B30 (0.20 g, 0.37 mmol) and Example A27 (100 mg, 0.37 mmol) were combined to afford tert-butyl 7-(5-(3-(4-(2-(1H-pyrazol-4-yl)pyridin-4-yloxy)-2-fluorophenyl)ureido)-3-tert-butyl-1H-pyrazol-1-yl)-3,4-dihydroisoquinoline-2(1H)-carboxylate (130 mg, 53% yield) which was treated with 4.0 M HCl/dioxane (2 mL) and it was stirred at RT for 4 hours. The solid was filtered, washed with ethyl acetate, and dried under vacuum to obtain 1-(4-(2-(1H-pyrazol-4-yl)pyridin-4-yloxy)-2-fluorophenyl)-3-(3-tert-butyl-1-(1,2,3,4-tetrahydroisoquinolin-7-yl)-1H-pyrazol-5-yl)urea HCl salt (120 mg, 96% yield). ¹H NMR (400 MHz, DMSO-d₆) δ 9.51 (brs, 2H), 9.27 (brs, 1H), 9.21 (brs, 1H), 8.69 (brs, 2H), 8.54 (d, J=7.2 Hz, 1H), 8.22 (t, J=9.2 Hz, 1H), 7.84 (m, 1H), 7.3-7.5 (m, 4H), 7.13 (m, 1H), 7.10 (dd, J=2.4, and 6.4 Hz, 1H), 6.37 (s, 1H), 4.38 (m, 2H), 3.38 (m, 2H), 3.05 (m, 2H), 1.28 (s, 9H); MS (ESI) m/z: 567.3 (M+H).

### Example 52

Using a procedure analogous to Example 1, Example A36 (110 mg, 0.363 mmol) and Example B10 (150 mg, 0.363 mmol) were combined and purified by chromatography (Si-25 column, methanol/ethyl acetate) to give 1-(3-ethyl-1-(quinolin-6-yl)-1H-pyrazol-5-yl)-3-(2-fluoro-3-methyl-4-(2-(-methyl-H-pyrazol-4-yl)pyridin-4-yloxy)phenyl)urea as a white foam (66 mg, 32% yield). ¹H NMR (400 MHz, dimethylsulfoxide-d₆) δ 1.27 (t, 3H), 2.65 (q, 2H), 3.89 (s, 3H), 6.46 (s, 1H), 6.74-6.76 (m, 1H), 7.22 (t, 1H), 7.29 (s, 1H), 7.65-7.68 (s, 1H), 7.97-8.02 (m, 3H), 8.20-8.22 (m, 2H), 8.31 (s, 1H), 8.40-8.42 (m, 1H), 8.50-8.53 (m, 1 H), 9.00-9.01 (m, 1H), 9.11 (s, 1H), 9.19 (s, 1H); MS (ESI) m/z: 567.0 (M+H⁺).

### Example 53

Using a procedure analogous to Example 1, Example A38 (108 mg, 0.363 mmol) and Example B10 (150 mg, 0.363 mmol) were combined and purified by chromatography (Si-25 column, methanol/ethyl acetate) to give 1-(2,3-difluoro-4-(2-(1-methyl-1H-pyrazol-4-yl)pyridin-4-yloxy)phenyl)-3-(3-ethyl-1-(quinolin-6-yl)-1H-pyrazol-5-yl)urea as a white foam (78 mg, 38% yield). ¹H NMR (400 MHz, dimethylsulfoxide-d₆) δ 1.29 (t, 3H), 2.09 (s, 3H), 2.67 (q, 2H), 3.91 (s, 3H), 6.47 (s, 1H), 6.59-6.61 (m, 1H), 7.00-7.02 (m, 1H), 7.22 (s, 1H), 7.67-7.70 (m, 1H), 7.99-8.10 (m, 3H), 8.22-8.24 (m, 2H), 8.30 (s, 1H), 8.39 (d, 1H), 8.53-8.55 (m, 1H), 9.00-9.03 (m, 2H), 9.10 (s, 1H); MS (ESI) m/z: 563.3 (M+H⁺).

### Example 54

Using a procedure analogous to Example 1, Example B3 (0.10 g, 0.23 mmol) and Example A32 (56 mg, 0.23 mmol) in the presence of DIEA (68 μL) were combined to afford 1-(3-tert-butyl-1-(quinolin-6-yl)-1H-pyrazol-5-yl)-3-(3-(5-chloropyridin-3-yloxy)-5-cyanophenyl)urea (39 mg, 32% yield). ¹H NMR (400 MHz, DMSO-d₆) δ 9.47 (s, 1H), 8.98 (dd, J=2.0 and 4.4 Hz, 1H), 8.82 (s, 1H), 8.53 (d, J=2.0 Hz, 1H), 8.49 (m, 1H), 8.45 (d, J=2.4 Hz, 1H), 8.17 (m, 2H), 7.97 (dd, J=2.8 and 9.2 Hz, 1H), 7.84 (t, J=2.0 Hz, 1H), 7.70 (t, J=1.6 Hz, 1H), 7.65 (dd, J=4.0 and 8.0 Hz, 1H), 7.45 (t, J=2.0 Hz, 1H), 7.31 (m, 1H), 6.48 (s, 1H), 2.50 (s, 3H), 1.34 (s, 9H); MS (ESI) m/z: 538.0 (M+H⁺).

### Example 55

Using a procedure analogous to Example 1, Example B3 (0.10 g, 0.23 mmol) and Example A33 (51 mg, 0.23 mmol) in presence of DIEA (68 μL) were combined to afford 1-(3-tert-butyl-1-(quinolin-6-yl)-1H-pyrazol-5-yl)-3-(3-cyano-5-(6-methylpyridin-3-yloxy)phenyl)urea (31 mg, 27% yield). ¹H NMR (400 MHz, DMSO-d₆) δ 9.43 (s, 1H), 8.98 (dd, J=2.0 and 4.4 Hz, 1H), 8.74 (s, 1H), 8.48 (m, 1H), 8.33 (d, J=2.8 Hz, 1H), 8.16 (m, 2H), 7.96 (dd, J=2.8 and 9.2 Hz, 1H), 7.63 (m, 2H), 7.50 (dd, J=2.8 and 8.0 Hz, 1H), 7.34 (d, J=8.4 Hz, 1H), 7.29 (t, J=2.0 Hz, 1H), 7.17 (m, 1H), 6.46 (s, 1H), 2.50 (s, 3H), 1.33 (s, 9H); MS (ESI) m/z: 518.0 (M+H⁺).

### Example 56

Using a procedure analogous to Example 1, Example A41 (15 mg, 0.055 mmol) and Example B9 (24 mg, 0.056 mmol) were combined to provide 1-(5-(4-(1H-pyrazol-4-yl)pyrimidin-2-yloxy)-2-fluorophenyl)-3-(3-isopropyl-1-(quinolin-6-yl)-1H-pyrazol-5-yl)urea (9 mg, 29% yield) ¹H NMR (400 MHz, DMSO-d₆) δ 13.36 (s, 1H), 9.09 (s, 1H), 9.07 (s, 1H), 8.95 (m, 1H), 8.50-8.45 (m, 2 H), 8.17-8.12 (m, 2H), 8.01 (dd, J=6.8, 2.9 Hz, 1H), 7.92 (dd, J=9.0, 2.1 Hz, 1H), 7.61 (dd, J=8.2, 4.1 Hz, 1H), 7.51 (d, J=5.0 Hz, 1H), 7.27 (dd, J=11.0, 8.9 Hz, 1 H), 6.85 (m, 1H), 6.40 (s, 1H), 2.89 (m, 1H), 1.22 (d, J=6.8 Hz, 6H); MS (ESI) m/z: 550.2 (M+H⁺).

The following examples were prepared by the methods described in Schemes 1-17, General Method A, the above Examples and the methods described in WO 2006/071940, filed Dec. 23, 2005, incorporated by reference: 1-(3-tert-butyl-1-(1,2,3,4-tetrahydroisoquinolin-6-yl)-1H-pyrazol-5-yl)-3-(3-fluoro-4-(2-(methylcarbamoyl)pyridin-4-yloxy)phenyl)urea, 1-(3-tert-butyl-1-(2-(methylamino)quinolin-6-yl)-1H-pyrazol-5-yl)-3-(2-fluoro-4-(2-(methylcarbamoyl)pyridin-4-yloxy)phenyl)urea, 1-(1-(4-(2-amino-2-oxoethyl)naphthalen-2-yl)-3-tert-butyl-1H-pyrazol-5-yl)-3-(2-chloro-5-(5-fluoropyridin-3-yloxy)phenyl)urea, 1-(3-tert-butyl-1-(quinolin-6-yl)-1H-pyrazol-5-yl)-3-(2-fluoro-5-(pyridin-3-yloxy)phenyl)urea, 1-(3-tert-butyl-1-(quinolin-6-yl)-1H-pyrazol-5-yl)-3-(2,4-difluoro-5-(pyridin-3-yloxy)phenyl)urea, 1-(3-tert-butyl-1-(1,2,3,4-tetrahydroisoquinolin-6-yl)-1H-pyrazol-5-yl)-3-(2,4-difluoro-5-(pyridin-3-yloxy)phenyl)urea, 1-(3-tert-butyl-1-(1H-indazol-5-yl)-1H-pyrazol-5-yl)-3-(2-fluoro-5-(pyridin-3-yloxy)phenyl)urea, 1-(5-tert-butyl-1-(quinolin-6-yl)-1H-pyrazol-3-yl)-3-(2-fluoro-4-(2-(methylcarbamoyl)pyridin-4-yloxy)phenyl)urea, 1-(3-tert-butyl-1-(quinolin-6-yl)-1H-pyrazol-5-yl)-3-(2-fluoro-4-(2-(2-hydroxyethylamino)pyridin-4-yloxy)phenyl)urea, 1-(3-tert-butyl-1-(quinolin-6-yl)-1H-pyrazol-5-yl)-3-(4-chloro-5-(6-cyanopyridin-3-yloxy)-2-fluorophenyl)urea, 1-(2-fluoro-4-(2-(methylcarbamoyl)pyridin-4-yloxy)phenyl)-3-(1-(quinolin-6-yl)-3-(trifluoromethyl)-1H-pyrazol-5-yl)urea, 1-(3-cyclopentyl-1-(quinolin-6-yl)-1H-pyrazol-5-yl)-3-(2-fluoro-4-(2-(methylcarbamoyl)pyridin-4-yloxy)phenyl)urea, 1-(3-cyclobutyl-1-(quinolin-6-yl)-1H-pyrazol-5-yl)-3-(2-fluoro-4-(2-(methylcarbamoyl)pyridin-4-yloxy)phenyl)urea, 1-(3-tert-butyl-1-(quinolin-6-yl)-1H-pyrazol-5-yl)-3-(5-(6-cyanopyridin-3-yloxy)-2-fluorophenyl)urea, 1-(3-tert-butyl-1-(quinolin-6-yl)-1H-pyrazol-5-yl)-3-(3-fluoro-4-(2-(methylamino)pyridin-4-yloxy)phenyl)urea, 1-(3-tert-butyl-1-(1,2,3,4-tetrahydroisoquinolin-6-yl)-1H-pyrazol-5-yl)-3-(4-methyl-3-(pyridin-3-yloxy)phenyl)urea, 1-(2-fluoro-5-(6-methylpyridin-3-yloxy)phenyl)-3-(3-isopropyl-1-(quinolin-6-yl)-1H-pyrazol-5-yl)urea, 1-(3-ethyl-1-(quinolin-6-yl)-1H-pyrazol-5-yl)-3-(2-fluoro-4-(2-(methylamino)pyridin-4-yloxy)phenyl)urea, 1-(3-tert-butyl-1-(1H-indazol-5-yl)-1H-pyrazol-5-yl)-3-(2-fluoro-5-(2-(methylamino)pyrimidin-4-yloxy)phenyl)urea, 1-(4-(2-carbamoylpyridin-4-yloxy)-2-fluorophenyl)-3-(4-chloro-2-(quinolin-6-yl)phenyl)urea, 1-(1-(1H-indazol-5-yl)-3-isopropyl-1H-pyrazol-5-yl)-3-(4-(2-carbamoylpyridin-4-yloxy)-2-fluorophenyl)urea, 1-(3-tert-butyl-1-(2-methylquinolin-6-yl)-1H-pyrazol-5-yl)-3-(4-(2-carbamoylpyridin-4-yloxy)-2-fluorophenyl)urea, 1-(4-(2-carbamoylpyridin-4-yloxy)-3-methylphenyl)-3-(3-isopropyl-1-(quinolin-6-yl)-1H-pyrazol-5-yl)urea, 1-(2-fluoro-4-(2-(methylcarbamoyl)pyridin-4-yloxy)phenyl)-3-(3-isopropyl-1-(2-methylquinolin-6-yl)-1H-pyrazol-5-yl)urea, 1-(3-isopropyl-1-(quinolin-6-yl)-1H-pyrazol-5-yl)-3-(3-methyl-4-(2-(methylcarbamoyl)pyridin-4-yloxy)phenyl)urea, 1-(4-(2-carbamoylpyridin-4-yloxy)-2-fluorophenyl)-3-(3-isopropyl-1-(2-methylquinolin-6-yl)-1H-pyrazol-5-yl)urea, 1-(4-(2-(dimethylamino)pyridin-4-yloxy)-2-fluorophenyl)-3-(3-isopropyl-1-(quinolin-6-yl)-1H-pyrazol-5-yl)urea, 1-(3-isopropyl-1-(quinolin-6-yl)-1H-pyrazol-5-yl)-3-(3-methyl-4-(2-(methylamino)pyridin-4-yloxy)phenyl)urea, 1-(3-tert-butyl-1-(quinolin-6-yl)-1H-pyrazol-5-yl)-3-(4-(2-carbamoylpyridin-4-yloxy)-3-methylphenyl)urea, 1-(5-(2-aminopyrimidin-4-yloxy)-2-fluorophenyl)-3-(3-isopropyl-1-(2-methylquinolin-6-yl)-1H-pyrazol-5-yl)urea, 1-(2-fluoro-4-(2-(methylamino)pyrimidin-4-yloxy)phenyl)-3-(3-isopropyl-1-(quinolin-6-yl)-1H-pyrazol-5-yl)urea, 1-(2-fluoro-5-(6-(methylcarbamoyl)pyridin-3-yloxy)phenyl)-3-(3-isopropyl-1-(quinolin-6-yl)-1H-pyrazol-5-yl)urea, 1-(3-isopropyl-1-(quinolin-6-yl)-1H-pyrazol-5-yl)-3-(2-methyl-4-(2-(methylcarbamoyl)pyridin-4-yloxy)phenyl)urea, 1-(3-tert-butyl-1-(2-methylquinolin-6-yl)-1H-pyrazol-5-yl)-3-(2-fluoro-4-(2-(methylamino)pyridin-4-yloxy)phenyl)urea, 1-(4-(2-(1H-pyrazol-4-yl)pyridin-4-yloxy)-2-fluorophenyl)-3-(3-methyl-1-(quinolin-6-yl)-1H-pyrazol-5-yl)urea, 1-(2-fluoro-4-(2-(methylcarbamoyl)pyridin-4-yloxy)phenyl)-3-(3-isopropyl-1-(quinoxalin-6-yl)-1H-pyrazol-5-yl)urea, 1-(4-(2-carbamoylpyridin-4-yloxy)-2-fluorophenyl)-3-(3-isopropyl-1-(quinoxalin-6-yl)-1H-pyrazol-5-yl)urea, 1-(1-(benzo[d]oxazol-5-yl)-3-tert-butyl-1H-pyrazol-5-yl)-3-(2-fluoro-4-(2-(methylcarbamoyl)pyridin-4-yloxy)phenyl)urea, 1-(4-(2-(1H-pyrazol-4-yl)pyridin-4-yloxy)-3-methylphenyl)-3-(3-isopropyl-1-(quinolin-6-yl)-1H-pyrazol-5-yl)urea, 1-(4-(2-(1H-pyrazol-4-yl)pyridin-4-yloxy)-2-fluorophenyl)-3-(3-tert-butyl-1-(1,2,3,4-tetrahydroisoquinolin-6-yl)-1H-pyrazol-5-yl)urea, 1-(3-fluoro-4-(2-(isopropylamino)pyridin-4-yloxy)phenyl)-3-(3-isopropyl-1-(quinolin-6-yl)-1H-pyrazol-5-yl)urea, 1-(3-isopropyl-1-(quinolin-6-yl)-1H-pyrazol-5-yl)-3-(4-(2-(isopropylamino)pyridin-4-yloxy)-3-methylphenyl)urea, 1-(4-(2-(cyclopentylamino)pyridin-4-yloxy)-2-fluorophenyl)-3-(3-isopropyl-1-(quinolin-6-yl)-1H-pyrazol-5-yl)urea, 1-(2-fluoro-4-(2-(1-methyl-1H-pyrazol-4-yl)pyridin-4-yloxy)phenyl)-3-(3-methyl-1-(quinolin-6-yl)-1H-pyrazol-5-yl)urea, 1-(2-fluoro-4-((2-(1-methyl-1H-pyrazol-4-yl)pyridin-4-yl)oxy)phenyl)-3-(1-isopropyl-3-(quinolin-6-yl)-1H-pyrazol-4-yl)urea, 4-(3-fluoro-4-(3-(1-isopropyl-3-(quinolin-6-yl)-1H-pyrazol-4-yl)ureido)phenoxy)-N-methylpicolinamide, 1-(4-((2-(ethylamino)pyridin-4-yl)oxy)-2,3-difluorophenyl)-3-(3-isopropyl-1-(quinolin-6-yl)-1H-pyrazol-5-yl)urea, N-(4-(2-fluoro-4-(3-(3-isopropyl-1-(quinolin-6-yl)-1H-pyrazol-5-yl)ureido)phenoxy)pyridin-2-yl)acetamide, 4-(4-(3-(1-(4-(aminomethyl)naphthalen-2-yl)-3-(tert-butyl)-1H-pyrazol-5-yl)ureido)-3-fluorophenoxy)-N-methylpicolinamide, and 4-(3-fluoro-4-(3-(3-(1-hydroxy-2-methylpropan-2-yl)-1-(quinolin-6-yl)-1H-pyrazol-5-yl)ureido)phenoxy)-N-methylpicolinamide.

The following examples are prepared by the methods described in Schemes 1-17, General Method A, the above Examples and the methods described in WO 2006/071940, filed Dec. 23, 2005, incorporated by reference: 1-(3-tert-butyl-1-(1,2,3,4-tetrahydroisoquinolin-6-yl)-1H-pyrazol-5-yl)-3-(4-methyl-3-(4-(pyridin-3-yl)pyrimidin-2-yloxy)phenyl)urea, 1-(5-(4-(1H-pyrazol-4-yl)pyrimidin-2-yloxy)-2-fluorophenyl)-3-(3-isopropyl-1-(quinolin-6-yl)-1H-pyrazol-5-yl)urea, 1-(2-fluoro-4-methyl-5-(4-(1-methyl-1H-pyrazol-4-yl)pyrimidin-2-yloxy)phenyl)-3-(3-isopropyl-1-(quinolin-6-yl)-1H-pyrazol-5-yl)urea, 1-(2-fluoro-5-(4-(1-methyl-1H-pyrazol-4-yl)pyrimidin-2-yloxy)phenyl)-3-(3-isopropyl-1-(quinolin-6-yl)-1H-pyrazol-5-yl)urea, 1-(2-fluoro-4-(2-(1-methyl-1H-pyrazol-4-yl)pyridin-4-yloxy)phenyl)-3-(1-isopropyl-4-(quinolin-6-yl)-1H-pyrrol-3-yl)urea, 1-(2-fluoro-4-(2-(1-methyl-1H-pyrazol-4-yl)pyridin-4-yloxy)phenyl)-3-(1-isopropyl-5-methyl-3-(quinolin-6-yl)-1H-pyrazol-4-yl)urea, 1-(2-fluoro-4-(2-(1-methyl-1H-pyrazol-4-yl)pyridin-4-yloxy)phenyl)-3-(2-isopropyl-5-(quinolin-6-yl)oxazol-4-yl)urea, 1-(2-fluoro-4-(2-(1-methyl-1H-pyrazol-4-yl)pyridin-4-yloxy)phenyl)-3-(2-isopropyl-5-(quinolin-6-yl)thiazol-4-yl)urea, 1-(2-fluoro-4-(2-(1-methyl-1H-pyrazol-4-yl)pyridin-4-yloxy)phenyl)-3-(5-isopropyl-2-(quinolin-6-yl)furan-3-yl)urea, 1-(2-fluoro-4-(2-(1-methyl-1H-pyrazol-4-yl)pyridin-4-yloxy)phenyl)-3-(5-isopropyl-2-(quinolin-6-yl)thiophen-3-yl)urea, 1-(2-fluoro-4-(2-(1-methyl-1H-pyrazol-4-yl)pyridin-4-yloxy)phenyl)-3-(4-isopropyl-1-(quinolin-6-yl)-1H-imidazol-2-yl)urea, 1-(2-fluoro-4-(2-(1-methyl-H-pyrazol-4-yl)pyridin-4-yloxy)phenyl)-3-(5-isopropyl-2-(quinolin-6-yl)-1H-pyrrol-3-yl)urea, 1-(2-fluoro-4-(2-(1-methyl-1H-pyrazol-4-yl)pyridin-4-yloxy)phenyl)-3-(4-isopropyl-1-(quinolin-6-yl)-1H-pyrrol-2-yl)urea, 1-(2-fluoro-4-(2-(1-methyl-1H-pyrazol-4-yl)pyridin-4-yloxy)phenyl)-3-(5-methyl-2-(quinolin-6-yl)pyridin-3-yl)urea, 1-(2-fluoro-4-(2-(1-methyl-1H-pyrazol-4-yl)pyridin-4-yloxy)phenyl)-3-(1-isopropyl-3-(1,2,3,4-tetrahydroisoquinolin-6-yl)-1H-pyrazol-4-yl)urea, 1-(2-fluoro-4-(2-(1-methyl-1H-pyrazol-4-yl)pyridin-4-yloxy)phenyl)-3-(1-isopropyl-4-(1,2,3,4-tetrahydroisoquinolin-6-yl)-1H-pyrrol-3-yl)urea, 1-(2-fluoro-4-(2-(1-methyl-1H-pyrazol-4-yl)pyridin-4-yloxy)phenyl)-3-(2-isopropyl-5-(1,2,3,4-tetrahydroisoquinolin-6-yl)oxazol-4-yl)urea, 1-(2-fluoro-4-(2-(1-methyl-1H-pyrazol-4-yl)pyridin-4-yloxy)phenyl)-3-(2-isopropyl-5-(1,2,3,4-tetrahydroisoquinolin-6-yl)thiazol-4-yl)urea, 1-(2-fluoro-4-(2-(1-methyl-1H-pyrazol-4-yl)pyridin-4-yloxy)phenyl)-3-(5-isopropyl-2-(1,2,3,4-tetrahydroisoquinolin-6-yl)furan-3-yl)urea, 1-(2-fluoro-4-(2-(1-methyl-1H-pyrazol-4-yl)pyridin-4-yloxy)phenyl)-3-(5-isopropyl-2-(1,2,3,4-tetrahydroisoquinolin-6-yl)thiophen-3-yl)urea, 1-(2-fluoro-4-(2-(1-methyl-1H-pyrazol-4-yl)pyridin-4-yloxy)phenyl)-3-(4-isopropyl-1-(1,2,3,4-tetrahydroisoquinolin-6-yl)-1H-imidazol-2-yl)urea, 1-(2-fluoro-4-(2-(1-methyl-1H-pyrazol-4-yl)pyridin-4-yloxy)phenyl)-3-(5-isopropyl-2-(1,2,3,4-tetrahydroisoquinolin-6-yl)-1H-pyrrol-3-yl)urea, 1-(2-fluoro-4-(2-(1-methyl-1H-pyrazol-4-yl)pyridin-4-yloxy)phenyl)-3-(4-isopropyl-1-(1,2,3,4-tetrahydroisoquinolin-6-yl)-1H-pyrrol-2-yl)urea, 1-(2-fluoro-4-(2-(1-methyl-1H-pyrazol-4-yl)pyridin-4-yloxy)phenyl)-3-(5-methyl-2-(1,2,3,4-tetrahydroisoquinolin-6-yl)pyridin-3-yl)urea, 4-(3-fluoro-4-(3-(1-isopropyl-3-(quinolin-6-yl)-1H-pyrazol-4-yl)ureido)phenoxy)-N-methylpicolinamide, 4-(3-fluoro-4-(3-(1-isopropyl-4-(quinolin-6-yl)-1H-pyrrol-3-yl)ureido)phenoxy)-N-methylpicolinamide, 4-(3-fluoro-4-(3-(2-isopropyl-5-(quinolin-6-yl)oxazol-4-yl)ureido)phenoxy)-N-methylpicolinamide, 4-(3-fluoro-4-(3-(2-isopropyl-5-(quinolin-6-yl)thiazol-4-yl)ureido)phenoxy)-N-methylpicolinamide, 4-(3-fluoro-4-(3-(5-isopropyl-2-(quinolin-6-yl)thiophen-3-yl)ureido)phenoxy)-N-methylpicolinamide, 4-(3-fluoro-4-(3-(4-isopropyl-1-(quinolin-6-yl)-1H-imidazol-2-yl)ureido)phenoxy)-N-methylpicolinamide, 4-(3-fluoro-4-(3-(5-isopropyl-2-(quinolin-6-yl)-1H-pyrrol-3-yl)ureido)phenoxy)-N-methylpicolinamide, 4-(3-fluoro-4-(3-(4-isopropyl-1-(quinolin-6-yl)-1H-pyrrol-2-yl)ureido)phenoxy)-N-methylpicolinamide, 4-(3-fluoro-4-(3-(5-methyl-2-(quinolin-6-yl)pyridin-3-yl)ureido)phenoxy)-N-methylpicolinamide, 4-(3-fluoro-4-(3-(5-isopropyl-2-(quinolin-6-yl)furan-3-yl)ureido)phenoxy)-N-methylpicolinamide, 1-(5-(4-(1H-pyrazol-4-yl)pyrimidin-2-yloxy)-2-fluoro-4-methylphenyl)-3-(3-isopropyl-1-(quinolin-6-yl)-1H-pyrazol-5-yl)urea, 1-(2-fluoro-5-(4-(1-methyl-1H-pyrazol-4-yl)pyrimidin-2-yloxy)phenyl)-3-(3-isopropyl-1-(quinolin-6-yl)-1H-pyrazol-5-yl)urea, 1-(5-(4-(1H-pyrazol-4-yl)pyrimidin-2-yloxy)-2-fluoro-4-methylphenyl)-3-(1-(benzo[d]oxazol-5-yl)-3-isopropyl-1H-pyrazol-5-yl)urea, 1-(2-fluoro-4-methyl-5-(4-(1-methyl-1H-pyrazol-4-yl)pyrimidin-2-yloxy)phenyl)-3-(3-isopropyl-1-(quinolin-6-yl)-1H-pyrazol-5-yl)urea, 1-(1-(benzo[d]oxazol-5-yl)-3-isopropyl-1H-pyrazol-5-yl)-3-(2-fluoro-5-(4-(1-methyl-1H-pyrazol-4-yl)pyrimidin-2-yloxy)phenyl)urea, 1-(5-(4-(1H-pyrazol-4-yl)pyrimidin-2-yloxy)-2-fluoro-4-methylphenyl)-3-(1-(imidazo[1,2-a]pyridin-6-yl)-3-isopropyl-1H-pyrazol-5-yl)urea, 1-(2-fluoro-5-(4-(1-methyl-1H-pyrazol-4-yl)pyrimidin-2-yloxy)phenyl)-3-(1-(imidazo[1,2-a]pyridin-6-yl)-3-isopropyl-1H-pyrazol-5-yl)urea, and 1-(3-tert-butyl-1-(quinolin-6-yl)-1H-pyrazol-5-yl)-3-(2-fluoro-4-(2-(trideuteriomethylcarbamoyl)pyridin-4-yloxy)phenyl)urea.

### Section 3

c-ABL Kinase (Seq. ID no. 1) Assay

Activity of c-ABL kinase (Seq. ID no. 1) was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A_{340 nm}) was continuously monitored spectrophometrically. The reaction mixture (100 μl) contained c-ABL kinase (1 nM. c-ABL from deCode Genetics), peptide substrate (EAIYAAPFAKKK, 0.2 mM), MgCl₂ (10 mM), pyruvate kinase (4 units), lactate dehydrogenase (0.7 units), phosphoenol pyruvate (1 mM), and NADH (0.28 mM) in 90 mM Tris buffer containing 0.2% octyl-glucoside and 3.5% DMSO, pH 7.5. Test compounds were incubated with c-ABL (Seq. ID no. 1) and other reaction reagents at 30 C for 2 h before ATP (500 M) was added to start the reaction. The absorption at 340 nm was monitored continuously for 2 hours at 30° C. on Polarstar Optima plate reader (BMG). The reaction rate was calculated using the 1.0 to 2.0 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package.

pABL Kinase Assay

Activity of pABL kinase (Seq. ID no. 1) was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A_{340 nm}) was continuously monitored spectrophometrically. The reaction mixture (1001) contained pABL kinase (2 nM. pABL from deCode Genetics), peptide substrate (EAIYAAPFAKKK, 0.2 mM), MgCl₂ (10 mM), pyruvate kinase (4 units), lactate dehydrogenase (0.7 units), phosphoenol pyruvate (1 mM), and NADH (0.28 mM) in 90 mM Tris buffer containing 0.2% octyl-glucoside and 3.5% DMSO, pH 7.5. Test compounds were incubated with pABL (Seq. ID no. 1) and other reaction reagents at 30 C for 2 h before ATP (500 μM) was added to start the reaction. The absorption at 340 nm was monitored continuously for 2 hours at 30° C. on Polarstar Optima plate reader (BMG). The reaction rate was calculated using the 1.0 to 2.0 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package. pABL was obtained as a phosphorylated form of the enzyme used in the c-ABL assay (see above).

c-ABL(T315I) (Seq. ID no. 2) Kinase Assay

Activity of c-ABL(T315I) kinase (Seq. ID no. 2) was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A_{340 nm}) was continuously monitored spectrophometrically. The reaction mixture (1001) contained c-ABL(T315I) kinase (Seq. ID no. 2) (6 nM. c-ABL(T315I) from decode Genetics), peptide substrate (EAIYAAPFAKKK, 0.2 mM), MgCl₂ (10 mM), pyruvate kinase (4 units), lactate dehydrogenase (0.7 units), phosphoenol pyruvate (1 mM), and NADH (0.28 mM) in 90 mM Tris buffer containing 0.2% octyl-glucoside and 3.5% DMSO, pH 7.5. Test compounds were incubated with c-ABL(T315I) and other reaction reagents at 30° C. for 2 h before ATP (500 M) was added to start the reaction. The absorption at 340 nm was monitored continuously for 2 hours at 30° C. on Polarstar Optima plate reader (BMG). The reaction rate was calculated using the 1.0 to 2.0 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package.

Biological Data Summary. Biochemical IC₅₀ Values of Compounds of Formula Ia.

In general, Examples 1-56 disclosed herein exhibited >50% inhibition activity at 0.1-2 μM concentration against ABL kinase and T315I ABL kinase.

Biochemical Assay for FLT-3 D835Y (Seq. ID no. 9)

Activity of FLT-3 D835Y kinase was determined by following the production of ADP from the kinase reaction through coupling with the pyruvate kinase/lactate dehydrogenase system (e.g., Schindler et al. Science (2000) 289: 1938-1942). In this assay, the oxidation of NADH (thus the decrease at A340 nm) was continuously monitored spectrophometrically. The reaction mixture (100 μl) contained FLT-3 D835Y (purchased from Invitrogen) (1.2 nM), polyE4Y (1 mg/ml), MgCl₂ (10 mM), pyruvate kinase (4 units), lactate dehydrogenase (0.7 units), phosphoenol pyruvate (1 mM), and NADH (0.28 mM) and ATP (500 μM) in 90 mM Tris buffer containing 0.2% octyl-glucoside and 1% DMSO, pH 7.5. The inhibition reaction was started by mixing serial diluted test compound with the above reaction mixture. The absorption at 340 nm was monitored continuously for 4 hours at 30° C. on Synergy 2 plate reader (BioTeK). The reaction rate was calculated using the 2 to 3 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package. Examples 1, 4, 5 and 15 disclosed herein exhibited >50% inhibition activity at 0.1-2 μM concentration against D835Y FLT-3 kinase.

Biochemical Assay for TRK-A (Seq. ID no. 10)

Activity of TRK-A kinase was determined by following the production of ADP from the kinase reaction through coupling with the pyruvate kinase/lactate dehydrogenase system (e.g., Schindler et al. Science (2000) 289: 1938-1942). In this assay, the oxidation of NADH (thus the decrease at A340 nm) was continuously monitored spectrophometrically. The reaction mixture (100 μl) contained TRK-A (Invitrogen) (5 nM), polyE4Y (1 mg/ml), MgCl₂ (10 mM), pyruvate kinase (4 units), lactate dehydrogenase (0.7 units), phosphoenol pyruvate (1 mM), and NADH (0.28 mM) and ATP (500 M) in 90 mM Tris buffer containing 0.2% octyl-glucoside and 1% DMSO, pH 7.5. The inhibition reaction was started by mixing serial diluted test compound with the above reaction mixture. The absorption at 340 nm was monitored continuously for 4 hours at 30° C. on Synergy 2 plate reader (BioTeK). The reaction rate was calculated using the 3 to 4 h time frame. Percent inhibition was obtained by comparison of reaction rate with that of a control (i.e. with no test compound). IC50 values were calculated from a series of percent inhibition values determined at a range of inhibitor concentrations using software routines as implemented in the GraphPad Prism software package. Example 1 disclosed herein exhibited >50% inhibition activity at 0.1 M concentration against TRK-A kinase.

Biochemical Assay for TIE-2 (Seq. ID no. 11)

Inhibition of TIE-2 kinase activity was carried out with Transcreener Fluorescence Polarization Assay (Part #3004-1K) developed and marketed by BellBrook Labs, Madison, Wis.). In this assay ADP formed from the kinase reaction is quantitatively determined with the Transcreener ADP ADP-specific antibody. The antibody can also bind a florescence tracer (AlexaFluor), which competes with ADP. The extent of the tracer's binding to the antibody, which reflects the amount of ADP formed from the reaction, is followed with the method of fluorescence polarization. In the TIE-2 assay, test compound was incubated with 16 nM TIE-2 (Invitrogen) for 1 h at room temperature, followed by adding ATP (final 50 uM) and incubation for 2 h. At the end of the incubation, Tracer and Anti ADP antibody were added and 30 min later the plate was read with excitation at 620 nm and emission at 680 nm according to the protocol by the manufacture. % inhibition at each concentration of an inhibitor was calculated from the values of mP, from which IC50 was calculated with GraphPad Prism. Examples 1 and 5 disclosed herein exhibited >50% inhibition activity at 0.1 μM concentration against TIE-2 kinase.

ABL kinase
(Seq. ID no. 1)
MSYYHHHHHHDYDIPTTENLYFQGAMDPSSPNYDKWEMERTDITMKHKLGGGQYGEV
YEGVWKKYSLTVAVKTLKEDTMEVEEFLKEAAVMKEIKHPNLVQLLGVCTREPPFYI
ITEFMTYGNLLDYLRECNRQEVNAVVLLYMATQISSAMEYLEKKNFIHRDLAARNCL
VGENHLVKVADFGLSRLMTGDTYTAHAGAKFPIKWTAPESLAYNKFSIKSDVWAFGV
LLWEIATYGMSPYPGIDLSQVYELLEKDYRMERPEGCPEKVYELMRACWQWNPSDRP
SFAEIHQAFETMFQESSISDEVEKELGKRGT
c-ABL(T315I) kinase
(Seq. ID no. 2)
MEEYMPTEHHHHHHENLYFQGTSMDPSSPNYDKWEMERTDITMKHKLGGGQYGEVYE
GVWKKYSLTVAVKTLKEDTMEVEEFLKEAAVMKEIKHPNLVQLLGVCTREPPFYIII
EFMTYGNLLDYLRECNRQEVNAVVLLYMATQISSAMEYLEKKNFIHRDLAARNCLVG
ENHLVKVADFGLSRLMTGDTYTAHAGAKFPIKWTAPESLAYNKFSIKSDVWAFGVLL
WEIATYGMSPYPGIDLSQVYELLEKDYRMERPEGCPEKVYELMRACWQWNPSDRPSF
AEIHQAFETMFQE
BCR-ABL p210-e14a2
(Seq. ID no. 3)
MVDPVGFAEAWKAQFPDSEPPRMELRSVGDIEQELERCKASIRRLEQEVNQERFRMI
YLQTLLAKEKKSYDRQRWGFRRAAQAPDGASEPRASASRPQPAPADGADPPPAEEPE
ARPDGEGSPGKARPGTARRPGAAASGERDDRGPPASVAALRSNFERIRKGHGQPGAD
AEKPFYVNVEFHHERGLVKVNDKEVSDRISSLGSQAMQMERKKSQHGAGSSVGDASR
PPYRGRSSESSCGVDGDYEDAELNPRFLKDNLIDANGGSRPPWPPLEYQPYQSIYVG
GIMEGEGKGPLLRSQSTSEQEKRLTWPRRSYSPRSFEDCGGGYTPDCSSNENLTSSE
EDFSSGQSSRVSPSPTTYRMFRDKSRSPSQNSQQSFDSSSPPTPQCHKRHRHCPVVV
SEATIVGVRKTGQIWPNDDEGAFHGDADGSFGTPPGYGCAADRAEEQRRHQDGLPYI
DDSPSSSPHLSSKGRGSRDALVSGALKSTKASELDLEKGLEMRKWVLSGILASEETY
LSHLEALLLPMKPLKAAATTSQPVLTSQQIETIFFKVPELYEIHKESYDGLFPRVQQ
WSHQQRVGDLFQKLASQLGVYRAFVDNYGVAMEMAEKCCQANAQFAEISENLRARSN
KDAKDPTTKNSLETLLYKPVDRVTRSTLVLHDLLKHTPASHPDHPLLQDALRISQNF
LSSINEEITPRRQSMTVKKGEHRQLLKDSFMVELVEGARKLRHVFLFTDLLLCTKLK
KQSGGKTQQYDCKWYIPLTDLSFQMVDELEAVPNIPLVPDEELDALKIKISQIKSDI
QREKRANKGSKATERLKKKLSEQESLLLLMSPSMAFRVHSRNGKSYTFLISSDYERA
EWRENIREQQKKCFRSFSLTSVELQMLTNSCVKLQTVHSIPLTINKEDDESPGLYGF
LNVIVHSATGFKQSSKALQRPVASDFEPQGLSEAARWNSKENLLAGPSENDPNLFVA
LYDFVASGDNTLSITKGEKLRVLGYNHNGEWCEAQTKNGQGWVPSNYITPVNSLEKH
SWYHGPVSRNAAEYPLSSGINGSFLVRESESSPSQRSISLRYEGRVYHYRINTASDG
KLYVSSESRFNTLAELVHHHSTVADGLITTLHYPAPKRNKPTVYGVSPNYDKWEMER
TDITMKHKLGGGQYGEVYEGVWKKYSLTVAVKTLKEDTMEVEEFLKEAAVMKEIKHP
NLVQLLGVCTREPPFYIITEFMTYGNLLDYLRECNRQEVNAVVLLYMATQISSAMEY
LEKKNFIHRDLAARNCLVGENHLVKVADFGLSRLMTGDTYTAHAGAKFPIKWTAPES
LAYNKFSIKSDVWAFGVLLWEIATYGMSPYPGIDRSQVYELLEKDYRMKRPEGCPEK
VYELMRACWQWNPSDRPSFAEIHQAFETMFQESSISDEVEKELGKQGVRGAVTTLLQ
APELPTKTRTSRRAAEHRDTTDVPEMPHSKGQGESDPLDHEPAVSPLLPRKERGPPE
GGLNEDERLLPKDKKTNLFSALIKKKKKTAPTPPKRSSSFREMDGQPERRGAGEEEG
RDISNGALAFTPLDTADPAKSPKPSNGAGVPNGALRESGGSGFRSPHLWKKSSTLTS
SRLATGEEEGGGSSSKRFLRSCSVSCVPHGAKDTEWRSVTLPRDLQSTGRQFDSSTF
GGHKSEKPALPRKRAGENRSDQVTRGTVTPPPRLVKKNEEAADEVFKDIMESSPGSS
PPNLTPKPLRRQVTVAPASGLPHKEEAWKGSALGTPAAAEPVTPTSKAGSGAPRGTS
KGPAEESRVRRHKHSSESPGRDKGKLSKLKPAPPPPPAASAGKAGGKPSQRPGQEAA
GEAVLGAKTKATSLVDAVNSDAAKPSQPAEGLKKPVLPATPKPHPAKPSGTPISPAP
VPLSTLPSASSALAGDQPSSTAFIPLISTRVSLRKTRQPPERASGAITKGVVLDSTE
ALCLAISGNSEQMASHSAVLEAGKNLYTFCVSYVDSIQQMRNKFAFREAINKLENNL
RELQICPASAGSGPAATQDFSKLLSSVKEISDIVQR
BCR-ABL p210-e13a2
(Seq. ID no. 4)
MVDPVGFAEAWKAQFPDSEPPRMELRSVGDIEQELERCKASIRRLEQEVNQERFRMI
YLQTLLAKEKKSYDRQRWGFRRAAQAPDGASEPRASASRPQPAPADGADPPPAEEPE
ARPDGEGSPGKARPGTARRPGAAASGERDDRGPPASVAALRSNFERIRKGHGQPGAD
AEKPFYVNVEFHHERGLVKVNDKEVSDRISSLGSQAMQMERKKSQHGAGSSVGDASR
PPYRGRSSESSCGVDGDYEDAELNPRFLKDNLIDANGGSRPPWPPLEYQPYQSIYVG
GIMEGEGKGPLLRSQSTSEQEKRLTWPRRSYSPRSFEDCGGGYTPDCSSNENLTSSE
EDFSSGQSSRVSPSPTTYRMFRDKSRSPSQNSQQSFDSSSPPTPQCHKRHRHCPVVV
SEATIVGVRKTGQIWPNDDEGAFHGDADGSFGTPPGYGCAADRAEEQRRHQDGLPYI
DDSPSSSPHLSSKGRGSRDALVSGALKSTKASELDLEKGLEMRKWVLSGILASEETY
LSHLEALLLPMKPLKAAATTSQPVLTSQQIETIFFKVPELYEIHKESYDGLFPRVQQ
WSHQQRVGDLFQKLASQLGVYRAFVDNYGVAMEMAEKCCQANAQFAEISENLRARSN
KDAKDPTTKNSLETLLYKPVDRVTRSTLVLHDLLKHTPASHPDHPLLQDALRISQNF
LSSINEEITPRRQSMTVKKGEHRQLLKDSFMVELVEGARKLRHVFLFTDLLLCTKLK
KQSGGKTQQYDCKWYIPLTDLSFQMVDELEAVPNIPLVPDEELDALKIKISQIKSDI
QREKRANKGSKATERLKKKLSEQESLLLLMSPSMAFRVHSRNGKSYTFLISSDYERA
EWRENIREQQKKCFRSFSLTSVELQMLTNSCVKLQTVHSIPLTINKEEALQRPVASD
FEPQGLSEAARWNSKENLLAGPSENDPNLFVALYDFVASGDNTLSITKGEKLRVLGY
NHNGEWCEAQTKNGQGWVPSNYITPVNSLEKHSWYHGPVSRNAAEYPLSSGINGSFL
VRESESSPSQRSISLRYEGRVYHYRINTASDGKLYVSSESRFNTLAELVHHHSTVAD
GLITTLHYPAPKRNKPTVYGVSPNYDKWEMERTDITMKHKLGGGQYGEVYEGVWKKY
SLTVAVKTLKEDTMEVEEFLKEAAVMKEIKHPNLVQLLGVCTREPPFYIITEFMTYG
NLLDYLRECNRQEVNAVVLLYMATQISSAMEYLEKKNFIHRDLAARNCLVGENHLVK
VADFGLSRLMTGDTYTAHAGAKFPIKWTAPESLAYNKFSIKSDVWAFGVLLWEIATY
GMSPYPGIDRSQVYELLEKDYRMKRPEGCPEKVYELMRACWQWNPSDRPSFAEIHQA
FETMFQESSISDEVEKELGKQGVRGAVTTLLQAPELPTKTRTSRRAAEHRDTTDVPE
MPHSKGQGESDPLDHEPAVSPLLPRKERGPPEGGLNEDERLLPKDKKTNLFSALIKK
KKKTAPTPPKRSSSFREMDGQPERRGAGEEEGRDISNGALAFTPLDTADPAKSPKPS
NGAGVPNGALRESGGSGFRSPHLWKKSSTLTSSRLATGEEEGGGSSSKRFLRSCSVS
CVPHGAKDTEWRSVTLPRDLQSTGRQFDSSTFGGHKSEKPALPRKRAGENRSDQVTR
GTVTPPPRLVKKNEEAADEVFKDIMESSPGSSPPNLTPKPLRRQVTVAPASGLPHKE
EAWKGSALGTPAAAEPVTPTSKAGSGAPRGTSKGPAEESRVRRHKHSSESPGRDKGK
LSKLKPAPPPPPAASAGKAGGKPSQRPGQEAAGEAVLGAKTKATSLVDAVNSDAAKP
SQPAEGLKKPVLPATPKPHPAKPSGTPISPAPVPLSTLPSASSALAGDQPSSTAFIP
LISTRVSLRKTRQPPERASGAITKGVVLDSTEALCLAISGNSEQMASHSAVLEAGKN
LYTFCVSYVDSIQQMRNKFAFREAINKLENNLRELQICPASAGSGPAATQDFSKLLS
SVKEISDIVQR
BCR-ABL p190-e1a2
(Seq. ID no. 5)
MVDPVGFAEAWKAQFPDSEPPRMELRSVGDIEQELERCKASIRRLEQEVNQERFRMI
YLQTLLAKEKKSYDRQRWGFRRAAQAPDGASEPRASASRPQPAPADGADPPPAEEPE
ARPDGEGSPGKARPGTARRPGAAASGERDDRGPPASVAALRSNFERIRKGHGQPGAD
AEKPFYVNVEFHHERGLVKVNDKEVSDRISSLGSQAMQMERKKSQHGAGSSVGDASR
PPYRGRSSESSCGVDGDYEDAELNPRFLKDNLIDANGGSRPPWPPLEYQPYQSIYVG
GIMEGEGKGPLLRSQSTSEQEKRLTWPRRSYSPRSFEDCGGGYTPDCSSNENLTSSE
EDFSSGQSSRVSPSPTTYRMFRDKSRSPSQNSQQSFDSSSPPTPQCHKRHRHCPVVV
SEATIVGVRKTGQIWPNDDEGAFHGDAEALQRPVASDFEPQGLSEAARWNSKENLLA
GPSENDPNLFVALYDFVASGDNTLSITKGEKLRVLGYNHNGEWCEAQTKNGQGWVPS
NYITPVNSLEKHSWYHGPVSRNAAEYPLSSGINGSFLVRESESSPSQRSISLRYEGR
VYHYRINTASDGKLYVSSESRFNTLAELVHHHSTVADGLITTLHYPAPKRNKPTVYG
VSPNYDKWEMERTDITMKHKLGGGQYGEVYEGVWKKYSLTVAVKTLKEDTMEVEEFL
KEAAVMKEIKHPNLVQLLGVCTREPPFYIITEFMTYGNLLDYLRECNRQEVNAVVLL
YMATQISSAMEYLEKKNFIHRDLAARNCLVGENHLVKVADFGLSRLMTGDTYTAHAG
AKFPIKWTAPESLAYNKFSIKSDVWAFGVLLWEIATYGMSPYPGIDRSQVYELLEKD
YRMKRPEGCPEKVYELMRACWQWNPSDRPSFAEIHQAFETMFQESSISDEVEKELGK
QGVRGAVTTLLQAPELPTKTRTSRRAAEHRDTTDVPEMPHSKGQGESDPLDHEPAVS
PLLPRKERGPPEGGLNEDERLLPKDKKTNLFSALIKKKKKTAPTPPKRSSSFREMDG
QPERRGAGEEEGRDISNGALAFTPLDTADPAKSPKPSNGAGVPNGALRESGGSGFRS
PHLWKKSSTLTSSRLATGEEEGGGSSSKRFLRSCSVSCVPHGAKDTEWRSVTLPRDL
QSTGRQFDSSTFGGHKSEKPALPRKRAGENRSDQVTRGTVTPPPRLVKKNEEAADEV
FKDIMESSPGSSPPNLTPKPLRRQVTVAPASGLPHKEEAWKGSALGTPAAAEPVTPT
SKAGSGAPRGTSKGPAEESRVRRHKHSSESPGRDKGKLSKLKPAPPPPPAASAGKAG
GKPSQRPGQEAAGEAVLGAKTKATSLVDAVNSDAAKPSQPAEGLKKPVLPATPKPHP
AKPSGTPISPAPVPLSTLPSASSALAGDQPSSTAFIPLISTRVSLRKTRQPPERASG
AITKGVVLDSTEALCLAISGNSEQMASHSAVLEAGKNLYTFCVSYVDSIQQMRNKFA
FREAINKLENNLRELQICPASAGSGPAATQDFSKLLSSVKEISDIVQR
BCR-ABL p210-e14a2 T315I
(Seq. ID no. 6)
MVDPVGFAEAWKAQFPDSEPPRMELRSVGDIEQELERCKASIRRLEQEVNQERFRMI
YLQTLLAKEKKSYDRQRWGFRRAAQAPDGASEPRASASRPQPAPADGADPPPAEEPE
ARPDGEGSPGKARPGTARRPGAAASGERDDRGPPASVAALRSNFERIRKGHGQPGAD
AEKPFYVNVEFHHERGLVKVNDKEVSDRISSLGSQAMQMERKKSQHGAGSSVGDASR
PPYRGRSSESSCGVDGDYEDAELNPRFLKDNLIDANGGSRPPWPPLEYQPYQSIYVG
GIMEGEGKGPLLRSQSTSEQEKRLTWPRRSYSPRSFEDCGGGYTPDCSSNENLTSSE
EDFSSGQSSRVSPSPTTYRMFRDKSRSPSQNSQQSFDSSSPPTPQCHKRHRHCPVVV
SEATIVGVRKTGQIWPNDDEGAFHGDADGSFGTPPGYGCAADRAEEQRRHQDGLPYI
DDSPSSSPHLSSKGRGSRDALVSGALKSTKASELDLEKGLEMRKWVLSGILASEETY
LSHLEALLLPMKPLKAAATTSQPVLTSQQIETIFFKVPELYEIHKESYDGLFPRVQQ
WSHQQRVGDLFQKLASQLGVYRAFVDNYGVAMEMAEKCCQANAQFAEISENLRARSN
KDAKDPTTKNSLETLLYKPVDRVTRSTLVLHDLLKHTPASHPDHPLLQDALRISQNF
LSSINEEITPRRQSMTVKKGEHRQLLKDSFMVELVEGARKLRHVFLFTDLLLCTKLK
KQSGGKTQQYDCKWYIPLTDLSFQMVDELEAVPNIPLVPDEELDALKIKISQIKSDI
QREKRANKGSKATERLKKKLSEQESLLLLMSPSMAFRVHSRNGKSYTFLISSDYERA
EWRENIREQQKKCFRSFSLTSVELQMLTNSCVKLQTVHSIPLTINKEDDESPGLYGF
LNVIVHSATGFKQSSKALQRPVASDFEPQGLSEAARWNSKENLLAGPSENDPNLFVA
LYDFVASGDNTLSITKGEKLRVLGYNHNGEWCEAQTKNGQGWVPSNYITPVNSLEKH
SWYHGPVSRNAAEYPLSSGINGSFLVRESESSPSQRSISLRYEGRVYHYRINTASDG
KLYVSSESRFNTLAELVHHHSTVADGLITTLHYPAPKRNKPTVYGVSPNYDKWEMER
TDITMKHKLGGGQYGEVYEGVWKKYSLTVAVKTLKEDTMEVEEFLKEAAVMKEIKHP
NLVQLLGVCTREPPFYIIIEFMTYGNLLDYLRECNRQEVNAVVLLYMATQISSAMEY
LEKKNFIHRDLAARNCLVGENHLVKVADFGLSRLMTGDTYTAHAGAKFPIKWTAPES
LAYNKFSIKSDVWAFGVLLWEIATYGMSPYPGIDRSQVYELLEKDYRMKRPEGCPEK
VYELMRACWQWNPSDRPSFAEIHQAFETMFQESSISDEVEKELGKQGVRGAVTTLLQ
APELPTKTRTSRRAAEHRDTTDVPEMPHSKGQGESDPLDHEPAVSPLLPRKERGPPE
GGLNEDERLLPKDKKTNLFSALIKKKKKTAPTPPKRSSSFREMDGQPERRGAGEEEG
RDISNGALAFTPLDTADPAKSPKPSNGAGVPNGALRESGGSGFRSPHLWKKSSTLTS
SRLATGEEEGGGSSSKRFLRSCSVSCVPHGAKDTEWRSVTLPRDLQSTGRQFDSSTF
GGHKSEKPALPRKRAGENRSDQVTRGTVTPPPRLVKKNEEAADEVFKDIMESSPGSS
PPNLTPKPLRRQVTVAPASGLPHKEEAWKGSALGTPAAAEPVTPTSKAGSGAPRGTS
KGPAEESRVRRHKHSSESPGRDKGKLSKLKPAPPPPPAASAGKAGGKPSQRPGQEAA
GEAVLGAKTKATSLVDAVNSDAAKPSQPAEGLKKPVLPATPKPHPAKPSGTPISPAP
VPLSTLPSASSALAGDQPSSTAFIPLISTRVSLRKTRQPPERASGAITKGVVLDSTE
ALCLAISGNSEQMASHSAVLEAGKNLYTFCVSYVDSIQQMRNKFAFREAINKLENNL
RELQICPASAGSGPAATQDFSKLLSSVKEISDIVQR
BCR-ABL p210-e13a2 T315I
(Seq. ID no. 7)
MVDPVGFAEAWKAQFPDSEPPRMELRSVGDIEQELERCKASIRRLEQEVNQERFRMI
YLQTLLAKEKKSYDRQRWGFRRAAQAPDGASEPRASASRPQPAPADGADPPPAEEPE
ARPDGEGSPGKARPGTARRPGAAASGERDDRGPPASVAALRSNFERIRKGHGQPGAD
AEKPFYVNVEFHHERGLVKVNDKEVSDRISSLGSQAMQMERKKSQHGAGSSVGDASR
PPYRGRSSESSCGVDGDYEDAELNPRFLKDNLIDANGGSRPPWPPLEYQPYQSIYVG
GIMEGEGKGPLLRSQSTSEQEKRLTWPRRSYSPRSFEDCGGGYTPDCSSNENLTSSE
EDFSSGQSSRVSPSPTTYRMFRDKSRSPSQNSQQSFDSSSPPTPQCHKRHRHCPVVV
SEATIVGVRKTGQIWPNDDEGAFHGDADGSFGTPPGYGCAADRAEEQRRHQDGLPYI
DDSPSSSPHLSSKGRGSRDALVSGALKSTKASELDLEKGLEMRKWVLSGILASEETY
LSHLEALLLPMKPLKAAATTSQPVLTSQQIETIFFKVPELYEIHKESYDGLFPRVQQ
WSHQQRVGDLFQKLASQLGVYRAFVDNYGVAMEMAEKCCQANAQFAEISENLRARSN
KDAKDPTTKNSLETLLYKPVDRVTRSTLVLHDLLKHTPASHPDHPLLQDALRISQNF
LSSINEEITPRRQSMTVKKGEHRQLLKDSFMVELVEGARKLRHVFLFTDLLLCTKLK
KQSGGKTQQYDCKWYIPLTDLSFQMVDELEAVPNIPLVPDEELDALKIKISQIKSDI
QREKRANKGSKATERLKKKLSEQESLLLLMSPSMAFRVHSRNGKSYTFLISSDYERA
EWRENIREQQKKCFRSFSLTSVELQMLTNSCVKLQTVHSIPLTINKEEALQRPVASD
FEPQGLSEAARWNSKENLLAGPSENDPNLFVALYDFVASGDNTLSITKGEKLRVLGY
NHNGEWCEAQTKNGQGWVPSNYITPVNSLEKHSWYHGPVSRNAAEYPLSSGINGSFL
VRESESSPSQRSISLRYEGRVYHYRINTASDGKLYVSSESRFNTLAELVHHHSTVAD
GLITTLHYPAPKRNKPTVYGVSPNYDKWEMERTDITMKHKLGGGQYGEVYEGVWKKY
SLTVAVKTLKEDTMEVEEFLKEAAVMKEIKHPNLVQLLGVCTREPPFYIIIEFMTYG
NLLDYLRECNRQEVNAVVLLYMATQISSAMEYLEKKNFIHRDLAARNCLVGENHLVK
VADFGLSRLMTGDTYTAHAGAKFPIKWTAPESLAYNKFSIKSDVWAFGVLLWEIATY
GMSPYPGIDRSQVYELLEKDYRMKRPEGCPEKVYELMRACWQWNPSDRPSFAEIHQA
FETMFQESSISDEVEKELGKQGVRGAVTTLLQAPELPTKTRTSRRAAEHRDTTDVPE
MPHSKGQGESDPLDHEPAVSPLLPRKERGPPEGGLNEDERLLPKDKKTNLFSALIKK
KKKTAPTPPKRSSSFREMDGQPERRGAGEEEGRDISNGALAFTPLDTADPAKSPKPS
NGAGVPNGALRESGGSGFRSPHLWKKSSTLTSSRLATGEEEGGGSSSKRFLRSCSVS
CVPHGAKDTEWRSVTLPRDLQSTGRQFDSSTFGGHKSEKPALPRKRAGENRSDQVTR
GTVTPPPRLVKKNEEAADEVFKDIMESSPGSSPPNLTPKPLRRQVTVAPASGLPHKE
EAWKGSALGTPAAAEPVTPTSKAGSGAPRGTSKGPAEESRVRRHKHSSESPGRDKGK
LSKLKPAPPPPPAASAGKAGGKPSQRPGQEAAGEAVLGAKTKATSLVDAVNSDAAKP
SQPAEGLKKPVLPATPKPHPAKPSGTPISPAPVPLSTLPSASSALAGDQPSSTAFIP
LISTRVSLRKTRQPPERASGAITKGVVLDSTEALCLAISGNSEQMASHSAVLEAGKN
LYTFCVSYVDSIQQMRNKFAFREAINKLENNLRELQICPASAGSGPAATQDFSKLLS
SVKEISDIVQR
BCR-ABL p190-e1a2
(Seq. ID no. 8)
MVDPVGFAEAWKAQFPDSEPPRMELRSVGDIEQELERCKASIRRLEQEVNQERFRMI
YLQTLLAKEKKSYDRQRWGFRRAAQAPDGASEPRASASRPQPAPADGADPPPAEEPE
ARPDGEGSPGKARPGTARRPGAAASGERDDRGPPASVAALRSNFERIRKGHGQPGAD
AEKPFYVNVEFHHERGLVKVNDKEVSDRISSLGSQAMQMERKKSQHGAGSSVGDASR
PPYRGRSSESSCGVDGDYEDAELNPRFLKDNLIDANGGSRPPWPPLEYQPYQSIYVG
GIMEGEGKGPLLRSQSTSEQEKRLTWPRRSYSPRSFEDCGGGYTPDCSSNENLTSSE
EDFSSGQSSRVSPSPTTYRMFRDKSRSPSQNSQQSFDSSSPPTPQCHKRHRHCPVVV
SEATIVGVRKTGQIWPNDDEGAFHGDAEALQRPVASDFEPQGLSEAARWNSKENLLA
GPSENDPNLFVALYDFVASGDNTLSITKGEKLRVLGYNHNGEWCEAQTKNGQGWVPS
NYITPVNSLEKHSWYHGPVSRNAAEYPLSSGINGSFLVRESESSPSQRSISLRYEGR
VYHYRINTASDGKLYVSSESRFNTLAELVHHHSTVADGLITTLHYPAPKRNKPTVYG
VSPNYDKWEMERTDITMKHKLGGGQYGEVYEGVWKKYSLTVAVKTLKEDTMEVEEFL
KEAAVMKEIKHPNLVQLLGVCTREPPFYIIIEFMTYGNLLDYLRECNRQEVNAVVLL
YMATQISSAMEYLEKKNFIHRDLAARNCLVGENHLVKVADFGLSRLMTGDTYTAHAG
AKFPIKWTAPESLAYNKFSIKSDVWAFGVLLWEIATYGMSPYPGIDRSQVYELLEKD
YRMKRPEGCPEKVYELMRACWQWNPSDRPSFAEIHQAFETMFQESSISDEVEKELGK
QGVRGAVTTLLQAPELPTKTRTSRRAAEHRDTTDVPEMPHSKGQGESDPLDHEPAVS
PLLPRKERGPPEGGLNEDERLLPKDKKTNLFSALIKKKKKTAPTPPKRSSSFREMDG
QPERRGAGEEEGRDISNGALAFTPLDTADPAKSPKPSNGAGVPNGALRESGGSGFRS
PHLWKKSSTLTSSRLATGEEEGGGSSSKRFLRSCSVSCVPHGAKDTEWRSVTLPRDL
QSTGRQFDSSTFGGHKSEKPALPRKRAGENRSDQVTRGTVTPPPRLVKKNEEAADEV
FKDIMESSPGSSPPNLTPKPLRRQVTVAPASGLPHKEEAWKGSALGTPAAAEPVTPT
SKAGSGAPRGTSKGPAEESRVRRHKHSSESPGRDKGKLSKLKPAPPPPPAASAGKAG
GKPSQRPGQEAAGEAVLGAKTKATSLVDAVNSDAAKPSQPAEGLKKPVLPATPKPHP
AKPSGTPISPAPVPLSTLPSASSALAGDQPSSTAFIPLISTRVSLRKTRQPPERASG
AITKGVVLDSTEALCLAISGNSEQMASHSAVLEAGKNLYTFCVSYVDSIQQMRNKFA
FREAINKLENNLRELQICPASAGSGPAATQDFSKLLSSVKEISDIVQR
FLT-3 D835Y kinase
(Seq. ID no. 9)
MHKYKKQFRYESQLQMVQVTGSSDNEYFYVDFREYEYDLKWEFPRENLEFGKVLGSG
AFGKVMNATAYGISKTGVSIQVAVKMLKEKADSSEREALMSELKMMTQLGSHENIVN
LLGACTLSGPIYLIFEYCCYGDLLNYLRSKREKFHRTWTEIFKEHNFSFYPTFQSHP
NSSMPGSREVQIHPDSDQISGLHGNSFHSEDEIEYENQKRLEEEEDLNVLTFEDLLC
FAYQVAKGMEFLEFKSCVHRDLAARNVLVTHGKVVKICDFGLARYIMSDSNYVVRGN
ARLPVKWMAPESLFEGIYTIKSDVWSYGILLWEIFSLGVNPYPGIPVDANFYKLIQN
GFKMDQPFYATEEIYIIMQSCWAFDSRKRPSFPNLTSFLGCQLADAEEAMYQNVKGV
EACQLGTDDYDIPTTHHHHHH
TRK-A kinase
(Seq. ID no. 10)
MKCGRRNKFGINRPAVLAPEDGLAMSLHFMTLGGSSLSPTEGKGSGLQGHIIENPQY
FSDACVHHIKRRDIVLKWELGEGAFGKVFLAECHNLLPEQDKMLVAVKALKEASESA
RQDFQREAELLTMLQHQHIVRFFGVCTEGRPLLMVFEYMRHGDLNRFLRSHGPDAKL
LAGGEDVAPGPLGLGQLLAVASQVAAGMVYLAGLHFVHRDLATRNCLVGQGLVVKIG
DFGMSRDIYSTDYYRVGGRTMLPIRWMPPESILYRKFTTESDVWSFGVVLWEIFTYG
KQPWYQLSNTEAIDCITQGRELERPRACPPEVYAIMRGCWQREPQQRHSIKDVHARL
QALAQAPPVYLDVLGKGVEACQLGTDDYDIPTTHHHHHH
TIE-2 kinase
(Seq. ID no. 11)
MAPILGYWKIKGLVQPTRLLLEYLEEKYEEHLYERDEGDKWRNKKFELGLEFPNLPY
YIDGDVKLTQSMAIIRYIADKHNMLGGCPKERAEISMLEGAVLDIRYGVSRIAYSKD
FETLKVDFLSKLPEMLKMFEDRLCHKTYLNGDHVTHPDFMLYDALDVVLYMDPMCLD
AFPKLVCFKKRIEAIPQIDKYLKSSKYIAWPLQGWQATFGGGDHPPKSDLVPRHNQT
SLYKKAGSAAALFNFKKEPFTPVLDWNDIKFQDVIGEGNFGQVLKARIKKDGLRMDA
AIKRMKEYASKDDHRDFAGELEVLCKLGHHPNIINLLGACEHRGYLYLAIEYAPHGN
LLDFLRKSRVLETDPAFAIANSTASTLSSQQLLHFAADVARGMDYLSQKQFIHRDLA
ARNILVGENYVAKIADFGLSRGQEVYVKKTMGRLPVRWMAIESLNYSVYTTNSDVWS
YGVLLWEIVSLGGTPYCGMTCAELYEKLPQGYRLEKPLNCDDEVYDLMRQCWREKPY
ERPSFAQILVSLNRMLEERKT

### BaF3 Cell Culture

BaF3 cells (parental or transfected with the following: wild type BCR-ABL or BCR-ABL point mutants T3151, E255K, Y253F, M351T) were obtained from Professor Richard Van Etten (New England Medical Center, Boston, Mass.). Briefly, cells were grown in RPMI 1640 supplemented with 10% characterized fetal bovine serum (HyClone, Logan, Utah) at 37 degrees Celsius, 5% CO₂, 95% humidity. Cells were allowed to expand until reaching 80% saturation at which point they were subcultured or harvested for assay use.

### BCR-ABL Cell Proliferation Assay

A serial dilution of test compound was dispensed into a 96 well black clear bottom plate (Corning, Corning, N.Y.). For each cell line, three thousand cells were added per well in complete growth medium. Plates were incubated for 72 hours at 37 degrees Celsius, 5% CO2, 95% humidity. At the end of the incubation period Cell Titer Blue (Promega, Madison, Wis.) was added to each well and an additional 4.5 hour incubation at 37 degrees Celsius, 5% CO2, 95% humidity was performed. Plates were then read on a BMG Fluostar Optima (BMG, Durham, N.C.) using an excitation of 544 nM and an emission of 612 nM. Data was analyzed using Prism software (Graphpad, San Diego, Calif.) to calculate IC50's.

Biological Data Summary. Whole Cell IC₅₀ Values of Compounds of Formula Ia.

In general, compounds 1-56 disclosed herein exhibited >50% inhibition of proliferation at 1-10 uM concentration against BaF/3 cells harboring wt BCR-ABL and or BCR-ABLpoint mutants including T315I, E255K, Y253F, and M351T.

### MV-4-11 Cell Culture

MV-4-11 cells (catalog #CRL-9591) were obtained from the American Type Culture Collection (ATCC, Manassas, Va.). Briefly, cells were grown in suspension in IMDM medium supplemented with 10% characterized fetal bovine serum (Invitrogen, Carlsbad, Calif.), 100 units/mL penicillin G, 100 g/ml streptomycin, and 0.29 mg/mL L-glutamine (Invitrogen, Carlsbad, Calif.) at 37 degrees Celsius, 5% CO2, and 95% humidity. Cells were allowed to expand until reaching saturation at which point they were subcultured or harvested for assay use.

### MV-4-11 Cell Proliferation Assay

A serial dilution of test compound was dispensed into a 96-well black clear bottom plate (Corning, Corning, N.Y.). Ten thousand cells were added per well in 200 L complete growth medium. Plates were incubated for 64 hours at 37 degrees Celsius, 5% CO2, and 95% humidity. At the end of the incubation period 40 L of a 440 M solution of resazurin (Sigma, St. Louis, Mo.) in PBS was added to each well and incubated for an additional 8 hours at 37 degrees Celsius, 5% CO2, and 95% humidity. Plates were read on a Synergy2 reader (Biotek, Winooski, Vt.) using an excitation of 540 nM and an emission of 600 nM. Data was analyzed using Prism software (Graphpad, San Diego, Calif.) to calculate IC50 values. Examples 1, 5, and 15 disclosed herein exhibited >50% inhibition activity at <0.1 M concentration against MV-4-11 cells.

### MV-4-11 Phospho-FLT3 Western Blot Assay

MV-4-11 cells suspended in complete medium were added to 12-well tissue culture treated plates (1×10⁶ cells/well). Test compound or DMSO was added to the wells (0.5% final DMSO concentration). The plates were then incubated for 4 hours at 37° C./5% CO₂. The cells were collected and lysed using MPER lysis buffer (Pierce, Rockford, Ill.) containing Halt Phosphatase and Protease Inhibitors (Pierce, Rockford, Ill.) and Phosphatase inhibitor cocktail 2 (Sigma, St. Louis, Mo.) at 4° C. for minutes with shaking. Cleared lysates were separated by SDS-PAGE on a 4-12% Novex NuPage Bis-Tris gel (Invitrogen, Carlsbad, Calif.) and then transferred to PVDF (Invitrogen, Carlsbad, Calif.). After transfer, the PVDF membrane was blocked with BSA (Santa Cruz Biotechnology, Santa Cruz, Calif.) and then probed with an antibody for phospho-FLT3 (Cell Signaling Technology, Beverly, Mass.) and an antibody for eIF4E (Cell Signaling Technology, Beverly, Mass.) as a control. A secondary anti-rabbit antibody conjugated to horseradish peroxidase (Cell Signaling Technology, Beverly, Mass.) was used to detect phospho-FLT3 and eIF4E. ECL Plus (GE Healthcare, Piscataway, N.J.), a substrate for horseradish peroxidase that generates a fluorescent product, was added. Fluorescence was detected using a Storm 840 phosphorimager (GE Healthcare, Piscataway, N.J.) in fluorescence mode. The 160 kDa phospho-FLT3 band was quantified using ImageQuant software (GE Healthcare, Piscataway, N.J.). Data was analyzed using Prism software (Graphpad, San Diego, Calif.) to calculate IC50 values. Example 1 disclosed herein exhibited >50% inhibition of phospho FLT-3 levels in MV-4-11 cells at <0.1 μM concentration.

### K562 Cell Culture

K562 cells (catalog #CCL-243) were obtained from the American Type Culture Collection (ATCC, Manassas, Va.). Briefly, cells were grown in suspension in IMDM medium supplemented with 10% characterized fetal bovine serum (Invitrogen, Carlsbad, Calif.), 100 units/mL penicillin G, 100 g/ml streptomycin, and 0.29 mg/mL L-glutamine (Invitrogen, Carlsbad, Calif.) at 37 degrees Celsius, 5% CO2, and 95% humidity. Cells were allowed to expand until reaching saturation at which point they were subcultured or harvested for assay use.

### K562 Phospho-TRK-A Western Blot Assay

K562 cells suspended in serum-free IMDM medium were added to 24-well tissue culture treated plates (1.5×10⁶ cells/well). Test compound or DMSO was added to the wells (0.5% final DMSO concentration). The plates were then incubated for 4 hours at 37° C./5% CO₂. Following compound incubation, the cells were stimulated with 100 ng/mL NGF (R&D Systems, Minneapolis, Minn.) for 10 min (one DMSO control (-NGF) was not stimulated). The cells were collected and lysed using MPER lysis buffer (Pierce, Rockford, Ill.) containing Halt Phosphatase and Protease Inhibitors (Pierce, Rockford, Ill.) and Phosphatase inhibitor cocktail 2 (Sigma, St. Louis, Mo.) at 4° C. for 10 minutes with shaking. Cleared lysates were separated by SDS-PAGE on a 4-12% Novex NuPage Bis-Tris gel (Invitrogen, Carlsbad, Calif.) and then transferred to PVDF (Invitrogen, Carlsbad, Calif.). After transfer, the PVDF membrane was blocked with BSA (Santa Cruz Biotechnology, Santa Cruz, Calif.) and then probed with an antibody for phospho-TRK-A (Cell Signaling Technology, Beverly, Mass.) and an antibody for eIF4E (Cell Signaling Technology, Beverly, Mass.) as a control. A secondary anti-rabbit antibody conjugated to horseradish peroxidase (Cell Signaling Technology, Beverly, Mass.) was used to detect phospho-TRK-A and eIF4E. ECL Plus (GE Healthcare, Piscataway, N.J.), a substrate for horseradish peroxidase that generates a fluorescent product, was added. Fluorescence was detected using a Storm 840 phosphorimager (GE Healthcare, Piscataway, N.J.) in fluorescence mode. The 140 kDa phospho-TRK-A band was quantified using ImageQuant software (GE Healthcare, Piscataway, N.J.). Data was analyzed using Prism software (Graphpad, San Diego, Calif.) to calculate IC50 values. Example 1 disclosed herein exhibited >50% inhibition of phospho FLT-3 levels in MV-4-11 cells at <0.1 μM concentration.

### CHO-K1 Cell Culture

CHO-K1 cells (catalog #CCL-61) were obtained from the American Type Culture Collection (ATCC, Manassas, Va.). Briefly, cells were grown in F12K medium supplemented with 10% characterized fetal bovine serum (Invitrogen, Carlsbad, Calif.), 100 units/mL penicillin G, 100 μg/ml streptomycin, and 0.29 mg/mL L-glutamine (Invitrogen, Carlsbad, Calif.) at 37 degrees Celsius, 5% CO2, and 95% humidity. Cells were allowed to expand until reaching 70-95% confluence at which point they were subcultured or harvested for assay use.

### TIE-2-Transfected CHO K1 Phospho-TIE-2 Western Blot Assay

CHO K1 cells (1×10⁵ cells/well) were added to a 24-well tissue-culture treated plate in 1 mL of RPMI1640 medium supplemented with 10% characterized fetal bovine serum and 1× non-essential amino acids (Invitrogen, Carlsbad, Calif.). Cells were then incubated overnight at 37 degrees Celsius, 5% CO₂, 95% humidity. Medium was aspirated, and 0.5 mL of medium was added to each well. Transfection-grade plasmid DNA (TIE-2 gene Gateway cloned into pcDNA3.2™/V5-DEST expression vector, Invitrogen, Carlsbad, Calif.) was diluted to 5 μg/mL in room temperature Opti-MEM® I Reduced Serum Medium without serum (Invitrogen, Carlsbad, Calif.). Two L of Lipofectamine LTX Reagent (Invitrogen, Carlsbad, Calif.) was added per 0.5 g of plasmid DNA. The tube was mixed gently and incubated for minutes at room temperature to allow DNA-Lipofectamine LTX complex formation. 100 L of the DNA-Lipofectamine LTX complex was added directly to each well containing cells and mixed gently. Twenty-four hours post-transfection, medium containing DNA-Lipofectamine complexes was aspirated, cells were washed with PBS, and F12K medium supplemented with 10% characterized fetal bovine serum (Invitrogen, Carlsbad, Calif.), 100 units/mL penicillin G, 100 μg/ml streptomycin, and 0.29 mg/mL L-glutamine (Invitrogen, Carlsbad, Calif.) was added. Test compound or DMSO was added to the wells (0.5% final DMSO concentration). The plates were then incubated for 4 hours at 37° C./5% CO₂. Following the incubation, the media was aspirated and the cells were washed with PBS. The cells were lysed using MPER lysis buffer (Pierce, Rockford, Ill.) containing Halt Phosphatase and Protease Inhibitors (Pierce, Rockford, Ill.) and Phosphatase inhibitor cocktail 2 (Sigma, St. Louis, Mo.) at 4° C. for 10 minutes with shaking. Cleared lysates were separated by SDS-PAGE on a 4-12% Novex NuPage Bis-Tris gel (Invitrogen, Carlsbad, Calif.) and then transferred to PVDF (Invitrogen, Carlsbad, Calif.). After transfer, the PVDF membrane was blocked with BSA (Santa Cruz Biotechnology, Santa Cruz, Calif.) and then probed with an antibody for phospho-TIE-2 (Cell Signaling Technology, Beverly, Mass.). A secondary anti-rabbit antibody conjugated to horseradish peroxidase (Cell Signaling Technology, Beverly, Mass.) was used to detect phospho-TIE-2. ECL Plus (GE Healthcare, Piscataway, N.J.), a substrate for horseradish peroxidase that generates a fluorescent product, was added. Fluorescence was detected using a Storm 840 phosphorimager (GE Healthcare, Piscataway, N.J.) in fluorescence mode. The 160 kDa phospho-TIE-2 band was quantified using ImageQuant software (GE Healthcare, Piscataway, N.J.). Data was analyzed using Prism software (Graphpad, San Diego, Calif.) to calculate IC50 values. Example 1 disclosed herein exhibited >50% inhibition of phospho TIE-2 levels in TIE-2-transfected CHO K1 cells at <0.1 M concentration.

Section 4—Important Structural Comparisons vs. Biological Activity

WO 2006/071940A2 describes inhibitors of kinases, including c-ABL kinase, B-RAF kinase, c-MET, VEGF kinase, and the HER family wherein a central phenyl ring is unsubstituted. An example of these inhibitors is shown below, wherein the central phenyl ring is unsubstituted (R16 and R18=H). Compounds A, B and C, discussed below, are taken from WO 2006/071940A2.

### Representative Key Structures

| | |
|---|---|
| Example 1 (R16 = 2-F, R18 = H), | Example 15 (R16 = 2-F, R18 = H) |
| Example 5 (R16 = 2-Me, R18 = H) | Compound B (R16 = H, R18 = H) |
| Compound A (R16 = H, R18 = H) | |

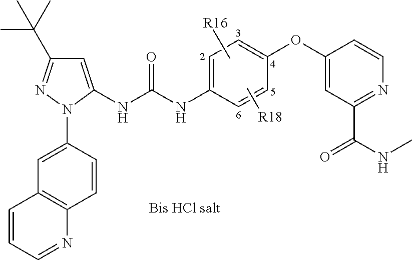

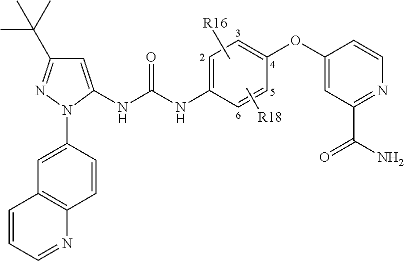

It has unexpectedly been found that inhibitors that contain R16 substituents other than H have superior potency as measured by in vitro kinase inhibition and also as measured by in vivo whole cell anti-proliferation potencies in cancer cells. By way of illustration in Table 1, Example 1 of the present invention containing a 2-F moiety as the R16 substituent is 5.5-times more potent vs. phosphorylated-Abl kinase (p-ABL) than the unsubstituted Compound A containing R16=H. Example 1 is 6.3 times more potent than Compound A vs. the T315I mutant ABLkinase, a clinical isolate of oncogenic ABLkinase found in patients with chronic myelogenous leukemia and in whom treatment is resistant to currently available therapies including Gleevec® (M. E. Gorre et al, Science (2001) 293: 876; S. Branford et al, Blood (2002) 99: 3472; N. von Bubnoff et al, Lancet (2002) 359: 487) and dasatinib (N. P. Shah et al, Science (2004) 305: 399). Example 5 containing a 3-methyl moiety as the R16 substituent is 4 times more potent vs. p-ABL kinase than the unsubstituted (R16=H) Compound A. Example 15 containing a 2-F moiety as the R16 substituent is 8-times more potent vs. unphosphorylated-ABLkinase (u-ABL) than the unsubstituted (R16=H) Compound B (from WO 2006/071940A2). Example 15 is >14-times more potent than Compound B vs. p-ABL kinase, and 18 times more potent than Compound B vs. the T3151 mutant ABLkinase.

TABLE 1

| | | | | | | T315I ABL | |
|---|---|---|---|---|---|---|---|
| | R16 | u-ABL IC₅₀ | | p-ABL IC₅₀ | | IC₅₀ | |
| Example 1 | 2-F | 0.8 | nM | 4 | nM | 6 | nM |
| Example 5 | 5-Me | 0.7 | nM | 6 | nM | 250 | nM |
| Compound A | H | 1 | nM | 22 | | 38 | |
| Example 15 | 2-F | 1 | nM | 35 | nM | 56 | nM |
| CompoundB | H | 8 | nM | >500 | nM | 1,000 | nM |
| Example 4 | 2-F | 0.7 | nM | 20 | nM | 12 | nM |
| Compound C | H | 1.6 | nM | 350 | nM | 160 | nM |

### Structures of Example 4 (R16=2-F, R18=H) and Compound C(R16, R18=H)

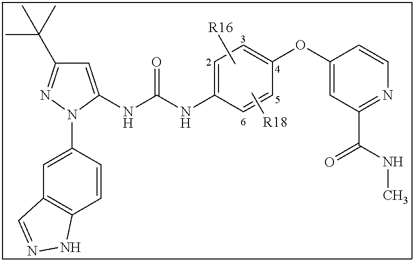

This trend is also evident in other analogs related to those mentioned above. As shown in Table 1, the indazolyl-containing compound Example 4 containing a 2-F moiety as the R16 substituent is 2.2 times more potent than the unsubstituted (R16=H) Compound C vs. u-ABLkinase, 18 times more potent than Compound C vs. p-ABLkinase, and 13 times more potent than Compound C vs. T315I mutant ABLkinase.

This unexpected increase in potency vs. these kinases is also revealed in whole cell assays which measure the effectiveness of these ABLkinase inhibitors to block proliferation of cells containing oncogenic forms of ABLkinase: the fusion protein BCR-ABL kinases (C. L. Sawyers, New England Journal of Medicine (1999) 340: 1330; S. Faderl et al, New England Journal of Medicine (1999) 341: 164; J. B. Konopka et al, Proceeding of the National Academy of Sciences USA (1985) 82: 1810). Table 2 illustrates the increased potency of substituted R16-containing compounds of Examples 1, 5, and 15 vs. their unsubstituted analogs Compounds A and B. The R16-substituted analogs are 2.6-4.5 times more potent than the unsubstituted analogs in BaF3 cells expressing oncogenic BCR-ABL kinase, 1.5-3.5 times more potent in BaF3 cells expressing the T315I mutant oncogenic form of BCR-ABL kinase, 3.5-7.2 times more potent in BaF3 cells expressing the Y253F mutant oncogenic form of BCR-ABL kinase, 4.4-6 times more potent in BaF3 cells expressing the E255K mutant oncogenic form of BCR-ABL kinase, and 3.2-4.2 times more potent in BaF3 cells expressing the M351T mutant oncogenic form of BCR-ABL kinase. These five forms of BCR-ABL kinase are oncogenic and are causative of human chronic myelogenous leukemia. Moreover, the four mutant forms of BCR-ABL kinase are resistant to the currently available BCR-ABLinhibitor Gleevec®.

TABLE 2

| | | | | | BaF3 | BaF3 |
|---|---|---|---|---|---|---|
| | | BaF3 wt | BaF3 T315I | BaF3 Y253F | E255K | M351T |
| | | BCR-ABL | BCR-ABL | BCR-ABL | BCR-ABL | BCR-ABL |
| | R16 | IC₅₀ | IC₅₀ | IC₅₀ | IC₅₀ | IC₅₀ |
| Example 1 | 2-F | 6 nM | 8 nM | 26 nM | 83 nM | 11 nM |
| Example 5 | 5-Me | 8 nM | 25 nM | 15 nM | 62 nM | 10 nM |
| Compound A | H | 16 nM | 12 nM | 108 nM | 368 nM | 35 nM |
| Example 15 | 2-F | 11 nM | 25 nM | 86 nM | 238 nM | 13 nM |
| Compound B | H | 49 nM | 87 nM | 297 nM | 1,109 nM | 54 nM |

1. A method of modulating a kinase activity of a wild-type kinase species, oncogenic forms thereof, aberrant fusion proteins thereof and polymorphs of any of the foregoing, comprising the step of contacting said species with a compound of formula Ia′: 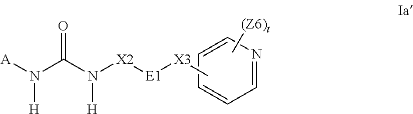
or a pharmaceutically acceptable salt thereof,
wherein
E1 is phenyl and wherein the E1 ring is substituted with one to three R16 moieties and one to three R18 moieties;
A is selected from the group consisting of pyrazolyl and imidazolyl;
G1 is a heteroaryl taken from the group consisting of pyrazolyl, imidazolyl, pyrrolyl, furyl, thienyl, oxazolyl, thiazolyl, isoxazolyl, isothiazolyl, oxadiazolyl, thiadiazolyl, triazolyl, tetrazolyl, pyrazinyl, pyridazinyl, triazinyl, pyridinyl, and pyrimidinyl;
G4 is a heterocyclyl taken from the group consisting of oxetanyl, azetadinyl, tetrahydrofuranyl, pyrrolidinyl, oxazolinyl, oxazolidinyl, imidazolonyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl;
the A ring is substituted at any substitutable position with one A1 moiety, wherein A1 is selected from the group consisting of: 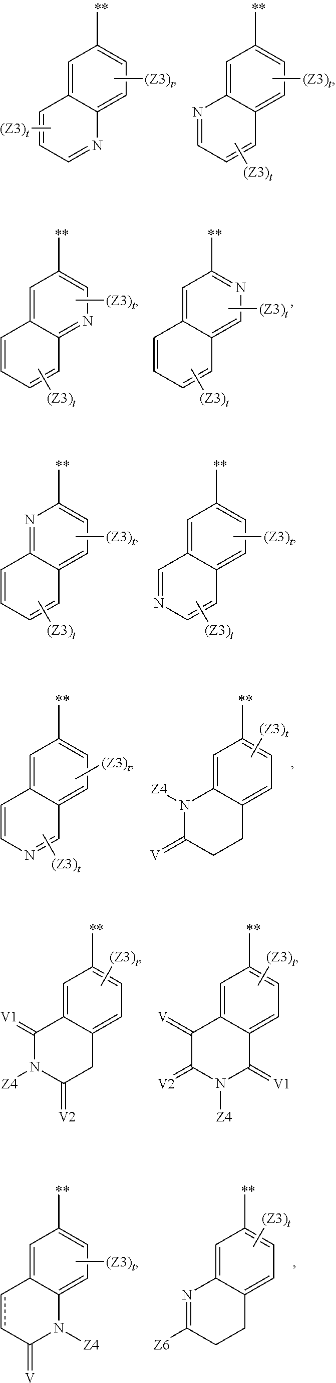 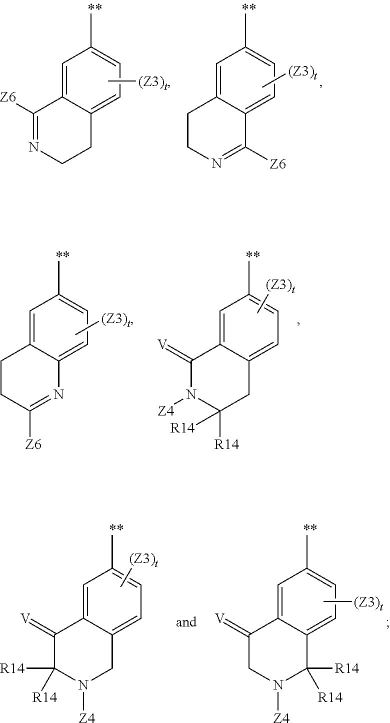
and wherein the symbol (**) is the point of attachment to the A ring of formula Ia;
and wherein - - - - indicates either a saturated or unsaturated bond;
the A ring is optionally substituted with one or more R2 moieties;
X2 is a direct bond, wherein E1 is directly linked to the NH group of formula Ia;
X3 is —O—;
V, V1 and V2 are each independently O or represent two hydrogens attached to the methylene carbon to which the V, V1, and V2 is attached;
each Z3 is independently and individually selected from the group consisting of H, C1-C6alkyl, branched C3-C7alkyl, C3-C8carbocyclyl, halogen, fluoroC1-C6alkyl wherein the alkyl moiety can be partially or fully fluorinated, cyano, hydroxyl, methoxy, oxo, (R3)2NC(O)—, (R4)2NC(O)—, —N(R4)C(O)R8, (R3)2NSO2-, (R4)2NSO2-, —N(R4)SO2R5, —N(R4)SO2R8, —(CH2)N(R3)2, —(CH2)nN(R4)2, —O(CH2)qN(R4)2, —O(CH2)qO—C1-C6alkyl, —N(R3)(CH2)qO—C1-C6alkyl, —N(R3)(CH2)qN(R4)2, —O(CH2)qR5, —N(R3)(CH2)qR5, —C(O)R5, —C(O)R8, —R5, and nitro;
in the event that Z3 contains an alkyl or alkylene moiety, such moieties may be further substituted by one or more C1-C6alkyl;
each Z4 is independently and individually selected from the group consisting of H, C1-C6alkyl, hydroxyC2-C6alkyl, C1-C6alkoxyC2-C6alkyl, (R4)2N—C2-C6alkyl, (R4)2N—C2-C6alkylN(R4)-C2-C6alkyl, (R4)2N—C2-C6alkyl-O—C2-C6alkyl, (R4)2NC(O)—C1-C6alkyl, carboxyC1-C6alkyl-, C1-C6alkoxycarbonylC1-C6alkyl-, —C2-C6alkylN(R4)C(O)R8, R8-C(═NR3)-, —SO2R8, —C(O)R8, and —(CH2)qR5;
in the event that Z4 contains an alkyl or alkylene moiety, such moieties may be further substituted by one or more C1-C6alkyl;
each Z6 is independently and individually selected from the group consisting of C(O)N(R3)2, —C(O)N(R4)2, —(CH2)nG1, (R4)2N—, (R3)2N—, —N(R3)C(O)R8, N(R4)C(O)R8, H, C1-C6alkyl, branched C3-C7alkyl, hydroxyl, hydroxyC1-C6alkyl, hydroxyC2-C6 branched alkyl, C1-C6alkoxy, C1-C6alkoxyC1-C6alkyl-, C1-C6alkoxyC2-C6 branched alkyl-, C2-C6 branched alkoxy-, C1-C6alkylthio-, —R5, —N(R3)SO2R6, —C(O)R5, —SO2N(R4)2, —SO2N(R5)2, halogen, fluoroC1-C6alkyl wherein the alkyl is fully or partially fluorinated, cyano, fluoroC1-C6alkoxy wherein the alkyl is fully or partially fluorinated, —O(CH2)qN(R4)2, —N(R3)(CH2)qN(R4)2, —O(CH2)qO—C1-C6alkyl, —O(CH2)qN(R4)2, —N(R3)(CH2)qO—C1-C6alkyl, —N(R3)(CH2)qN(R4)2, —O(CH2)qR5, and —N(R3)(CH2)qR5, —(NR3)rR17, —(O)rR17, —(S)rR17, —(CH2)nR17, —R17, —(CH2)nG4, —(CH2)n—O—(CH2)nG1, —(CH2)n—O—(CH2)nG4, —(CH2)nN(R3)(CH2)nG1, and —(CH2)nN(R3)(CH2)nG4;
each R2 is selected from the group consisting of branched C3-C8alkyl, C1-C6alkyl, fluoroC1-C6alkyl wherein the alkyl is fully or partially fluorinated, R19 substituted C3-C8carbocyclyl, Z3-substituted aryl, Z3-substituted G1-, Z3-substituted G4-, hydroxyC1-C6alkyl-, hydroxy branched C3-C6alkyl-, hydroxy substituted C3-C8carbocyclyl-, cyanoC1-C6alkyl-, cyano substituted branched C3-C6alkyl, cyano substituted C3-C8carbocyclyl, (R4)2NC(O)C1-C6alkyl-, (R4)2NC(O) substituted branched C3-C6alkyl-, (R4)2NC(O) substituted C3-C8carbocyclyl-, halogen, cyano, C1-C6alkoxy, and fluoroC1-C6alkoxy wherein the alkyl is fully or partially fluorinated;
wherein each R3 is independently and individually selected from the group consisting of H, C1-C6alkyl, branched C3-C7alkyl, C3-C8carbocyclyl, and Z3-substituted phenyl;
each R4 is independently and individually selected from the group consisting of H, C1-C6alkyl, hydroxyC1-C6alkyl-, dihydroxyC1-C6alkyl-, C1-C6alkoxyC1-C6alkyl-, branched C3-C7alkyl-, branched hydroxyC1-C6alkyl-, branched C1-C6alkoxyC1-C6alkyl-, branched dihydroxyC2-C6alkyl-, —(CH2)pN(R7)2, —(CH2)pR5, —(CH2)pC(O)N(R7)2, —(CH2)nC(O)R5, —(CH2)nC(O)OR3, C3-C8carbocyclyl, hydroxy substituted C3-C8carbocyclyl-, alkoxy substituted C3-C8carbocyclyl-, dihydroxy substituted C3-C8carbocyclyl-, and —(CH2)nR17;
each R5 is independently and individually selected from the group consisting of 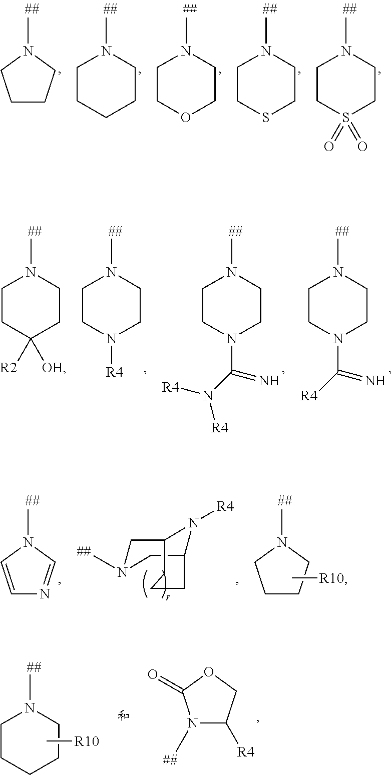
and wherein the symbol (##) is the point of attachment of the R5 moiety;
each R6 is independently and individually selected from the group consisting of C1-C6alkyl, branched C3-C7alkyl, C3-C8carbocyclyl, phenyl, G1, and G4;
each R7 is independently and individually selected from the group consisting of H, C1-C6alkyl, hydroxyC2-C6alkyl-, dihydroxyC2-C6alkyl-, C2-C6alkoxyC2-C6alkyl-, branched C3-C7alkyl-, branched hydroxyC2-C6alkyl-, branched C2-C6alkoxyC2-C6alkyl-, branched dihydroxyC2-C6alkyl-, —(CH2)qR5, —(CH2)nC(O)R5, —(CH2)nC(O)OR3, C3-C8carbocyclyl, hydroxy substituted C3-C8carbocyclyl-, alkoxy substituted C3-C8carbocyclyl-, dihydroxy substituted C3-C8carbocyclyl, and —(CH2)nR17;
each R8 is independently and individually selected from the group consisting of C1-C6alkyl, branched C3-C7alkyl, fluoroC1-C6alkyl wherein the alkyl moiety is partially or fully fluorinated, C3-C8carbocyclyl, Z3-substituted phenyl-, Z3-substituted phenylC1-C6alkyl-, Z3-substituted G1, Z3-substituted G1-C1-C6alkyl-, Z2-substituted G4, Z2-substituted G4-C1-C6alkyl-, OH, C1-C6alkoxy, N(R3)2, N(R4)2, and R5;
each R10 is independently and individually selected from the group consisting of CO2H, CO2C1-C6alkyl, —C(O)N(R4)2, OH, C1-C6alkoxy, and —N(R4)2;
each R14 is independently and respectively selected from the group consisting of H, C1-C6alkyl, branched C3-C6alkyl, and C3-C8carbocyclyl;
R16 is independently and individually selected from the group consisting of halogen, C1-C6alkyl, branched C3-C7alkyl, C3-C8carbocyclyl, fluoroC1-C6alkyl wherein the alkyl moiety can be partially or fully fluorinated, cyano, hydroxyl, C1-C6alkoxy, fluoroC1-C6alkoxy wherein the alkyl moiety can be partially or fully fluorinated, —N(R3)2, —N(R4)2, C2-C3alkynyl, and nitro;
each R17 is selected from the group consisting of phenyl, naphthyl, pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyrazinyl, pyridazinyl, triazinyl, oxetanyl, azetadinyl, tetrahydrofuranyl, oxazolinyl, oxazolidinyl, pyranyl, thiopyranyl, tetrahydropyranyl, dioxalinyl, azepinyl, oxepinyl, diazepinyl, pyrrolidinyl, and piperidinyl;
wherein R17 can be further substituted with one or more Z2, Z3 or Z4 moieties;
R18 is independently and individually selected from the group consisting of hydrogen, C1-C6alkyl, branched C3-C7alkyl, C3-C8carbocyclyl, halogen, fluoroC1-C6alkyl wherein the alkyl moiety can be partially or fully fluorinated, cyano, hydroxyl, C1-C6alkoxy, fluoroC1-C6alkoxy wherein the alkyl moiety can be partially or fully fluorinated, —N(R3)2, —N(R4)2, C2-C3alkynyl, and nitro;
R19 is H or C1-C6alkyl;
n is 0-6; p is 1-4; q is 2-6; r is 0 or 1; t is 1-3; and v is 1 or 2; and
at least one other pharmaceutically active agent.

2. A method of treating mammalian disease wherein the disease etiology or progression is at least partially mediated by the kinase activity of c-ABL kinase, BCR-ABL kinase, FLT-3 kinase, TIE-2 kinase, TRK-A kinase, TRK-B kinase, TRK-C kinase, VEGFR-2 kinases, c-MET kinase, PDGFR-alpha kinase, PDGFR-beta kinase, HER-1 kinase, HER-2 kinase, HER-3 kinase, HER-4 kinase, FGFR kinases, c-KIT kinase, RET kinase, c-FMS kinase, oncogenic forms thereof, aberrant fusion proteins thereof and polymorphs of any of the foregoing, comprising the step of administering to the mammal a therapeutically effective amount of a pharmaceutical composition comprising a compound of claim 1; and
further comprising administering at least one other pharmaceutically active agent.

3. The method of claim 2 wherein said kinase is selected from the group consisting of BCR-ABL fusion protein kinases p210, BCR-ABL fusion protein kinases p190, BCR-ABL fusion protein kinases bearing the T315I gatekeeper mutant in the ABL kinase domain of p210, BCR-ABL fusion protein kinases bearing the T3151 gatekeeper mutant in the ABL kinase domain of p190, and other BCR-ABL polymorphs of any of the foregoing kinases.

4. The method of claim 3, wherein said BCR-ABL fusion protein kinases p210 have Seq. IDs 3 & 4, wherein said BCR-ABL fusion protein kinase p190 has Seq. ID 5, wherein said BCR-ABL fusion protein kinases p210 bearing the T315I mutation in the ABL kinase domain have Seq. IDs 6 & 7, and wherein said BCR-ABL fusion protein kinase p190 bearing the T315I mutation in the ABL kinase domain has Seq. ID 8.

5. The method of claim 2 wherein said kinase is selected from the group consisting of c-KIT protein kinase, PDGFR-alpha kinase, PDGFR-beta kinase, c-FMS kinase, and any fusion protein, mutation and polymorph of any of the foregoing.

6. The method of claim 2 wherein said kinase is selected from the group consisting of c-MET protein kinase, RET kinase, FGFR kinases, HER kinases, and any fusion protein, mutation and polymorph of any of the foregoing.

7. The method of claim 2 wherein said kinase is selected from the group consisting of FLT-3 kinase, TIE-2 kinase, TRK kinases, and any fusion protein, mutation and polymorph of any of the foregoing.

8. A method of treating an individual suffering from a condition selected from the group consisting of cancer, secondary cancer growth arising from metastasis, hyperproliferative diseases, diseases characterized by hyper-vascularization, inflammation, osteoarthritis, respiratory diseases, stroke, systemic shock, immunological diseases, autoimmune diseases, bone resorptive diseases, cardiovascular disease and diseases characterized by angiogenesis, comprising the step of administering to such individual a therapeutically effective amount of a pharmaceutical composition comprising a compound of claim 1; and
further comprising administering at least one other pharmaceutically active agent.

9. A method of treating an individual suffering from a disease caused by c-ABL kinase, oncogenic forms thereof, aberrant fusion proteins thereof including BCR-ABL kinase and polymorphs thereof; a disease caused by FLT-3 kinase, oncogenic forms thereof, aberrant fusion proteins thereof and polymorphs thereof; a disease caused by TIE-2 kinase, oncogenic forms thereof, aberrant fusion proteins thereof and polymorphs thereof; a disease caused by TRK kinases, oncogenic forms thereof, aberrant fusion proteins thereof and polymorphs thereof; a disease caused by cMET kinase, oncogenic forms thereof, aberrant fusion proteins thereof including TPR-MET; a disease caused by KDR kinase or PDGFR kinases; a disease caused by HER kinases, oncogenic forms thereof and polymorphs thereof; a disease caused by RET kinase, oncogenic forms thereof, aberrant fusion proteins thereof; a disease caused by c-FMS kinase, oncogenic forms thereof and polymorphs thereof; a disease caused by a c-KIT kinase, oncogenic forms thereof, aberrant fusion proteins thereof and polymorphs thereof; and diseases caused by any of the foregoing kinases, oncogenic forms thereof, and aberrant fusion proteins thereof, including but not limited to, chronic myelogenous leukemia, acute lymphocytic leukemia, acute myeloid leukemia, other myeloproliferative disorders, a disease caused by metastasis of primary solid tumors to secondary sites, glioblastomas, ovarian cancer, pancreatic cancer, prostate cancer, lung cancers, mesothelioma, hypereosinophilic syndrome, a disease caused or maintained by pathological vascularization, ocular diseases characterized by hyperproliferation leading to blindness including various retinopathies, i.e. diabetic retinopathy and age-related macular degeneration, non small cell lung cancer, breast cancers, kidney cancers, colon cancers, cervical carcinomas, papillary thyroid carcinoma, melanomas, autoimmune diseases including rheumatoid arthritis, multiple sclerosis, lupus, asthma, human inflammation, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic obstructive pulmonarydisease, bone resorptive diseases, bone cancer, graft-versus-host reaction, Crohn's disease, ulcerative colitis, inflammatory bowel disease, pyresis, gastrointestinal stromal tumors, and combinations, comprising the step of administering to such individual a therapeutically effective amount of a pharmaceutical composition comprising a compound of Claim 1; and
further comprising administering at least one other pharmaceutically active agent.

10. The method of claim 8 or 9, said compound being administered by a method selected from the group consisting of oral, parenteral, inhalation, and subcutaneous.

11. The method of claim 1, 2, 8, 9, or 10 wherein the compound is 1-(3-tert-butyl-1-(quinolin-6-yl)-1H-pyrazol-5-yl)-3-(2-fluoro-4-(2-(methylcarbamoyl)pyridin-4-yloxy)phenyl)urea.

12. The method of claim 8 or 9, wherein the pharmaceutical composition further comprises at least one other pharmaceutically active agent.

13. The method of claim 12, wherein the at least one other pharmaceutically active agent is useful for treating cancer.

14. The method of claim 13, wherein the other pharmaceutically active agent is selected from the group consisting of imatinib, nilotinib, dasatinib, ponatinib, and bosutinib.

15. The method of claim 14 wherein the compound of formula Ia′ is 1-(3-tert-butyl-1-(quinolin-6-yl)-1H-pyrazol-5-yl)-3-(2-fluoro-4-(2-(methylcarbamoyl)pyridin-4-yloxy)phenyl)urea.

16. The method of claim 14, wherein the other pharmaceutically active agent is imatinib.

17. The method of claim 16 wherein the compound of formula Ia′ is 1-(3-tert-butyl-1-(quinolin-6-yl)-1H-pyrazol-5-yl)-3-(2-fluoro-4-(2-(methylcarbamoyl)pyridin-4-yloxy)phenyl)urea.

18. The method of claim 14, wherein the other therapeutic agent is dasatinib.

19. The method of claim 18 wherein the compound of formula Ia is 1-(3-tert-butyl-1-(quinolin-6-yl)-1H-pyrazol-5-yl)-3-(2-fluoro-4-(2-(methylcarbamoyl)pyridin-4-yloxy)phenyl)urea.

20. The method of claim 12, 15, 17, or 19, wherein the other pharmaceutically active agent is combined with the compound of formula Ia′ in a single dosage form.

21. The method of claim 12, 15, 17, or 19, wherein the other pharmaceutically active agent is in a separate dosage form than the compound of formula Ia′.

22. The method of claim 21, wherein the compound of formula Ia and the other pharmaceutically active agent are dosed simultaneously or sequentially within a period of time from one hour to less than two weeks.

23. The method of claim 22, wherein the compound of formula Ia and the other pharmaceutically active agent are dosed alternately, wherein the compound of formula Ia is administered for a period of time ranging from two weeks to six months, followed by administration of the other pharmaceutically active agent for a second period of time ranging from two weeks to six months.

24. The method of claim 23, wherein the alternate dosing of the compound of formula Ia and the other pharmaceutically active agent is repeated multiple times.

25. The method of claim 24, wherein a drug holiday is implemented between the dosing of the compound of formula Ia′ and the other pharmaceutically active agent, wherein neither agent is dosed during the drug holiday.

26. The method of claim 25, wherein the drug holiday is a period of time ranging from one day to one month.

27. The method of claim 12, wherein at least one other pharmaceutically active agent is useful for treating autoimmune diseases or inflammatory diseases.

28. The method of claim 27, wherein the other pharmaceutically active agent is selected from the group consisting of methotrexate or other anti-folate agent.

29. The method of claim 27, wherein the other pharmaceutically active agent is an anti-TNF agent.

30. The method of claim 27, wherein the other pharmaceutically active agent is selected from the group consisting of Humira®, Enbrel®, and Remicade®.
